(19) United States
(12) Patent Application Publication
(10) Pub. No.: US 2021/0299107 A1
(43) Pub. Date: Sep. 30, 2021

(54) COMPOUNDS, COMPOSITIONS, AND METHODS FOR MODULATING FERROPTOSIS AND TREATING EXCITOTOXIC DISORDERS

(71) Applicant: THE TRUSTEES OF COLUMBIA UNIVERSITY IN THE CITY OF NEW YORK, New York, NY (US)

(72) Inventors: Brent R. Stockwell, New York, NY (US); Arie Zask, New York, NY (US); Hui Tan, New York, NY (US); Jacob Daniels, New York, NY (US)

(21) Appl. No.: 17/330,386

(22) Filed: May 25, 2021

Related U.S. Application Data
(63) Continuation-in-part of application No. PCT/US2019/063640, filed on Nov. 27, 2019.
(60) Provisional application No. 62/771,841, filed on Nov. 27, 2018.

(51) Int. Cl. A61K 31/4245; C07D 271/10; C07D 263/32; C07C 229/60; C07C 233/65; C07D 295/13; C07D 213/74; C07D 413/04; C07D 213/80; C07D 413/12; A61P 39/06; A61P 31/06; A61K 31/421; A61K 31/245; A61K 31/166; A61K 31/5375; A61K 31/44; A61K 31/4439

(52) U.S. Cl. CPC A61K 31/4245; A61K 31/4439; C07D 263/32; C07C 229/60; C07C 233/65; C07D 295/13; C07D 213/74; C07D 413/04; C07D 213/80; C07D 413/12; A61P 39/06; A61P 31/06; A61K 31/421; A61K 31/245; A61K 31/166; A61K 31/5375; A61K 31/44; C07D 271/10

(57) ABSTRACT

The present disclosure provides, inter alia, a compound having the structure:

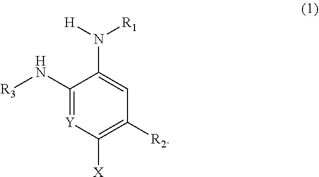

Also provided are compositions containing a pharmaceutically acceptable carrier and one or more compounds according to the present disclosure. Further provided are methods for treating or ameliorating the effects of an excitotoxic disorder in a subject, methods of modulating ferroptosis in a subject, methods of reducing reactive oxygen species (ROS) in a cell, methods for treating or ameliorating the effects of a neurodegenerative disease, methods for alleviating side effects in a subject undergoing radiotherapy and/or immunotherapy, and methods for treating or ameliorating the effects of an infection associated with ferroptosis in a subject.

### CROSS REFERENCE TO RELATED APPLICATIONS

The present application is a continuation-in-part of PCT international application no. PCT/US2019/063640, filed on Nov. 27, 2019, which claims benefit of U.S. Provisional Patent Application Ser. No. 62/771,841, filed on Nov. 27, 2018, which applications are incorporated by reference herein in their entireties.

### GOVERNMENT FUNDING

This disclosure was made with government support under grant nos. CA097061, CA209896 and NS109407, awarded by National Institutes of Health. The government has certain rights in the disclosure.

### FIELD OF DISCLOSURE

The present disclosure provides, inter alia, compounds having the structure:

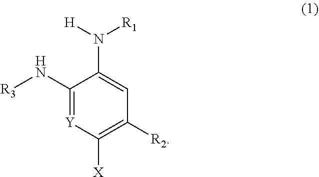

Also provided are pharmaceutical compositions containing the compounds of the present disclosure, as well as methods of using such compounds and compositions.

### BACKGROUND OF THE DISCLOSURE

Cell death is crucial for normal development, homeostasis and the prevention of hyper-proliferative diseases such as cancer (Fuchs and Steller, 2011; Thompson, 1995). It was once thought that almost all regulated cell death in mammalian cells resulted from the activation of caspase-dependent apoptosis (Fuchs and Steller, 2011; Thompson, 1995). More recently this view has been challenged by the discovery of several regulated non-apoptotic cell death pathways activated in specific disease states, including poly(ADP-ribose) polymerase-1 (PARP-1) and apoptosis inducing factor 1 (AIF1)-dependent parthanatos, caspase-1-dependent pyroptosis and receptor interacting protein kinase 1 (RIPK1)-dependent necroptosis (Bergsbaken et al., 2009; Christofferson and Yuan, 2010; Wang et al., 2009). It is believed that additional regulated forms of non-apoptotic cell death likely remain to be discovered that mediate cell death in other developmental or pathological circumstances.

The RAS family of small GTPases (HRAS, NRAS and KRAS) is mutated in about 30% of all cancers (Vigil et al., 2010). Finding compounds that are selectively lethal to RAS-mutant tumor cells is, therefore, a high priority. Two structurally unrelated small molecules, named erastin and RSL3, were previously identified. These molecules were selectively lethal to oncogenic RAS-mutant cell lines, and together, they were referred to as RAS-selective lethal (RSL) compounds (Dolma et al., 2003; Yang and Stockwell, 2008). Using affinity purification, voltage dependent anion channels 2 and 3 (VDAC2/3) were identified as direct targets of erastin (Yagoda et al., 2007), but not RSL3. ShRNA and cDNA overexpression studies demonstrated that VDAC2 and VDAC3 are necessary, but not sufficient, for erastin-induced death (Yagoda et al., 2007), indicating that additional unknown targets are required for this process.

The type of cell death activated by the RSLs has been enigmatic. Classic features of apoptosis, such as mitochondrial cytochrome c release, caspase activation and chromatin fragmentation, are not observed in RSL-treated cells (Dolma et al., 2003; Yagoda et al., 2007; Yang and Stockwell, 2008). RSL-induced death is, however, associated with increased levels of intracellular reactive oxygen species (ROS) and is prevented by iron chelation or genetic inhibition of cellular iron uptake (Yagoda et al., 2007; Yang and Stockwell, 2008). In a recent systematic study of various mechanistically unique lethal compounds, the prevention of cell death by iron chelation was a rare phenomenon (Wolpaw et al., 2011), suggesting that few triggers can access iron-dependent lethal mechanisms.

Accordingly, there is a need for the exploration of various pathways of regulated cell death, as well as for compositions and methods for preventing the occurrence of regulated cell death. This disclosure is directed to meeting these and other needs.

### SUMMARY OF THE DISCLOSURE

Without being bound to a particular theory, the inventors hypothesized that RSLs, such as erastin, activate a lethal pathway that is different from apoptosis, necrosis and other well-characterized types of regulated cell death. It was found that erastin-induced death involves a unique constellation of morphological, biochemical and genetic features, which led to the name “ferroptosis” as a description for this phenotype. Small molecule inhibitors of ferroptosis that prevent ferroptosis in cancer cells, as well as glutamate-induced cell death in postnatal rat brain slices have been identified and disclosed herein. The inventors have found an underlying similarity between diverse forms of iron-dependent, non-apoptotic death and that the manipulation of ferroptosis may be exploited to selectively destroy RAS-mutant tumor cells or to preserve neuronal cells exposed to specific oxidative conditions.

Accordingly, one embodiment of the present disclosure is a compound according to formula (1):

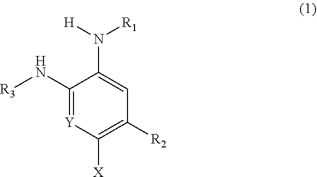

wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups; and
X is selected from the group consisting of H, optionally substituted alkyl, and halo;
Y is —CH or N;
or an N-oxide, crystalline form, hydrate, or a pharmaceutically acceptable salt thereof,
with the proviso that:
when R₁ and X are both H, Y is —CH and R₃ is

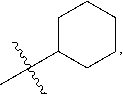 [SMILES: *C1CCCCC1]

R₂ cannot be

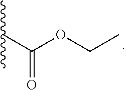 [SMILES: *C(=O)OCC]

Another embodiment of the present disclosure is a compound selected from the group consisting of:

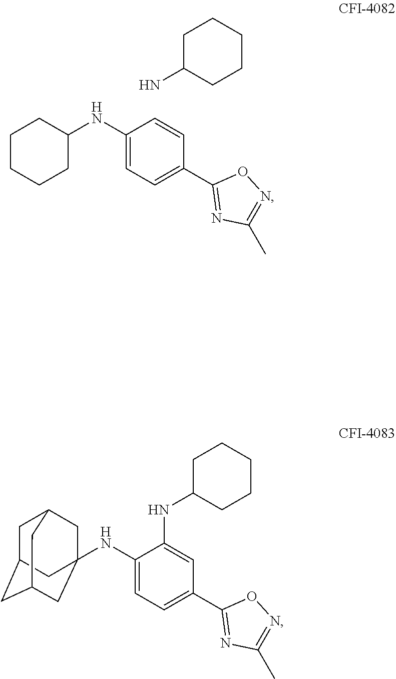

and combinations thereof, or an N-oxide, crystalline form, hydrate, or a pharmaceutically acceptable salt thereof.

Another embodiment of the present disclosure is a pharmaceutical composition. This pharmaceutical composition comprises a pharmaceutically acceptable carrier or diluent and one or more compounds according to formula (1):

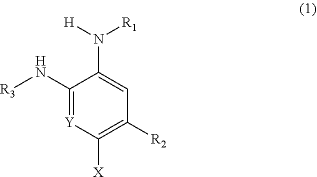

wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

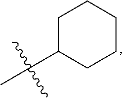 [SMILES: *C1CCCCC1]

R₂ cannot be

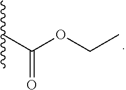 [SMILES: *C(=O)OCC]

A further embodiment of the present disclosure is a kit. This kit comprises a compound or a pharmaceutical composition according to the present disclosure with instructions for the use of the compound or the pharmaceutical composition, respectively.

Another embodiment of the present disclosure is a method for treating or ameliorating the effects of a disorder in a subject in need thereof. This method comprises administering to the subject an effective amount of one or more compounds having the structure of formula (1):

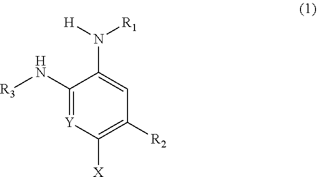

wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

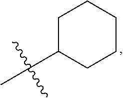 [SMILES: *C1CCCCC1]

R₂ cannot be

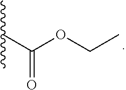 [SMILES: *C(=O)OCC]

An additional embodiment of the present disclosure is a method for treating or ameliorating the effects of a disorder in a subject in need thereof. This method comprises administering to the subject an effective amount of a pharmaceutical composition comprising a pharmaceutically acceptable carrier or diluent and one or more compounds having the structure of formula (1):

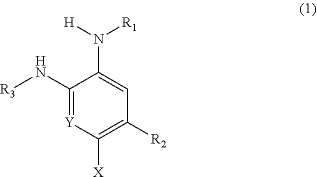

wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

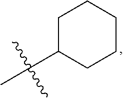 [SMILES: *C1CCCCC1]

R₂ cannot be

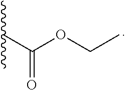 [SMILES: *C(=O)OCC]

Another embodiment of the present disclosure is a method of modulating ferroptosis in a subject in need thereof. This method comprises administering to the subject an effective amount of a ferroptosis inhibitor, which comprises one or more compounds having the structure of formula (1):

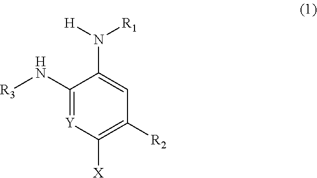

wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

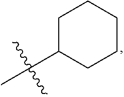 [SMILES: *C1CCCCC1]

R₂ cannot be

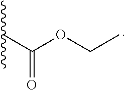 [SMILES: *C(=O)OCC]

A further embodiment of the present disclosure is a method of reducing reactive oxygen species (ROS) in a cell. This method comprises contacting a cell with a ferroptosis modulator, which comprises one or more compounds having the structure of formula (1):

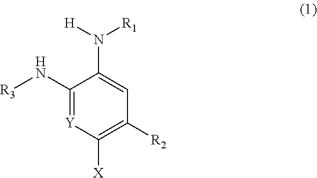

wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

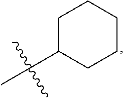 [SMILES: *C1CCCCC1]

R₂ cannot be

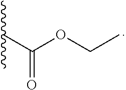 [SMILES: *C(=O)OCC]

An additional embodiment of the present disclosure is a method for treating or ameliorating the effects of a neurodegenerative disease in a subject in need thereof. This method comprises administering to the subject an effective amount of one or more compounds having the structure of formula (1):

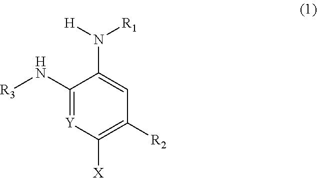

wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

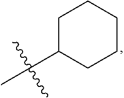 [SMILES: *C1CCCCC1]

R₂ cannot be

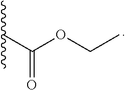 [SMILES: *C(=O)OCC]

A further embodiment of the present disclosure is a compound according to formula (2):

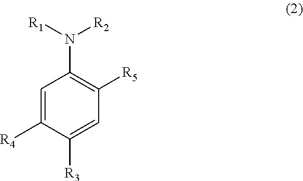

wherein:
R₁ and R₂ are independently selected from the group consisting of H, aryl, C₁₋₆alkyl-aryl, C₁₋₆ alkyl-phenolyl, C₁₋₆ alkyl-bicycle, and C₃₋₁₀carbocycle, wherein each of the aryl, C₁₋₆ alkyl-aryl, C₁₋₆ alkyl-phenolyl, C₁₋₆ alkyl-bicycle, and C₃₋₁₀carbocycle are optionally substituted with one or more atoms or groups; or together, with the nitrogen attached, form a cyclic or bicyclic structure, wherein the cyclic or bicyclic structure is optionally substituted with one or more atoms or groups;
R₃ is selected from the group consisting of hydroxyl, alkoxy, and alcohol, wherein each of the hydroxyl, alkoxy, and alcohol are optionally substituted with one or more atoms or groups;
R₄ is selected from the group consisting of H, alkyl, and alkoxy; or together with R₃, form a ring structure, wherein the ring structure is optionally substituted with one or more atoms or groups; and
R₅ is selected from the group consisting of H, and alkoxy;
or an N-oxide, crystalline form, hydrate, or a pharmaceutically acceptable salt thereof.

Still another embodiment of the present disclosure is a compound according to formula (3):

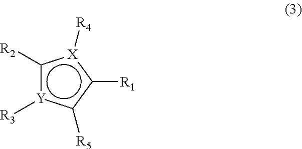

wherein:
X is selected from N, O, and S;
Y is C or N;
R₁ and R₅ are independently selected from the group consisting of H, alkenyl, ester, amino, and aryl, wherein each of the alkenyl, ester, amino, and aryl are optionally substituted with one or more atoms or groups; or together form a ring structure, wherein the ring structure is optionally substituted with one or more atoms or groups;
R₂ and R₃ together form a saturated or unsaturated ring structure, wherein the ring structure is optionally substituted with one or more atoms or groups; and
R₄ is selected from the group consisting of no atom, H, alkyl, alkenyl, and ketone;
or an N-oxide, crystalline form, hydrate, or a pharmaceutically acceptable salt thereof.

Another embodiment of the present disclosure is a compound selected from the group consisting of:

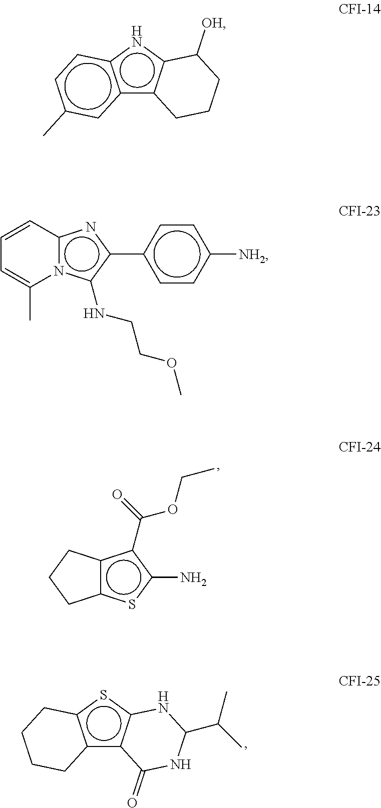

and combinations thereof, or an N-oxide, crystalline form, hydrate, or a pharmaceutically acceptable salt thereof.

Another embodiment of the present disclosure is a pharmaceutical composition. This pharmaceutical composition comprises a pharmaceutically acceptable carrier or diluent and one or more compounds according to formula (2):

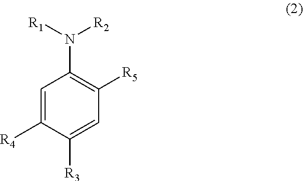

wherein:
R₁ and R₂ are independently selected from the group consisting of H, aryl, C₁₋₆alkyl-aryl, C₁₋₆ alkyl-phenolyl, C₁₋₆ alkyl-bicycle, and C₃₋₁₀carbocycle, wherein each of the aryl, C₁₋₆ alkyl-aryl, C₁₋₆ alkyl-phenolyl, C₁₋₆ alkyl-bicycle, and C₃₋₁₀carbocycle are optionally substituted with one or more atoms or groups; or together, with the nitrogen attached, form a cyclic or bicyclic structure, wherein the cyclic or bicyclic structure is optionally substituted with one or more atoms or groups;
R₃ is selected from the group consisting of hydroxyl, alkoxy, and alcohol, wherein each of the hydroxyl, alkoxy, and alcohol are optionally substituted with one or more atoms or groups;
R₄ is selected from the group consisting of H, alkyl, and alkoxy; or together with R₃, form a ring structure, wherein the ring structure is optionally substituted with one or more atoms or groups; and
R₅ is selected from the group consisting of H, and alkoxy;
or an N-oxide, crystalline form, hydrate, or a pharmaceutically acceptable salt thereof.

Another embodiment of the present disclosure is a pharmaceutical composition. This pharmaceutical composition comprises a pharmaceutically acceptable carrier or diluent and one or more compounds according to formula (3):

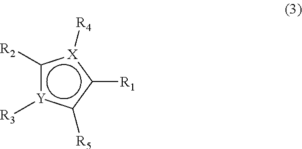

wherein:
X is selected from N, O, and S;
Y is C or N;
R₁ and R₅ are independently selected from the group consisting of H, alkenyl, ester, amino, and aryl, wherein each of the alkenyl, ester, amino, and aryl are optionally substituted with one or more atoms or groups; or together form a ring structure, wherein the ring structure is optionally substituted with one or more atoms or groups;
R₂ and R₃ together form a saturated or unsaturated ring structure, wherein the ring structure is optionally substituted with one or more atoms or groups; and
R₄ is selected from the group consisting of no atom, H, alkyl, alkenyl, and ketone;
or an N-oxide, crystalline form, hydrate, or a pharmaceutically acceptable salt thereof.

An additional embodiment of the present disclosure is a method for treating or ameliorating the effects of a neurodegenerative disease in a subject in need thereof. This method comprises administering to the subject an effective amount of a compound having the structure selected from the group consisting of:

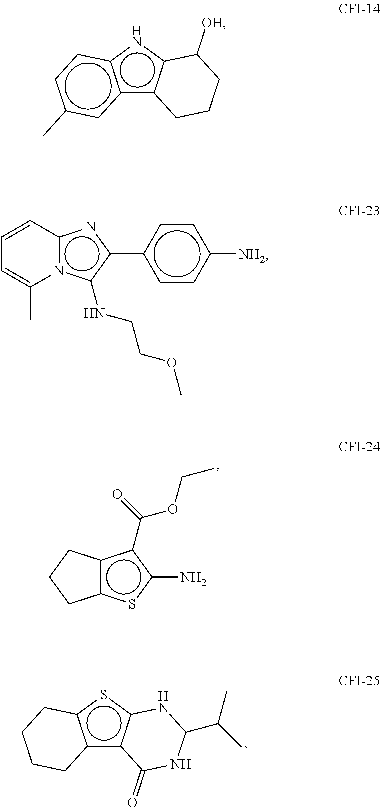

and combinations thereof, or an N-oxide, crystalline form, hydrate, or a pharmaceutically acceptable salt thereof.

An additional embodiment of the present disclosure is a method of modulating ferroptosis in a subject in need thereof. This method comprises administering to the subject an effective amount of a ferroptosis inhibitor, which comprises a compound having the structure selected from the group consisting of:

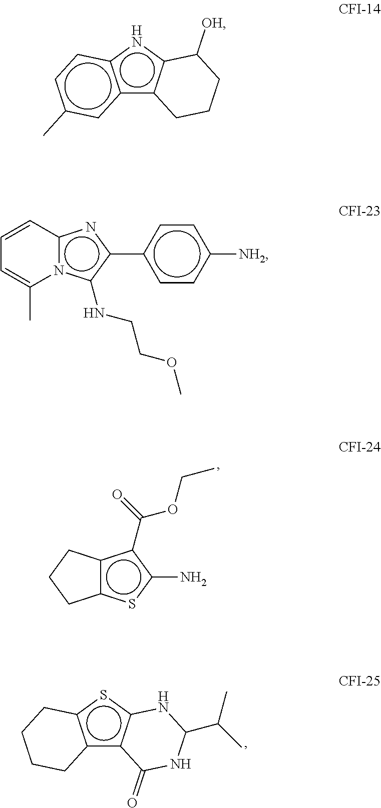

and combinations thereof, or an N-oxide, crystalline form, hydrate, or a pharmaceutically acceptable salt thereof.

An additional embodiment of the present disclosure is a method of reducing reactive oxygen species (ROS) in a cell. This method comprises contacting a cell with a ferroptosis modulator, which comprises a compound having the structure selected from the group consisting of:

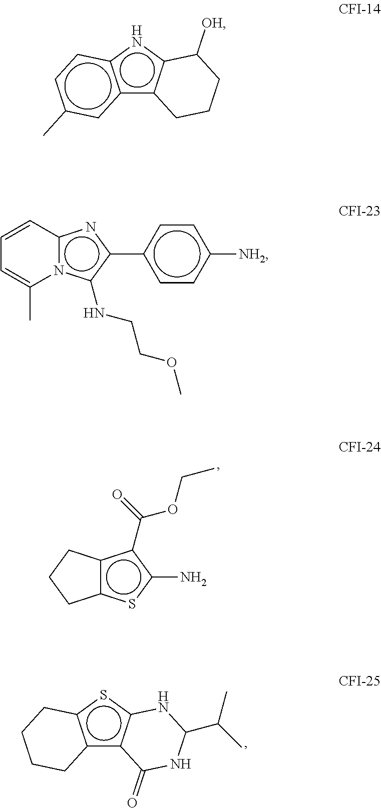

and combinations thereof, or an N-oxide, crystalline form, hydrate, or a pharmaceutically acceptable salt thereof.

An additional embodiment of the present disclosure is a method for treating or ameliorating the effects of a neurodegenerative disease in a subject in need thereof. This method comprises administering to the subject an effective amount of a compound having the structure selected from the group consisting of:

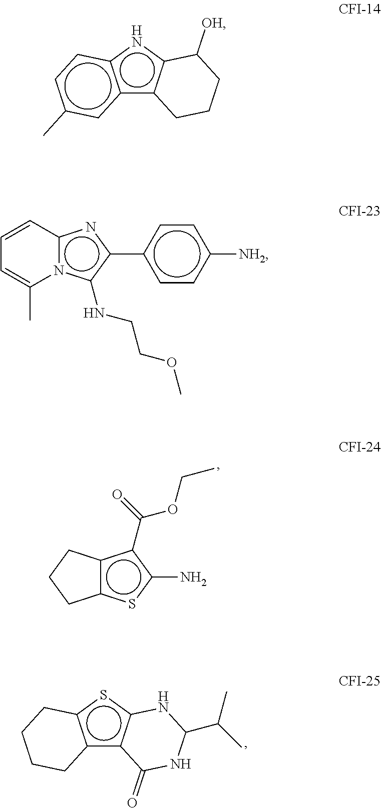

and combinations thereof, or an N-oxide, crystalline form, hydrate, or a pharmaceutically acceptable salt thereof.

Still another embodiment of the present disclosure is a method for alleviating side effects in a subject undergoing radiotherapy and/or immunotherapy, comprising administering to the subject an effective amount of one or more compounds disclosed herein.

A further embodiment of the present disclosure is a method for treating or ameliorating the effects of an infection associated with ferroptosis in a subject, comprising administering to the subject an effective amount of one or more compounds disclosed herein.

### BRIEF DESCRIPTION OF THE DRAWINGS

The application file contains at least one drawing executed in color. Copies of this patent application with color drawing(s) will be provided by the Office upon request and payment of the necessary fee.

FIGS. 1A-1C show the biological activities of Ferrostatin-1 and analogs. FIG. 1A shows the dose-response relationship for inhibition of erastin (10 μM, 24 hours)-induced death in HT-1080 cells by Fer-1 and analogs. FIG. 1B shows the dose-response relationship for inhibition of IKE or RSL3-induced death in HT-1080 cells by Fer-1 and analogs. FIG. 1C shows the structure of various compounds listed in FIGS. 1A and 1B.

FIG. 2 shows the microsomal stability of Fer-1, CFI-102 and TH-2-9-1 in mouse.

FIG. 3 shows the metabolic stability of CFI-4082 in plasma, brain, liver and kidney.

FIG. 4 shows the structure of selected Fer-1 analogs further tested in Example 4.

FIG. 5A shows the dose-response curves of TH-2-9-1, TH-2-5, and Fer-1 at a concentration range from 20 μM-0 μM against 3 μM IKE and 0.2 μM RSL3.

FIG. 5B shows the dose-response curves of TH-2-9-1, TH-2-5, and Fer-1 at a concentration range from 10 μM-0 μM against 3 μM IKE and 0.2 μM RSL3.

FIG. 5C shows the dose-response curves of TH-2-9-1, TH-2-5, and Fer-1 at a concentration range from 1 μM-0 μM against 10 μM Erastin, 3 μM IKE and 0.2 μM RSL3. Asterisk (*) indicates standardized result.

FIG. 5D shows the dose-response curves of TH-2-9-1, TH-2-5, and Fer-1 at a concentration range from 1 μM-0 μM against 10 μM Erastin, 3 μM IKE and 0.2 μM RSL3, from a second set of experiments.

FIG. 6A shows the dose-response curves of CFI-102 and TH-2-30 at a concentration range from 10 μM-0 μM against 3 μM IKE and 0.2 μM RSL3.

FIG. 6B shows the dose-response curves of CFI-102 and TH-2-30 at a concentration range from 2.5 μM-0 μM against 3 μM IKE and 0.2 μM RSL3.

FIG. 6C shows the dose-response curves of CFI-102 and TH-2-30 at a concentration range from 5 μM-0 μM against 3 μM IKE and 0.2 μM RSL3. HT-1080 cells were incubated for 51 hours.

FIG. 7 shows the dose-response curves of CFI-102, TH-2-30, TH-2-9-1 and Fer-1 at a concentration range from 5 μM-0 μM against 3 μM IKE and 0.2 μM RSL3. HT-1080 cells were incubated for 49 hours.

FIG. 8 shows the structures of the optimized analogs and the corresponding inactive analogs.

FIG. 9 shows the structures and representative dose-response curves of active ferrostatins TH-2-31, TH-4-55-2, and TH-4-67 (N=3).

FIG. 10 shows the structures and dose-response curves of inactive controls TH-4-50-2, TH-4-46-2, and TH-4-58-2 (N=3).

FIG. 11 shows microsomal stability of 3 active analogs (n=2 wells/compound/experiment).

FIG. 12 shows pharmacokinetics in plasma and brain of three active ferrostatins administered via IP, IV and PO.

FIG. 13 shows BBB permeabilities calculated as log₁₀(brain/plasma) values for each compound at each time point.

FIG. 14 shows brain concentration of each compound over time.

FIG. 15 shows Cₘₐₓ/EC₅₀ for brain and plasma of each optimized compounds and R.O.A.

FIG. 16 shows plasma stability (mouse) curves of each optimized analogs (n=2 wells/compound/experiment, N=2).

### DETAILED DESCRIPTION OF THE DISCLOSURE

In the present disclosure, new analogs of Fer-1 are provided. Certain of the analogs have improved microsomal stability and solubility while still maintaining good inhibition potency of ferroptosis. Accordingly, one embodiment of the present disclosure is a compound according to formula (1):

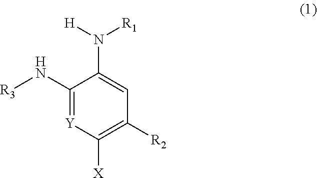

wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

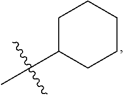 [SMILES: *C1CCCCC1]

R₂ cannot be

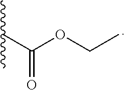 [SMILES: *C(=O)OCC]

In one aspect of this embodiment, the compound has the structure of formula (1a):

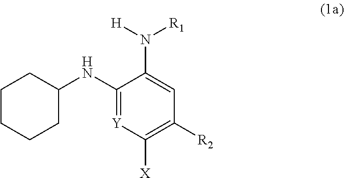

wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R1 and X are both H and Y is —CH, R₂ cannot be

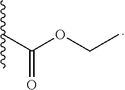 [SMILES: *C(=O)OCC]

In another aspect of this embodiment, the compound has the structure of formula (1b):

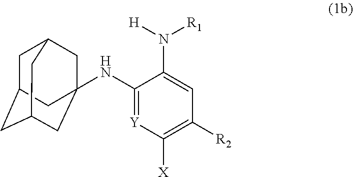

wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups; and
X is selected from the group consisting of H, optionally substituted alkyl, and halo; and
Y is —CH or N;
or an N-oxide, crystalline form, hydrate, or a pharmaceutically acceptable salt thereof.

In another aspect of this embodiment, the compound is selected from the group consisting of:

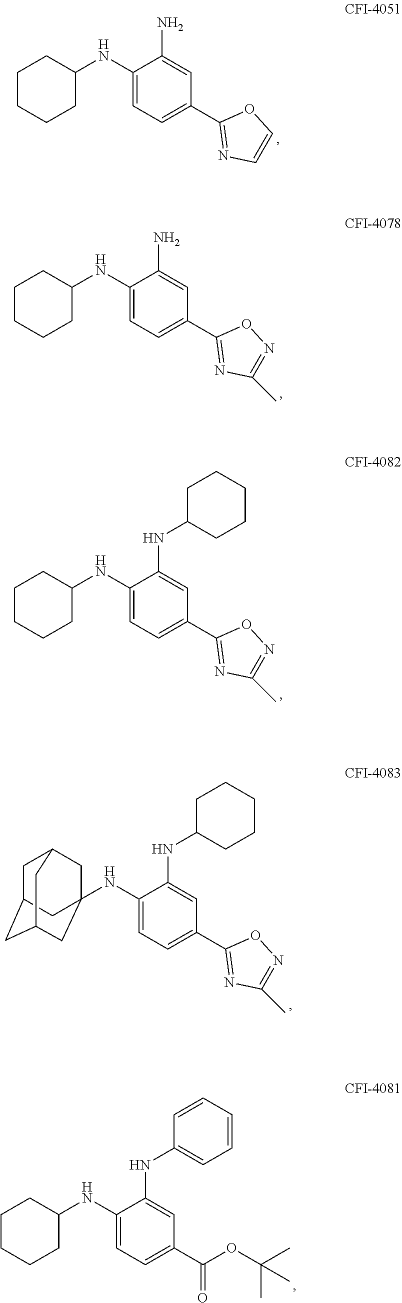 [SMILES: Nc1cc(-c2ncco2)ccc1NC1CCCCC1 ; Cc1noc(-c2ccc(NC3CCCCC3)c(N)c2)n1 ; Cc1noc(-c2ccc(NC3CCCCC3)c(NC3CCCCC3)c2)n1 ; Cc1noc(-c2ccc(NC34CC5CC(CC(C5)C3)C4)c(NC3CCCCC3)c2)n1 ; CC(C)(C)OC(=O)c1ccc(NC2CCCCC2)c(Nc2ccccc2)c1]

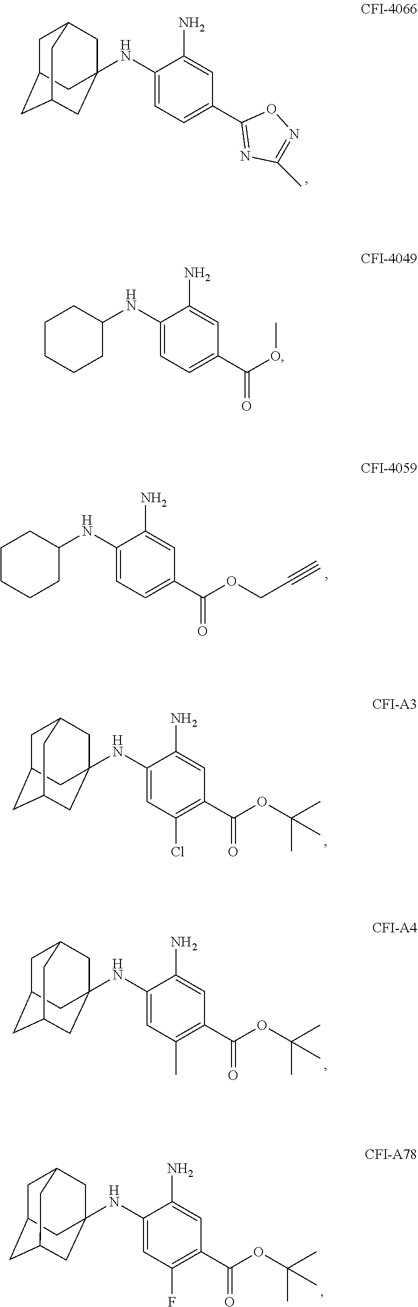 [SMILES: Cc1noc(-c2ccc(NC34CC5CC(CC(C5)C3)C4)c(N)c2)n1 ; COC(=O)c1ccc(NC2CCCCC2)c(N)c1 ; C#CCOC(=O)c1ccc(NC2CCCCC2)c(N)c1 ; CC(C)(C)OC(=O)c1cc(N)c(NC23CC4CC(CC(C4)C2)C3)cc1Cl ; Cc1cc(NC23CC4CC(CC(C4)C2)C3)c(N)cc1C(=O)OC(C)(C)C ; CC(C)(C)OC(=O)c1cc(N)c(NC23CC4CC(CC(C4)C2)C3)cc1F]

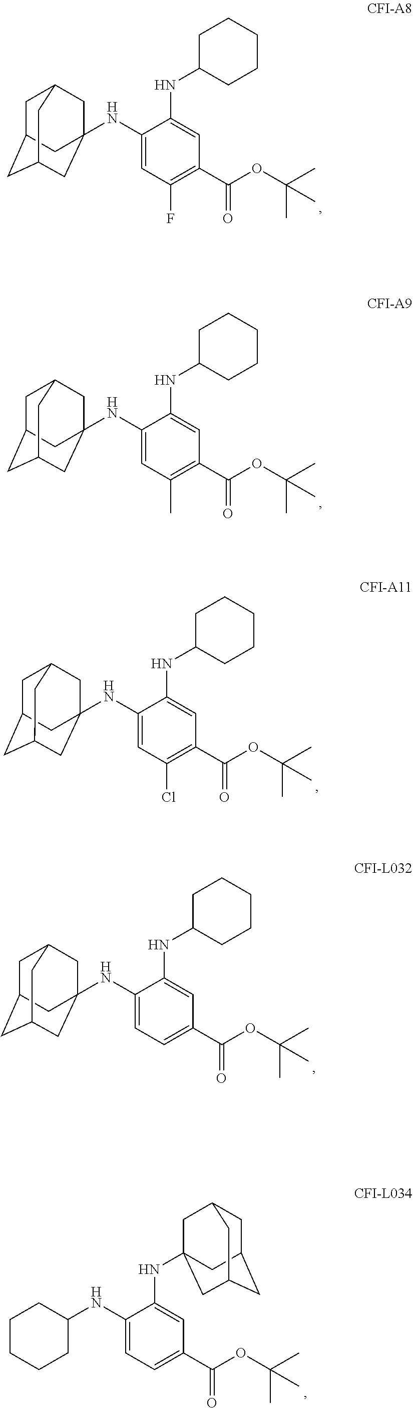 [SMILES: CC(C)(C)OC(=O)c1cc(NC2CCCCC2)c(NC23CC4CC(CC(C4)C2)C3)cc1F ; Cc1cc(NC23CC4CC(CC(C4)C2)C3)c(NC2CCCCC2)cc1C(=O)OC(C)(C)C ; CC(C)(C)OC(=O)c1cc(NC2CCCCC2)c(NC23CC4CC(CC(C4)C2)C3)cc1Cl ; CC(C)(C)OC(=O)c1ccc(NC23CC4CC(CC(C4)C2)C3)c(NC2CCCCC2)c1 ; CC(C)(C)OC(=O)c1ccc(NC2CCCCC2)c(NC23CC4CC(CC(C4)C2)C3)c1]

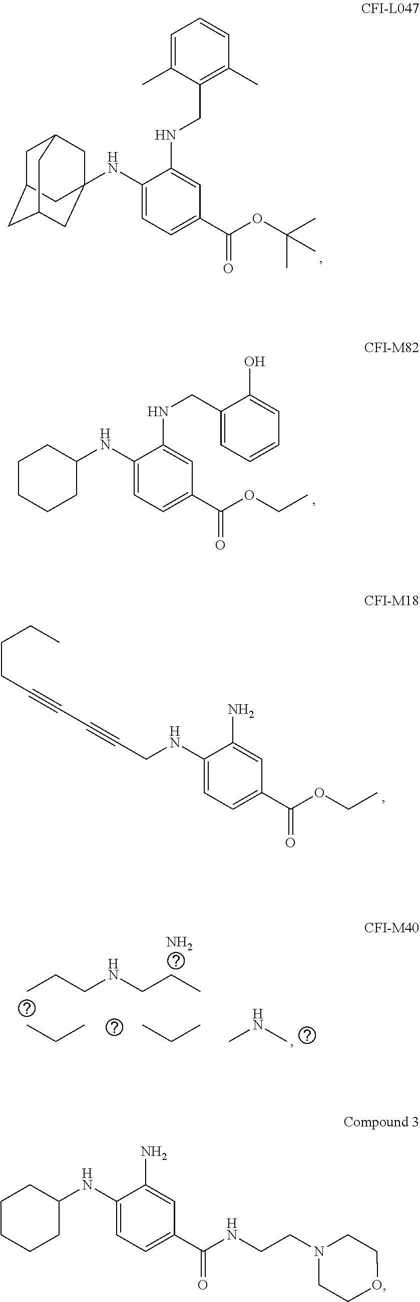

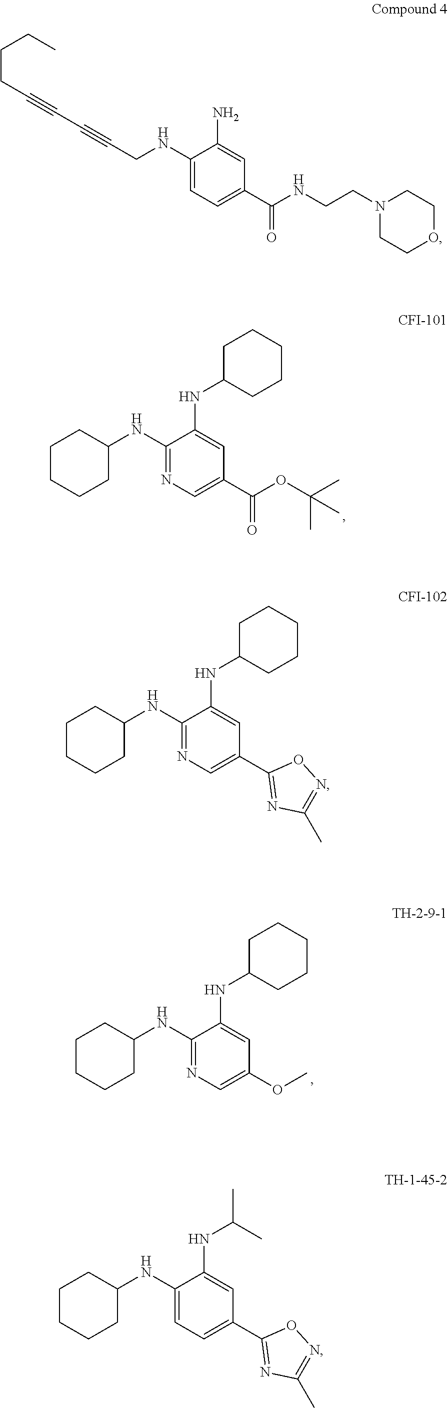 [SMILES: CCCCC#CC#CCNc1ccc(C(=O)NCCN2CCOCC2)cc1N ; CC(C)(C)OC(=O)c1cnc(NC2CCCCC2)c(NC2CCCCC2)c1 ; Cc1noc(-c2cnc(NC3CCCCC3)c(NC3CCCCC3)c2)n1 ; COc1cnc(NC2CCCCC2)c(NC2CCCCC2)c1 ; Cc1noc(-c2ccc(NC3CCCCC3)c(NC(C)C)c2)n1]

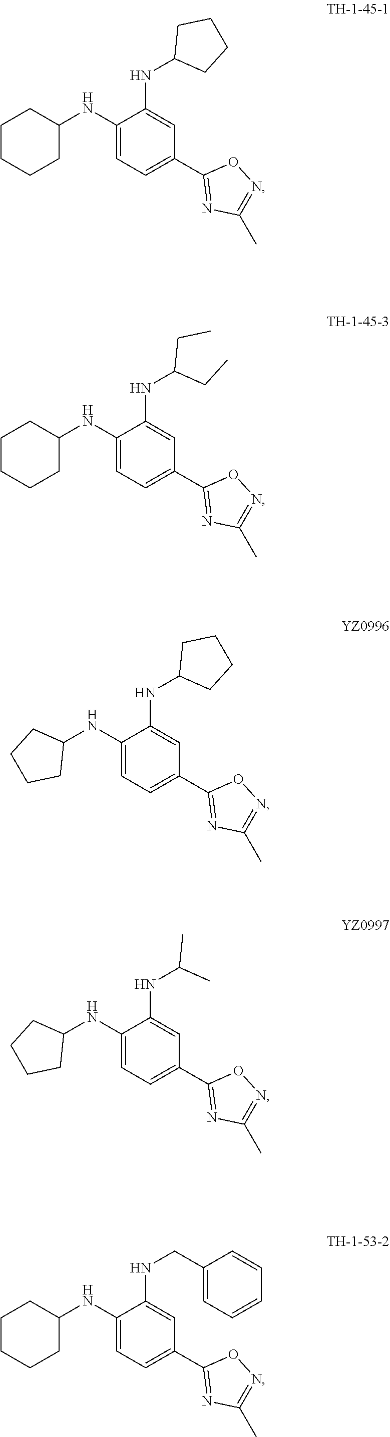 [SMILES: Cc1noc(-c2ccc(NC3CCCCC3)c(NC3CCCC3)c2)n1 ; CCC(CC)Nc1cc(-c2nc(C)no2)ccc1NC1CCCCC1 ; Cc1noc(-c2ccc(NC3CCCC3)c(NC3CCCC3)c2)n1 ; Cc1noc(-c2ccc(NC3CCCC3)c(NC(C)C)c2)n1 ; Cc1noc(-c2ccc(NC3CCCCC3)c(NCc3ccccc3)c2)n1]

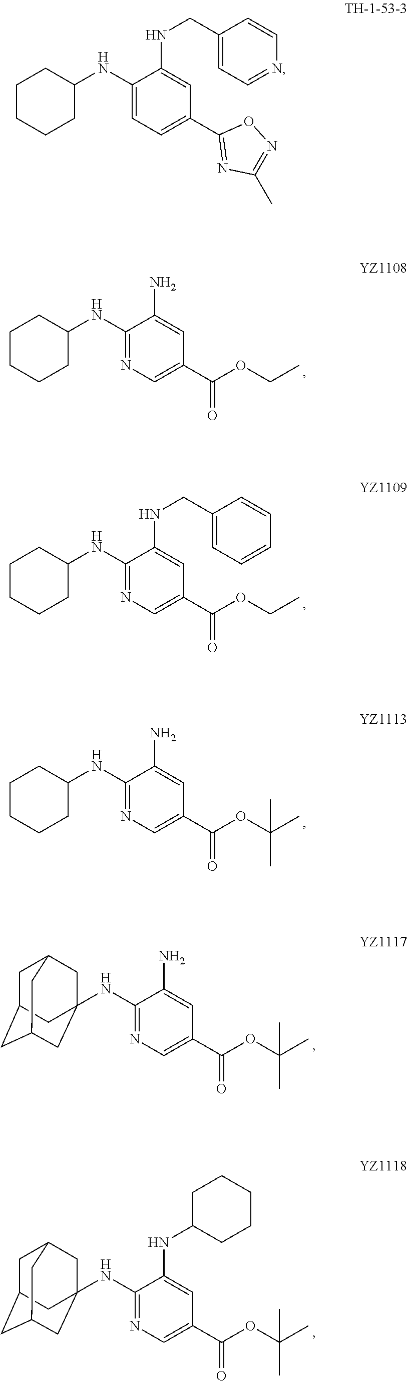

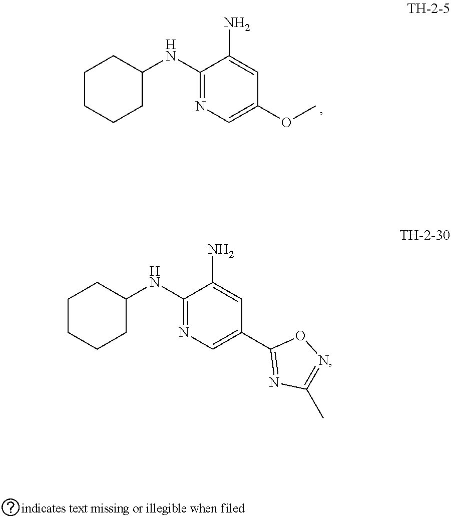 [SMILES: COc1cnc(NC2CCCCC2)c(N)c1 ; Cc1noc(-c2cnc(NC3CCCCC3)c(N)c2)n1]

or an N-oxide, crystalline form, hydrate, or a pharmaceutically acceptable salt thereof.

Preferably, the compound is selected from the group consisting of:

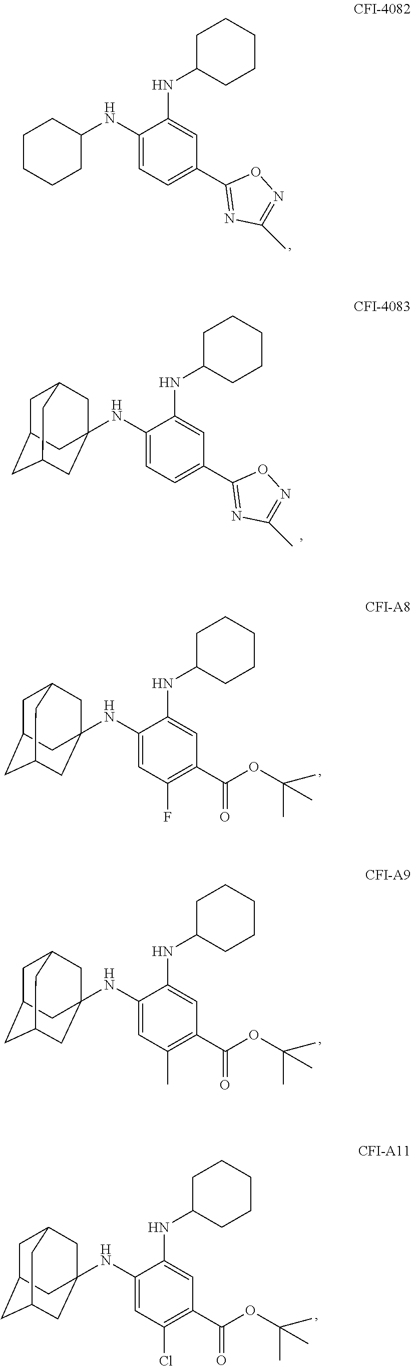 [SMILES: Cc1noc(-c2ccc(NC3CCCCC3)c(NC3CCCCC3)c2)n1 ; Cc1noc(-c2ccc(NC34CC5CC(CC(C5)C3)C4)c(NC3CCCCC3)c2)n1 ; CC(C)(C)OC(=O)c1cc(NC2CCCCC2)c(NC23CC4CC(CC(C4)C2)C3)cc1F ; Cc1cc(NC23CC4CC(CC(C4)C2)C3)c(NC2CCCCC2)cc1C(=O)OC(C)(C)C ; CC(C)(C)OC(=O)c1cc(NC2CCCCC2)c(NC23CC4CC(CC(C4)C2)C3)cc1Cl]

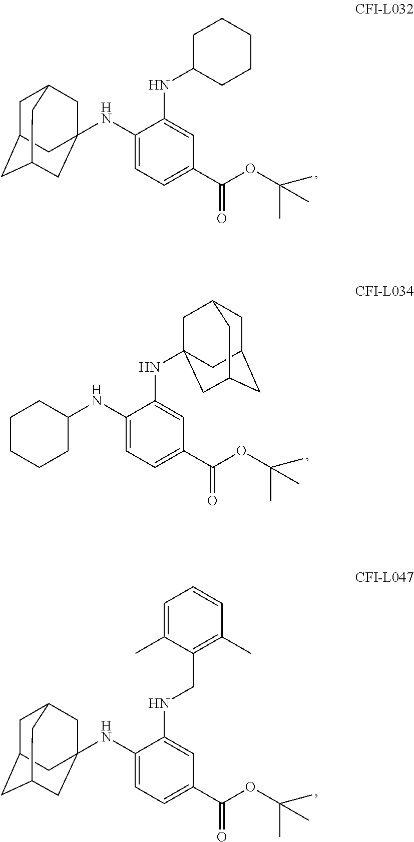 [SMILES: CC(C)(C)OC(=O)c1ccc(NC23CC4CC(CC(C4)C2)C3)c(NC2CCCCC2)c1 ; CC(C)(C)OC(=O)c1ccc(NC2CCCCC2)c(NC23CC4CC(CC(C4)C2)C3)c1 ; Cc1cccc(C)c1CNc1cc(C(=O)OC(C)(C)C)ccc1NC12CC3CC(CC(C3)C1)C2]

and combinations thereof, or an N-oxide, crystalline form, hydrate, or a pharmaceutically acceptable salt thereof.

More preferably, the compound is selected from the group consisting of:

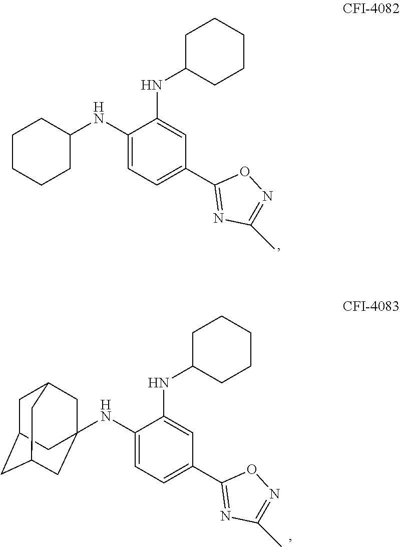 [SMILES: Cc1noc(-c2ccc(NC3CCCCC3)c(NC3CCCCC3)c2)n1 ; Cc1noc(-c2ccc(NC34CC5CC(CC(C5)C3)C4)c(NC3CCCCC3)c2)n1]

or an N-oxide, crystalline form, hydrate, or a pharmaceutically acceptable salt thereof.

Another embodiment of the present disclosure is a compound according to formula (2):

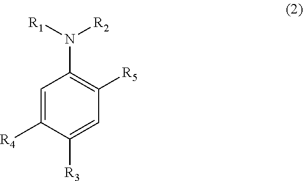

wherein:
R₁ and R₂ are independently selected from the group consisting of H, aryl, C₁₋₆alkyl-aryl, C₁₋₆ alkyl-phenolyl, C₁₋₆ alkyl-bicycle, and C₃₋₁₀carbocycle, wherein each of the aryl, C₁₋₆ alkyl-aryl, C₁₋₆ alkyl-bicycle, and C₃₋₁₀carbocycle are optionally substituted with one or more atoms or groups; or together, with the nitrogen attached, form a cyclic or bicyclic structure, wherein the cyclic or bicyclic structure is optionally substituted with one or more atoms or groups;
R₃ is selected from the group consisting of hydroxyl, alkoxy, and alcohol, wherein each of the hydroxyl, alkoxy, and alcohol are optionally substituted with one or more atoms or groups;
R₄ is selected from the group consisting of H, alkyl, and alkoxy; or together with R₃, form a ring structure, wherein the ring structure is optionally substituted with one or more atoms or groups; and
R₅ is selected from the group consisting of H, and alkoxy;
or an N-oxide, crystalline form, hydrate, or a pharmaceutically acceptable salt thereof.

Preferably, the compound is selected from the group consisting of:

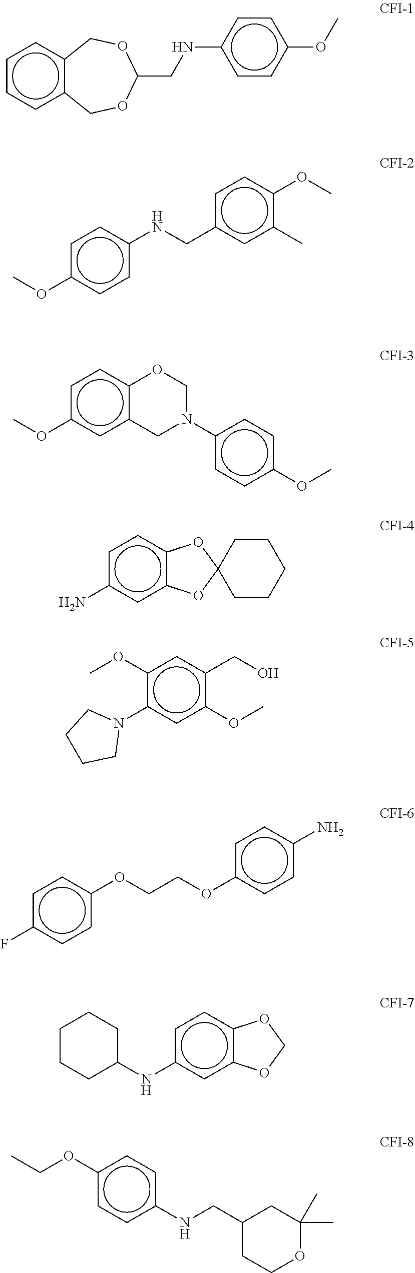

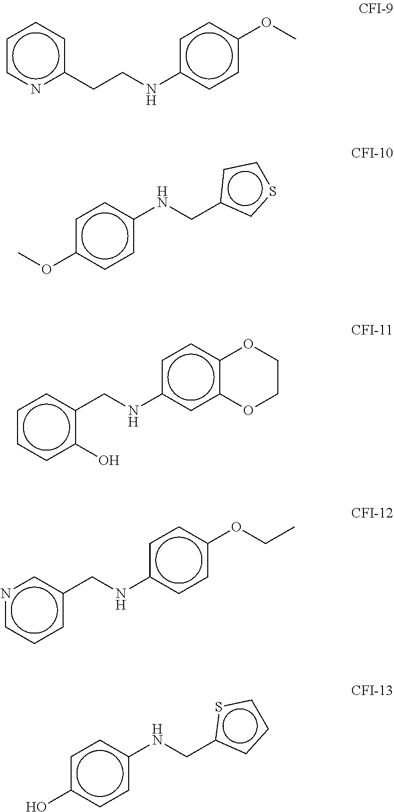

or an N-oxide, crystalline form, hydrate, or a pharmaceutically acceptable salt thereof.

Another embodiment of the present disclosure is a compound according to formula (3):

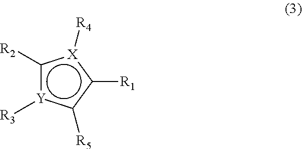

wherein:
X is selected from N, O, and S;
Y is C or N;
R₁ and R₅ are independently selected from the group consisting of H, alkenyl, ester, amino, and aryl, wherein each of the alkenyl, ester, amino, and aryl are optionally substituted with one or more atoms or groups; or together form a ring structure, wherein the ring structure is optionally substituted with one or more atoms or groups;
R₂ and R₃ together form a saturated or unsaturated ring structure, wherein the ring structure is optionally substituted with one or more atoms or groups; and
R₄ is selected from the group consisting of no atom, H, alkyl, alkenyl, and ketone;
or an N-oxide, crystalline form, hydrate, or a pharmaceutically acceptable salt thereof.

In one aspect of this embodiment, the compound has the structure of formula (3a):

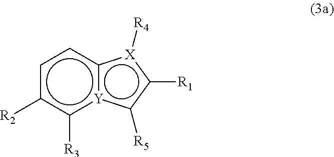

wherein:
X is selected from N, O, and S;
Y is C or N;
R₁ and R₅ are independently selected from the group consisting of H, alkenyl, ester, amino, and aryl, wherein each of the alkenyl, ester, amino, and aryl are optionally substituted with one or more atoms or groups; or together form a ring structure, wherein the ring structure is optionally substituted with one or more atoms or groups;
R₂ and R₃ are independently selected from the group consisting of H, alkyl, amino, and halo; and
R₄ is selected from the group consisting of no atom, H, alkyl, alkenyl, and ketone;
or an N-oxide, crystalline form, hydrate, or a pharmaceutically acceptable salt thereof.

In another aspect of this embodiment, the compound is selected from the group consisting of:

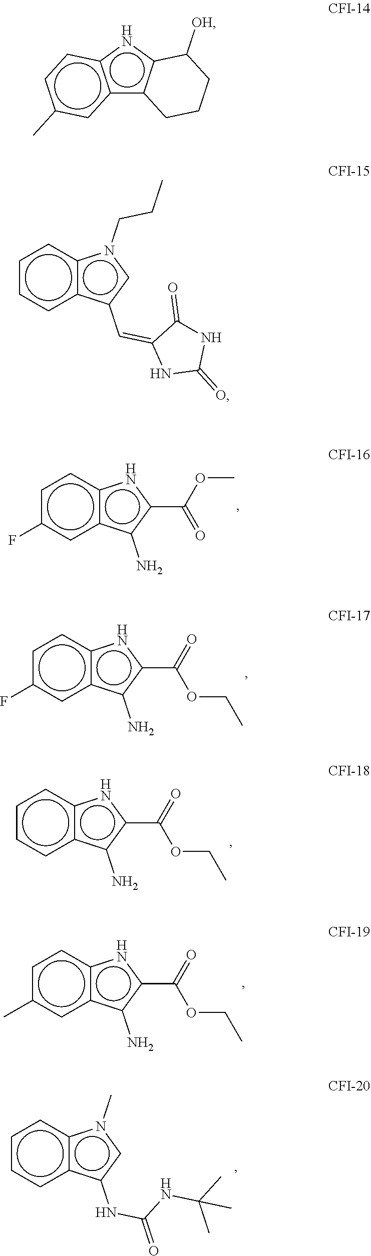

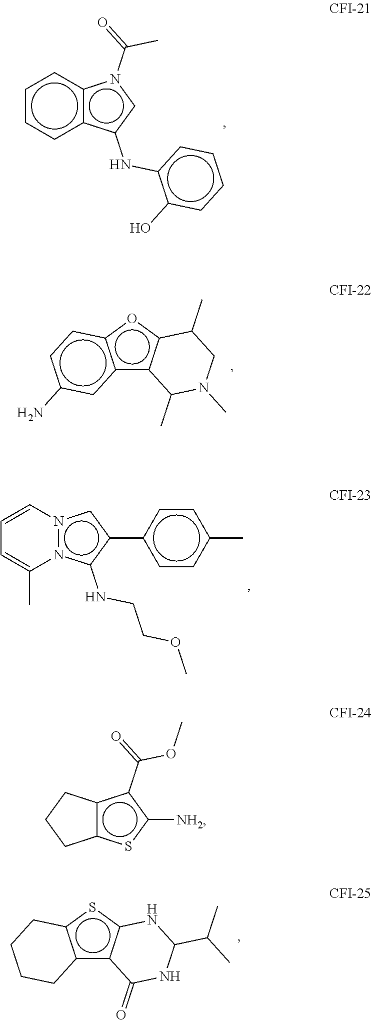

or an N-oxide, crystalline form, hydrate, or a pharmaceutically acceptable salt thereof.

Preferably, the compound is selected from the group consisting of:

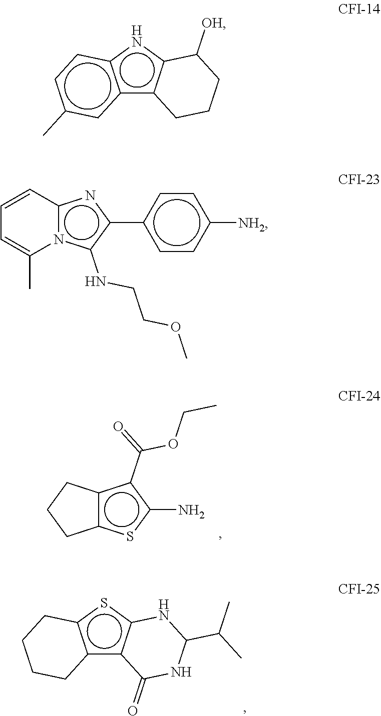

or an N-oxide, crystalline form, hydrate, or a pharmaceutically acceptable salt thereof.

Another embodiment of the present disclosure is a pharmaceutical composition. This pharmaceutical composition comprises a pharmaceutically acceptable carrier or diluent and one or more compounds according to formula (1):

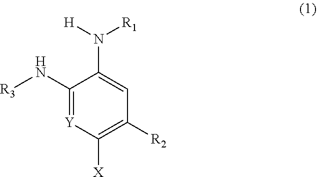

wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:

when R₁ and X are both H, Y is —CH and R₃ is

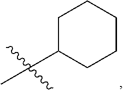 [SMILES: *C1CCCCC1]

R₂ cannot be

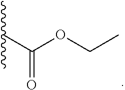 [SMILES: *C(=O)OCC]

Another embodiment of the present disclosure is a pharmaceutical composition. This pharmaceutical composition comprises a pharmaceutically acceptable carrier or diluent and one or more compounds according to formula (2):

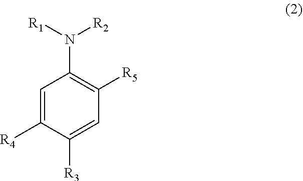

wherein:
R₁ and R₂ are independently selected from the group consisting of H, aryl, C₁₋₆alkyl-aryl, C₁₋₆ alkyl-phenolyl, C₁₋₆ alkyl-bicycle, and C₃₋₁₀carbocycle, wherein each of the aryl, C₁₋₆ alkyl-aryl, C₁₋₆ alkyl-phenolyl, C₁₋₆ alkyl-bicycle, and C₃₋₁₀carbocycle are optionally substituted with one or more atoms or groups; or together, with the nitrogen attached, form a cyclic or bicyclic structure, wherein the cyclic or bicyclic structure is optionally substituted with one or more atoms or groups;
R₃ is selected from the group consisting of hydroxyl, alkoxy, and alcohol, wherein each of the hydroxyl, alkoxy, and alcohol are optionally substituted with one or more atoms or groups;
R₄ is selected from the group consisting of H, alkyl, and alkoxy; or together with R₃, form a ring structure, wherein the ring structure is optionally substituted with one or more atoms or groups; and
R₅ is selected from the group consisting of H, and alkoxy;
or an N-oxide, crystalline form, hydrate, or a pharmaceutically acceptable salt thereof.

Another embodiment of the present disclosure is a pharmaceutical composition. This pharmaceutical composition comprises a pharmaceutically acceptable carrier or diluent and one or more compounds according to formula (3):

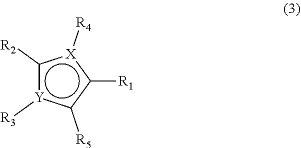

wherein:
X is selected from N, O, and S;
Y is C or N;
R₁ and R₅ are independently selected from the group consisting of H, alkenyl, ester, amino, and aryl, wherein each of the alkenyl, ester, amino, and aryl are optionally substituted with one or more atoms or groups; or together form a ring structure, wherein the ring structure is optionally substituted with one or more atoms or groups;
R₂ and R₃ together form a saturated or unsaturated ring structure, wherein the ring structure is optionally substituted with one or more atoms or groups; and
R₄ is selected from the group consisting of no atom, H, alkyl, alkenyl, and ketone;
or an N-oxide, crystalline form, hydrate, or a pharmaceutically acceptable salt thereof.

Suitable and preferred compounds that are used in the pharmaceutical compositions of the present disclosure are disclosed above in formulas (1), (1a), (1 b), (2), (3) and (3a), including the particular compounds also identified above.

A further embodiment of the present disclosure is a kit. This kit comprises a compound or a pharmaceutical composition disclosed herein with instructions for the use of the compound or the pharmaceutical composition, respectively.

The kits may also include suitable storage containers, e.g., ampules, vials, tubes, etc., for each compound of the present disclosure (which, e.g., may be in the form of pharmaceutical compositions) and other reagents, e.g., buffers, balanced salt solutions, etc., for use in administering the active agents to subjects. The compounds and/or pharmaceutical compositions of the disclosure and other reagents may be present in the kits in any convenient form, such as, e.g., in a solution or in a powder form. The kits may further include a packaging container, optionally having one or more partitions for housing the compounds and/or pharmaceutical compositions and other optional reagents.

Another embodiment of the present disclosure is a method for treating or ameliorating the effects of a disorder in a subject in need thereof. This method comprises administering to the subject an effective amount of one or more compounds having the structure of formula (1):

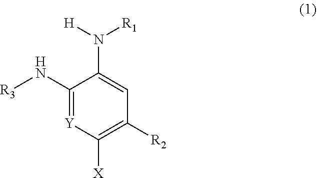

wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups; and
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

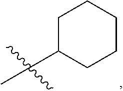 [SMILES: *C1CCCCC1]

R₂ cannot be

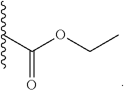 [SMILES: *C(=O)OCC]

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present disclosure may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

As used herein, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc.

Suitable and preferred compounds and pharmaceutical compositions for use in this method are as disclosed above in formulas (1), (1a), (1b), (2), (3) and (3a), including the particular compounds identified above.

In one aspect of this embodiment, the disorder is a degenerative disease that involves lipid peroxidation. As used herein, “lipid peroxidation” means the oxidative degradation of fats, oils, waxes, sterols, triglycerides, and the like. Lipid peroxidation has been linked with many degenerative diseases, such as atherosclerosis, ischemia-reperfusion, heart failure, Alzheimer's disease, rheumatic arthritis, cancer, and other immunological disorders. (Ramana et al., 2013).

In another aspect of this embodiment, the disorder is an excitotoxic disease involving oxidative cell death. As used herein, an “excitotoxic disorder” means a disease related to the death of central neurons that are mediated by excitatory amino acids (such as glutamate). Excitotoxic disorders within the scope of the present disclosure include diseases involving oxidative cell death. As used herein, “oxidative” cell death means cell death associated with increased levels of intracellular reactive oxygen species (ROS). In the present disclosure, “reactive oxygen species” means chemically reactive molecules, such as free radicals, containing oxygen. Non-limiting examples of ROS include oxygen ions and peroxides.

Non-limiting examples of disorders according to the present disclosure include epilepsy, kidney disease, stroke, myocardial infarction, type I diabetes, traumatic brain injury (TBI), periventricular leukomalacia (PVL), and neurodegenerative disease. Non-limiting examples of neurodegenerative diseases according to the present disclosure include Alzheimer's, Parkinson's, Amyotrophic lateral sclerosis, Friedreich's ataxia, Multiple sclerosis, Huntington's Disease, Transmissible spongiform encephalopathy, Charcot-Marie-Tooth disease, Dementia with Lewy bodies, Corticobasal degeneration, Progressive supranuclear palsy, Chronic Traumatic Encephalopathy (CTE), and Hereditary spastic paraparesis.

In another aspect of this embodiment, the method further comprises co-administering, together with one or more compounds or pharmaceutical compositions of the present disclosure, to the subject an effective amount of one or more of additional therapeutic agents such as 5-hydroxytryptophan, Activase, AFQ056 (Novartis Corp., New York, N.Y.), Aggrastat, Albendazole, alpha-lipoic acid/L-acetyl carnitine, Alteplase, Amantadine (Symmetrel), amlodipine, Ancrod, Apomorphine (Apokyn), Arimoclomol, Arixtra, Armodafinil, Ascorbic acid, Ascriptin, Aspirin, atenolol, Avonex, baclofen (Lioresal), Banzel, Benztropine (Cogentin), Betaseron, BGG492 (Novartis Corp., New York, N.Y.), Botulinum toxin, Bufferin, Carbatrol®, Carbidopa/levodopa immediate-release (Sinemet), Carbidopa/levodopa oral disintegrating (Parcopa), Carbidopa/levodopa/Entacapone (Stalevo), CERE-110: Adeno-Associated Virus Delivery of NGF (Ceregene, San Diego, Calif.), cerebrolysin, CinnoVex, citalopram, citicoline, Clobazam, Clonazepam, Clopidogrel, clozapine (Clozaril), Coenzyme Q, Creatine, dabigatran, dalteparin, Dapsone, Davunetide, Deferiprone, Depakene®, Depakote ER®, Depakote®, Desmoteplase, Diastat, Diazepam, Digoxin, Dilantin®, Dimebon, dipyridamole, divalproex (Depakote), Donepezil (Aricept), EGb 761, Eldepryl, ELND002 (Elan Pharmaceuticals, Dublin, Ireland), Enalapril, enoxaparin, Entacapone (Comtan), epoetin alfa, Eptifibatide, Erythropoietin, Escitalopram, Eslicarbazepine acetate, Esmolol, Ethosuximide, Ethyl-EPA (Miraxion™), Exenatide, Extavia, Ezogabine, Felbamate, Felbatol®, Fingolimod (Gilenya), fluoxetine (Prozac), fondaparinux, Fragmin, Frisium, Gabapentin, Gabitril®, Galantamine, Glatiramer (Copaxone), haloperidol (Haldol), Heparin, human chorionic gonadotropin (hCG), Idebenone, Inovelon®, insulin, Interferon beta 1a, Interferon beta 1 b, ioflupane 1231 (DATSCAN®), IPX066 (Impax Laboratories Inc., Hayward, Calif.), JNJ-26489112 (Johnson and Johnson, New Brunswick, N.J.), Keppra®, Klonopin, Lacosamide, L-Alpha glycerylphosphorylcholine, Lamictal®, Lamotrigine, Levetiracetam, liraglutide, Lisinopril, Lithium carbonate, Lopressor, Lorazepam, losartan, Lovenox, Lu AA24493, Luminal, LY450139 (Eli Lilly, Indianapolis, Ind.), Lyrica, Masitinib, Mecobalamin, Memantine, methylprednisolone, metoprolol tartrate, Minitran, Minocycline, mirtazapine, Mitoxantrone (Novantrone), Mysoline®, Natalizumab (Tysabri), Neurontin®, Niacinamide, Nitro-Bid, Nitro-Dur, nitroglycerin, Nitrolingual, Nitromist, Nitrostat, Nitro-Time, Norepinephrine (NOR), Carbamazepine, octreotide, Onfi®, Oxcarbazepine, Oxybutinin chloride, PF-04360365 (Pfizer, New York, N.Y.), Phenobarbital, Phenytek®, Phenytoin, piclozotan, Pioglitazone, Plavix, Potiga, Pram ipexole (Mirapex), pramlintide, Prednisone, Prim idone, Prinivil, probenecid, Propranolol, PRX-00023 (EPIX Pharmaceuticals Inc.), PXT3003, Quinacrine, Ramelteon, Rasagiline (Azilect), Rebif, ReciGen, remacemide, Resveratrol, Retavase, reteplase, riluzole (Rilutek), Rivastigmine (Exelon), Ropinirole (Requip), Rotigotine (Neupro), Rufinamide, Sabril, safinamide (EMD Serono, Rockland, Mass.), Salagen, Sarafem, Selegiline (1-deprenyl, Eldepryl), SEN0014196 (Siena Biotech, Siena, Italy), sertraline (Zoloft), Simvastatin, Sodium Nitroprussiate (NPS), sodium phenylbutyrate, Stanback Headache Powder, Tacrine (Cognex), Tamoxifen, tauroursodeoxycholic acid (TUDCA), Tegretol®, Tenecteplase, Tenormin, Tetrabenazine (Xenazine), THR-18 (Thrombotech Ltd.), Tiagabine, Tideglusib, tirofiban, tissue plasminogen activator (tPA), tizanidine (Zanaflex), TNKase, Tolcapone (Tasmar), Tolterodine, Topamax®, Topiramate, Trihexyphenidyl (formerly Artane), Trileptal®, ursodiol, Valproic Acid, valsartan, Varenicline (Pfizer), Vimpat, Vitamin E, Warfarin, Zarontin®, Zestril, Zonegran®, Zonisamide, Zydis selegiline HCL Oral disintegrating (Zelapar), and combinations thereof.

For example, to treat or ameliorate the effects of epilepsy, a subject may be administered an effective amount of one or more compounds or pharmaceutical compositions of the present disclosure and, e.g., one or more of the following: Albendazole, Banzel, BGG492 (Novartis Corp., New York, N.Y.) Carbamazepine, Carbatrol®, Clobazam, Clonazepam, Depakene®, Depakote®, Depakote ER®, Diastat, Diazepam, Dilantin®, Eslicarbazepine acetate, Ethosuximide, Ezogabine, Felbatol®, Felbamate, Frisium, Gabapentin, Gabitril®, Inovelon®, JNJ-26489112 (Johnson and Johnson, New Brunswick, N.J.) Keppra®, Keppra XR™, Klonopin, Lacosamide, Lamictal®, Lamotrigine, Levetiracetam, Lorazepam, Luminal, Lyrica, Mysoline®, Memantine, Neurontin®, Onfi®, Oxcarbazepine, Phenobarbital, Phenytek®, Phenytoin, Potiga, Primidone, probenecid, PRX-00023 (EPIX Pharmaceuticals Inc, Lexington, Mass.), Rufinamide, Sabril, Tegretol®, Tegretol XR®, Tiagabine, Topamax®, Topiramate, Trileptal®, Valproic Acid, Vimpat, Zarontin®, Zonegran®, and Zonisamide.

To treat or ameliorate the effects of stroke, a subject may be administered an effective amount of one or more compounds or pharmaceutical compositions of the present disclosure and, e.g., one or more of the following: Aspirin, dipyridamole, Clopidogrel, tissue plasminogen activator (tPA), Warfarin, dabigatran, Heparin, Lovenox, citicoline, L-Alpha glycerylphosphorylcholine, cerebrolysin, Eptifibatide, Escitalopram, Tenecteplase, Alteplase, Minocycline, Esmolol, Sodium Nitroprussiate (NPS), Norepinephrine (NOR), Dapsone, valsartan, Simvastatin, piclozotan, Desmoteplase, losartan, amlodipine, Ancrod, human chorionic gonadotropin (hCG), epoetin alfa (EPO), Galantamine, and THR-18 (Thrombotech Ltd., Ness Ziona, Israel).

To treat or ameliorate the effects of myocardial infarction, a subject may be administered an effective amount of one or more compounds or pharmaceutical compositions of the present disclosure and, e.g., one or more of the following: lisinopril, atenolol, Plavix, metoprolol tartrate, Lovenox, Lopressor, Zestril, Tenormin, Prinivil, aspirin, Arixtra, clopidogrel, Salagen, nitroglycerin, metoprolol tartrate, heparin, Nitrostat, Nitro-Bid, Stanback Headache Powder, nitroglycerin, Activase, Nitrolingual, nitroglycerin, fondaparinux, Lopressor, heparin, nitroglycerin TL, Nitro-Time, Nitromist, Ascriptin, alteplase, Retavase, TNKase, Bufferin, Nitro-Dur, Minitran, reteplase, tenecteplase, clopidogrel, Fragmin, enoxaparin, dalteparin, tirofiban, and Aggrastat.

To treat or ameliorate the effects of type I diabetes, a subject may be administered an effective amount of one or more compounds or pharmaceutical compositions of the present disclosure and, e.g., one or more of the following: insulin, such as regular insulin (Humulin R, Novolin R, others), insulin isophane (Humulin N, Novolin N), insulin lispro (Humalog), insulin aspart (NovoLog), insulin glargine (Lantus) and insulin detemir (Levemir), octreotide, pramlintide, and liraglutide.

To treat or ameliorate the effects of Alzheimer's disease, a subject may be administered an effective amount of one or more compounds or pharmaceutical compositions of the present disclosure and, e.g., one or more of the following: Donepezil (Aricept), Rivastigmine (Exelon), Galantamine (Razadyne), Tacrine (Cognex), Memantine (Namenda), Vitamin E, CERE-110: Adeno-Associated Virus Delivery of NGF (Ceregene), LY450139 (Eli Lilly), Exenatide, Varenicline (Pfizer), PF-04360365 (Pfizer), Resveratrol, and Donepezil (Eisai Korea).

To treat or ameliorate the effects of Parkinson's disease, a subject may be administered an effective amount of one or more compounds or pharmaceutical compositions of the present disclosure and, e.g., one or more of the following: Carbidopa/levodopa immediate-release (Sinemet), Carbidopa/levodopa oral disintegrating (Parcopa), Carbidopa/levodopa/Entacapone (Stalevo), Ropinirole (Requip), Pram ipexole (Mirapex), Rotigotine (Neupro), Apomorphine (Apokyn), Selegiline (1-deprenyl, Eldepryl), Rasagiline (Azilect), Zydis selegiline HCL Oral disintegrating (Zelapar), Entacapone (Comtan), Tolcapone (Tasmar), Amantadine (Symmetrel), Trihexyphenidyl (formerly Artane), Benztropine (Cogentin), IPX066 (Impax Laboratories Inc.), Rasagiline (Teva Neuroscience, Inc.), ioflupane 1231 (DATSCAN®), safinamide (EMD Serono), and Pioglitazone.

To treat or ameliorate the effects of amyotrophic lateral sclerosis, a subject may be administered an effective amount of one or more compounds or pharmaceutical compositions of the present disclosure and, e.g., one or more of the following: riluzole (Rilutek), Lithium carbonate, Arimoclomol, Creatine, Tamoxifen, Mecobalam in, Memantine (Ebixa), and tauroursodeoxycholic acid (TUDCA).

To treat or ameliorate the effects of Friedreich's ataxia, a subject may be administered an effective amount of one or more compounds or pharmaceutical compositions of the present disclosure and, e.g., one or more of the following: Idebenone, Coenzyme Q, 5-hydroxytryptophan, Propranolol, Enalapril, Lisinopril, Digoxin, Erythropoietin, Lu AA24493, Deferiprone, Varenicline, IVIG, Pioglitazone, and EGb 761.

To treat or ameliorate the effects of multiple sclerosis, a subject may be administered an effective amount of one or more compounds or pharmaceutical compositions of the present disclosure and, e.g., one or more of the following: Avonex, Betaseron, Extavia, Rebif, Glatiramer (Copaxone), Fingolimod (Gilenya), Natalizumab (Tysabri), Mitoxantrone (Novantrone), baclofen (Lioresal), tizanidine (Zanaflex), methylprednisolone, CinnoVex, ReciGen, Masitinib, Prednisone, Interferon beta 1a, Interferon beta 1 b, and ELND002 (Elan Pharmaceuticals).

To treat or ameliorate the effects of Huntington's disease, a subject may be administered an effective amount of one or more compounds or pharmaceutical compositions of the present disclosure and, e.g., one or more of the following: Tetrabenazine (Xenazine), haloperidol (Haldol), clozapine (Clozaril), clonazepam (Klonopin), diazepam (Valium), escitalopram (Lexapro), fluoxetine (Prozac, Sarafem), sertraline (Zoloft), valproic acid (Depakene), divalproex (Depakote), lamotrigine (Lamictal), Dimebon, AFQ056 (Novartis), Ethyl-EPA (Miraxion™), SEN0014196 (Siena Biotech), sodium phenylbutyrate, citalopram, ursodiol, minocycline, remacemide, and mirtazapine.

To treat or ameliorate the effects of transmissible spongiform encephalopathy, a subject may be administered an effective amount of one or more compounds or pharmaceutical compositions of the present disclosure and e.g., Quinacrine.

To treat or ameliorate the effects of Charcot-Marie-Tooth disease, a subject may be administered an effective amount of one or more compounds or pharmaceutical compositions of the present disclosure and, e.g., one or more of the following: ascorbic acid and PXT3003.

To treat or ameliorate the effects of dementia with Lewy bodies, a subject may be administered an effective amount of one or more compounds or pharmaceutical compositions of the present disclosure and, e.g., one or more of the following: Aricept, Galantamine, Memantine, Armodafinil, Donepezil, and Ramelteon.

To treat or ameliorate the effects of corticobasal degeneration, a subject may be administered an effective amount of one or more compounds or pharmaceutical compositions of the present disclosure and, e.g., one or more of the following: Davunetide and Coenzyme Q10.

To treat or ameliorate the effects of progressive supranuclear palsy, a subject may be administered an effective amount of one or more compounds or pharmaceutical compositions of the present disclosure and, e.g., one or more of the following: Tideglusib, Rasagiline, alpha-lipoic acid/L-acetyl carnitine, Riluzole, Niacinamide, and Rivastigmine.

To treat or ameliorate the effects of hereditary spastic paraparesis, a subject may be administered an effective amount of one or more compounds or pharmaceutical compositions of the present disclosure and, e.g., one or more of the following: Baclofen, Tizanidine, Oxybutinin chloride, Tolterodine, and Botulinum toxin.

In the present disclosure, one or more compounds or pharmaceutical compositions may be co-administered to a subject in need thereof together in the same composition, simultaneously in separate compositions, or as separate compositions administered at different times, as deemed most appropriate by a physician.

An additional embodiment of the present disclosure is a method for treating or ameliorating the effects of a disorder in a subject in need thereof. This method comprises administering to the subject an effective amount of a pharmaceutical composition comprising a pharmaceutically acceptable carrier or diluent and one or more compounds having the structure of formula (1):

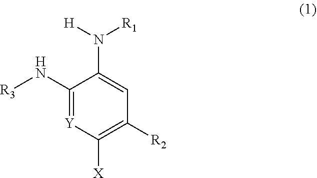

wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

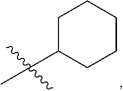 [SMILES: *C1CCCCC1]

R₂ cannot be

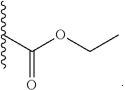 [SMILES: *C(=O)OCC]

Suitable and preferred pharmaceutical compositions for use in this method are as disclosed above in formulas (1), (1a), (1 b), (2), (3) and (3a), including pharmaceutical compositions containing the particular compounds identified above. Suitable and preferred subjects who may be treated in accordance with this method are as disclosed above. In this embodiment, the methods may be used to treat disorders set forth above, including degenerative diseases that involve lipid peroxidation and excitotoxic diseases that involve oxidative cell death.

In another aspect of this embodiment, the method further comprises co-administering to the subject an effective amount of one or more additional therapeutic agents disclosed herein.

Another embodiment of the present disclosure is a method of modulating ferroptosis in a subject in need thereof. This method comprises administering to the subject an effective amount of a ferroptosis inhibitor, which comprises one or more compounds having the structure of formula (1):

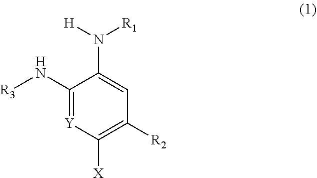

wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

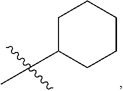 [SMILES: *C1CCCCC1]

R₂ cannot be

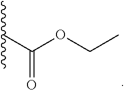 [SMILES: *C(=O)OCC]

As used herein, “ferroptosis” means regulated cell death that is iron-dependent. Ferroptosis is characterized by the overwhelming, iron-dependent accumulation of lethal lipid reactive oxygen species. (Dixon et al., 2012) Ferroptosis is distinct from apoptosis, necrosis, and autophagy. (Id.) Assays for ferroptosis are as disclosed herein, for instance, in the Examples section.

Suitable and preferred compounds for use in this method are as disclosed above in formulas (1), (1a), (1b), (2), (3) and (3a), including the particular compounds identified above. Suitable and preferred subjects who may be treated in accordance with this method are as disclosed above. In this embodiment, the methods may be used to treat the disorders set forth above, including degenerative diseases that involve lipid peroxidation and excitotoxic diseases that involve oxidative cell death.

In another aspect of this embodiment, the method further comprises co-administering to the subject an effective amount of one or more additional therapeutic agents disclosed herein.

A further embodiment of the present disclosure is a method of reducing reactive oxygen species (ROS) in a cell. This method comprises contacting a cell with a ferroptosis modulator, which comprises one or more compounds having the structure of formula (1):

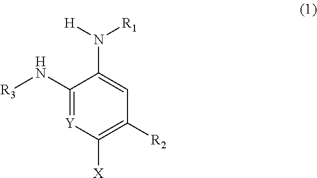

wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

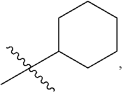 [SMILES: *C1CCCCC1]

R₂ cannot be

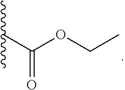 [SMILES: *C(=O)OCC]

As used herein, the terms “modulate”, “modulating”, “modulator” and grammatical variations thereof mean to change, such as decreasing or reducing the occurrence of ferroptosis. In this embodiment, “contacting” means bringing the compound and optionally one or more additional therapeutic agents into close proximity to the cells in need of such modulation. This may be accomplished using conventional techniques of drug delivery to the subject or in the in vitro situation by, e.g., providing the compound and optionally other therapeutic agents to a culture media in which the cells are located.

Suitable and preferred compounds for use in this method are as disclosed above in formulas (1), (1a), (1 b), (2), (3) and (3a), including the particular compounds identified above. In this embodiment, reducing ROS may be accomplished in cells obtained from a subject having a disorder as disclosed herein. Suitable and preferred subjects of this embodiment are as disclosed above.

In one aspect of this embodiment, the cell is a mammalian cell. Preferably, the mammalian cell is obtained from a mammal selected from the group consisting of humans, primates, farm animals, and domestic animals. More preferably, the mammalian cell is a human cancer cell.

In another aspect of this embodiment, the method further comprises contacting the cell with at least one additional therapeutic agent as disclosed herein.

An additional embodiment of the present disclosure is a method for treating or ameliorating the effects of a neurodegenerative disease in a subject in need thereof. This method comprises administering to the subject an effective amount of one or more compounds having the structure of formula (1):

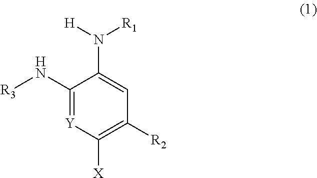

wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

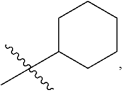 [SMILES: *C1CCCCC1]

R₂ cannot be

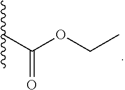 [SMILES: *C(=O)OCC]

Suitable and preferred compounds for use in this method are as disclosed above in formulas (1), (1a), (1 b), (2), (3) and (3a), including the particular compounds identified above. In this embodiment, the method may be used to treat the disorders set forth above.

Suitable and preferred subjects are as disclosed herein. In this embodiment, the methods may be used to treat the neurodegenerative disorders set forth above.

In one aspect of this embodiment, the method further comprises co-administering to the subject an effective amount of one or more therapeutic agents disclosed herein.

An additional embodiment of the present disclosure is a compound having the structure selected from the group consisting of:

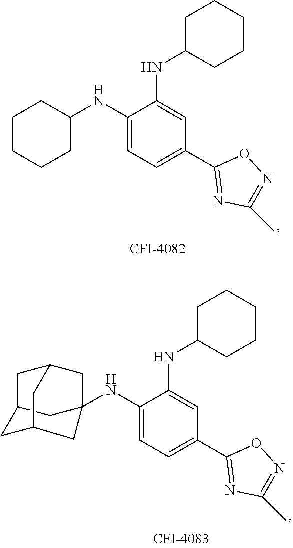 [SMILES: Cc1noc(-c2ccc(NC3CCCCC3)c(NC3CCCCC3)c2)n1 ; Cc1noc(-c2ccc(NC34CC5CC(CC(C5)C3)C4)c(NC3CCCCC3)c2)n1]

and combinations thereof, or an N-oxide, crystalline form, hydrate, or a pharmaceutically acceptable salt thereof.

An additional embodiment of the present disclosure is a pharmaceutical composition comprising a pharmaceutically acceptable carrier or diluent and a compound having the structure selected from the group consisting of:

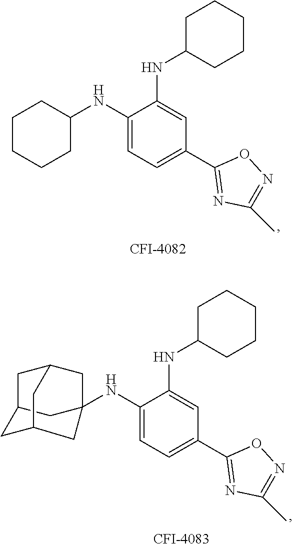 [SMILES: Cc1noc(-c2ccc(NC3CCCCC3)c(NC3CCCCC3)c2)n1 ; Cc1noc(-c2ccc(NC34CC5CC(CC(C5)C3)C4)c(NC3CCCCC3)c2)n1]

and combinations thereof, or an N-oxide, crystalline form, hydrate, or a pharmaceutically acceptable salt thereof.

An additional embodiment of the present disclosure is a method for treating or ameliorating the effects of a disorder in a subject in need thereof comprising administering to the subject an effective amount of a compound having the structure selected from the group consisting of:

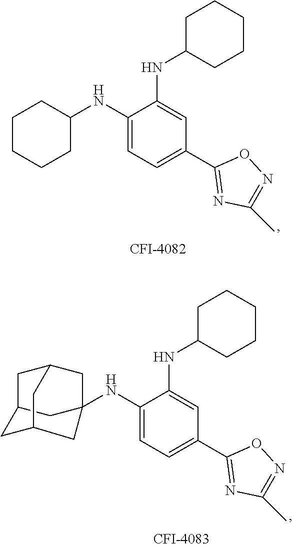 [SMILES: Cc1noc(-c2ccc(NC3CCCCC3)c(NC3CCCCC3)c2)n1 ; Cc1noc(-c2ccc(NC34CC5CC(CC(C5)C3)C4)c(NC3CCCCC3)c2)n1]

and combinations thereof, or an N-oxide, crystalline form, hydrate, or a pharmaceutically acceptable salt thereof.

An additional embodiment of the present disclosure is a method of modulating ferroptosis in a subject in need thereof comprising administering to the subject an effective amount of a ferroptosis inhibitor, which comprises a compound having the structure selected from the group consisting of:

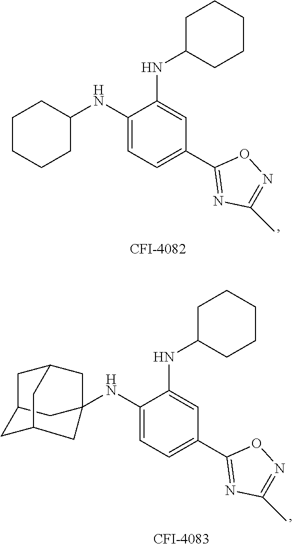 [SMILES: Cc1noc(-c2ccc(NC3CCCCC3)c(NC3CCCCC3)c2)n1 ; Cc1noc(-c2ccc(NC34CC5CC(CC(C5)C3)C4)c(NC3CCCCC3)c2)n1]

and combinations thereof, or an N-oxide, crystalline form, hydrate, or a pharmaceutically acceptable salt thereof.

An additional embodiment of the present disclosure is a method of reducing reactive oxygen species (ROS) in a cell comprising contacting a cell with a ferroptosis modulator, which comprises a compound having the structure selected from the group consisting of:

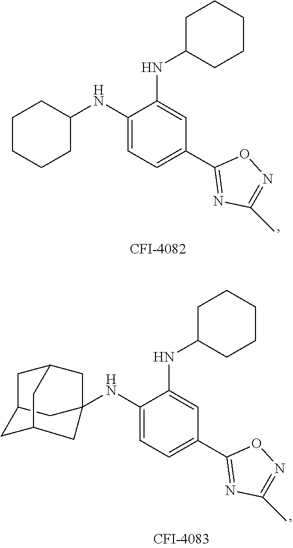 [SMILES: Cc1noc(-c2ccc(NC3CCCCC3)c(NC3CCCCC3)c2)n1 ; Cc1noc(-c2ccc(NC34CC5CC(CC(C5)C3)C4)c(NC3CCCCC3)c2)n1]

and combinations thereof, or an N-oxide, crystalline form, hydrate, or a pharmaceutically acceptable salt thereof.

An additional embodiment of the present disclosure is a method for treating or ameliorating the effects of a neurodegenerative disease in a subject in need thereof comprising administering to the subject an effective amount of a compound having the structure selected from the group consisting of:

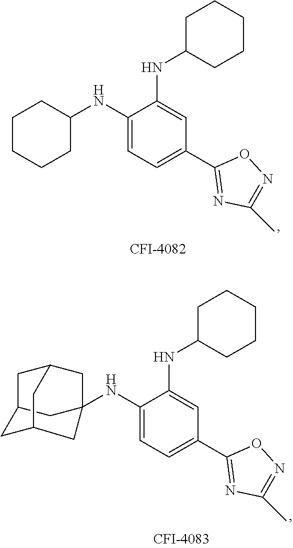 [SMILES: Cc1noc(-c2ccc(NC3CCCCC3)c(NC3CCCCC3)c2)n1 ; Cc1noc(-c2ccc(NC34CC5CC(CC(C5)C3)C4)c(NC3CCCCC3)c2)n1]

and combinations thereof, or an N-oxide, crystalline form, hydrate, or a pharmaceutically acceptable salt thereof.

An additional embodiment of the present disclosure is a compound having the structure selected from the group consisting of:

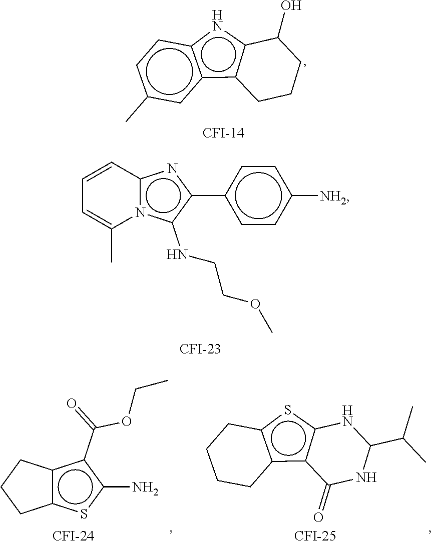

and combinations thereof, or an N-oxide, crystalline form, hydrate, or a pharmaceutically acceptable salt thereof.

An additional embodiment of the present disclosure is a pharmaceutical composition comprising a pharmaceutically acceptable carrier or diluent and a compound having the structure selected from the group consisting of:

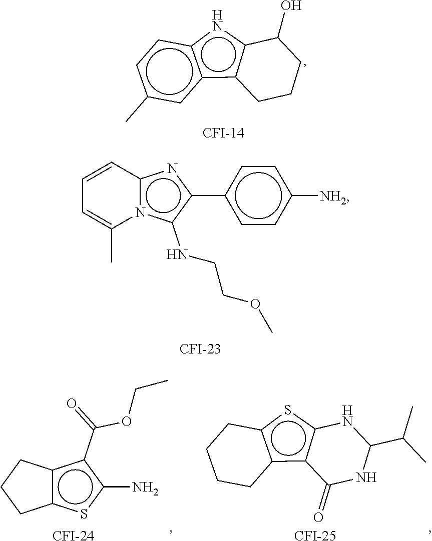

and combinations thereof, or an N-oxide, crystalline form, hydrate, or a pharmaceutically acceptable salt thereof.

An additional embodiment of the present disclosure is a method for treating or ameliorating the effects of a disorder in a subject in need thereof comprising administering to the subject an effective amount of a compound having the structure selected from the group consisting of:

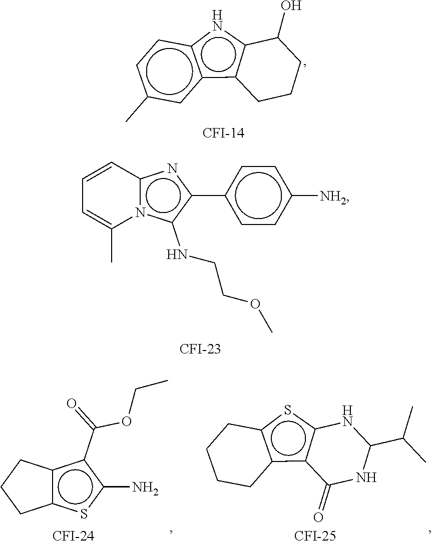

and combinations thereof, or an N-oxide, crystalline form, hydrate, or a pharmaceutically acceptable salt thereof.

An additional embodiment of the present disclosure is a method of modulating ferroptosis in a subject in need thereof comprising administering to the subject an effective amount of a ferroptosis inhibitor, which comprises a compound having the structure selected from the group consisting of:

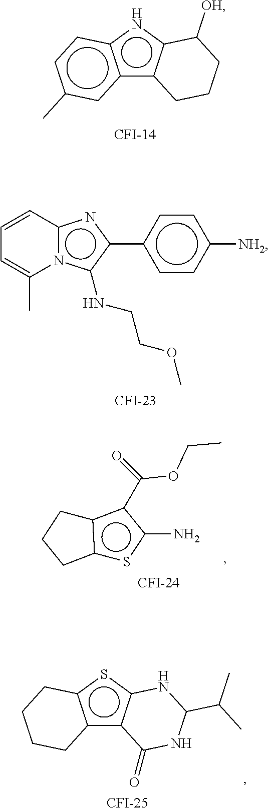

and combinations thereof, or an N-oxide, crystalline form, hydrate, or a pharmaceutically acceptable salt thereof.

An additional embodiment of the present disclosure is a method of reducing reactive oxygen species (ROS) in a cell comprising contacting a cell with a ferroptosis modulator, which comprises a compound having the structure selected from the group consisting of:

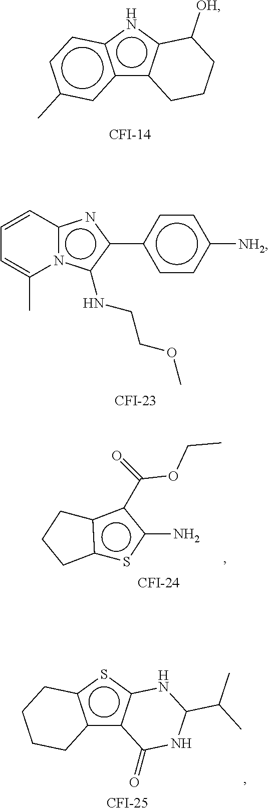

and combinations thereof, or an N-oxide, crystalline form, hydrate, or a pharmaceutically acceptable salt thereof.

An additional embodiment of the present disclosure is a method for treating or ameliorating the effects of a neurodegenerative disease in a subject in need thereof comprising administering to the subject an effective amount of a compound having the structure selected from the group consisting of:

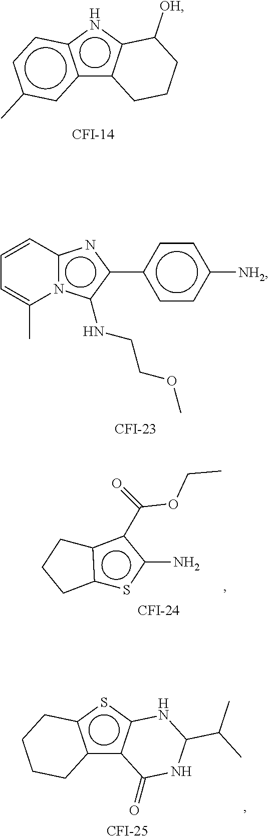

and combinations thereof, or an N-oxide, crystalline form, hydrate, or a pharmaceutically acceptable salt thereof.

Still another embodiment of the present disclosure is a method for alleviating side effects in a subject undergoing radiotherapy and/or immunotherapy, comprising administering to the subject an effective amount of one or more compounds disclosed herein.

As used herein, “radiotherapy” or “radiation therapy” refers to a therapy using ionizing radiation to control or kill malignant cells. Common side effects of radiotherapy include, but are not limited to, acute side effects (such as nausea, vomiting, damage to the epithelial surfaces, mouth, throat and stomach sores, intestinal discomfort, swelling, infertility, etc.), late side effects (such as fibrosis, epilation, dryness, lymphedema, cardiovascular disorder, cognitive decline, radiation enteropathy, radiation-induced polyneuropathy), and cumulative side effects.

As used herein, “immunotherapy” refers to the treatment of disease by activating or suppressing the immune system. It can be classified as an activation immunotherapy that elicits or amplifies an immune response, or a suppression immunotherapy that reduce or suppress an immune response. Common side effects of immunotherapy include, but are not limited to, skin problems (such as pain, swelling, soreness, redness, itchiness, rash, etc.), flu-like symptoms (such as fever, chills, weakness, dizziness, nausea or vomiting, muscle or joint aches, fatigue, headache, trouble breathing, low or high blood pressure, etc.), and other symptoms such as swelling and weight gain from retaining fluid, heart palpitations, sinus congestion, diarrhea, infection, organ inflammation, etc.

A further embodiment of the present disclosure is a method for treating or ameliorating the effects of an infection associated with ferroptosis in a subject, comprising administering to the subject an effective amount of one or more compounds disclosed herein. In some embodiments, the infection is caused by Mycobacterium tuberculosis.

As used herein, a “pharmaceutically acceptable salt” means a salt of the compounds of the present disclosure which are pharmaceutically acceptable, as defined herein, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

In the present disclosure, an “effective amount” or “therapeutically effective amount” of a compound or pharmaceutical composition is an amount of such a compound or composition that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of the subject, and like factors well known in the arts of, e.g., medicine and veterinary medicine. In general, a suitable dose of a compound or pharmaceutical composition according to the disclosure will be that amount of the compound or composition, which is the lowest dose effective to produce the desired effect with no or minimal side effects. The effective dose of a compound or pharmaceutical composition according to the present disclosure may be administered as two, three, four, five, six or more sub-doses, administered separately at appropriate intervals throughout the day.

A suitable, non-limiting example of a dosage of a compound or pharmaceutical composition according to the present disclosure or a composition comprising such a compound, is from about 1 ng/kg to about 1000 mg/kg, such as from about 1 mg/kg to about 100 mg/kg, including from about 5 mg/kg to about 50 mg/kg. Other representative dosages of a compound or a pharmaceutical composition of the present disclosure include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg.

A compound or pharmaceutical composition of the present disclosure may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, a compound or pharmaceutical composition of the present disclosure may be administered in conjunction with other treatments. A compound or pharmaceutical composition of the present disclosure may be encapsulated or otherwise protected against gastric or other secretions, if desired.

The pharmaceutical compositions of the disclosure are pharmaceutically acceptable and comprise one or more active ingredients in admixture with one or more pharmaceutically-acceptable carriers or diluents and, optionally, one or more other compounds, drugs, ingredients and/or materials. Regardless of the route of administration selected, the compounds/pharmaceutical compositions of the present disclosure are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, Pa.). More generally, “pharmaceutically acceptable” means that which is useful in preparing a composition that is generally safe, non-toxic, and neither biologically nor otherwise undesirable and includes that which is acceptable for veterinary use as well as human pharmaceutical use.

Pharmaceutically acceptable carriers and diluents are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier or diluent used in a composition of the disclosure must be “acceptable” in the sense of being compatible with the other ingredients of the formulation and not injurious to the subject. Carriers or diluents suitable for a selected dosage form and intended route of administration are well known in the art, and acceptable carriers or diluents for a chosen dosage form and method of administration can be determined using ordinary skill in the art.

The pharmaceutical compositions of the disclosure may, optionally, contain additional ingredients and/or materials commonly used in such compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Compounds or pharmaceutical compositions suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable carriers or diluents and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Compositions for rectal or vaginal administration may be presented as a suppository, which may be prepared by mixing one or more active ingredient(s) with one or more suitable nonirritating carriers which are solid at room temperature, but liquid at body temperature and, therefore, will melt in the rectum or vaginal cavity and release the active compound. Compositions which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable carrier or diluent. The ointments, pastes, creams and gels may contain excipients. Powders and sprays may contain excipients and propellants.

Compositions suitable for parenteral administrations comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier or diluent, for example water for injection, immediately prior to use. Extemporaneous injection solutions and suspensions may be prepared from sterile powders, granules and tablets of the type described above.

In the foregoing embodiments, the following definitions apply.

The term “aliphatic”, as used herein, refers to a group composed of carbon and hydrogen that do not contain aromatic rings. Accordingly, aliphatic groups include alkyl, alkenyl, alkynyl, and carbocyclyl groups. Additionally, unless otherwise indicated, the term “aliphatic” is intended to include both “unsubstituted aliphatics” and “substituted aliphatics”, the latter of which refers to aliphatic moieties having substituents replacing a hydrogen on one or more carbons of the aliphatic group. Such substituents can include, for example, a halogen, a deuterium, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aromatic, or heteroaromatic moiety.

The term “alkyl” refers to the radical of saturated aliphatic groups that does not have a ring structure, including straight-chain alkyl groups, and branched-chain alkyl groups. In certain embodiments, a straight chain or branched chain alkyl has 6 or fewer carbon atoms in its backbone (e.g., C₁-C₆ for straight chains, C₃-C₆ for branched chains). In other embodiments, the “alkyl” may include up to twelve carbon atoms, e.g., C₁, C₂, C₃, C₄, C₅, C₆, C₇, C₈, C₉, C₁₀, C₁₁ or C₁₂. Such substituents include all those contemplated for aliphatic groups, as discussed below, except where stability is prohibitive.

The term “alkenyl”, as used herein, refers to an aliphatic group containing at least one double bond and unless otherwise indicated, is intended to include both “unsubstituted alkenyls” and “substituted alkenyls”, the latter of which refers to alkenyl moieties having substituents replacing a hydrogen on one or more carbons of the alkenyl group. Such substituents include all those contemplated for aliphatic groups, as discussed below, except where stability is prohibitive. For example, substitution of alkenyl groups by one or more alkyl, carbocyclyl, aryl, heterocyclyl, or heteroaryl groups is contemplated.

Moreover, unless otherwise indicated, the term “alkyl” as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Indeed, unless otherwise indicated, all groups recited herein are intended to include both substituted and unsubstituted options.

The term “C_{x-y}” when used in conjunction with a chemical moiety, such as, alkyl and cycloalkyl, is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc.

The term “aryl” as used herein includes substituted or unsubstituted single-ring aromatic groups in which each atom of the ring is carbon. Preferably the ring is a 3- to 8-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “alkyl-aryl” refers to an alkyl group substituted with at least one aryl group.

The term “alkyl-heteroaryl” refers to an alkyl group substituted with at least one heteroaryl group.

The term “alkenyl-aryl” refers to an alkenyl group substituted with at least one aryl group.

The term “alkenyl-heteroaryl” refers to an alkenyl group substituted with at least one heteroaryl group.

The terms “carbocycle”, “carbocyclyl”, and “carbocyclic”, as used herein, refer to a non-aromatic saturated or unsaturated ring in which each atom of the ring is carbon. Preferably a carbocycle ring contains from 3 to 10 atoms, more preferably from 3 to 8 atoms, including 5 to 7 atoms, such as for example, 6 atoms. The term “cabocycle” also includes bicycles, tricycles and other multicyclic ring systems, including the adamantyl ring system.

The terms “halo” and “halogen” are used interchangeably herein and mean halogen and include chloro, fluoro, bromo, and iodo.

The term “heteroaryl” includes substituted or unsubstituted aromatic single ring structures, preferably 3- to 8-membered rings, more preferably 5- to 7-membered rings, even more preferably 5- to 6-membered rings, whose ring structures include at least one heteroatom, preferably one to four heteroatoms, more preferably one or two heteroatoms. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur; more preferably, nitrogen and oxygen.

The term “ketone” means an organic compound with the structure RC(═O)R′, wherein neither R nor R′ can be hydrogen atoms.

The term “ether” means an organic compound with the structure R—O—R′, wherein neither R nor R′ can be hydrogen atoms.

The term “ester” means an organic compound with the structure RC(═O)OR′, wherein neither R nor R′ can be hydrogen atoms.

The term “polyyne” means is an organic compound with alternating single and triple bonds; that is, a series of consecutive alkynes, (—C≡C—) n with n greater than 1.

The term “substituted” refers to moieties having substituents replacing a hydrogen on one or more carbons of the backbone. It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

As set forth previously, unless specifically stated as “unsubstituted,” references to chemical moieties herein are understood to include substituted variants. For example, reference to an “aryl” group or moiety implicitly includes both substituted and unsubstituted variants.

As used herein, the term “oxadiazole” means any compound or chemical group containing the following structure:

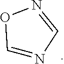

As used herein, the term “oxazole” means any compound or chemical group containing the following structure:

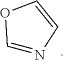

As used herein, the term “triazole” means any compound or chemical group containing the following structure:

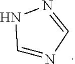

It is understood that the disclosure of a compound herein encompasses all stereoisomers of that compound. As used herein, the term “stereoisomer” refers to a compound made up of the same atoms bonded by the same bonds but having different three-dimensional structures which are not interchangeable. The three-dimensional structures are called configurations. Stereoisomers include enantiomers and diastereomers.

The terms “racemate” or “racemic mixture” refer to a mixture of equal parts of enantiomers. The term “chiral center” refers to a carbon atom to which four different groups are attached. The term “enantiomeric enrichment” as used herein refers to the increase in the amount of one enantiomer as compared to the other.

It is appreciated that to the extent compounds of the present disclosure have a chiral center, they may exist in and be isolated in optically active and racemic forms. Some compounds may exhibit polymorphism. It is to be understood that the present disclosure encompasses any racemic, optically-active, diastereomeric, polymorphic, or stereoisomeric form, or mixtures thereof, of a compound of the disclosure, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

Examples of methods to obtain optically active materials are known in the art, and include at least the following:
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved using chiral catalysts as disclosed in more detail herein or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

The stereoisomers may also be separated by usual techniques known to those skilled in the art including fractional crystallization of the bases or their salts or chromatographic techniques such as LC or flash chromatography. The (+) enantiomer can be separated from the (−) enantiomer using techniques and procedures well known in the art, such as that described by J. Jacques, et al., Enantiomers, Racemates, and Resolutions”, John Wiley and Sons, Inc., 1981. For example, chiral chromatography with a suitable organic solvent, such as ethanol/acetonitrile and Chiralpak AD packing, 20 micron can also be utilized to effect separation of the enantiomers.

The following examples are provided to further illustrate the methods of the present disclosure. These examples are illustrative only and are not intended to limit the scope of the disclosure in any way.

### EXAMPLES

The detailed experimental procedures applied to Ferrostatin-1 and its analogs have been described previously in the International Application No. PCT/US2014/067977, filed on Dec. 1, 2014, the entirety of which is incorporated herein by reference.

### Example 1

### Synthesis of Ferrostatin-1 Analogs

### Chemicals

Solvents, inorganic salts, and organic reagents were purchased from commercial sources such as Sigma and Fisher and used without further purification unless otherwise noted. Erastin was dissolved in DMSO to a final concentration of 73.1 mM and stored in aliquots at −20° C.

### Chromatography

Merck pre-coated 0.25 mm silica plates containing a 254 nm fluorescence indicator were used for analytical thin-layer chromatography. Flash chromatography was performed on 230-400 mesh silica (SiliaFlash® P60) from Silicycle.

### Spectroscopy

¹H, ¹³C and ¹⁹F NMR spectra were obtained on a Bruker DPX 400 MHz spectrometer. HRMS spectra were taken on double focusing sector type mass spectrometer HX-110A. Maker JEOL Ltd. Tokyo Japan (resolution of 10,000 and 10 KV accel. Volt. Ionization method; FAB (Fast Atom Bombardment) used Xe 3Kv energy. Used Matrix, NBA (m-Nitro benzyl alcohol)).

### General Procedure A (Esterification)

A representative example is the esterification of the 4-chloro-3-nitrobenzoic acid with tert-butanol. 4-dimethylaminopyridine (DMAP) (2.4607 g, 20.14 mmol, 0.4 equiv) and tert-butanol (24 mL, 250.94 mmol, 5.1 equiv) were added to a solution of 4-chloro-3-nitrobenzoic acid (10.0042 g, 49.63 mmol, 1.0 equiv) dissolved in dichloromethane (350 mL) at room temperature. N, N′-dicyclohexylcarbodiimide (DCC) (13.7853 g, 66.81 mmol, 1.4 equiv) was added to the solution at 0° C. The reaction mixture was allowed to warm to room temperature and stirred overnight under nitrogen atmosphere. The white precipitate was filtered off and the solution was purified by flash-column chromatography on silica gel (hexane, ethyl acetate gradient 40% max).

### General Procedure B (Nucleophilic Aromatic Substitution)

A representative example is the nucleophilic aromatic substitution of tert-butyl 4-chloro-3-nitrobenzoate with 1-admantylamine. Potassium carbonate (2.1570 g, 15.61 mmol, 1.9 equiv) was added to a solution of tert-butyl 4-chloro-3-nitrobenzoate (2.0784 g, 8.07 mmol, 1.0 equiv) dissolved in DMSO (13 mL). A solution of 1-adamantylamine (1.4273 g, 9.44 mmol, 1.2 equiv) dissolved in DMSO (13 mL) was added to the reaction mixture at room temperature. The reaction mixture was heated at 75° C. and stirred overnight under nitrogen atmosphere. After the reaction mixture was cooled to room temperature, water (200 mL) was added and the aqueous layer was extracted three times with ethyl acetate (100 mL). Combined organic layers were extracted with water (30 mL), dried (MgSO₄) and purified by flash-column chromatography on silica gel (hexane, ethyl acetate gradient 40% max).

### General Procedure C (Hydrogenation)

A representative example is the hydrogenation of tert-butyl 4-(1-adamantylamino)-3-nitrobenzoate. Pd(OH)₂ on charcoal (0.5048 g) was added to a solution of tert-butyl 4-(1-adamantylamino)-3-nitrobenzoate (1.0079 g, 2.71 mmol) dissolved in MeOH (100 mL) at room temperature. The reaction mixture was stirred at room temperature overnight under hydrogen atmosphere. The black solid was filtered out and the solution was purified by flash-column chromatography on silica gel (dichloromethane, methanol gradient).

### General Procedure D (Imine Formation)

A representative example is the imine formation reaction between tert-butyl 4-(1-adamantylamino)-3-aminobenzoate and pyrimidine-5-carboxaldehyde. Pyrimidine-5-carboxaldehyde (0.5653 g, 5.23 mmol, 2.9 equiv) and MgSO₄ (0.7850 g) were added to a solution of tert-butyl 4-(1-adamantylamino)-3-aminobenzoate (0.6097 g, 1.78 mmol, 1.0 equiv) dissolved in dichloromethane (122 mL) at room temperature. The reaction mixture was purged once with nitrogen and stirred at room temperature for two overnights under nitrogen atmosphere. The solution was purified by flash-column chromatography on silica gel (hexane, ethyl acetate gradient).

### General Procedure E (Oxidized Imine Formation)

A representative example is the oxidized imine formation reaction between tert-butyl 4-(1-adamantylamino)-3-aminobenzoate and pyrimidine-5-carboxaldehyde. Pyrimidine-5-carboxaldehyde (0.0415 g, 0.38 mmol, 1.3 equiv) was added to a solution of tert-butyl 4-(1-adamantylamino)-3-aminobenzoate (0.1008 g, 0.29 mmol, 1.0 equiv) dissolved in tert-butanol (6 mL). 4M HCl in dioxane (10 μL) was added to the solution at room temperature. The reaction mixture was stirred at 80° C. for 4 hours under nitrogen atmosphere. The solution was purified by flash-column chromatography on silica gel (dichloromethane, methanol gradient).

### General Procedure F (Reductive Amination)

A representative example is the reductive amination reaction between tert-butyl 3-(1-adamantylamino)-4-aminobenzoate and cyclohexanone. Cyclohexanone (0.5 mL, 4.83 mmol, 6.8 equiv) was added dropwise to a solution of tert-butyl 3-(1-adamantylamino)-4-aminobenzoate (0.2416 g, 0.706 mmol, 1 equiv) dissolved in 1,2-dichloroethane (24 mL) at room temperature. Sodium triacetoxyborohydride (0.8913 g, 4.21 mmol, 5.96 mmol) and glacial acetic acid (50 μL, 0.874 mmol, 1.24 equiv) were added to the solution at room temperature. The reaction mixture was stirred at room temperature overnight under nitrogen atmosphere. The solution was purified by flash-column chromatography on silica gel (hexane, ethyl acetate gradient).

### Design and Synthesis of Microsome and Plasma Stable Ferrostatin Analogs

A general route to obtain the compounds of formulas (I) to (III) follows a three-step synthesis (see below). An S_{N}Ar reaction between the commercially available ethyl 4-chloro-3-nitrobenzoate and cyclohexylamine, followed by catalytic hydrogenolysis of the nitro group, provided the desired ferrostatin derivatives. The anilines of the latter were reacted through reductive amination with arylaldehydes in the presence of sodium triacetoxyborohydride or through straightforward alkylation with arylalkylhalides in the presence of Hunig's base.

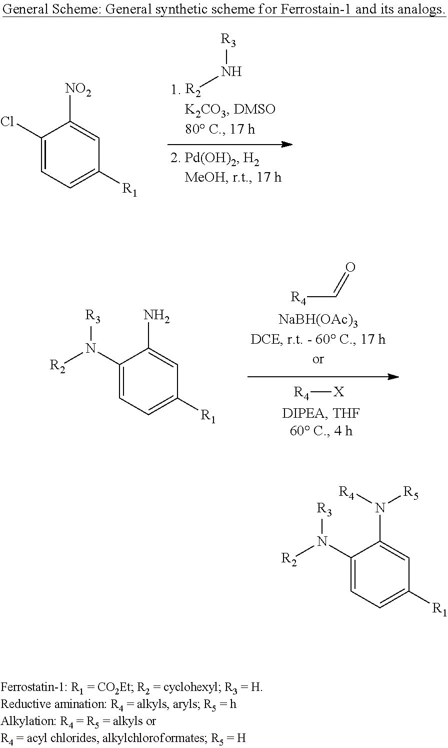

Experimental data pointed to the benzylic position of ferrostatin analogs as the site of metabolic liability in microsomes, and the ester group as the target of plasma esterases. Therefore, analog synthesis focuses on modification of these positions with the goal of improving microsomal and plasma stability in vitro and with the ultimate goal of producing analogs with improved in vivo properties for use in animal models of disease. Because in silico evaluation of Fer-1 analogs' P450 stability using the Schrodinger Suite P450_SOM program showed agreement with the experimental results with liver microsomes, this computer program is used to guide prioritization of compound synthesis and testing of analogs proposed based on modifications known to inhibit metabolism.

One of the most useful methods of blocking metabolism at a specific site is to use a steric shield—a bulky group that hinders oxidation at the position by cytochrome P450. An efficient synthesis of Fer-1 analogs with bulky, blocking groups incorporated at the benzylic site of oxidation is shown in Scheme 1.

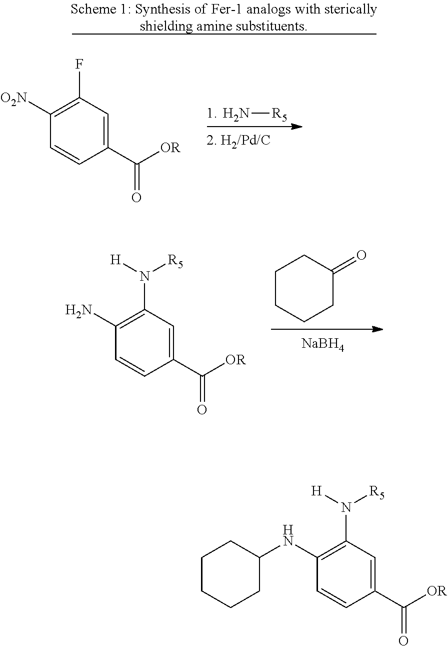

Treatment of commercially available 3-fluoro-4-nitrobenzoic acid with a benzylamine containing the desired bulky substituent at the benzylic position would displace fluoride via an SNAr reaction to give the corresponding aminonitro compound (Saitoh, et al., 2009). A wide range of benzyl amines are commercially available. Enantiomerically pure amines are important because cytochrome P450s are known to be enantioselective in their oxidations. Benzylically disubstituted amines would increase the amount of steric shielding and have the advantage of being achiral. The 2,6-dimethylbenzyl amine illustrates another mode of shielding the benzylic position.

The synthetic route shown in Scheme 1 also allows ready access to other substituted amine analogs that can be explored, and that may be more resistant to metabolism, as they do not have a benzylic position to react with P450s. Thus, aniline, cyclohexylamine, and adamantly amine may be used as starting materials to give the corresponding analogs.

The t-butyl ester is resistant to plasma esterases; however, this group may be acid labile, and may not be resistant to the acidic conditions in the stomach upon oral dosing. Bioisosteres, functionalities that are biologically equivalent to the functional group they are replacing, are commonly used to produce active analogs with improved properties, such as resistance to metabolism (Hamada, et al., 2012). A number of ester bioisosteres have been reported in the literature and can be incorporated into analogs of Fer-1. As shown in the synthetic route in Scheme 2, the acid or ester group of 3-fluoro-4-nitrobenzoic acid can be readily converted into ester bioisosteres, such as oxazoles (Wu, et al., 2004), oxadiazoles (Pipik, et al., 2004), triazoles (Passaniti, et al., 2002), or ketones (Genna, et al., 2011). These intermediates can then be used in the synthetic route outlined in Scheme 1 to produce the desired Fer-1 analogs with ester bioisosteres that are resistant to esterases.

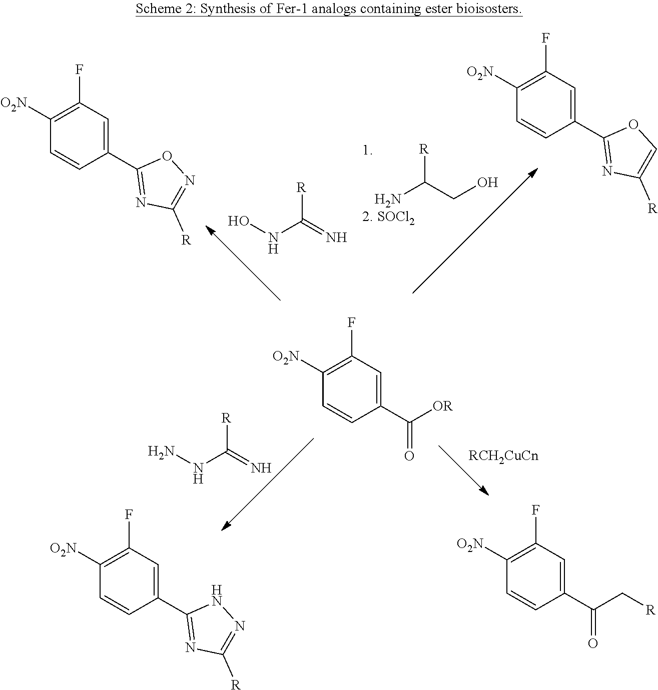

The synthetic routes of representative Fer-1 analogs are illustrated as follow:

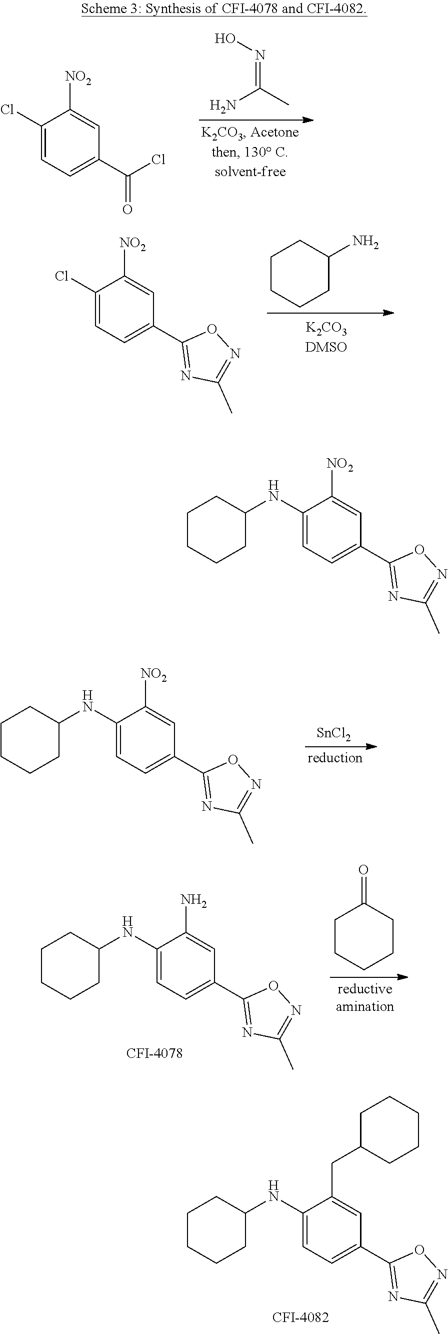

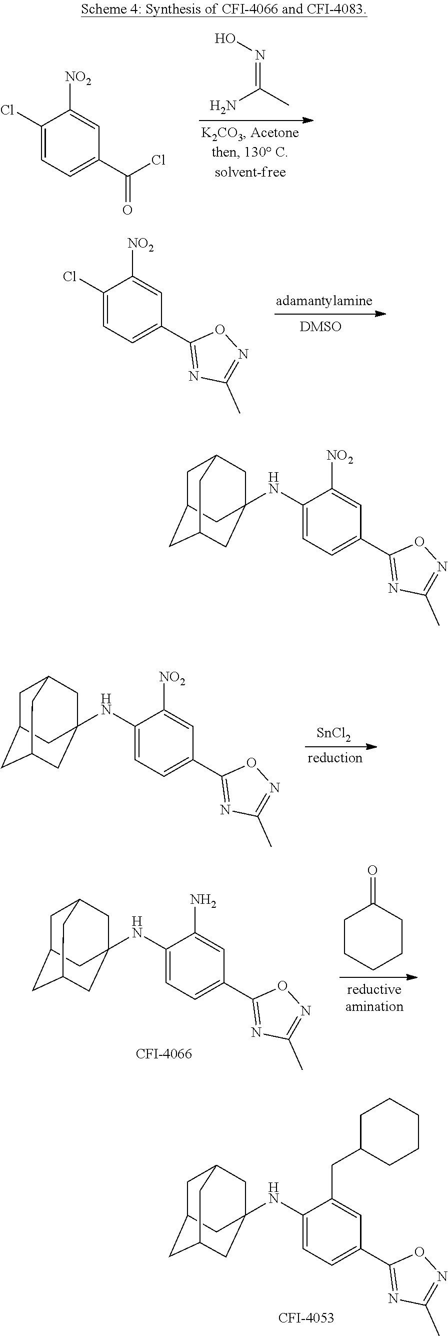

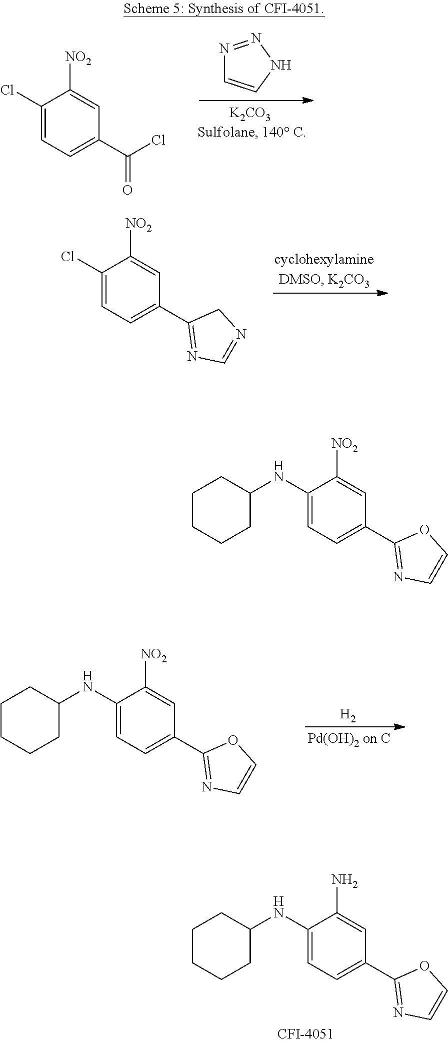

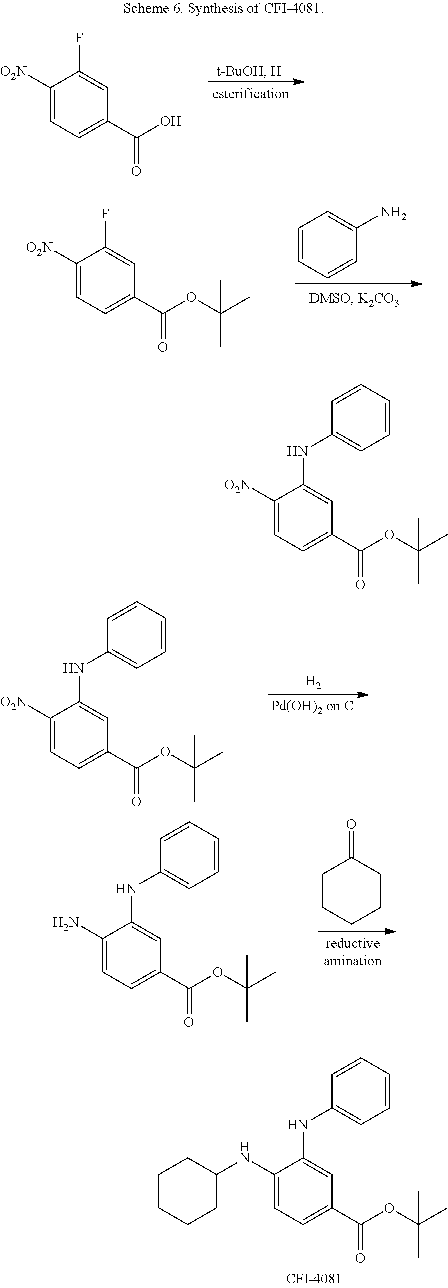

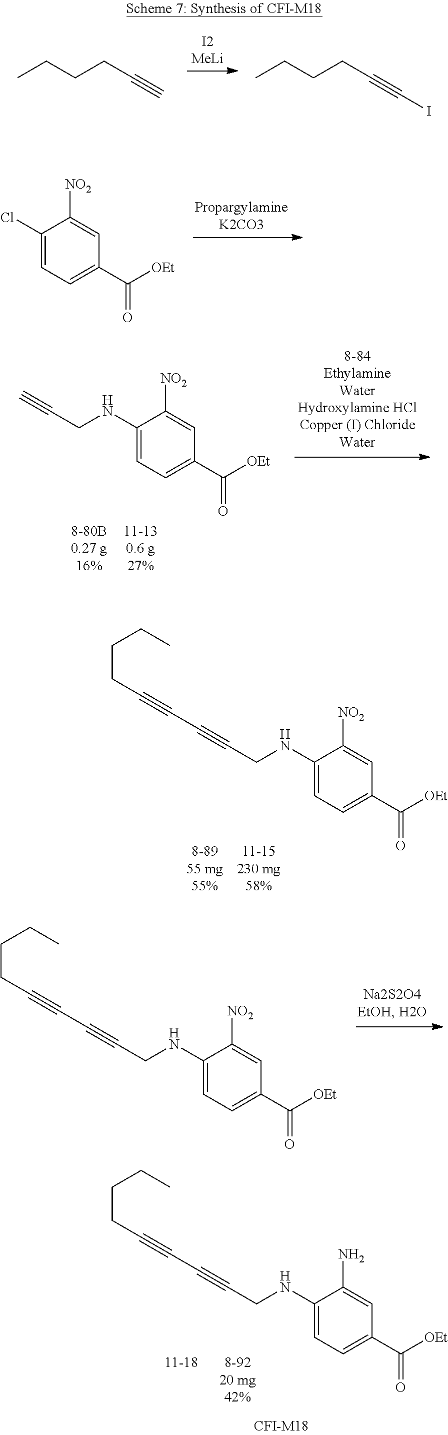

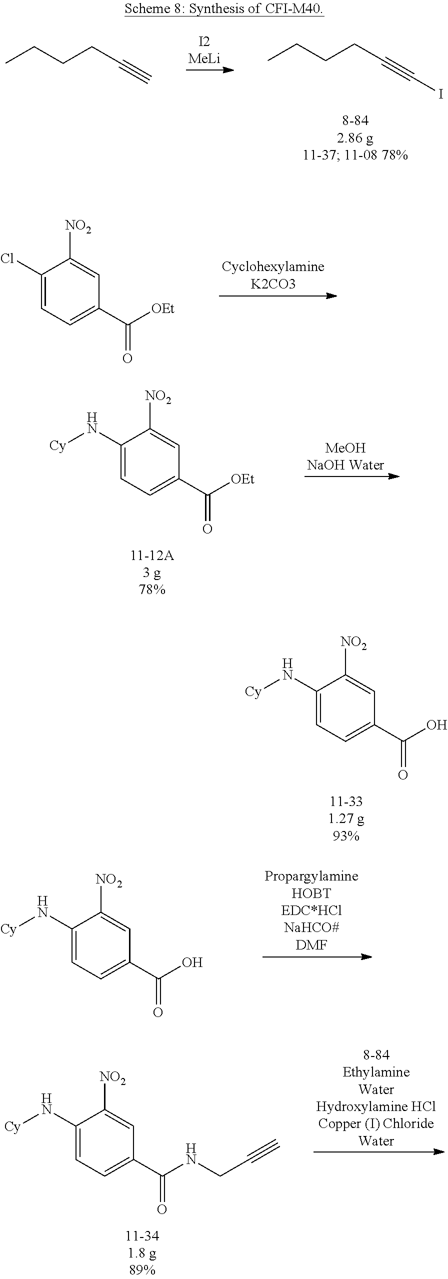

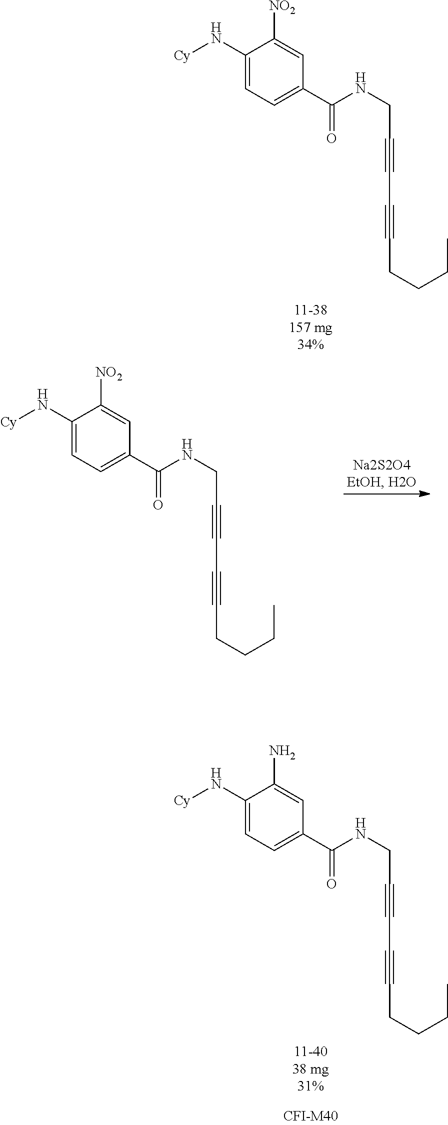

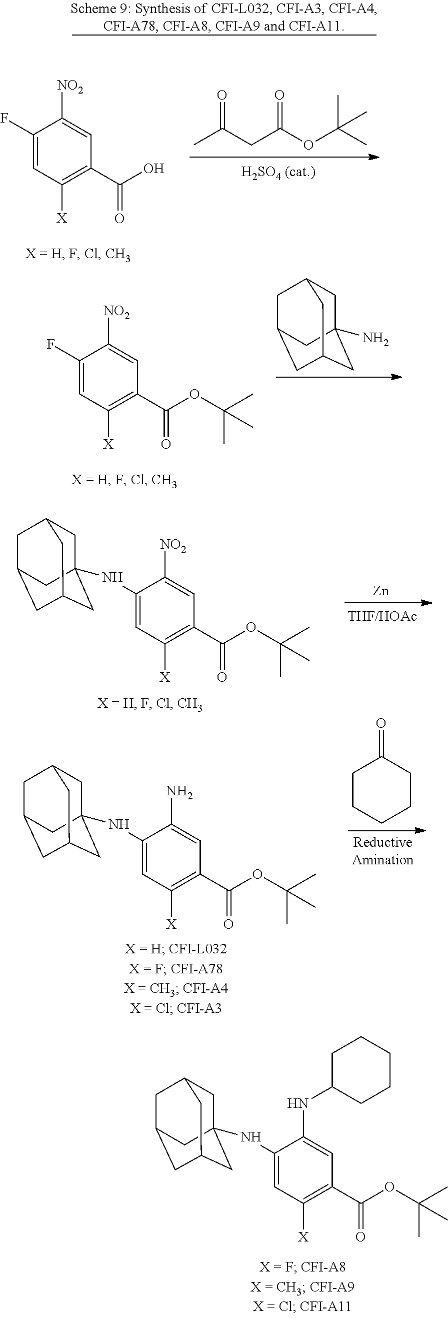

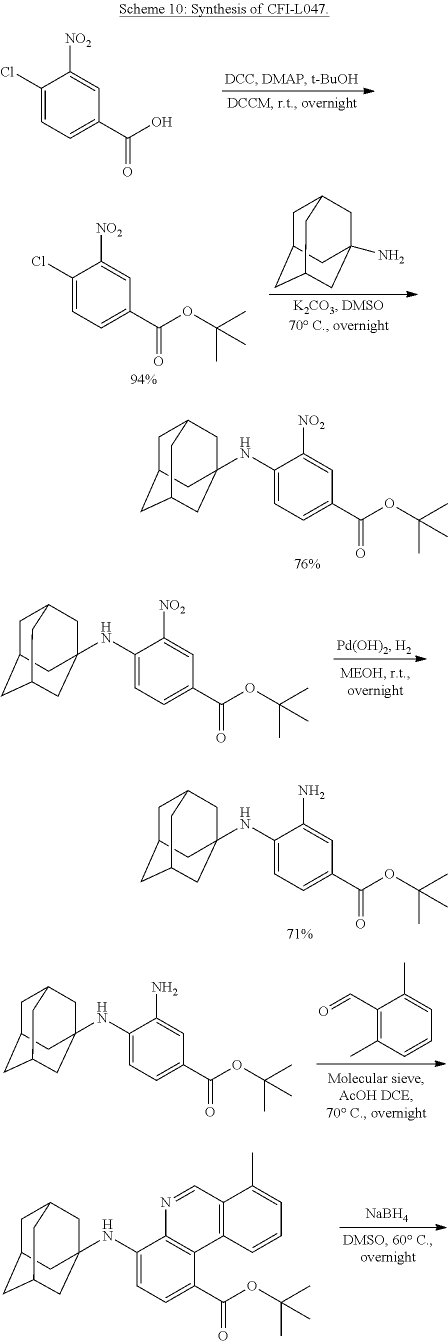

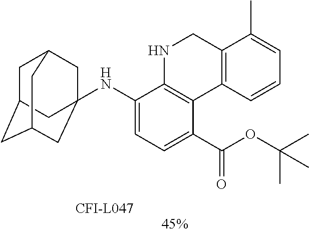 [SMILES: Cc1cccc2c1CNc1c(NC34CC5CC(CC(C5)C3)C4)ccc(C(=O)OC(C)(C)C)c1-2]

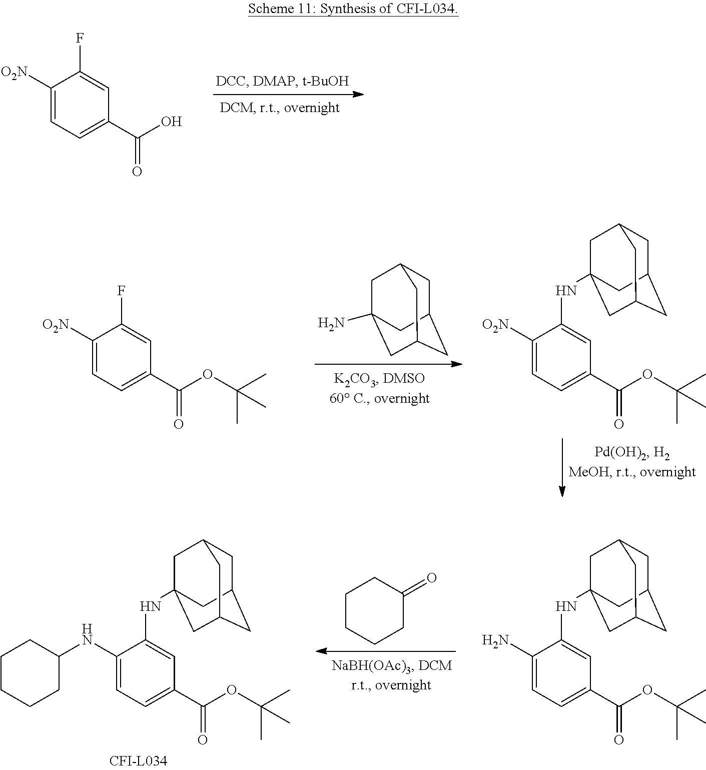

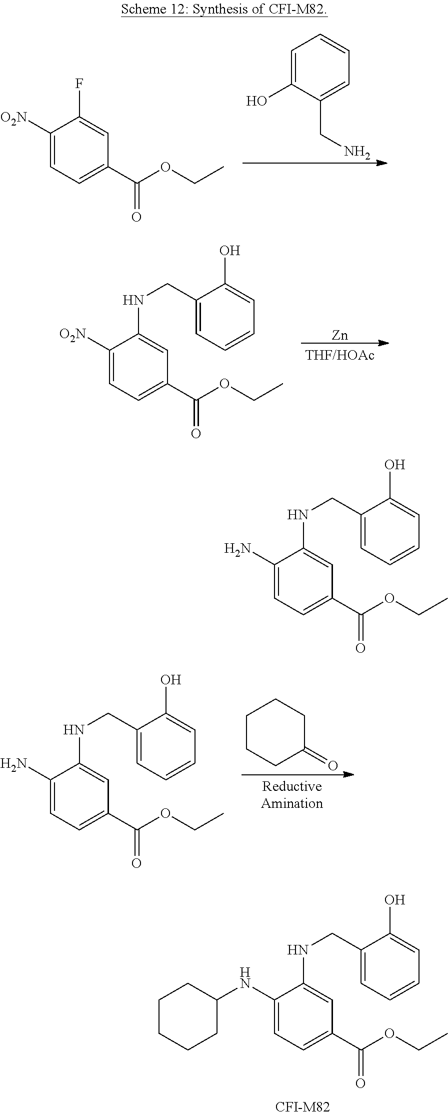

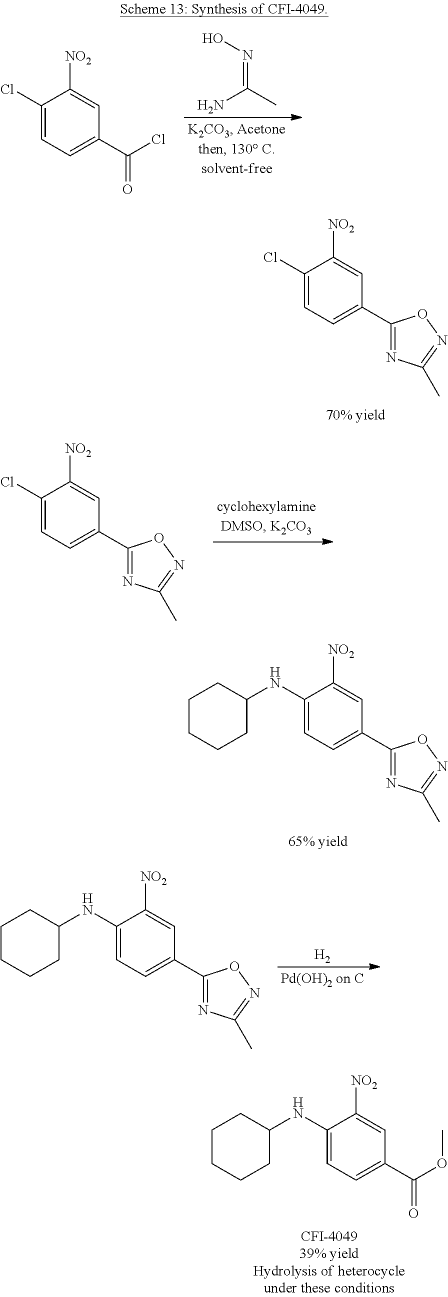

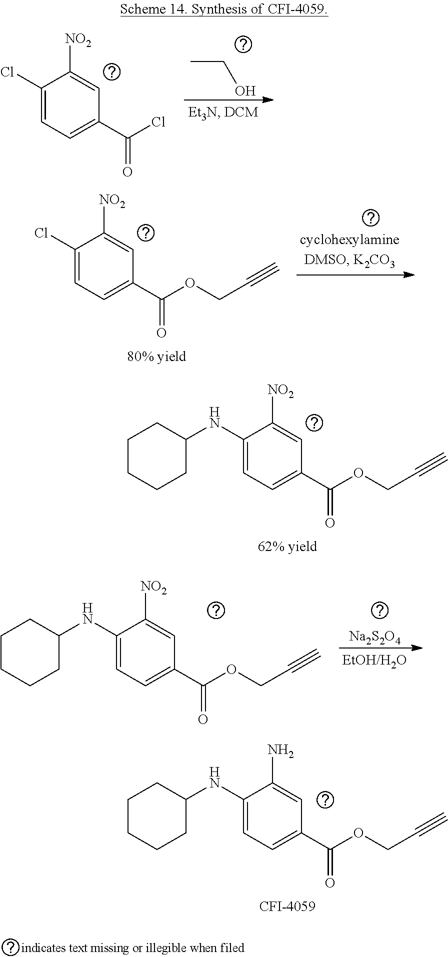

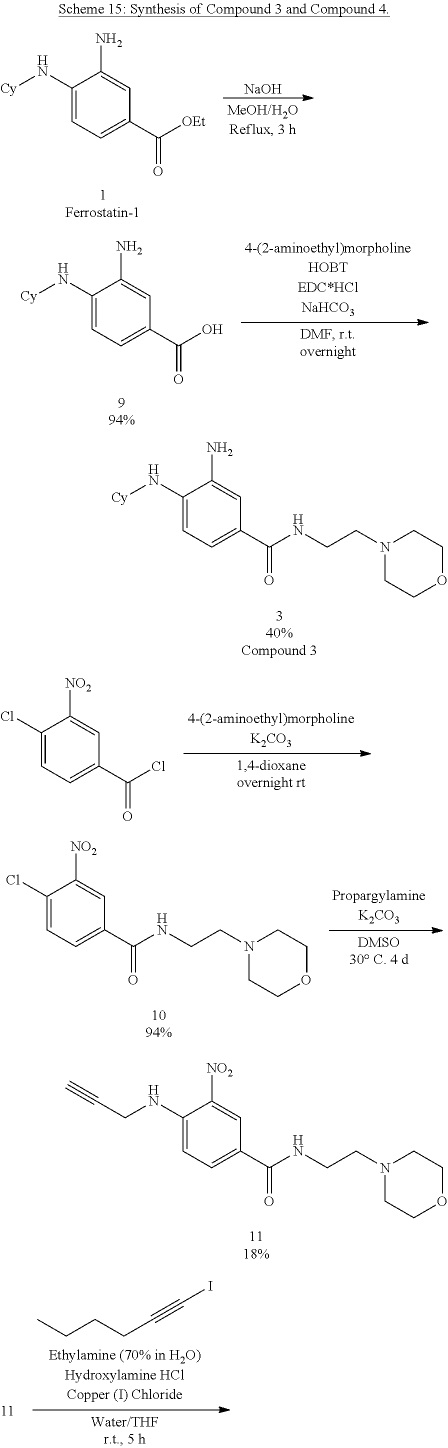

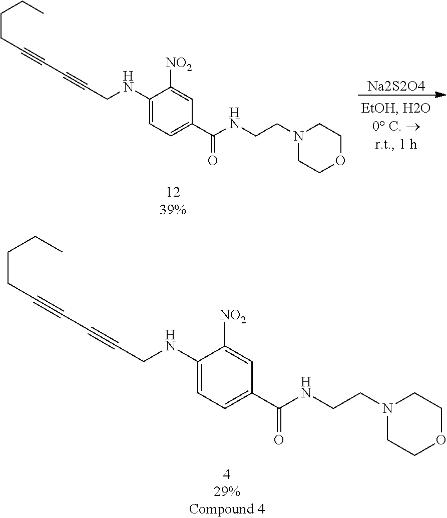 [SMILES: CCCCC#CC#CCNc1ccc(C(=O)NCCN2CCOCC2)cc1[N+](=O)[O-] ; "Na2S2O4" ; CCCCC#CC#CCNc1ccc(C(=O)NCCN2CCOCC2)cc1[N+](=O)[O-]]

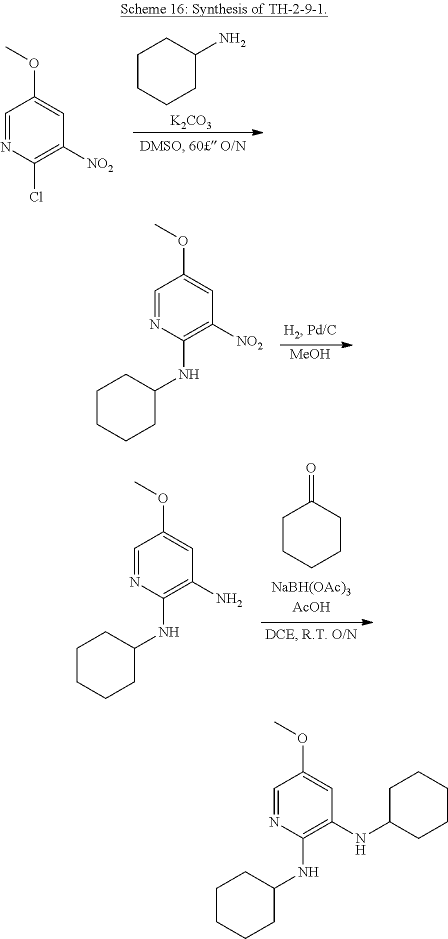

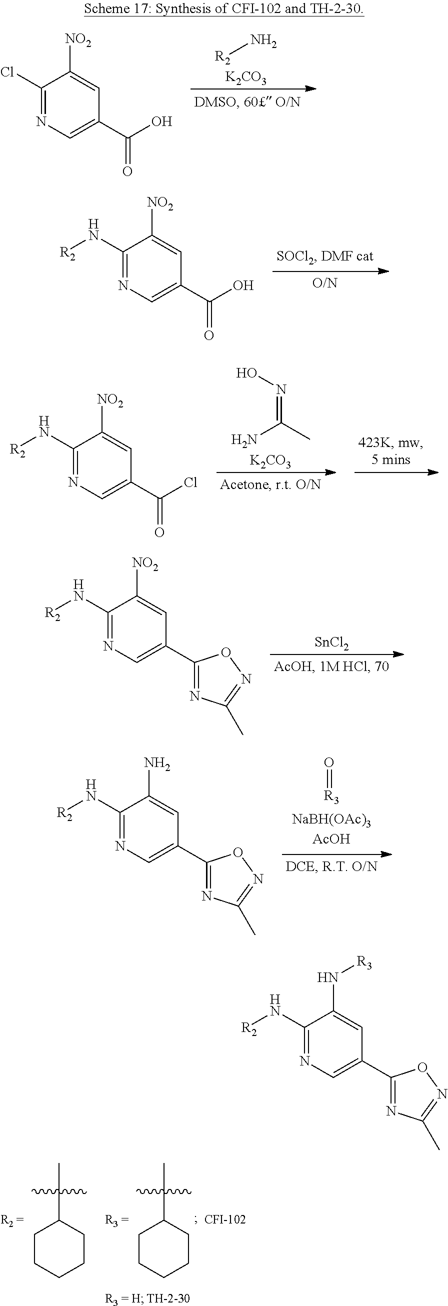

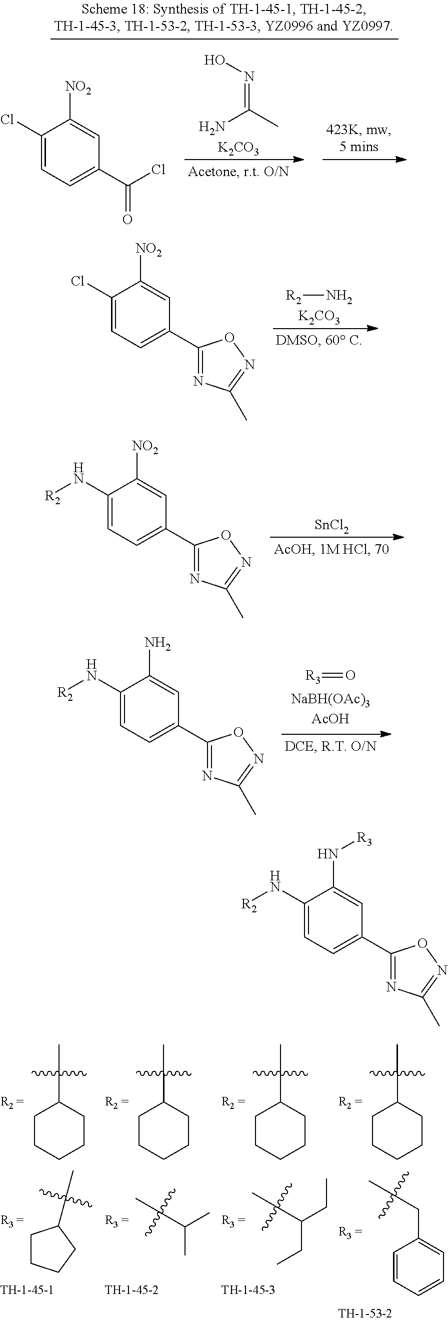

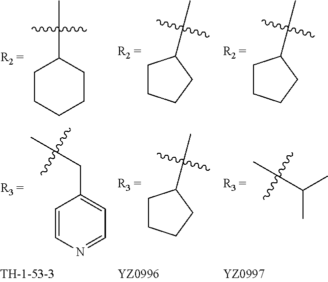

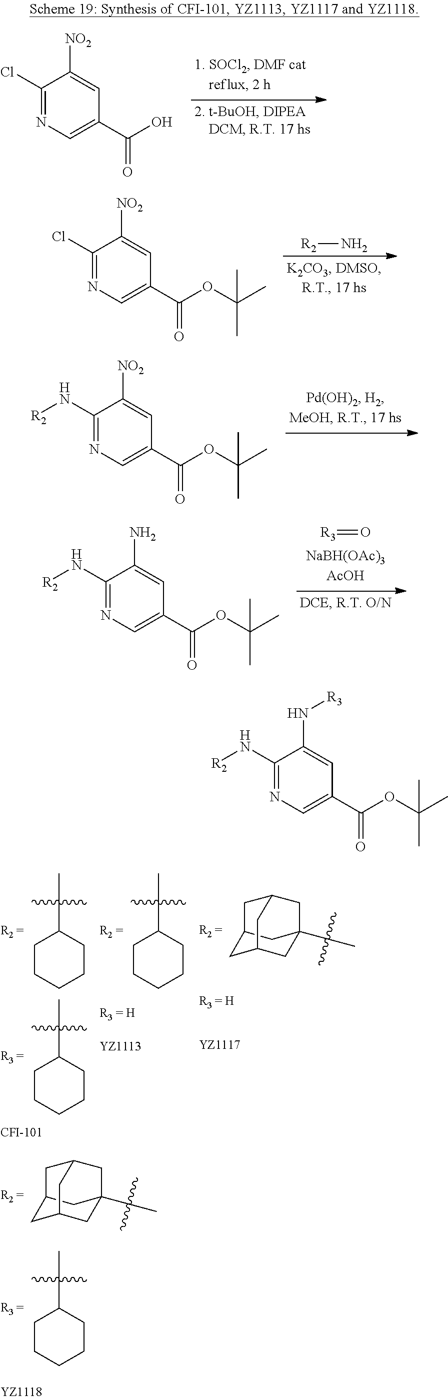

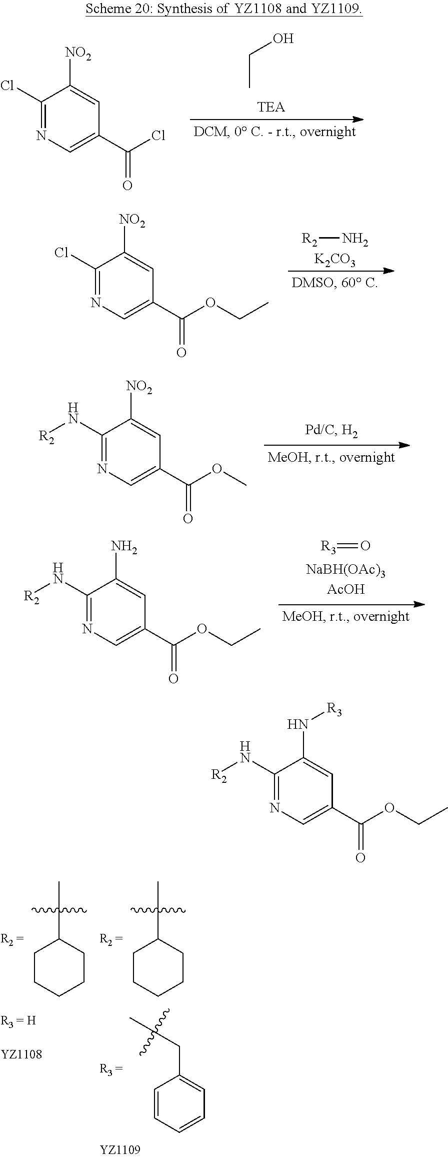

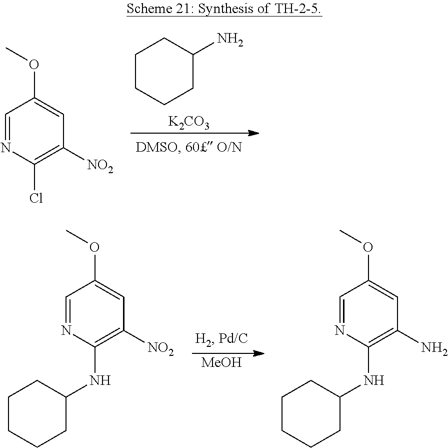

### Example 2

### Biological Activities of Ferrostatin-1 Analogs

All analogs are tested in vitro for their ability to inhibit erastin-induced ferroptosis in cells. Those with an IC₅₀ of <50 nM are tested for metabolic stability in mouse liver microsomes and plasma. Those analogs with T_{1/2}>30 minutes in those assays undergo pharmacokinetic analysis in mice. Those analogs with the best in vivo PK parameters are tested in the HD mouse model (see below).

### Rescue Activity of Fer-1 Analogs (Dixon, et al., 2012)

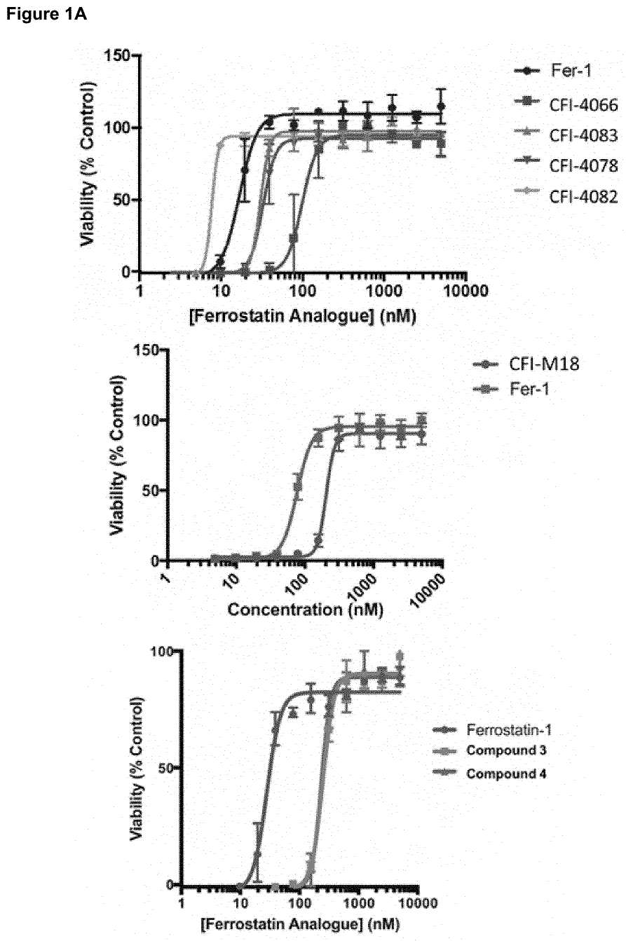

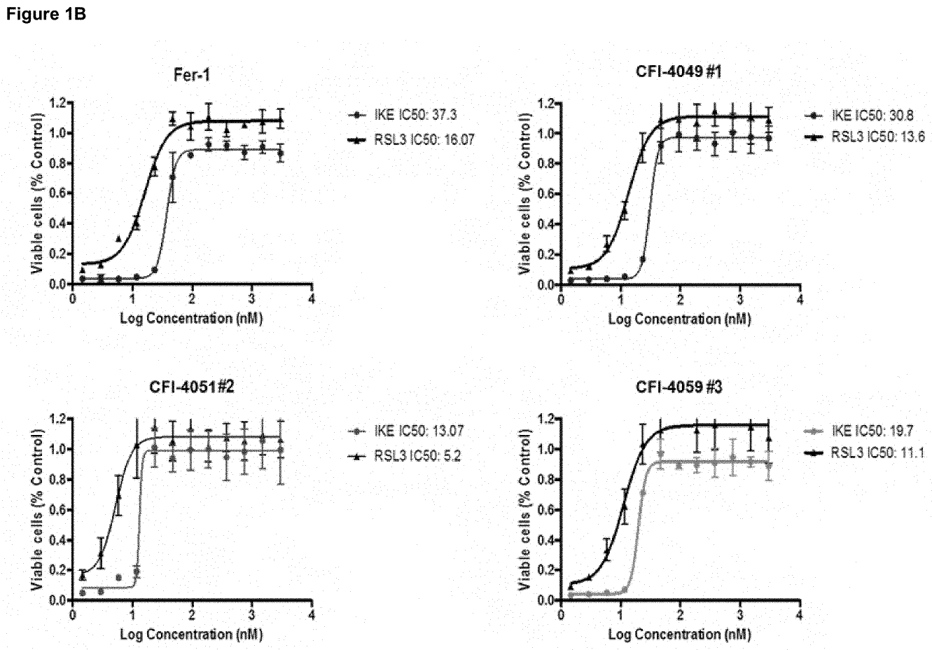

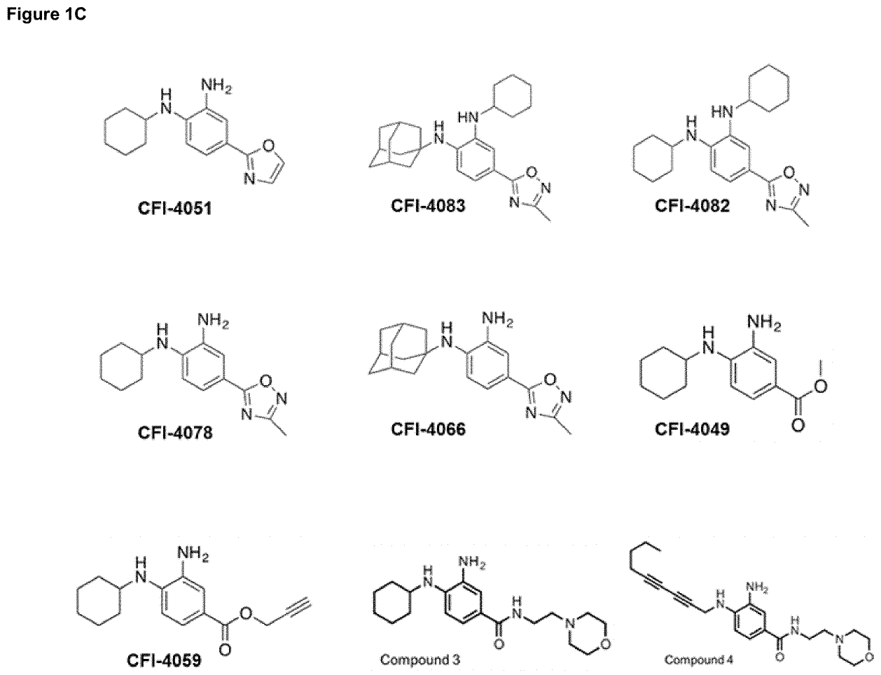

HT-1080 cells are cultured in DMEM containing 10% fetal bovine serum, 1% supplemented non-essential amino acids and 1% pen/strep mixture (Gibco) and maintained in a humidified environment at 37° C. with 5% CO₂ in a tissue culture incubator. 1,000 HT-1080 cells are seeded per well in duplicate 384-well plates (Corning) using a BioMek FX liquid handling robot (Beckman Coulter). The next day, the medium is replaced with 36 μL of medium containing 10 μM erastin with 4 μL of medium containing a dilution series (previously prepared) of DMSO, Fer-1 (positive control) or Fer-1 analogs. 24 hours later, 10 μL Alamar Blue (Invitrogen) cell viability solution is added to the growth media to a final concentration of 10%. Cells are incubated a further 6 hours and then the Alamar Blue fluorescence intensity recorded using a Victor 3 platereader (PerkinElmer)(ex/em 530/590). All experiments are performed at least twice and the background (no cells)-subtracted Alamar Blue values for each combination are averaged between replicates. The same procedure was repeated by replacing erastin (10 μM) with IKE (3 μM) or RSL3 (0.2 μM). From these data, sigmoidal dose-response viability curves (FIG. 1A for erastin, FIG. 1B for IKE and RSL3) and EC₅₀ values (Table 1) are computed using Prism 5.0 (GraphPad).

### Plasma and Metabolic Stability

Each compound (1 μM) is incubated with mouse plasma, for 4 hours at 37° C., with shaking at 100 rpm. The concentration of compound in the buffer and plasma chambers is determined using LC-MS/MS. Metabolism of each compound is predicted using Sites of Metabolism (Schrodinger Suite), which combines intrinsic reactivity analysis (Hammett-Taft) with induced fit docking against 2C9, 2D6 and 3A4. This approach identifies 90% of known metabolism sites and has a false positive rate of 17%. The in vitro metabolic stability of each compound in mouse liver microsomes is determined. Pooled mouse liver microsomes are prepared and stored at −80° C. until needed. Compound stability in liver microsomes is measured at 0, 15, 30, 45 and 60 minutes in duplicate, using LC-MS/MS analysis.

### Pharmacokinetic Evaluation of Compounds in Mice

To evaluate the PK profile of compounds, IV, IP, and PO administration of each compound is used in C57BL/6J wt mice. Mice are dosed IV at 10 mg/kg and sacrificed using Nembutal and CO₂ euthanasia. Six week old mice (Charles River) that have been acclimated to their environment for 2 weeks are used. All animals are observed for morbidity, mortality, injury, availability of food and water twice per day. Animals in poor health are euthanized. Blood samples are collected via cardiac puncture at each time point (0, 30 minutes, 2, 4, 8, 24 h). In addition, brains are collected, and compound concentration determined at each time point using LCO2N MS/MS. Standard PK parameters are calculated for each route of administration, including T_{1/2}, Cmax, AUC, clearance, Vd and % F.

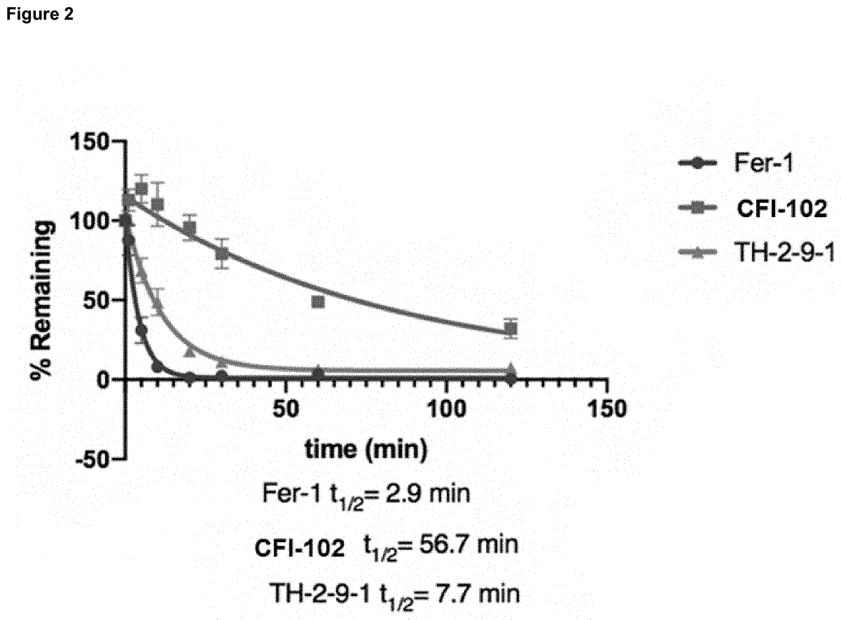

The properties of Ferrostatin-1 and analogs are summarized in Table 1. CFI-A8, CFI-A9, CFI-A11, CFI-L032, CFI-L034, CFI-L047, CFI-4082 and CFI-4083 show T_{1/2}>120 minutes in either mouse or human liver microsomes. Particularly, CFI-4082 and CFI-4083 show T_{1/2}>120 minutes in both mouse and human liver microsomes. The microsomal stability comparison (half-life measured in mouse) of Fer-1, CFI-102 and TH-2-9-1 is also provided in FIG. 2.

TABLE 1
Properties of Ferrostatin-1 and analogs.

| | | Plasma | | | | | | |
|---|---|---|---|---|---|---|---|---|
| | | Stability | | | | | | |
| | | at six | | | | | | |
| | Microsomal | hours (% | | | | donor | acceptor | |
| Compound and Structure | Stability t_{1/2} | remaining) | MW | ClogP | PSA | HB | HB | EC₅₀ |
| 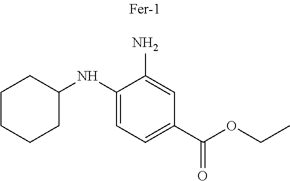 [SMILES: CCOC(=O)c1ccc(NC2CCCCC2)c(N)c1] | Mouse: 2.4 ± 0.6 min 1.9 ± 0.6 min¹ Human: 11.4 ± 0.2 min¹ 6.9 ± 0.2 min¹ Rat: 3.0 ± 11.1 min Dog: 49.5 ± 9.6% remaining t = | Mouse: 0-2% Human: 100% Rat: 0-4% [symbol] | 262.351 | 2.694 | 71.607 | 2.5 | 4 | 50^{a} |
| | 120 | | | | | | | |
| | Pig: 16.2 ± | Pig: 100% | | | | | | |
| | 3.9 min | | | | | | | |
| 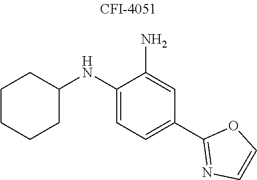 [SMILES: Nc1cc(-c2ncco2)ccc1NC1CCCCC1] | Mouse: 3.2- 3.3 min Human: >120 min Rat: 30 < t_{1/2} < 60 min Dog: >120 min Pig: — | Mouse: — Human: — Rat: — [symbol] Pig: — | 257.335 | 2.641 | 57.392 | 2.5 | 4 | 364^{a} 13^{b} 5^{c} |
| 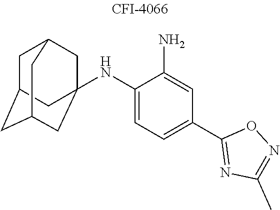 [SMILES: Cc1noc(-c2ccc(NC34CC5CC(CC(C5)C3)C4)c(N)c2)n1] | Mouse: 30.2- 32.4 min Human: — Rat: — Dog: — Pig: — | Mouse: — Human: — Rat: — [symbol] Pig: — | 324.425 | 2.854 | 74.416 | 2.5 | 5 | 123^{a} |
| 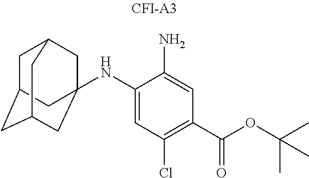 [SMILES: CC(C)(C)OC(=O)c1cc(N)c(NC23CC4CC(CC(C4)C2)C3)cc1Cl] | Mouse: 16.2- 16.6 min Human: >120 min Rat: 30 < t_{1/2} < 60 min Dog: >120 min Pig: — | Mouse: — Human: — Rat: — [symbol] Pig: — | 376.925 | 4.474 | 64.699 | 2.5 | 4 | 26^{b} 6^{c} |
| 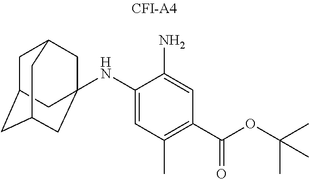 [SMILES: Cc1cc(NC23CC4CC(CC(C4)C2)C3)c(N)cc1C(=O)OC(C)(C)C] | Mouse: 12.5- 13.4 min Human: >120 min Rat: ~20 min Dog: 30 < t_{1/2} < 60 min Pig: — | Mouse: — Human: — Rat: — [symbol] Pig: — | 356.507 | 4.335 | 63.254 | 2.5 | 4 | 16^{b} 1^{c} |
| 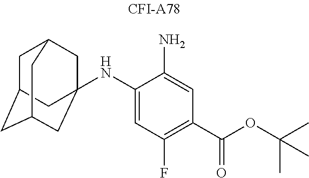 [SMILES: CC(C)(C)OC(=O)c1cc(N)c(NC23CC4CC(CC(C4)C2)C3)cc1F] | Mouse: 25.7- 27.7 min Human: — Rat: — [symbol] Pig: — | Mouse: — Human: — Rat: — [symbol] Pig: — | 360.471 | 4.272 | 65.511 | 2.5 | 4 | 26^{b} 18^{c} |
| 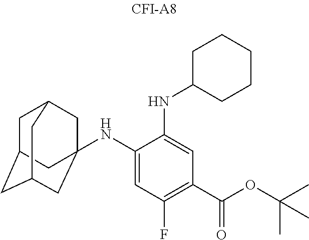 [SMILES: CC(C)(C)OC(=O)c1cc(NC2CCCCC2)c(NC23CC4CC(CC(C4)C2)C3)cc1F] | Mouse: > 120 min (76.4%) Human: TBD Rat: TBD [symbol] Pig: TBD | Mouse: 100% Human: TBD Rat: TBD [symbol] Pig: TBD | 442.616 | 6.598 | 50.622 | 2 | 4 | 20^{b} 19^{c} |
| 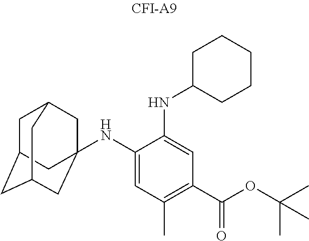 [SMILES: Cc1cc(NC23CC4CC(CC(C4)C2)C3)c(NC2CCCCC2)cc1C(=O)OC(C)(C)C] | Mouse: > 120 min (83.6%) Human: TBD Rat: TBD [symbol] Pig: TBD | Mouse: 100% Human: TBD Rat: TBD [symbol] Pig: TBD | 438.652 | 6.692 | 48.468 | 2 | 4 | 65^{a} |
| 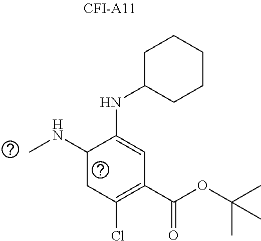 [SMILES: CNC1CC(Cl)=C(C(=O)OC(C)(C)C)C=C1NC1CCCCC1] | Mouse: > 120 min (69.1%) Human: TBD Rat: TBD Dog: TBD Pig: TBD | Mouse: 100% Human: TBD Rat: TBD [symbol] Pig: TBD | 459 .07 | 6.818 | 49.837 | 2 | 4 | 95^{a} |
| 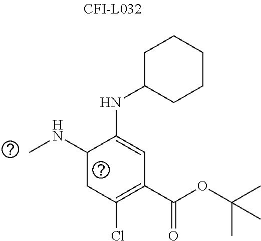 [SMILES: CNC1CC(Cl)=C(C(=O)OC(C)(C)C)C=C1NC1CCCCC1] | Mouse: > 120 min (78.7%) Human: TBD Rat: TBD [symbol] Pig: TBD | Mouse: 100% Human: TBD Rat: TBD [symbol] Pig: TBD | 424.625 | 6.346 | 50.91 | 2 | 4 | 43^{a} 77^{b} |
| 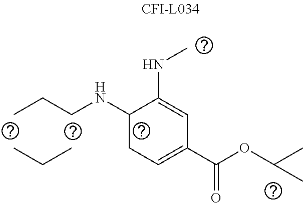 | Mouse: > 120 min (60.2%) Human: TBD Rat: TBD [symbol] Pig: TBD | Mouse: 100% Human: TBD Rat: TBD [symbol] Pig: TBD | 424.625 | 6.415 | 50.95 | 2 | 4 | 215^{a} 166^{b} |
| 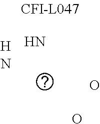 | Mouse: > 120 min (59.3%) Human: TBD Rat: TBD [symbol] Pig: TBD | Mouse: 100% Human: TBD Rat: TBD [symbol] Pig: TBD | 460.658 | 7.248 | 50.864 | 2 | 4 | 294^{a} |
| 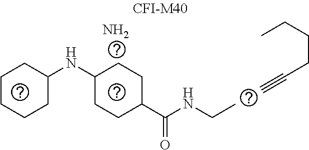 | Mouse: ~60 min Human: TBD Rat: TBD [symbol] Pig: TBD | Mouse: 100% Human: TBD Rat: TBD [symbol] Pig: TBD | 351.491 | 4.564 | 73.747 | 3.5 | 4.5 | 77^{a} |
| 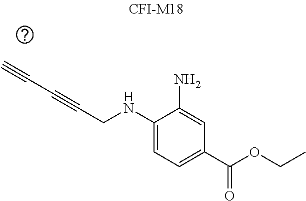 [SMILES: C#CC#CCNc1ccc(C(=O)OCC)cc1N] | Mouse: 20 < t_{1/2} < 30 min Human: TBD Rat: TBD [symbol] Pig: TBD | Mouse: — Human: — Rat: — [symbol] Pig: — | 298.384 | 3.78 | 73.366 | 2.5 | 4 | 203^{a} |
| 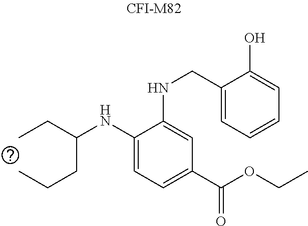 [SMILES: CCCC(CC)Nc1ccc(C(=O)OCC)cc1NCc1ccccc1O] | Mouse: 10 min Human: — Rat: — [symbol] Pig: — | Mouse: — Human:— Rat: — [symbol] Pig: — | 368.475 | 4.557 | 76.996 | 3 | 4.75 | 14^{a} |
| 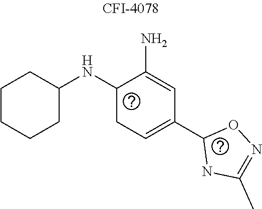 [SMILES: CC1=NOC(C2=CCC(NC3CCCCC3)C(N)=C2)N1] | Mouse: ~65 min Human: TBD Rat: TBD [symbol] TBD Pig: TBD | Mouse: — Human: — Rat: — [symbol] Pig: — | 272.349 | 2.202 | 75.449 | 2.5 | 5 | 102^{a} |
| 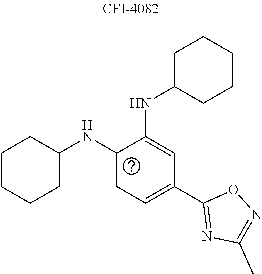 [SMILES: Cc1noc(C2=CCC(NC3CCCCC3)C(NC3CCCCC3)=C2)n1] | Mouse: ~120 min Human: >120 min Rat: 30 < t_{1/2} < 60 min [symbol] Pig: 60 < t_{1/2} < 120 min | Mouse: 100% Human: TBD Rat: TBD [symbol] Pig: TBD | 354.494 | 4.466 | 60.571 | 2 | 5 | 7^{a} |
| 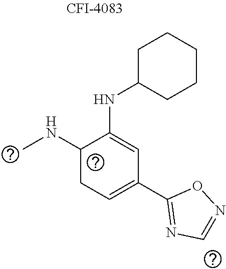 [SMILES: CNC1CC=C(c2ncno2)C=C1NC1CCCCC1] | Mouse: ~120 min Human: >120 min Rat: TBD [symbol] TBD Pig: TBD | Mouse: TBD Human: TBD Rat: TBD [symbol] Pig: TBD | 406.570 | 5.122 | 59.507 | 2 | 5 | 22^{a} |
| 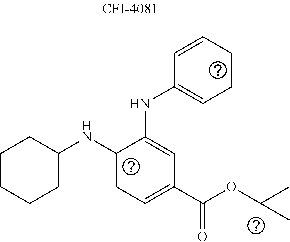 [SMILES: CC(C)OC(=O)C1=CCC(NC2CCCCC2)C(NC2=CCCC=C2)=C1] | Mouse: 30 < t_{1/2} < 60 min Human: >120 min Rat: 30 < t_{1/2} < 60 min [symbol] Pig: 60 < t_{1/2} < 120 min | Mouse: — Human: — Rat: — [symbol] Pig: — | 366.51 | 5.764 | 52.203 | 2 | 3.5 | 37^{a} |
| | | Plasma | | | | | | |
| | | Stability | | | | | | |
| | | at six | | | | | | |
| | Microsomal | hours (% | | | | donor | acceptor | EC₅₀ |
| Compound and Structure | Stability t_{1/2} | remaining) | MW | ClogP | PSA | HB | HB | (nM) |
| 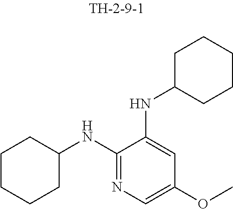 [SMILES: COc1cnc(NC2CCCCC2)c(NC2CCCCC2)c1] | Mouse: 7.7 min | | 303.23 | 4.519 | 39.778 | 2 | 3.25 | 0.1^{a} 0.21^{b} 0.2^{c} |
| 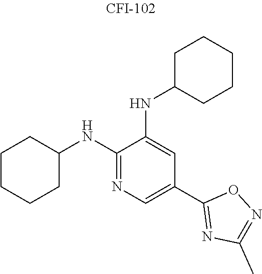 [SMILES: Cc1noc(-c2cnc(NC3CCCCC3)c(NC3CCCCC3)c2)n1] | Mouse: 30 < t_{1/2} < 60 min | | 355.24 | 4.132 | 70.599 | 2 | 5.5 | 0.9^{b} 0.9^{c} |
| 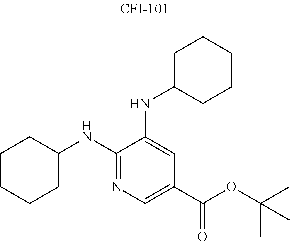 [SMILES: CC(C)(C)OC(=O)c1cnc(NC2CCCCC2)c(NC2CCCCC2)c1] | | | 373.541 | 5.109 | 59.291 | 2 | 4.5 | 354.2^{a} 170.6^{b} 162.6^{c} |
| 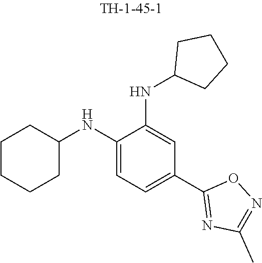 [SMILES: Cc1noc(-c2ccc(NC3CCCCC3)c(NC3CCCC3)c2)n1] | | | 340.471 | 3.702 | 64.136 | 2 | 5 | 33.82^{a} 17.3^{b} 5.1^{c} |
| 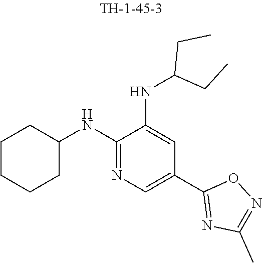 [SMILES: CCC(CC)Nc1cc(-c2nc(C)no2)cnc1NC1CCCCC1] | | | 342.487 | 4.081 | 62.834 | 2 | 5 | 37.8^{a} 26.9^{b} 6.6^{c} |
| 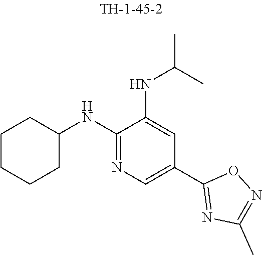 [SMILES: Cc1noc(-c2cnc(NC3CCCCC3)c(NC(C)C)c2)n1] | | | 314.433 | 3.313 | 63.472 | 2 | 5 | 22.3^{a} 16.2^{b} 4.2^{c} |
| 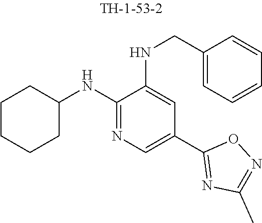 [SMILES: Cc1noc(-c2cnc(NC3CCCCC3)c(NCc3ccccc3)c2)n1] | | | 362.477 | 4.281 | 62.607 | 2 | 5 | 44.5^{a} 40.2^{b} 9.4^{c} |
| 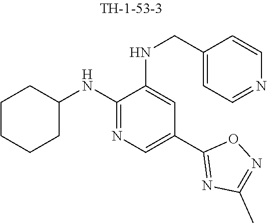 [SMILES: Cc1noc(-c2cnc(NC3CCCCC3)c(NCc3ccncc3)c2)n1] | | | 363.465 | 3.774 | 78.781 | 2 | 6.5 | 63.8^{a} 63.0^{b} 31.5^{c} |
| 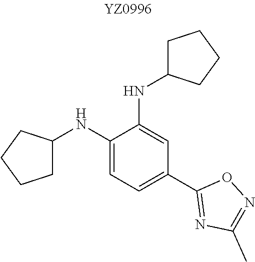 [SMILES: Cc1noc(-c2ccc(NC3CCCC3)c(NC3CCCC3)c2)n1] | | | 326.441 | 3.851 | 60.740 | 2 | 5 | 2^{a} |
| 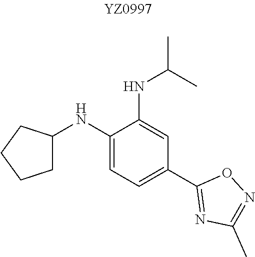 [SMILES: Cc1noc(-c2ccc(NC3CCCC3)c(NC(C)C)c2)n1] | | | 300.403 | 3.440 | 60.645 | 2 | 5 | 2.5^{a} |
| 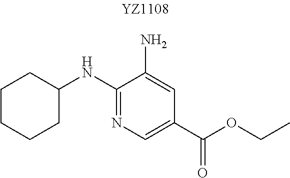 [SMILES: CCOC(=O)c1cnc(NC2CCCCC2)c(N)c1] | | | 263.341 | 2.072 | 78.354 | 2.5 | 4.5 | 314.6^{a} 639^{b} 1038^{c} |
| 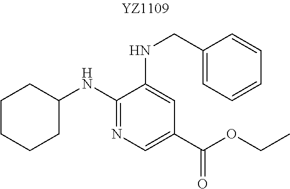 [SMILES: CCOC(=O)c1cnc(NC2CCCCC2)c(NCc2ccccc2)c1] | | | 353.466 | 4.438 | 65.172 | 2 | 4.5 | 317.2^{a} 165.5^{b} 115.2^{c} |
| 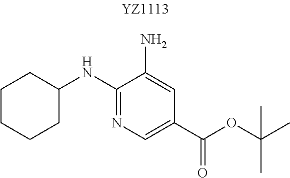 [SMILES: CC(C)(C)OC(=O)c1cnc(NC2CCCCC2)c(N)c1] | | | 291.395 | 2.9 | 73.657 | 2.5 | 4.5 | 338.2^{a} 311.8^{b} 156.2^{c} |
| 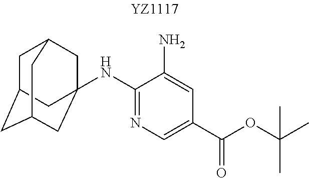 [SMILES: CC(C)(C)OC(=O)c1cnc(NC23CC4CC(CC(C4)C2)C3)c(N)c1] | | | 343.226 | 3.6 | 71.77 | 2.5 | 4.5 | 85.0^{a} 68.4^{b} 39.1^{c} |
| 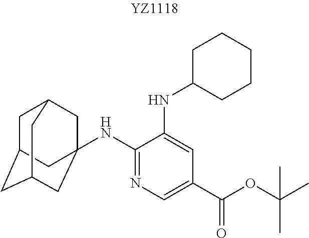 [SMILES: CC(C)(C)OC(=O)c1cnc(NC23CC4CC(CC(C4)C2)C3)c(NC2CCCCC2)c1] | | | 425.304 | 5.792 | 57.416 | 2 | 4.5 | 40.1^{b} 37.3^{c} |
| 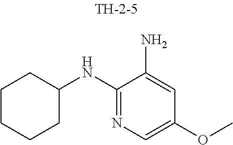 [SMILES: COc1cnc(NC2CCCCC2)c(N)c1] | | | 221.30 | 2.269 | 54.477 | 2.5 | 3.25 | >350 |
| 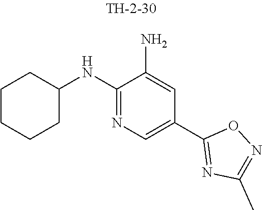 [SMILES: Cc1noc(-c2cnc(NC3CCCCC3)c(N)c2)n1] | | | 273.16 | 1.909 | 85.371 | 2.5 | 5.5 | 461.3^{b} 118.4^{c} |

¹Hofmans et al., 2016, J. Med. Chem, 59, 2041-2053
Mouse: CD1; for compounds with t_{1/2} > 120 min, the average % remaining after 120 minutes is provided in parentheses
Human: Pooled, 50 donors
Rat: Sprague Dawley
Dog: Beagle
Pig: Göttingen Minipig
TBD: to be determined
ClogP: Predicted octanol/water partition coefficient.
PSA: Total Van der Waals surface area of polar nitrogen and oxygen atoms and carbonyl carbon atoms.
donorHB: Estimated number of hydrogen bonds that would be donated by the solute to water molecules in an aqueous solution. Values are averages taken over a number of configurations, so they can be non-integer.
AccptHB: Estimated number of hydrogen bonds that would be accepted by the solute from water molecules in an aqueous solution. Values are averages taken over a number of configurations, so they can be non-integer.
EC₅₀: ^{a}Concentration (nM) of ferrostatin analogue required to achieve 50% viability against HT-1080 cells treated with 10 μM erastin.
^{b}Concentration (nM) of ferrostatin analogue required to achieve 50% viability against HT-1080 treated with 3 μM IKE.
^{c}Concentration (nM) of ferrostatin analogue required to achieve 50% viability against HT-1080 treated with 0.2 μM RSL3.

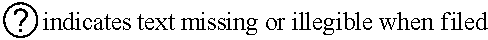

### Example 3

### Metabolic Stability of CFI-4082

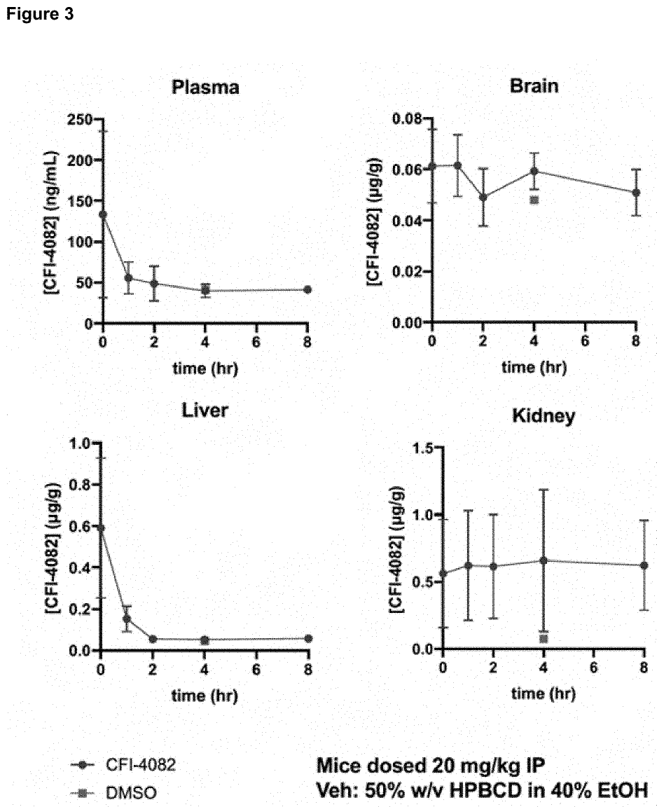

To determine the suitability of CFI-4082 for further in vivo applications, we administered a single dose of CFI-4082 (20 mg/kg in 50% 2-hydroxypropyl-β-cyclodextrin dissolved in 40% ethanol) to male and female C67BI/6 mice (Jackson Lab) via intraperitoneal injection over the course of eight hours, with the compound concentration in plasma and tissue determined by LC/MS-MS. CFI-4082 was found to have low in vivo plasma stability, but was found to stably accumulate in kidney over 8 hours (FIG. 3).

### Example 4

### Rescue Activity of Selected Fer-1 Analogs

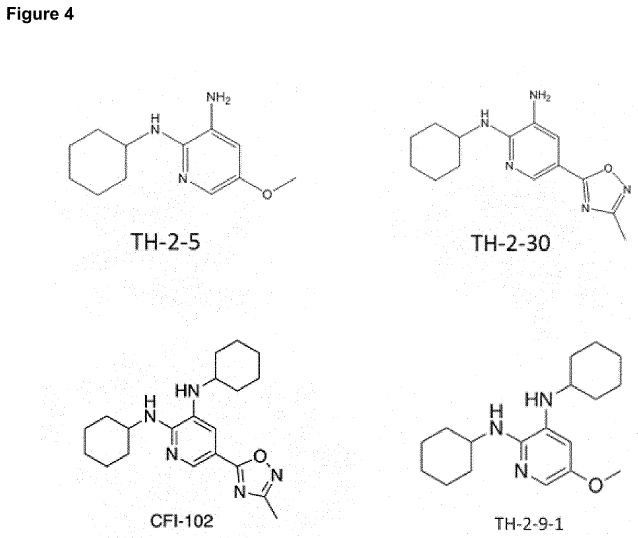

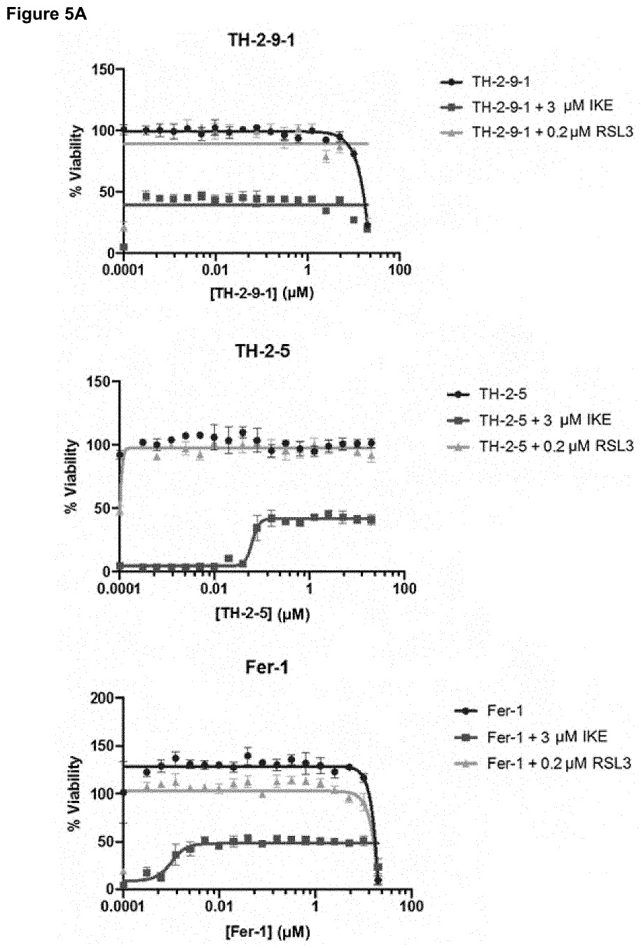

Selected Fer-1 analogs containing a pyridine moiety (FIG. 4) were tested to examine their efficacy and overall potency in inhibiting ferroptosis. For each of these compounds, dose-response curves were generated in HT-1080 cells looking at the effectiveness of the molecules in inhibiting ferroptosis induced by either 3 μM IKE or 0.2 μM RSL3, Fer-1 was used as a positive control. For each dose-response curve, 1,000 cells/well were seeded in a 384 well plate and allowed to adhere overnight prior to treating with compound from a daughter plate. Cells were treated for 48 hours, unless otherwise noted, prior to viability being analyzed using cell titer glo (40 μL per well). All liquid handling was performed using the BioMek. All samples were prepared in triplicate, unless otherwise noted.

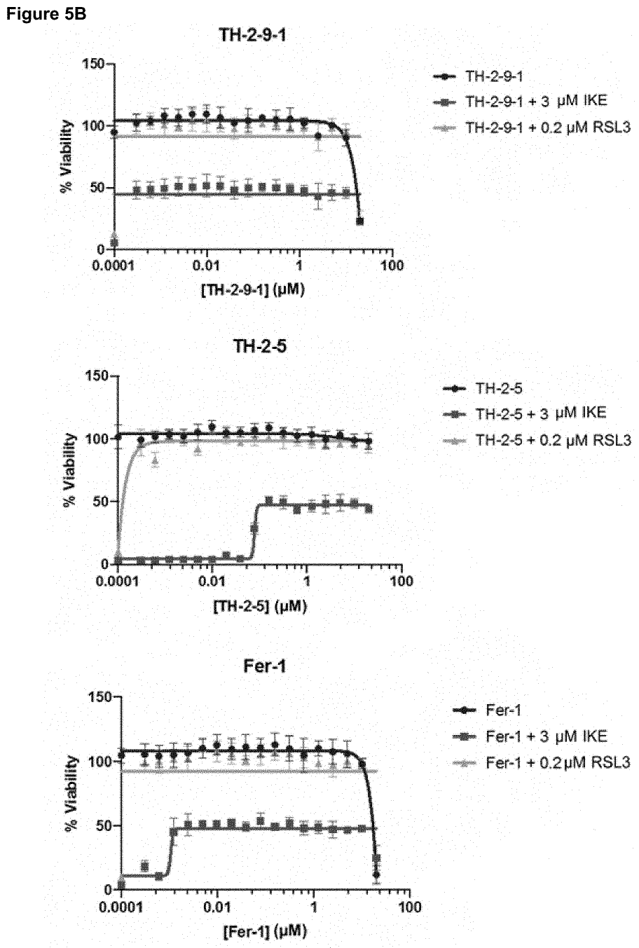

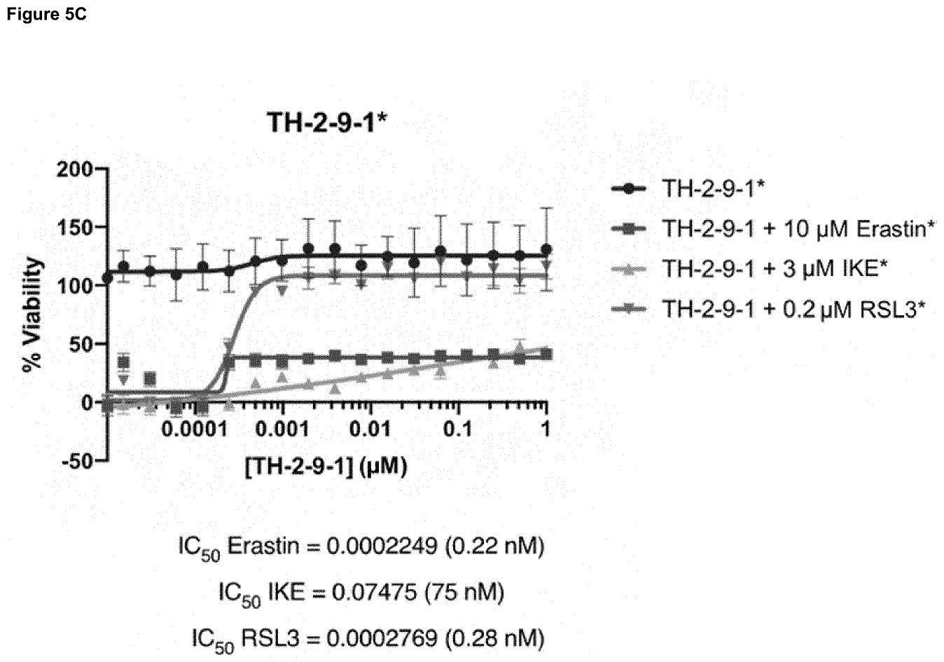

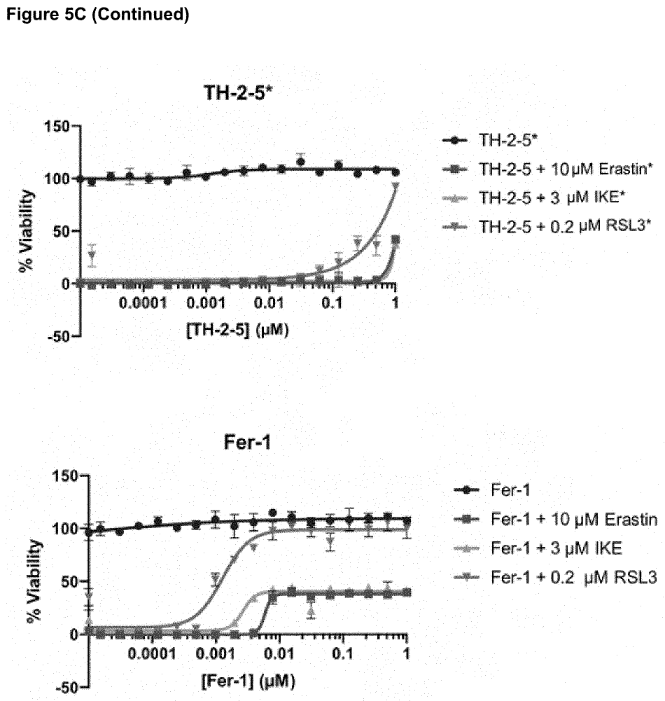

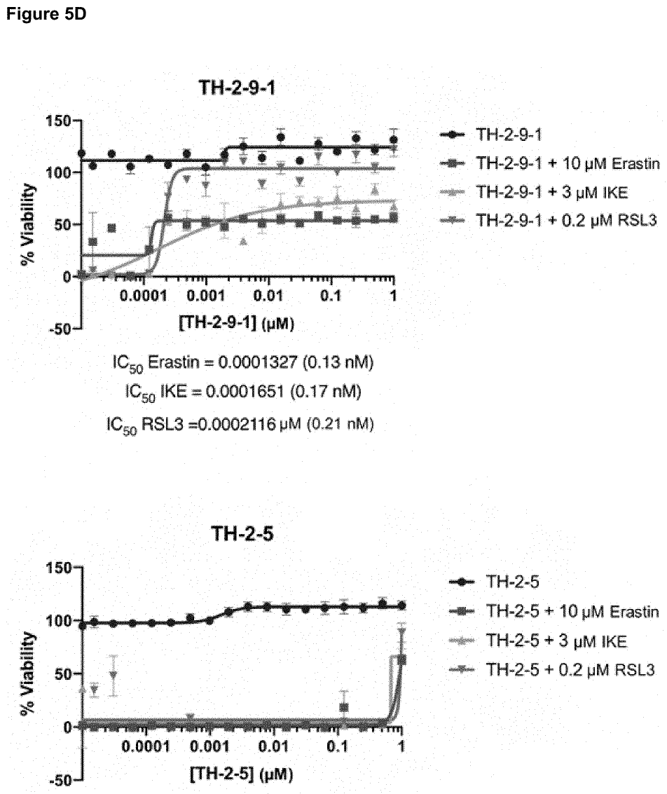

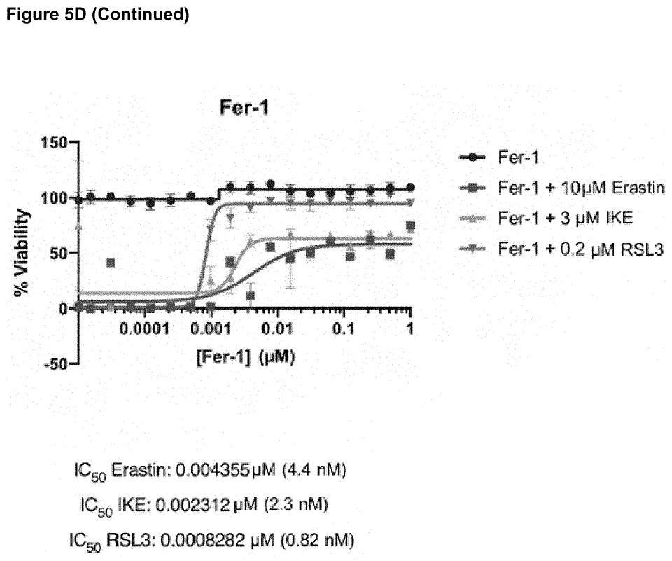

TH-2-9-1 and TH-2-5 compounds were first tested at a concentration range from 20 μM-0 μM. which was too high to capture any death at the lower concentrations, as evidenced by both compounds showing almost full rescue at most concentrations within the range (FIG. 5A).

The tests were repeated at a lower concentration range from 10 μM-0 μM, which was effective in capturing some of the earlier death. No death was observed with RSL3 for Fer-1, TH-2-9-1, and TH-2-5, suggesting that lower inhibitor concentrations were still needed (FIG. 5B).

By further lowering the concentration, compounds were tested at a range from 1 μM-0 μM. Erastin was also used in the test as a ferroptosis inducer. Following the same protocol, cells were treated with 10 μM erastin. As shown in FIG. 5C, TH-2-9-1 was protective against the cell death across the concentration range tested, indicating a higher potency than Fer-1 based on the leftward shift of the curves. Notably, both fer-1 and TH-2-9-1 were only able to produce ˜50% rescue against IKE and erastin. Another set of tests were repeated at the concentration range from 1 μM-0 μM, the results of which were largely consistent with the previous experiments (FIG. 5D). Fer-1 in this repeated experiment was much more potent than previously reported, with the potency nearly an order of magnitude higher than previously observed, while TH-2-9-1 was an order of magnitude more potent than Fer-1 for all inducers beyond RSL3, indicating that TH-2-9-1 can be a potential Fer-1 analog for in vivo applications.

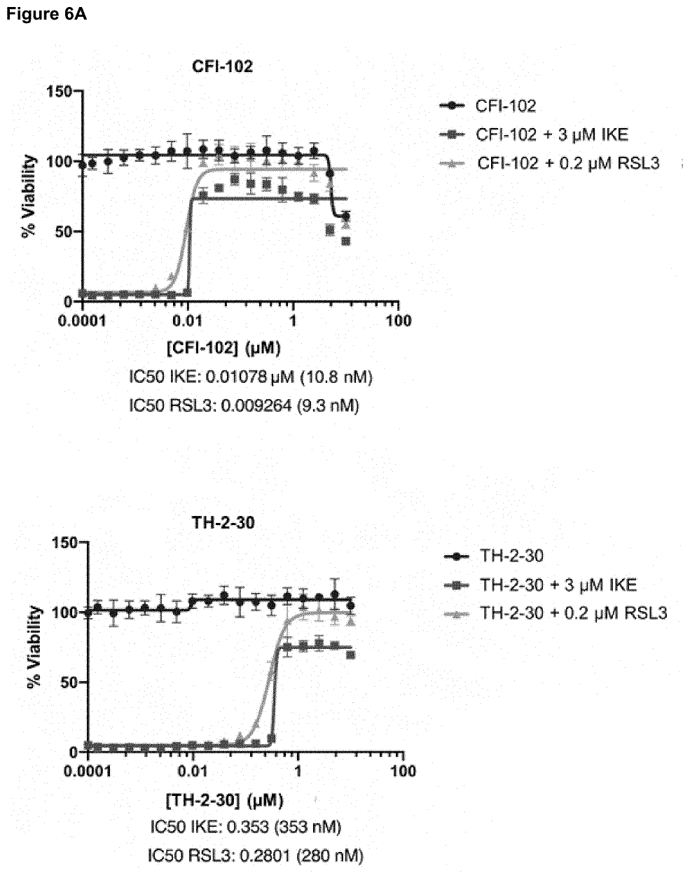

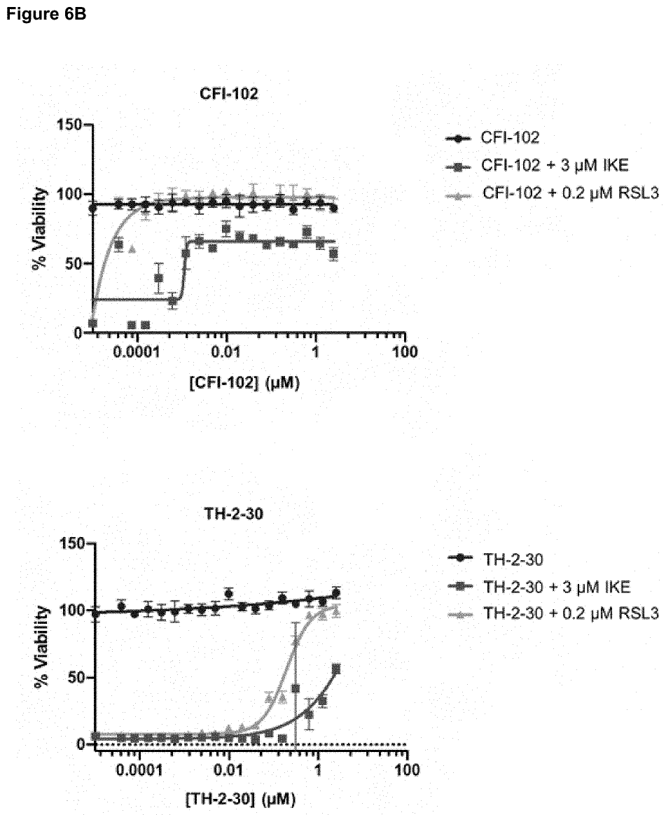

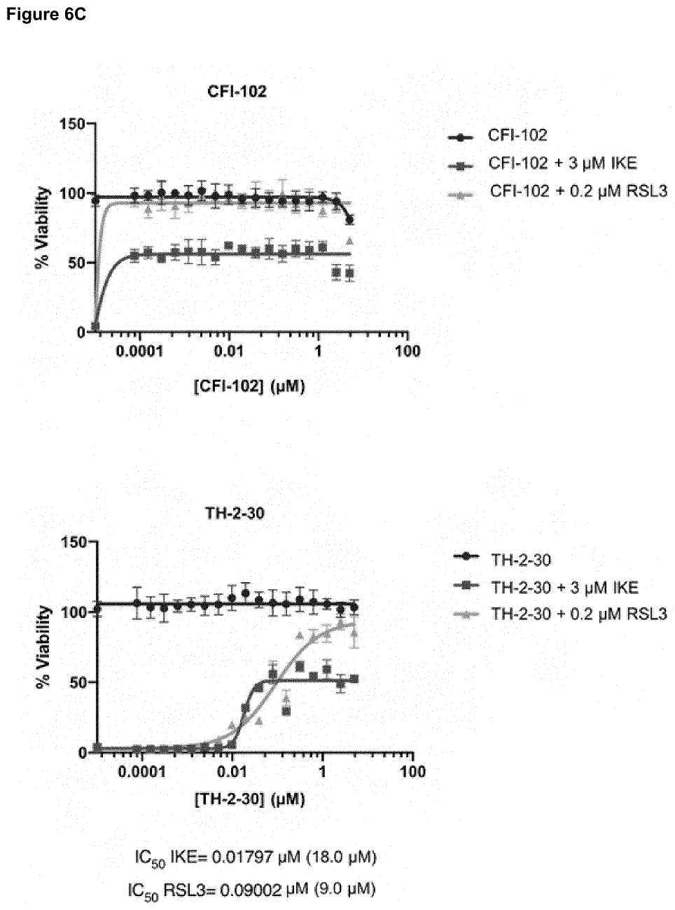

Two more compounds, CFI-102 and TH-2-30 were also tested for their anti-ferroptosis activities, using the the same protocol as described above. Starting with a concentration range from 10 μM-0 μM, both compounds demonstrated activity against both IKE and RSL3, with CFI-102 having an IC₅₀ of ˜10-20 nM against both IKE and RSL3. TH-2-30 was relatively less potent. At 10 μM, both compounds appeared to be toxic, as evidenced by the overall drop in viability for all treatment conditions at the concentration (FIG. 6A).

The tests were repeated at a lower concentration range from 2.5 μM-0 μM, While the toxicity issues at 10 μM was not present, it appeared that 2.5 μM was too low of a starting concentration for TH-2-30 to fully establish rescue (FIG. 6B). Therefore, another set of experiments was conducted with the staring concentration of 5 μM. For this set of experiments the samples were treated for 51 hours instead of 48 hours. As shown in FIG. 6C, no compound was able to achieve full rescue against IKE at the highest concentration; this might be due to this batch of IKE being more potent or some other factor. Both compounds showed activity against IKE and RSL3, and CFI-102 was more potent by achieving full rescue at around 0.0001 μM.

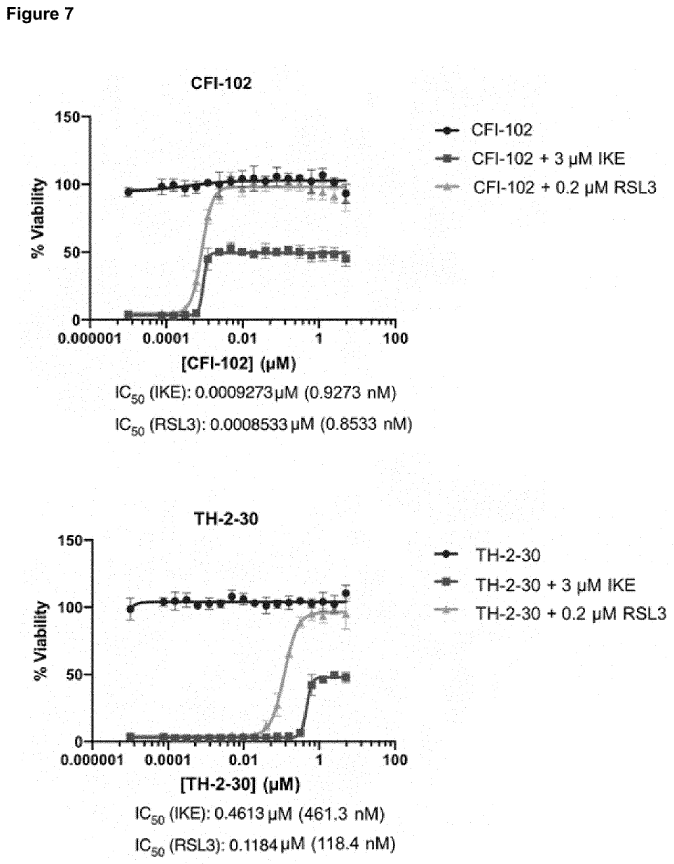

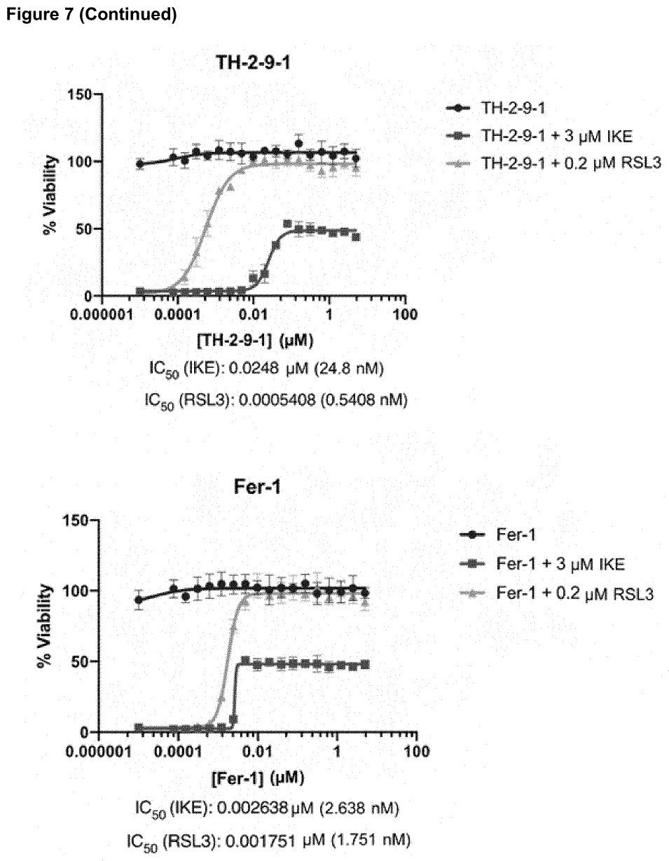

Further experiments were performed with a starting concentration of 5 μM to compare the potency between different compounds. According to the results shown in FIG. 7, CFI-102 was the most potent analog for both IKE an RSL3, TH-2-9-1 was the most potent analog for RSL3 alone, and TH-2-30 had potency comparable to Fer-1 against IKE and RSL3.

### Example 5

### Therapeutical Applications of Fer-1 Analogs

Patients receiving radiotherapy and/or immunotherapy usually suffer from various side effects including, but not limited to, skin reactions (e.g., redness, itching, peeling, blistering, and dryness) and flu-like symptoms (e.g., fatigue, fever, chills, weakness, nausea, vomiting, dizziness, body aches, and high or low blood pressure). There is evidence showing these side effects may be associated with undesired cell death through ferroptosis, which suggests therapeutic potential for molecules that inhibit/reduce ferroptosis.

To explore such applications, we will introduce the Fer-1 analogs disclosed herein into conventional radiotherapy/immunotherapy protocols. We will monitor patients' (animal and then human patient's) reaction to the combined treatment, and determine whether there is any improvement with respect to common side effects, for example, less or even no occurrence, reduced intensity, etc. We anticipate using in vitro models to inform our animal trials.

It is also believed that ferroptosis plays a critical role in bacteria-induced (e.g., Mycobacterium tuberculosis) cell death and tissue necrosis. In light of this, we expect that the Fer-1 analogs disclosed herein would have therapeutic application against various pathogens through inhibiting unwanted ferroptosis.

### Example 6

### Other Optimized Fer-1 Analogs as Ferroptosis Inhibitors

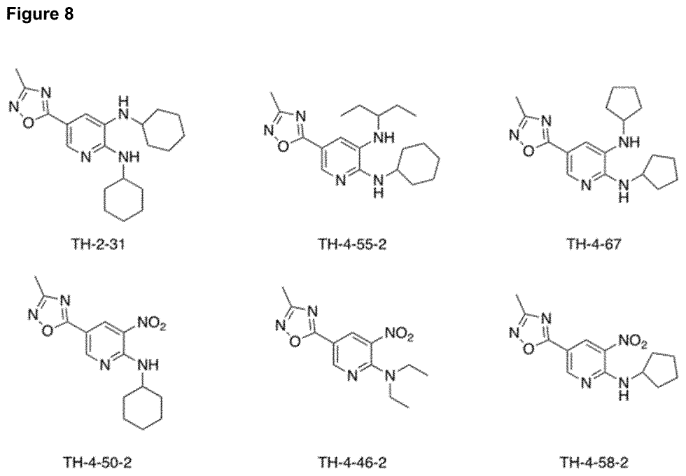

After synthesizing and characterizing a series of ferrostatin-1 analogs, three active compounds (TH-2-31 (i.e., CFI-102), TH-4-55-2, and TH-4-67) that meet all criteria for success were identified. Three inactive controls derived from the active compounds were also obtained for comparative studies (FIG. 8, compounds TH-4-50-2, TH-4-46-2, and TH-4-58-2). All active analogs can be synthesized on gram scale in high purity, and are suitable for in vivo efficacy studies. Detailed test results are shown below.

Potency in Suppressing Ferroptosis Induced by RSL3 in N27 Cells (20 nM, 48 Hours of Incubation) with EC₅₀<10 nM

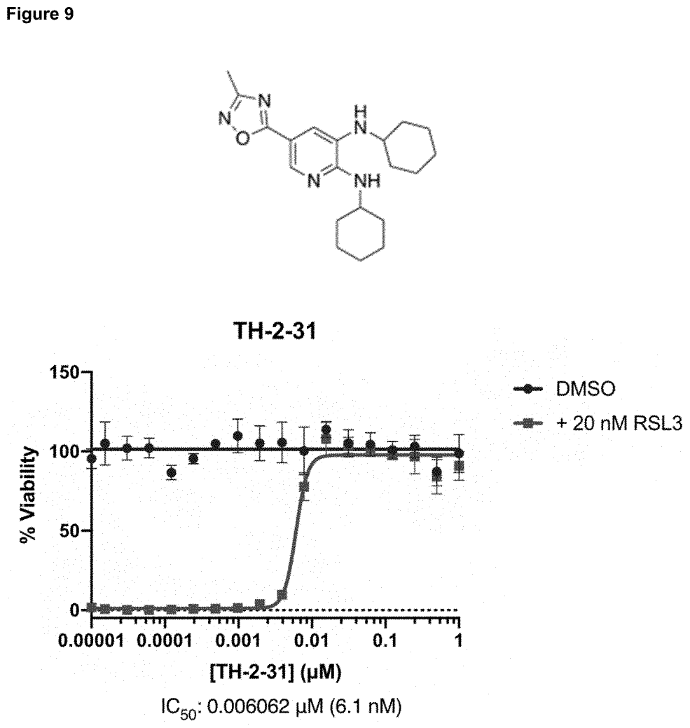

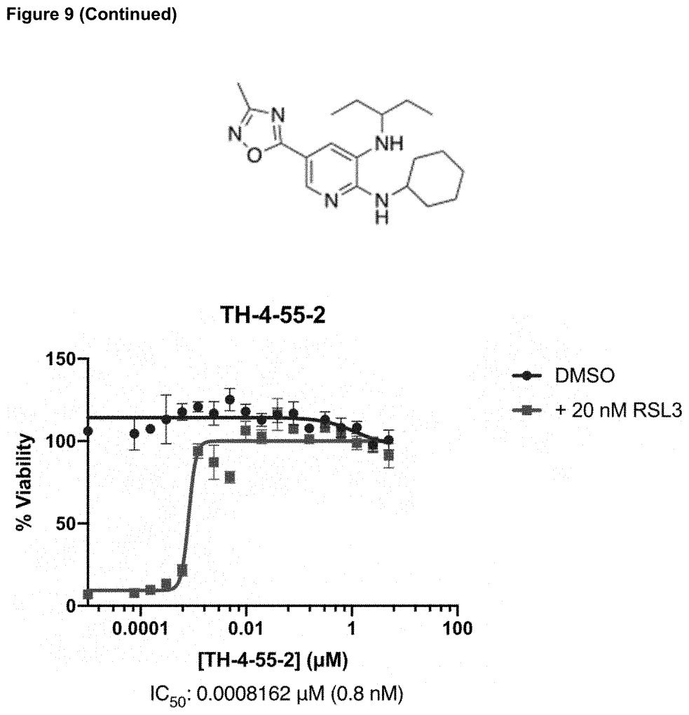

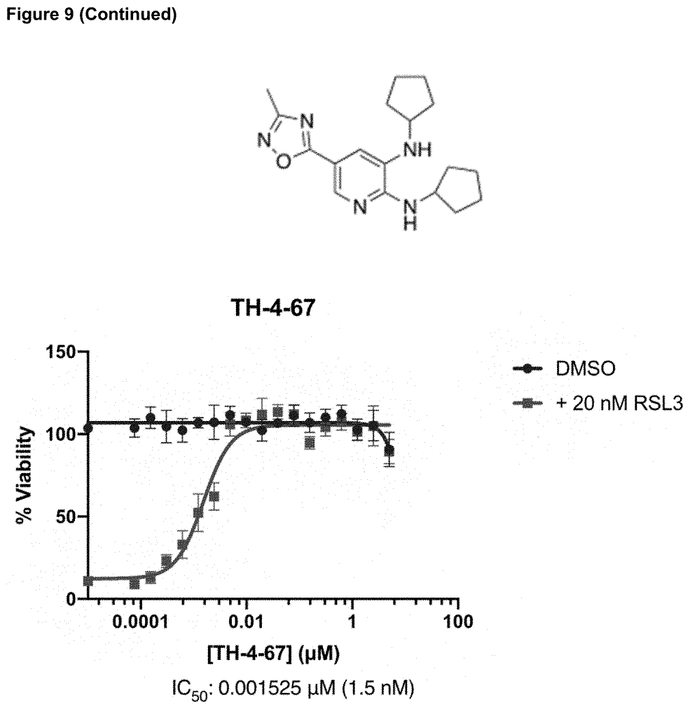

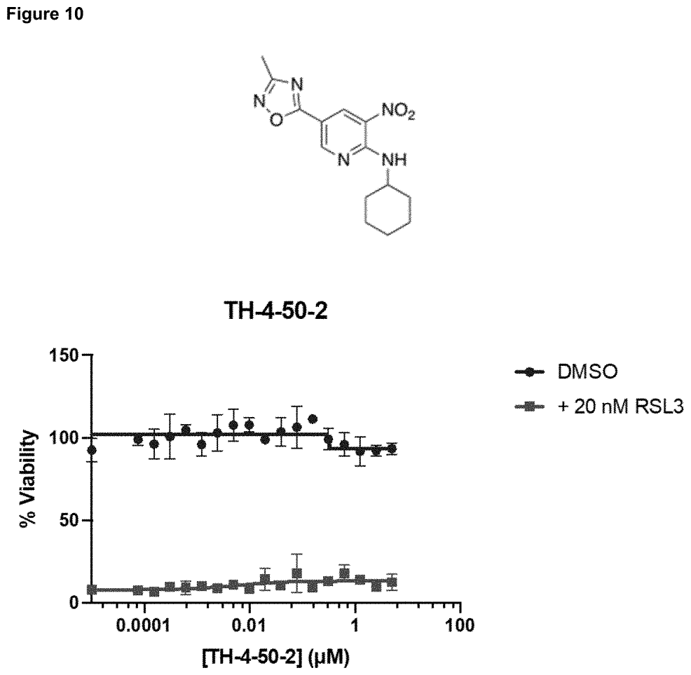

As shown in FIG. 9, there are representative dose-response curves demonstrating that three optimized ferrostatins (TH-2-31, TH-4-55-2, and TH-4-67) suppress ferroptosis induced by RSL3 in N27 cells (20 nM, 24 hours of treatment) with EC₅₀<10 nM. It was found that 20 nM RSL3 can effectively achieve 100% cell death within 24 hours, and potent ferrostatin-1 analogs are able to demonstrate complete ferroptosis rescue in a dose-dependent manner within the same time frame. Additionally, a 24-hour treatment allows for more efficient testing of analogs in a higher throughput fashion than 48-hour treatment.

EC₅₀ values for three separate experiment, n=3 wells/per compound/per condition, are provided in Table 2 below.

TABLE 2
EC₅₀s of TH-2-31, TH-4-55-2, and TH-4-67 in N27 cells.

| Compound | EC₅₀ I | EC₅₀ II | EC₅₀ III |
|---|---|---|---|
| TH-2-31 | 2.6 nM | 1.4 nM | 6.1 nM |
| TH-4-55-2 | 0.8 nM | 1.0 nM | 4.9 nM |
| TH-4-67 | 1.5 nM | 1.4 nM | 3.4 nM |

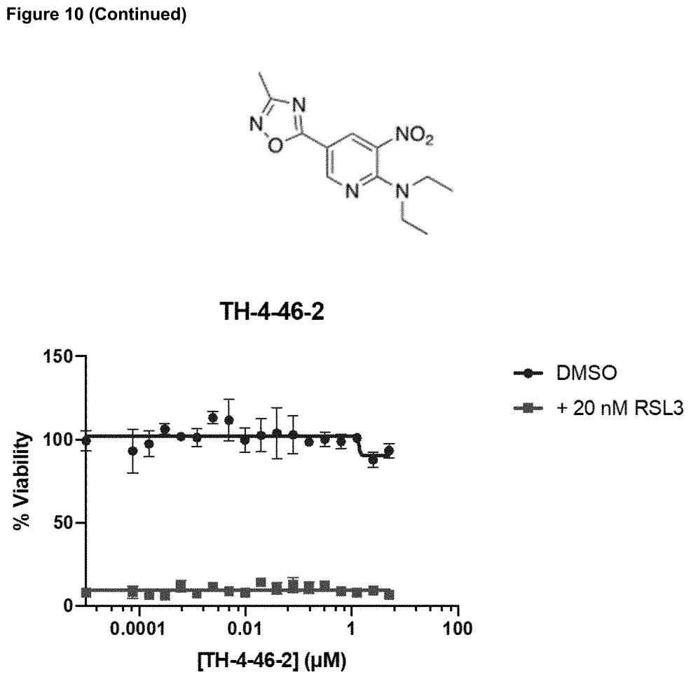

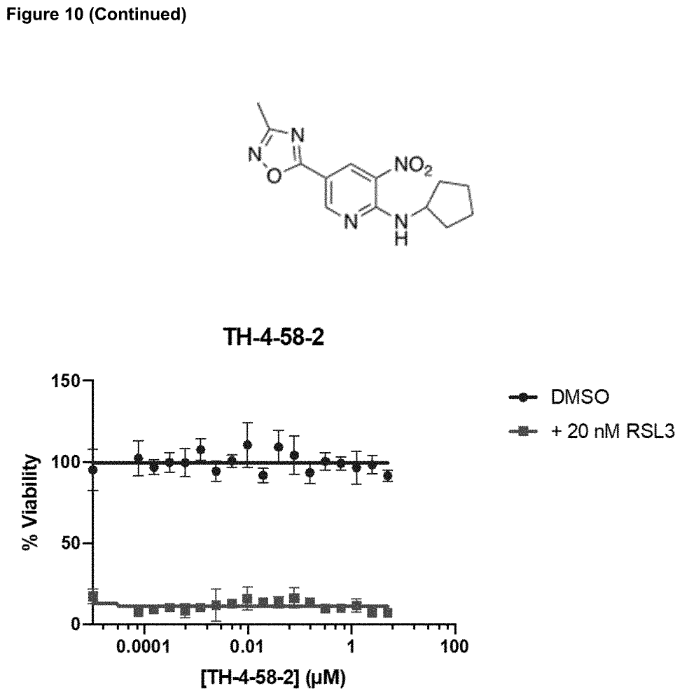

In addition to the optimized ferrostatin compounds described above, three inactive controls (TH-4-50-2, TH-4-58-2, and TH-4-46-2) were developed that are unable to suppress ferroptosis induced by RSL3 in N27 cells (20 nM, 24 hours of incubation). Their structures and representative dose-response curves are shown in FIG. 10.

Metabolic Stability in Mouse Liver Microsomes with Half-Life >60 Min

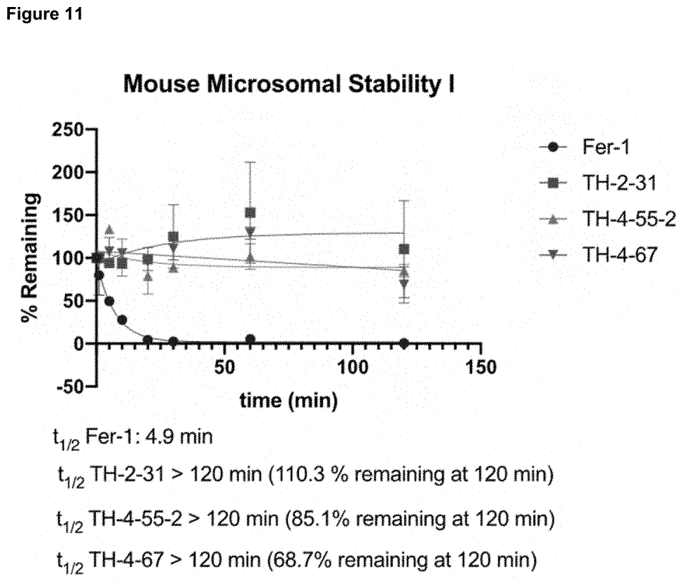

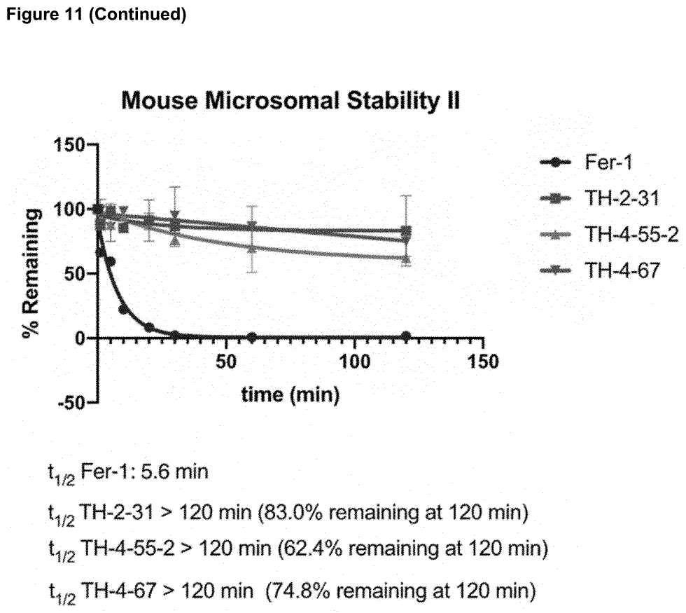

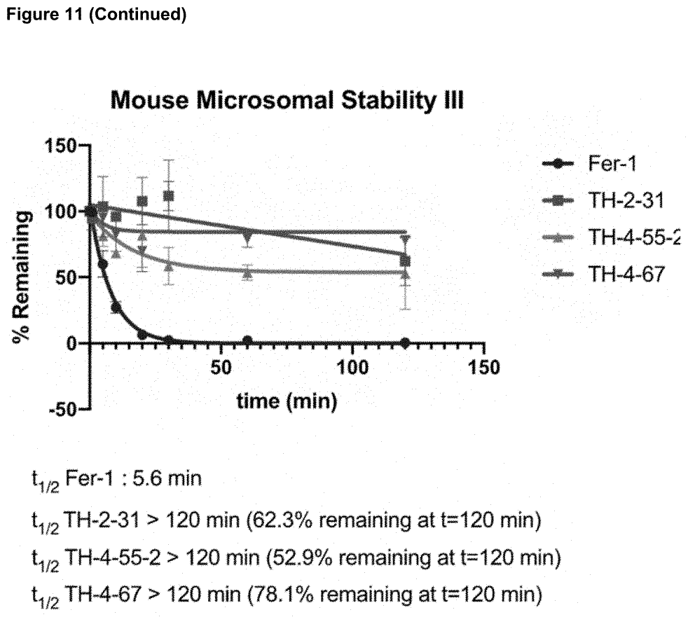

Results from three separate mouse microsomal stability experiments each performed in triplicate demonstrated that the three optimized ferrostatins (TH-2-31, TH-4-55-2, and TH-4-67) are stable in mouse liver microsomes with half-life greater than 60 minutes, with each compound indeed having a half-life greater than two hours (FIG. 11).

A summary of the half-lives from the three independent microsomal stability tests in mouse liver microsomes is provided below in Table 3.

TABLE 3
Results from three independent tests of ferrostatins TH-2-31,
TH-4-55-2 and TH-4-67 for microsomal stability
(n = 2 wells/compound/experiment).

| Compound | t_{1/2} I | t_{1/2} II | t_{1/2} III |
|---|---|---|---|
| TH-2-31 | >120 min | >120 min | >120 min |
| TH-4-55-2 | >120 min | >120 min | >120 min |
| TH-4-67 | >120 min | >120 min | >120 min |

For the in vivo studies, the results from which are detailed below. The optimized ferrostatins were administered to C57BL/6 mice at 8 weeks of age. Compounds were administered at a concentration of 20 mg/kg in a vehicle consisting of 1:1 (65% v/v of 25% w/v 2-hydroxypropyl-β-cyclodextrin dissolved in 20% ethanol, 30% v/v PEG-400, and 5% Tween-80): H₂O via intravenous injection (IV), intraperitoneal injection (IP), or oral gavage (PO). For each time-point and route of administration, two male and two female mice were used. Mice were euthanized, and plasma and brain samples were obtained from each mouse at 0, 1, 2, 4, 8, and 24 hours after compound administration. Compounds were extracted from plasma and brain homogenate in acetonitrile and analyzed via UHPLC-MS/MS against a standard curve to quantify compound concentrations.

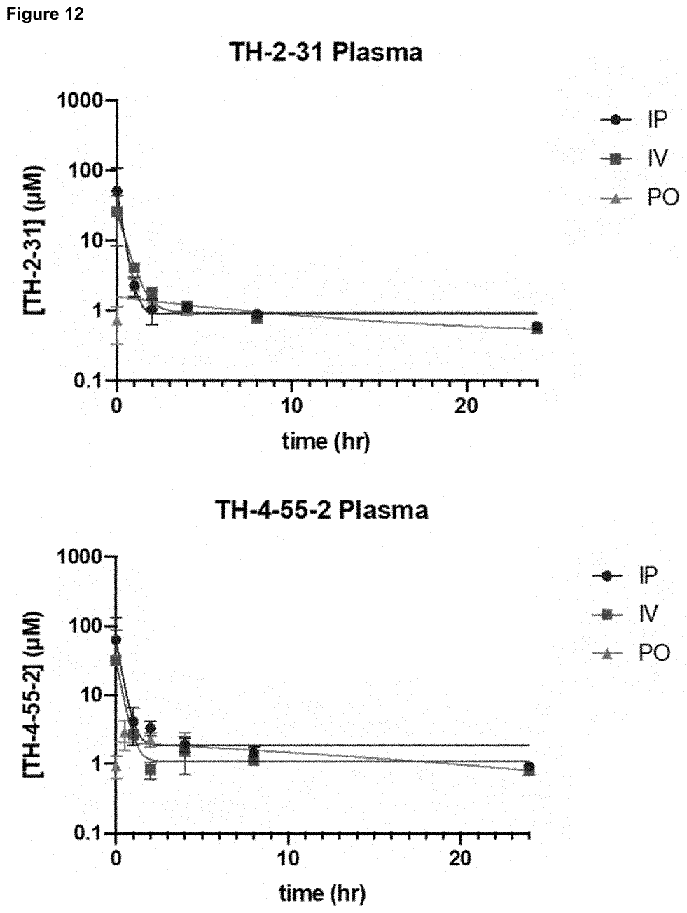

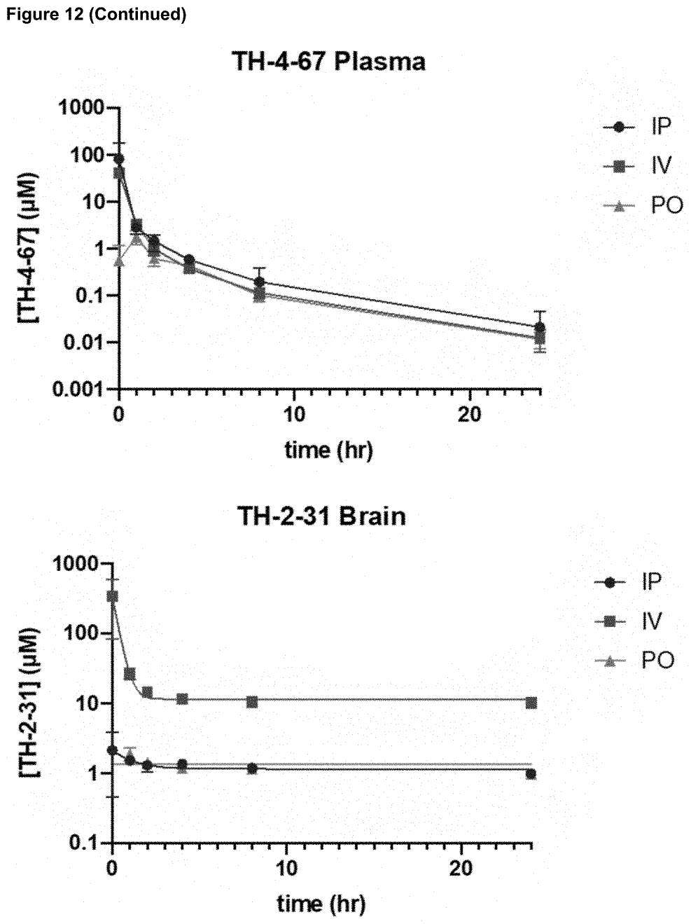

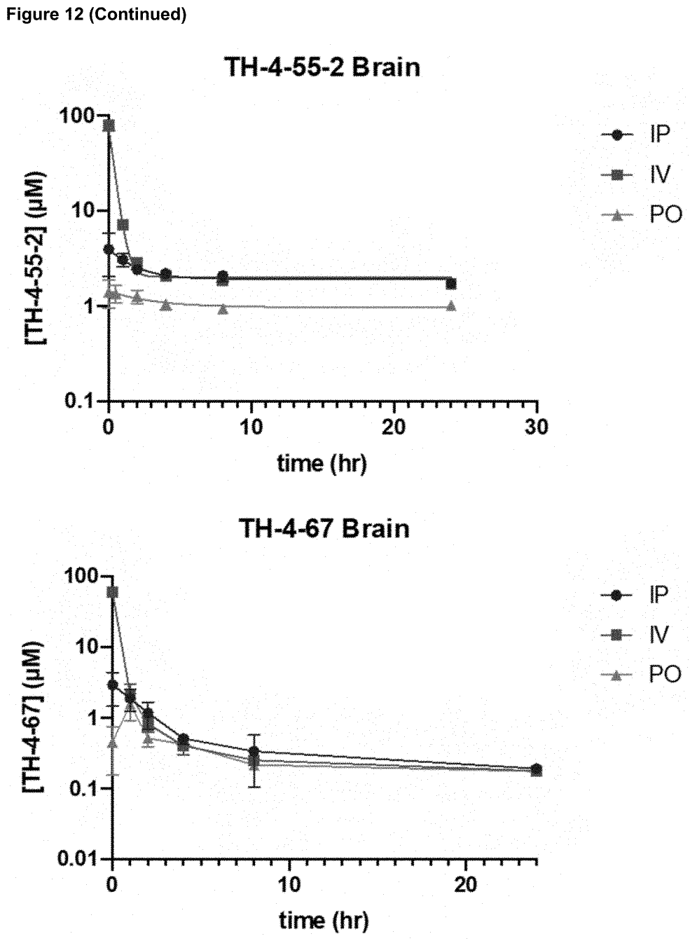

The concentration of each analog in plasma and brain is shown below in FIG. 12. Each analog accumulated at high nM to μM concentrations across the time-points analyzed. The metrics for various tests are described below.

BBB Permeability with Log (Brain/Plasma) Ratio >0

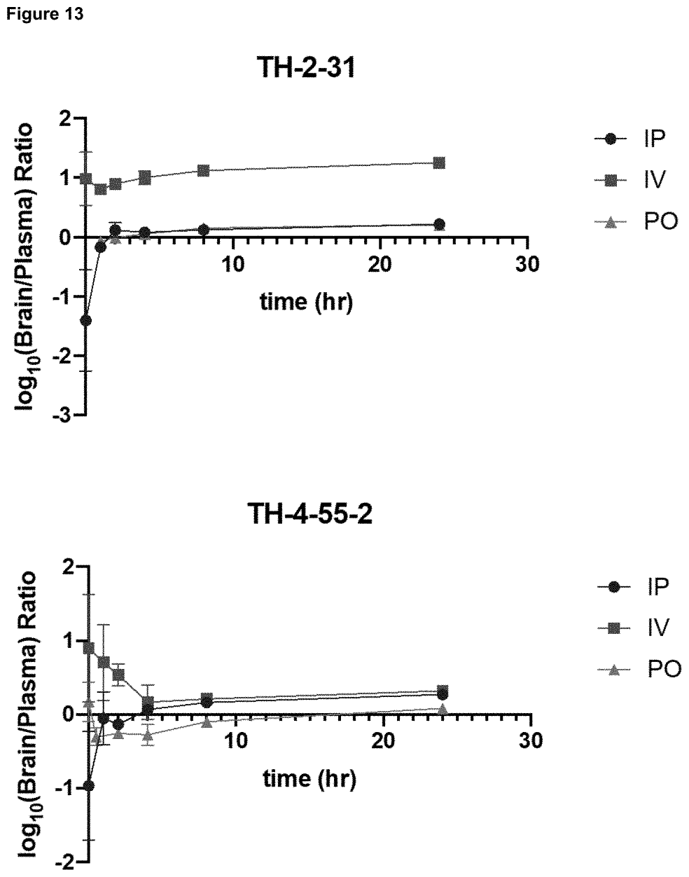

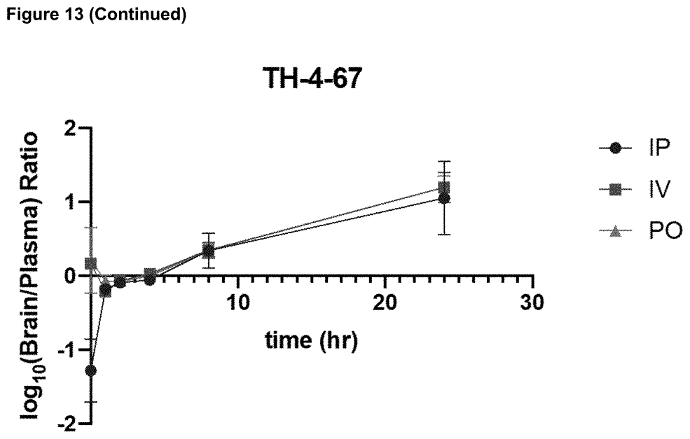

Calculated from the above PK data, BBB permeability was determined. As shown in FIG. 13, each optimized analog preferentially accumulates in the brain over time, with all three compounds having a log₁₀(brain/plasma) value >0 at 24 hours.

### In Vivo Brain Half-Life >3 h

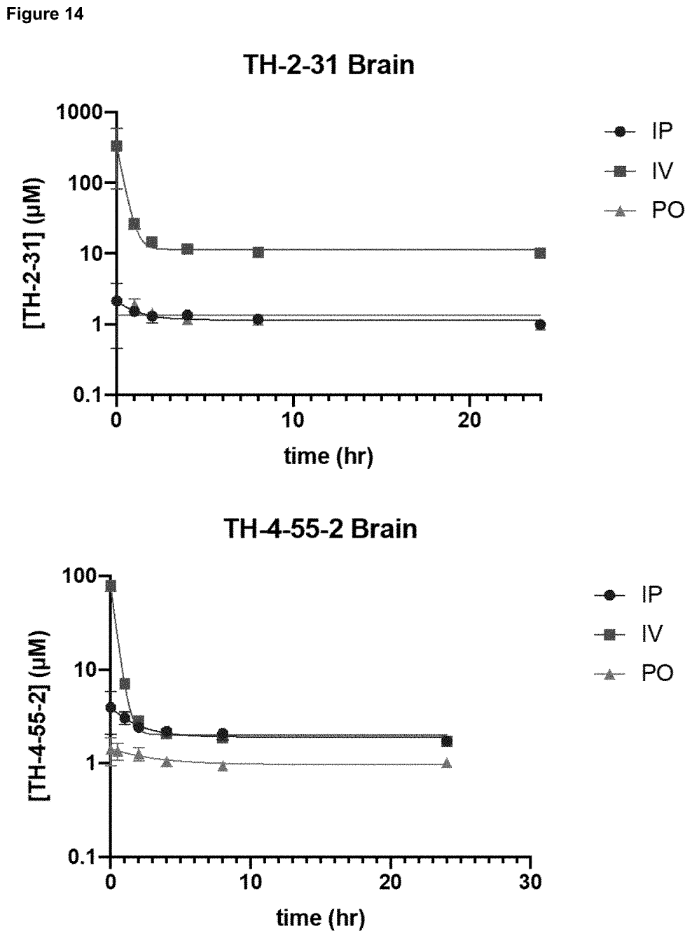

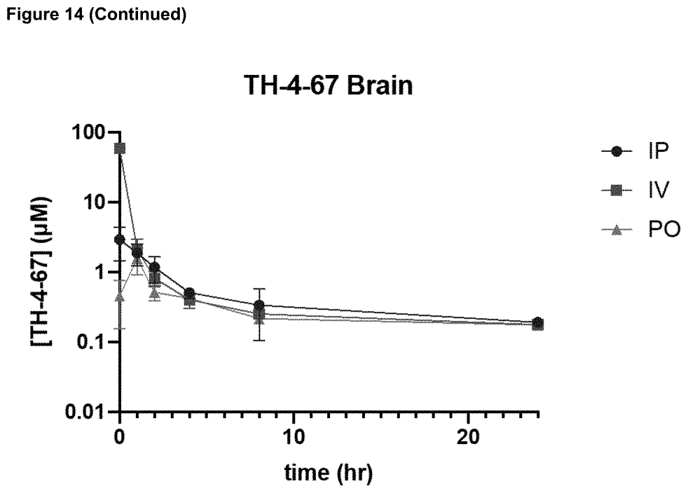

Each analog rapidly accumulated in brain following IV accumulation and decreases in concentration thereafter (FIG. 14).

A summary with the in vivo brain half-lives is provided in Table 4.

TABLE 4
in vivo brain half-live of each compound.

| Compound | R.O.A | t_{1/2} |
|---|---|---|
| TH-2-31 | IP | >8 h |
| | IV | <1 h |
| | PO | >24 h |
| TH-4-55-2 | IP | >8 h |
| | IV | <1 h |
| | PO | >24 h |
| TH-4-67 | IP | 1 h < t_{1/2} < 2 h |
| | IV | < 1 h |
| | PO | 1 h < t_{1/2} < 2 h |

For both TH-2-31 and TH-4-55-2, the IP and PO administrations satisfy the R33 transition criterion. For TH-4-67, the criterion is not met. However, as observed in the data provided below and the corresponding graphs, TH-4-67 accumulates in brain at orders of magnitude higher than the EC₅₀ values at 24 hours post compound administration, and it is expected to be potent irrespective of the half-live in the brain. Indeed, this compounds exceeds its 2 nM EC₅₀ value for the full 24 hours of treatment. Therefore, although the IP and PO half-lives are slightly under 3 hours, the compound is likely to exert a PD and therapeutic effect in mice, due to exceeding its effective concentration in the brain over a 24 hour period.

### Cmax>5× the EC₅₀ in N27 Cells

Table 5 below details the Cₘₐₓ in both plasma and brain and the EC₅₀ values for each analog and route of administration. All three of the optimized ferrostatins easily meet this criterion.

TABLE 5
Summary of in vivo Cₘₐₓ values in brain and plasma, and
Cₘₐₓ/EC₅₀ for each route of administration.

| | | Cₘₐₓ | Cₘₐₓ | | Cₘₐₓ | |
|---|---|---|---|---|---|---|
| | | plasma | brain | | plasma/ | Cₘₐₓ |
| Compound | R.O.A. | (nM) | (nM) | EC₅₀ | EC₅₀ | brain/EC₅₀ |
| TH-2-31 | IP | 50,923 | 2,133 | 3.37 | 15,111 | 633 |
| | IV | 25,791 | 334,759 | nM | 7,653 | 99,335 |
| | PO | 2,238 | 1,190 | | 664 | 353 |
| TH-4-55-2 | IP | 63,834 | 3,936 | 2.23 | 28,625 | 1,765 |
| | IV | 32,109 | 79,314 | nM | 14,399 | 35,567 |
| | PO | 2,929 | 1,410 | | 1,313 | 632 |
| TH-4-67 | IP | 82,120 | 2,916 | 2.10 | 39,105 | 1,389 |
| | IV | 40,640 | 52,121 | nM | 19,352 | 24,820 |
| | PO | 1,768 | 1,554 | | 842 | 740 |

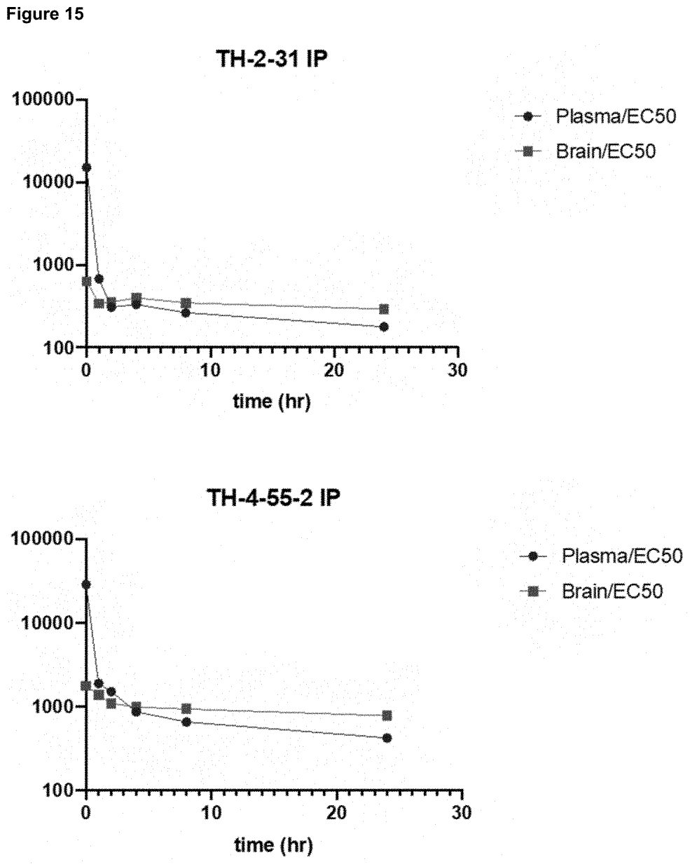

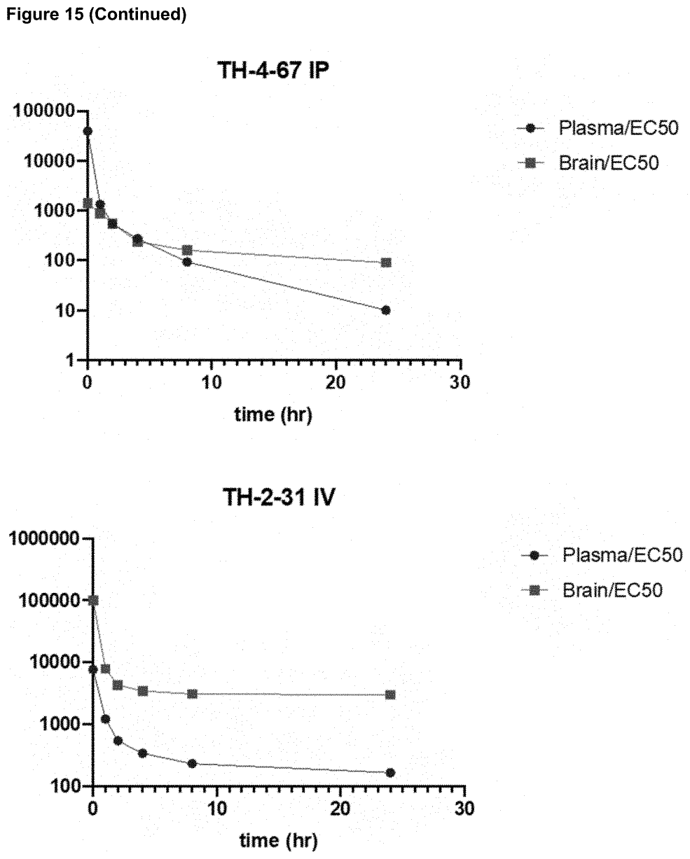

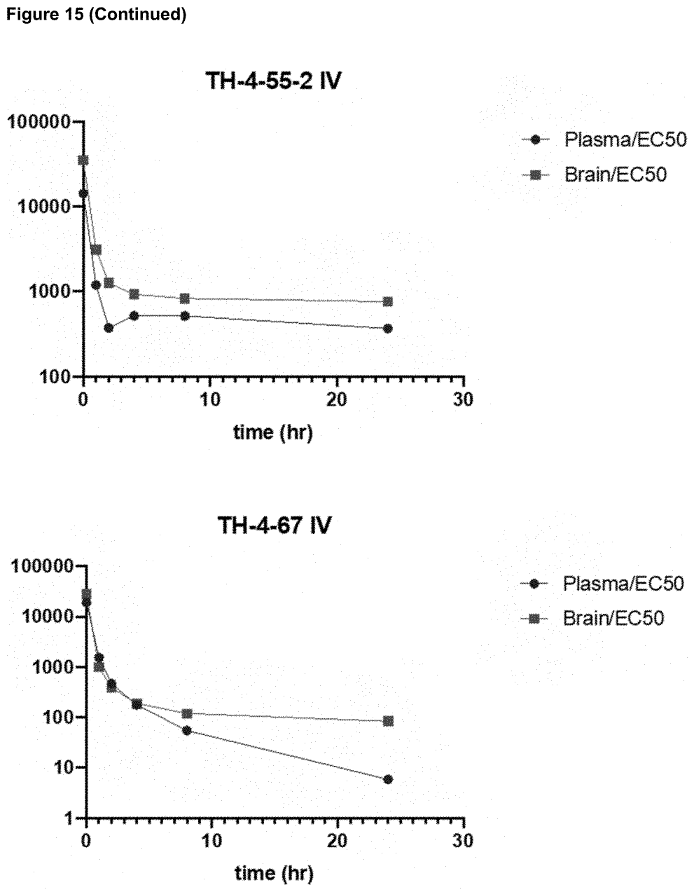

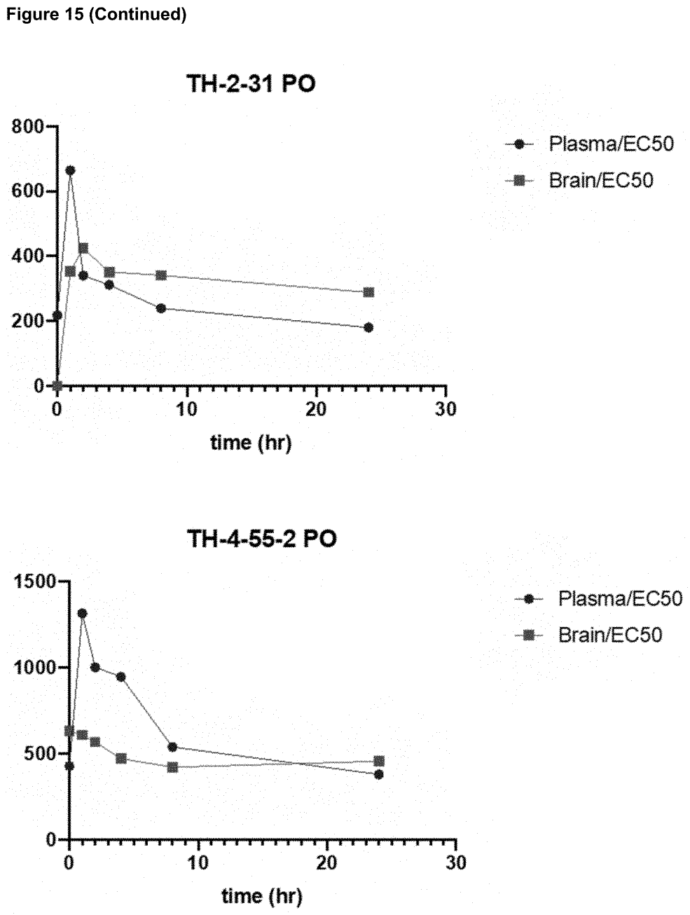

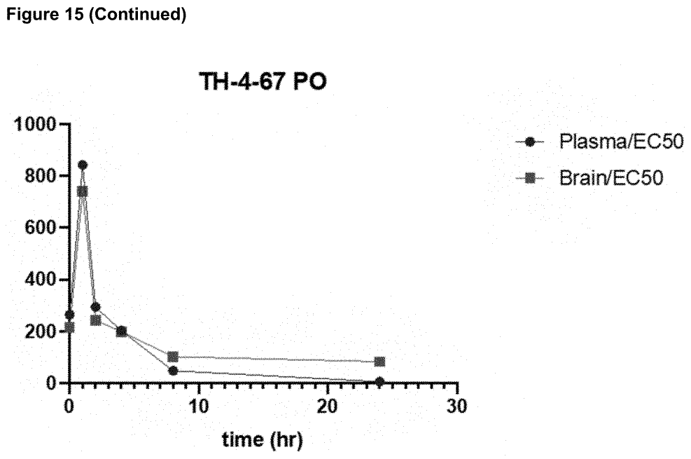

As shown in FIG. 15, the concentration of each optimized analog in brain and plasma was examined and compared to the EC₅₀ value for each time point and route of administration (R.O.A.). As indicated, all 3 compounds accumulate in both plasma and brain at ratios >5 for all time-points and route of administration.

### Solubility >1 mM

To achieve the 20 mg/kg dose for each compound, mice were injected with a 2 mg/mL solution in the vehicle described above. For all 3 optimized compounds, no precipitation was observed in the resulting 2 mg/mL solutions, even several days after preparation. As detailed in Table 6 below, each of the compounds meet this criterion, with solubility greater than concentrations needed for in vivo injections.

TABLE 6
Concentration of each optimized
compounds prepared for in vivo injection.

| | Molecular | Concentration | Concentration |
|---|---|---|---|
| Compound | Weight | (mg/mL) | (mM) |
| TH-2-31 | 355.49 | 2 | 5.6 |
| TH-4-55-2 | 343.48 | 2 | 5.8 |
| TH-4-67 | 327.43 | 2 | 6.1 |

Plasma Stability (Mouse) with Half-Life >120 Min

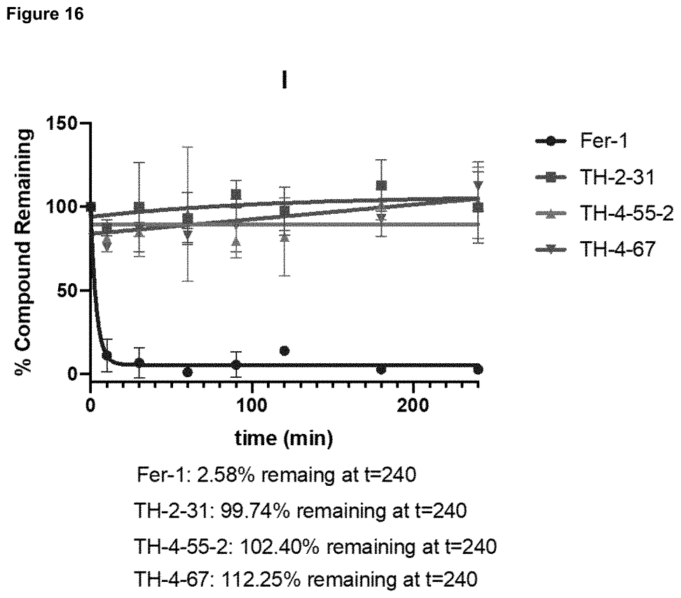

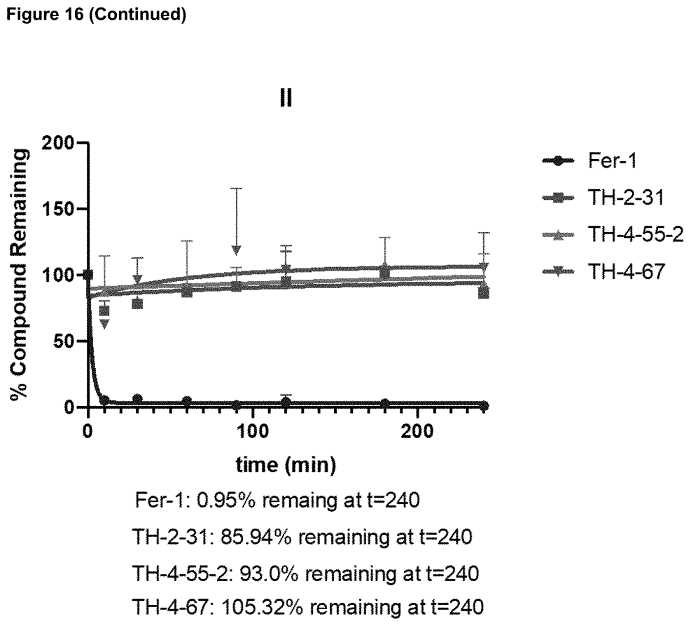

In two separate experiments, all three compounds were stable in mouse plasma with minimal-to-no degradation of the compounds after a 4 hour incubation (FIG. 16). Ferrostatin-1 is shown as comparison, which is fully degraded in mouse plasma in less than 15 min. Table 7 summarizes the observed plasma half-lives for all 3 analogs.

TABLE 7
Observed plasma half-lives for all 3 optimized analogs.

| Compound | t_{1/2} I | t_{1/2} II |
|---|---|---|
| TH-2-31 | >240 min | >240 min |
| TH-4-55-2 | >240 min | >240 min |
| TH-4-67 | >240 min | >240 min |

All optimized compounds were synthesized on a gram scale with high purity, ready for in vivo efficacy studies. >1 gram of each compound was synthesized.

### DOCUMENTS CITED

ABDEL-MAGID, A. F.; Carson, K. G.; Harris, B. D.; Maryanoff, C. A.; Shah, R. D. J. Org. Chem. 1996, 61, 3849-3862.
ANITHA, M., Nandhu, M. S., Anju, T. R., Jes, P. & Paulose, C. S. Targeting glutamate mediated excitotoxicity in Huntington's disease: neural progenitors and partial glutamate antagonist—memantine. Medical hypotheses 76, 138-140, doi:10.1016/j.mehy.2010.09.003 (2011).
BANJAC, A., Perisic, T., Sato, H., Seiler, A., Bannai, S., Weiss, N., Kolle, P., Tschoep, K., Issels, R. D., Daniel, P. T., et al. (2008). The cystine/cysteine cycle: a redox cycle regulating susceptibility versus resistance to cell death. Oncogene 27, 1618-1628.
BARTZOKIS, G., Cummings, J., Perlman, S., Hance, D. B. & Mintz, J. Increased basal ganglia iron levels in Huntington disease. Arch Neurol 56, 569-574 (1999).
BEAULIEU, P. L.; Hache, B.; Von Moos, E. Synthesis, 2003, 11, 1683-1692.
BEHL, C. Alzheimer's disease and oxidative stress: implications for novel therapeutic approaches. Prog Neurobiol 57, 301-323 (1999).
BERGSBAKEN, T., Fink, S. L., and Cookson, B. T. (2009). Pyroptosis: host cell death and inflammation. Nat Rev Microbiol 7, 99-109.
BLOIS, M. S. (1958). Antioxidant determinations by the use of a stable free radical. Nature 181, 1199-1200.
CATER, H. L., Gitterman, D., Davis, S. M., Benham, C. D., Morrison, B., 3rd, and Sundstrom, L. E. (2007). Stretch-induced injury in organotypic hippocampal slice cultures reproduces in vivo post-traumatic neurodegeneration: role of glutamate receptors and voltage-dependent calcium channels. J Neurochem 101, 434-447.
CATER, H. L., Gitterman, D., Davis, S. M., Benham, C. D., Morrison, B., 3rd, and Sundstrom, L. E. (2007). Stretch-induced injury in organotypic hippocampal slice cultures reproduces in vivo post-traumatic neurodegeneration: role of glutamate receptors and voltage-dependent calcium channels. J Neurochem 101, 434-447.
CHA, J. H. et al. Altered brain neurotransmitter receptors in transgenic mice expressing a portion of an abnormal human huntington disease gene. Proc Natl Acad Sci USA 95, 6480-6485. (1998).
CHEAH, J. H., Kim, S. F., Hester, L. D., Clancy, K. W., Patterson, S. E., 3rd, Papadopoulos, V., and Snyder, S. H. (2006). NMDA receptor-nitric oxide transmission mediates neuronal iron homeostasis via the GTPase Dexras1. Neuron 51, 431-440.
CHEN, J. C. et al. MR of human postmortem brain tissue: correlative study between T2 and assays of iron and ferritin in Parkinson and Huntington disease. AJNR. American journal of neuroradiology 14, 275-281 (1993).
CHEN, J. et al. Iron accumulates in Huntington's disease neurons: protection by deferoxamine. PLoS One 8, e77023, doi:10.1371/journal.pone.0077023 (2013).
CHOI, D. W. (1988). Glutamate neurotoxicity and diseases of the nervous system. Neuron 1, 623-634.
CHRISTOFFERSON, D. E., and Yuan, J. (2010). Necroptosis as an alternative form of programmed cell death. Current Opinion in Cell Biology 22, 263-268.
CHUNG, N., Zhang, X. D., Kreamer, A., Locco, L., Kuan, P. F., Bartz, S., Linsley, P. S., Ferrer, M., and Strulovici, B. (2008). Median absolute deviation to improve hit selection for genome-scale RNAi screens. J Biomol Screen 13, 149-158.
CRUZ-AGUADO, R., Turner, L. F., Diaz, C. M. & Pinero, J. Nerve growth factor and striatal glutathione metabolism in a rat model of Huntington's disease. Restorative neurology and neuroscience 17, 217-221 (2000).
DEGTEREV A, et al. (2005) Chemical inhibitor of nonapoptotic cell death with therapeutic potential for ischemic brain injury. Nat Chem Biol 1(2):112-119.
DESILVA, T. M. et al. Glutamate transporter EAAT2 expression is up-regulated in reactive astrocytes in human periventricular leukomalacia. J Comp Neurol 508, 238-248, doi:10.1002/cne.21667 (2008).
DILLON C P, et al. (2012) Survival function of the FADD-CASPASE-8-cFLIP(L) complex. Cell Reports 1(5):401-407.
DILLON C P, et al. (2014) RIPK1 blocks early postnatal lethality mediated by caspase-8 and RIPK3. Cell 157(5):1189-1202.
DIXON, D. J. et al., (2012) Ferroptosis: An Iron-Dependent Form of Nonapoptotic Cell Death. Cell, Vol. 149, Issue 5, pp. 1060-1072.
DOLMA, S., Lessnick, S. L., Hahn, W. C., and Stockwell, B. R. (2003). Identification of genotype-selective antitumor agents using synthetic lethal chemical screening in engineered human tumor cells. Cancer Cell 3, 285-296.
DOMMERGUES, M. A., Gallego, J., Evrard, P. & Gressens, P. Iron supplementation aggravates periventricular cystic white matter lesions in newborn mice. European journal of paediatric neurology: EJPN: official journal of the European Paediatric Neurology Society 2, 313-318 (1998).
DUCE, J. A., Tsatsanis, A., Cater, M. A., James, S. A., Robb, E., Wikhe, K., Leong, S. L., Perez, K., Johanssen, T., Greenough, M. A., et al. (2010). Iron-export ferroxidase activity of beta-amyloid precursor protein is inhibited by zinc in Alzheimer's disease. Cell 142, 857-867.
ESTRADA SANCHEZ, A. M., Mejia-Toiber, J. & Massieu, L. Excitotoxic neuronal death and the pathogenesis of Huntington's disease. Archives of medical research 39, 265-276, doi:10.1016/j. arcmed.2007.11.011 (2008).
FOLKERTH, R. D. Periventricular leukomalacia: overview and recent findings. Pediatric and developmental pathology: the official journal of the Society for Pediatric Pathology and the Paediatric Pathology Society 9, 3-13, doi:10.2350/06-01-0024.1 (2006).
FOLLETT, P. L. et al. Glutamate receptor-mediated oligodendrocyte toxicity in periventricular leukomalacia: a protective role for topiramate. J Neurosci 24, 4412-4420, doi:10.1523/JNEUROSCI.0477-04.2004 (2004).
FUCHS, Y., and Steller, H. (2011). Programmed cell death in animal development and disease. Cell 147, 742-758.
GALLUZZI L, Kepp O, Krautwald S, Kroemer G, Linkermann A (2014) Molecular mechanisms of regulated necrosis. Semin Cell Dev Biol 35C:24-32.
GENNA, D. T. & Posner, G. H. Cyanocuprates convert carboxylic acids directly into ketones. Org Lett 13, 5358-5361, doi:10.1021/ol202237j (2011).
GOUT, P. W., Buckley, A. R., Simms, C. R., and Bruchovsky, N. (2001). Sulfasalazine, a potent suppressor of lymphoma growth by inhibition of the x(c)-cystine transporter: a new action for an old drug. Leukemia 15, 1633-1640.
GUO, W., Wu, S., Liu, J., and Fang, B. (2008). Identification of a small molecule with synthetic lethality for K-ras and protein kinase C iota. Cancer Res 68, 7403-7408.
HAMADA, Y. & Kiso, Y. The application of bioisosteres in drug design for novel drug discovery: focusing on acid protease inhibitors. Expert opinion on drug discovery 7, 903-922, doi:10.1517/17460441.2012.712513 (2012).
HAYNES, R. L. et al. Oxidative and nitrative injury in periventricular leukomalacia: a review. Brain pathology 15, 225-233 (2005).
HAYNES, R. L. et al. Nitrosative and oxidative injury to premyelinating oligodendrocytes in periventricular leukomalacia. Journal of neuropathology and experimental neurology 62, 441-450 (2003).
HE S, et al. (2009) Receptor interacting protein kinase-3 determines cellular necrotic response to TNF-alpha. Cell 137(6):1100-1111.
HOFFSTROM, B. G. et al. Inhibitors of protein disulfide isomerase suppress apoptosis induced by misfolded proteins. Nat Chem Biol 6, 900-906, doi:10.1038/nchembio.467 (2010).
HUANG, D., Ou, B. & Prior, R. L. The chemistry behind antioxidant capacity assays. J Agric Food Chem 53, 1841-1856, doi:10.1021/jf030723c (2005).
ISHIDA, T., Suzuki, T., Hirashima, S., Mizutani, K., Yoshida, A., Ando, I., Ikeda, S., Adachi, T., and Hashimoto, H. (2006). Benzimidazole inhibitors of hepatitis C virus NS5B polymerase: identification of 2-[(4-diarylmethoxy)phenyl]-benzimidazole. Bioorg Med Chem Lett 16, 1859-1863.
ISHII, T., Bannai, S., and Sugita, Y. (1981). Mechanism of growth stimulation of L1210 cells by 2-mercaptoethanol in vitro. Role of the mixed disulfide of 2-mercaptoethanol and cysteine. The Journal of biological chemistry 256, 12387-12392.
JACOBSON, M. D., and Raff, M. C. (1995). Programmed cell death and Bcl-2 protection in very low oxygen. Nature 374, 814-816.
JOHRI, A. & Beal, M. F. Antioxidants in Huntington's disease. Biochim Biophys Acta 1822, 664-674, doi:10.1016/j.bbadis.2011.11.014 (2012).
KAMATA, T. (2009). Roles of Nox1 and other Nox isoforms in cancer development. Cancer Sci 100, 1382-1388.
KANAI, Y., and Endou, H. (2003). Functional properties of multispecific amino acid transporters and their implications to transporter-mediated toxicity. J Toxicol Sci 28, 1-17.
LALEU, B., Gaggini, F., Orchard, M., Fioraso-Cartier, L., Cagnon, L., Houngninou-Molango, S., Gradia, A., Duboux, G., Merlot, C., Heitz, F., et al. (2010). First in class, potent, and orally bioavailable NADPH oxidase isoform 4 (Nox4) inhibitors for the treatment of idiopathic pulmonary fibrosis. Journal of medicinal chemistry 53, 7715-7730.
LEI, P., Ayton, S., Finkelstein, D. I., Spoerri, L., Ciccotosto, G. D., Wright, D. K., Wong, B. X., Adlard, P. A., Cherny, R. A., Lam, L. Q., et al. (2012). Tau deficiency induces parkinsonism with dementia by impairing APP-mediated iron export. Nature medicine 18, 291-295.
LI, Y., Maher, P., and Schubert, D. (1997). A role for 12-lipoxygenase in nerve cell death caused by glutathione depletion. Neuron 19, 453-463.
LINKERMANN A, et al. (2012) Rip1 (receptor-interacting protein kinase 1) mediates necroptosis and contributes to renal ischemia/reperfusion injury. Kidney Int 81(8):751-761.
LINKERMANN A, et al. (2013A) The RIP1-kinase inhibitor necrostatin-1 prevents osmotic nephrosis and contrast-induced AKI in mice. J Am Soc Nephrol 24(10):1545-1557.
LINKERMANN A, et al. (2013B) Two independent pathways of regulated necrosis mediate ischemia-reperfusion injury. Proc Natl Acad Sci USA 110(29):12024-12029.
LINKERMANN A, Green D R (2014A) Necroptosis. N Engl J Med 370(5):455-465.
LINKERMANN A, et al. (2014B) Regulated Cell Death in AKI. J Am Soc Nephrol, ASN.2014030262.
LIPINSKI, C. A., Lombardo, F., Dominy, B. W., and Feeney, P. J. (2001). Experimental and computational approaches to estimate solubility and permeability in drug discovery and development settings. Advanced drug delivery reviews 46, 3-26.
LO, M., Ling, V., Wang, Y. Z., and Gout, P. W. (2008). The xc-cystine/glutamate antiporter: a mediator of pancreatic cancer growth with a role in drug resistance. British journal of cancer 99, 464-472.
LOSSI, L., Alasia, S., Salio, C., and Merighi, A. (2009). Cell death and proliferation in acute slices and organotypic cultures of mammalian CNS. Prog Neurobiol 88, 221-245.
LUEDDE M, et al. (2014) RIP3, a kinase promoting necroptotic cell death, mediates adverse remodelling after myocardial infarction. Cardiovasc Res 103(2):206-216.
MACARRON, R., Banks, M. N., Bojanic, D., Burns, D. J., Cirovic, D. A., Garyantes, T., Green, D. V., Hertzberg, R. P., Janzen, W. P., Paslay, J. W., et al. (2011). Impact of high-throughput screening in biomedical research. Nature reviews Drug discovery 10, 188-195.
MASON, R. P. et al. Glutathione peroxidase activity is neuroprotective in models of Huntington's disease. Nat Genet 45, 1249-1254, doi:10.1038/ng.2732 (2013).
MILLER, B. R. & Bezprozvanny, I. Corticostriatal circuit dysfunction in Huntington's disease: intersection of glutamate, dopamine and calcium. Future neurology 5, 735-756, doi:10.2217/fn1.10.41 (2010).
MOFFAT, J., Grueneberg, D. A., Yang, X., Kim, S. Y., Kloepfer, A. M., Hinkle, G., Piqani, B., Eisenhaure, T. M., Luo, B., Grenier, J. K., et al. (2006). A lentiviral RNAi library for human and mouse genes applied to an arrayed viral high-content screen. Cell 124, 1283-1298.
MORRISON, P. J. & Nevin, N. C. Serum iron, total iron binding capacity and ferritin in early Huntington disease patients. Irish journal of medical science 163, 236-237 (1994).
MORRISON, B., 3rd, Pringle, A. K., McManus, T., Ellard, J., Bradley, M., Signorelli, F., Iannotti, F., and Sundstrom, L. E. (2002). L-arginyl-3,4-spermidine is neuroprotective in several in vitro models of neurodegeneration and in vivo ischaemia without suppressing synaptic transmission. Br J Pharmacol 137, 1255-1268.
MULAY S R, et al. (2013) Calcium oxalate crystals induce renal inflammation by NLRP3-mediated IL-1β secretion. J Clin Invest 123(1):236-246.
MULLEN, A. R., Wheaton, W. W., Jin, E. S., Chen, P. H., Sullivan, L. B., Cheng, T., Yang, Y., Linehan, W. M., Chandel, N. S., and Deberardinis, R. J. (2011). Reductive carboxylation supports growth in tumour cells with defective mitochondria. Nature.
MURPHY, T. H., Miyamoto, M., Sastre, A., Schnaar, R. L., and Coyle, J. T. (1989). Glutamate toxicity in a neuronal cell line involves inhibition of cystine transport leading to oxidative stress. Neuron 2, 1547-1558.
NATIONAL RESEARCH COUNCIL (2011) Guide for the Care and Use of Laboratory Animals (National Academies Press, Washington, D.C.), 8th Ed.
NI CHONGHAILE, T., Sarosiek, K. A., Vo, T. T., Ryan, J. A., Tammareddi, A., Moore Vdel, G., Deng, J., Anderson, K. C., Richardson, P., Tai, Y. T., et al. (2011). Pretreatment mitochondrial priming correlates with clinical response to cytotoxic chemotherapy. Science 334, 1129-1133.
NORABERG, J., Poulsen, F. R., Blaabjerg, M., Kristensen, B. W., Bonde, C., Montero, M., Meyer, M., Gramsbergen, J. B., and Zimmer, J. (2005). Organotypic hippocampal slice cultures for studies of brain damage, neuroprotection and neurorepair. Curr Drug Targets CNS Neurol Disord 4, 435-452.
PAGLIARINI, D. J., Calvo, S. E., Chang, B., Sheth, S. A., Vafai, S. B., Ong, S. E., Walford, G. A., Sugiana, C., Boneh, A., Chen, W. K., et al. (2008). A mitochondrial protein compendium elucidates complex I disease biology. Cell 134, 112-123.
PARK, J. S., Pasupulati, R., Feldkamp, T., Roeser, N. F. & Weinberg, J. M. Cyclophilin D and the mitochondrial permeability transition in kidney proximal tubules after hypoxic and ischemic injury. American journal of physiology. Renal physiology 301, F134-150, doi:10.1152/ajprenal.00033.2011 (2011).
PASSANITI, P. et al. Synthesis, spectroscopic and electrochemical properties of mononuclear and dinuclear bis(bipy)ruthenium(II) complexes containing dimethoxyphenyl(pyridin-2-yl)-1,2,4-triazole ligands J. Chem. Soc., Dalton Transactions 8, 1740-1746 (2002).
PETR, G. T. et al. Glutamate transporter expression and function in a striatal neuronal model of Huntington's disease. Neurochem Int 62, 973-981, doi:10.1016/j.neuint.2013.02.026 (2013).
PINNIX, Z. K., Miller, L. D., Wang, W., D'Agostino, R., Jr., Kute, T., Willingham, M. C., Hatcher, H., Tesfay, L., Sui, G., Di, X., et al. (2010). Ferroportin and iron regulation in breast cancer progression and prognosis. Sci Transl Med 2, 43ra56.
PIPIK, B., Ho, G. J., Williams, J. M. & Conlon, D. A. A preferred synthesis of 1,2,4-oxadiazoles. Synthetic Communications 34, 1863-1870 (2004).
RAJ, L., Ide, T., Gurkar, A. U., Foley, M., Schenone, M., Li, X., Tolliday, N.J., Golub, T. R., Carr, S. A., Shamji, A. F., et al. (2011). Selective killing of cancer cells by a small molecule targeting the stress response to ROS. Nature 475, 231-234.
RAMANA, Kota V. et al., “Lipid Peroxidation Products in Human Health and Disease,” Oxidative Medicine and Cellular Longevity, vol. 2013, Article ID 583438, 3 pages, 2013. doi:10.1155/2013/583438.
RAMANATHAN, A., and Schreiber, S. L. (2009). Direct control of mitochondrial function by mTOR. Proc Natl Acad Sci USA 106, 22229-22232.
RATAN, R. R., Murphy, T. H., and Baraban, J. M. (1994). Oxidative stress induces apoptosis in embryonic cortical neurons. J Neurochem 62, 376-379.
RIBEIRO, F. M., Pires, R. G. & Ferguson, S. S. Huntington's disease and Group I metabotropic glutamate receptors. Molecular neurobiology 43, 1-11, doi:10.1007/s12035-010-8153-1 (2011).
RIBEIRO, M. et al. Glutathione redox cycle dysregulation in Huntington's disease knock-in striatal cells. Free Radic Biol Med 53, 1857-1867, doi:10.1016/j.freeradbiomed.2012.09.004 (2012).
SAITOH, M. et al. Design, synthesis and structure-activity relationships of 1,3,4-oxadiazole derivatives as novel inhibitors of glycogen synthase kinase-3beta. Bioorg Med Chem 17, 2017-2029, doi:10.1016/j.bmc.2009.01.019 (2009).
SALAHUDEEN, A. A., Thompson, J. W., Ruiz, J. C., Ma, H. W., Kinch, L. N., Li, Q., Grishin, N. V., and Bruick, R. K. (2009). An E3 ligase possessing an iron-responsive hemerythrin domain is a regulator of iron homeostasis. Science 326, 722-726.
SANCHEZ, M., Galy, B., Schwanhaeusser, B., Blake, J., Bahr-Ivacevic, T., Benes, V., Selbach, M., Muckenthaler, M. U., and Hentze, M. W. (2011). Iron regulatory protein-1 and -2: transcriptome-wide definition of binding mRNAs and shaping of the cellular proteome by iron regulatory proteins. Blood 118, e168-179.
SATO, H., Tamba, M., Ishii, T., and Bannai, S. (1999). Cloning and expression of a plasma membrane cystine/glutamate exchange transporter composed of two distinct proteins. The Journal of biological chemistry 274, 11455-11458.
SHAW, A. T., Winslow, M. M., Magendantz, M., Ouyang, C., Dowdle, J., Subramanian, A., Lewis, T. A., Maglathin, R. L., Tolliday, N., and Jacks, T. (2011). Selective killing of K-ras mutant cancer cells by small molecule inducers of oxidative stress. Proc Natl Acad Sci USA.
SKOUTA R, et al. (2014) Ferrostatins inhibit oxidative lipid damage and cell death in diverse disease models. J Am Chem Soc 136(12):4551-4556.
SMITH C C, et al. (2007) Necrostatin: A potentially novel cardioprotective agent? Cardiovasc Drugs Ther 21(4):227-233.
SOGABE, K., Roeser, N. F., Venkatachalam, M. A. & Weinberg, J. M. Differential cytoprotection by glycine against oxidant damage to proximal tubule cells. Kidney international 50, 845-854 (1996).
SUNDSTROM, L., Morrison, B., 3rd, Bradley, M., and Pringle, A. (2005). Organotypic cultures as tools for functional screening in the CNS. Drug discovery today 10, 993-1000.
TAN, S., Sagara, Y., Liu, Y., Maher, P., and Schubert, D. (1998). The regulation of reactive oxygen species production during programmed cell death. The Journal of Cell Biology 141, 1423-1432.
THOMPSON, C. B. (1995). Apoptosis in the pathogenesis and treatment of disease. Science 267, 1456-1462.
TRACHOOTHAM, D., Zhou, Y., Zhang, H., Demizu, Y., Chen, Z., Pelicano, H., Chiao, P. J., Achanta, G., Arlinghaus, R. B., Liu, J., et al. (2006). Selective killing of oncogenically transformed cells through a ROS-mediated mechanism by beta-phenylethyl isothiocyanate. Cancer Cell 10, 241-252.
TRAYKOVA-BRAUCH M, et al. (2008) An efficient and versatile system for acute and chronic modulation of renal tubular function in transgenic mice. Nat Med 14(9): 979-984.
TURMAINE, M. et al. Nonapoptotic neurodegeneration in a transgenic mouse model of Huntington's disease. Proc Natl Acad Sci USA 97, 8093-8097. (2000).
VARMA, H., Lo, D. C. & Stockwell, B. R. in Neurobiology of Huntington's Disease: Applications to Drug Discovery Frontiers in Neuroscience (eds D. C. Lo & R. E. Hughes) (2011).
VASHISHT, A. A., Zumbrennen, K. B., Huang, X., Powers, D. N., Durazo, A., Sun, D., Bhaskaran, N., Persson, A., Uhlen, M., Sangfelt, O., et al. (2009). Control of iron homeostasis by an iron-regulated ubiquitin ligase. Science 326, 718-721.
VIGIL, D., Cherfils, J., Rossman, K. L., and Der, C. J. (2010). Ras superfamily GEFs and GAPs: validated and tractable targets for cancer therapy? Nat Rev Cancer 10, 842-857.
WANG, Y., Dawson, V. L., and Dawson, T. M. (2009). Poly(ADP-ribose) signals to mitochondrial AIF: a key event in parthanatos. Exp Neurol 218, 193-202.
WATKINS, P. A., Maiguel, D., Jia, Z., and Pevsner, J. (2007). Evidence for 26 distinct acyl-coenzyme A synthetase genes in the human genome. J Lipid Res 48, 2736-2750.
WISE, D. R., DeBerardinis, R. J., Mancuso, A., Sayed, N., Zhang, X. Y., Pfeiffer, H. K., Nissim, I., Daikhin, E., Yudkoff, M., McMahon, S. B., et al. (2008). Myc regulates a transcriptional program that stimulates mitochondrial glutaminolysis and leads to glutamine addiction. Proceedings of the National Academy of Sciences of the United States of America 105, 18782-18787.
WOLPAW, A. J., Shimada, K., Skouta, R., Welsch, M. E., Akavia, U. D., Pe'er, D., Shaik, F., Bulinski, J. C., and Stockwell, B. R. (2011). Modulatory profiling identifies mechanisms of small molecule-induced cell death. Proceedings of the National Academy of Sciences of the United States of America.
WU, C. et al. Discovery, modeling, and human pharmacokinetics of N-(2-acetyl-4,6-dimethylphenyl)-3-(3,4-dimethylisoxazol-5-ylsulfamoyl)thiophene-2-carboxamide (TBC3711), a second generation, ETA selective, and orally bioavailable endothelin antagonist. J Med Chem 47, 1969-1986, doi:10.1021/jm030528p (2004).
YAGODA, N., von Rechenberg, M., Zaganjor, E., Bauer, A. J., Yang, W. S., Fridman, D. J., Wolpaw, A. J., Smukste, I., Peltier, J. M., Boniface, J. J., et al. (2007). RAS-RAF-MEK-dependent oxidative cell death involving voltage-dependent anion channels. Nature 447, 864-868.
YANG, W. S., and Stockwell, B. R. (2008). Synthetic lethal screening identifies compounds activating iron-dependent, nonapoptotic cell death in oncogenic-RAS-harboring cancer cells. Chemistry & biology 15, 234-245.
YONEZAWA, M., Back, S. A., Gan, X., Rosenberg, P. A., and Volpe, J. J. (1996). Cystine deprivation induces oligodendroglial death: rescue by free radical scavengers and by a diffusible glial factor. J Neurochem 67, 566-573.
ZERON, M. M. et al. Increased sensitivity to N-methyl-D-aspartate receptor-mediated excitotoxicity in a mouse model of Huntington's disease. Neuron 33, 849-860. (2002).
ZHANG D W, et al. (2009) RIP3, an energy metabolism regulator that switches TNFinduced cell death from apoptosis to necrosis. Science 325(5938):332-336.

All documents cited in this application are hereby incorporated by reference as if recited in full herein.

Although illustrative embodiments of the present disclosure have been described herein, it should be understood that the disclosure is not limited to those described, and that various other changes or modifications may be made by one skilled in the art without departing from the scope or spirit of the disclosure.

What is claimed is:

1. A compound according to formula (1): 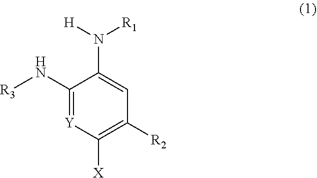
wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is 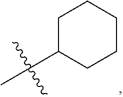 [SMILES: *C1CCCCC1]
R₂ cannot be 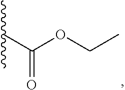 [SMILES: *C(=O)OCC]
the compound is not

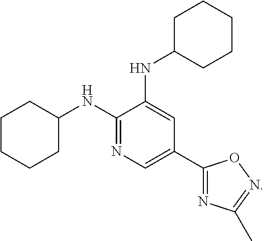 [SMILES: Cc1noc(-c2cnc(NC3CCCCC3)c(NC3CCCCC3)c2)n1]

2. A compound according to claim 1 having the structure of formula (1a): 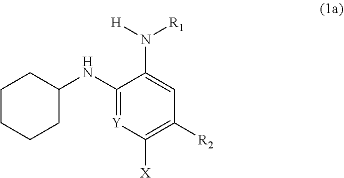
wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H and Y is —CH, R₂ cannot be 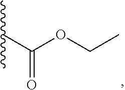 [SMILES: *C(=O)OCC]
the compound is not

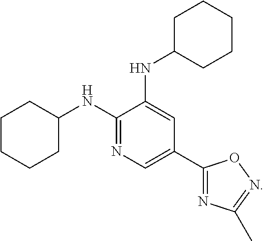 [SMILES: Cc1noc(-c2cnc(NC3CCCCC3)c(NC3CCCCC3)c2)n1]

3. A compound according to claim 1 having the structure of formula (1b): 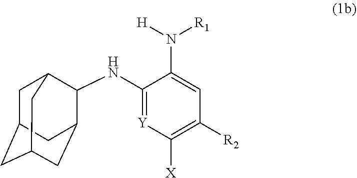
wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof.

4. A compound according to claim 1, which is selected from the group consisting of: 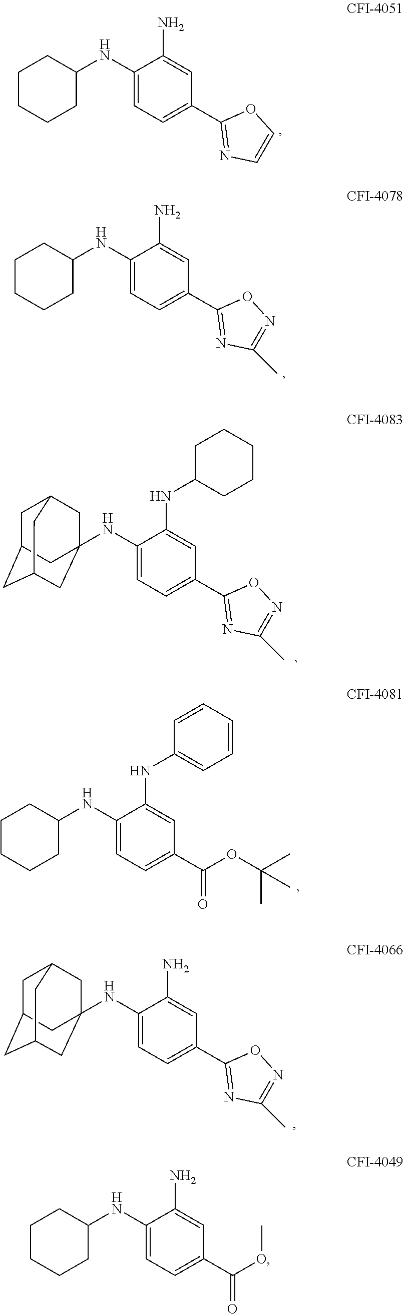 [SMILES: Nc1cc(-c2ncco2)ccc1NC1CCCCC1 ; Cc1noc(-c2ccc(NC3CCCCC3)c(N)c2)n1 ; Cc1noc(-c2ccc(NC34CC5CC(CC(C5)C3)C4)c(NC3CCCCC3)c2)n1 ; CC(C)(C)OC(=O)c1ccc(NC2CCCCC2)c(Nc2ccccc2)c1 ; Cc1noc(-c2ccc(NC34CC5CC(CC(C5)C3)C4)c(N)c2)n1 ; COC(=O)c1ccc(NC2CCCCC2)c(N)c1] 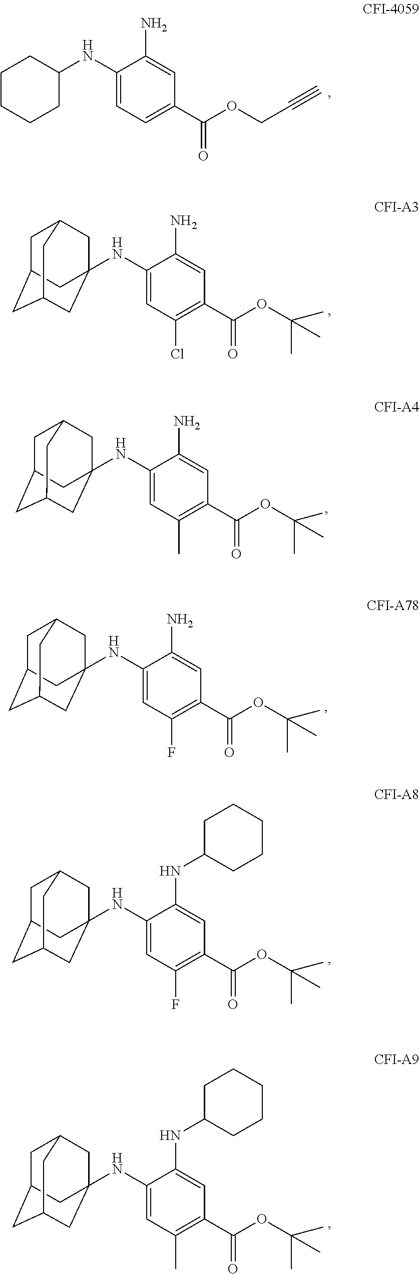 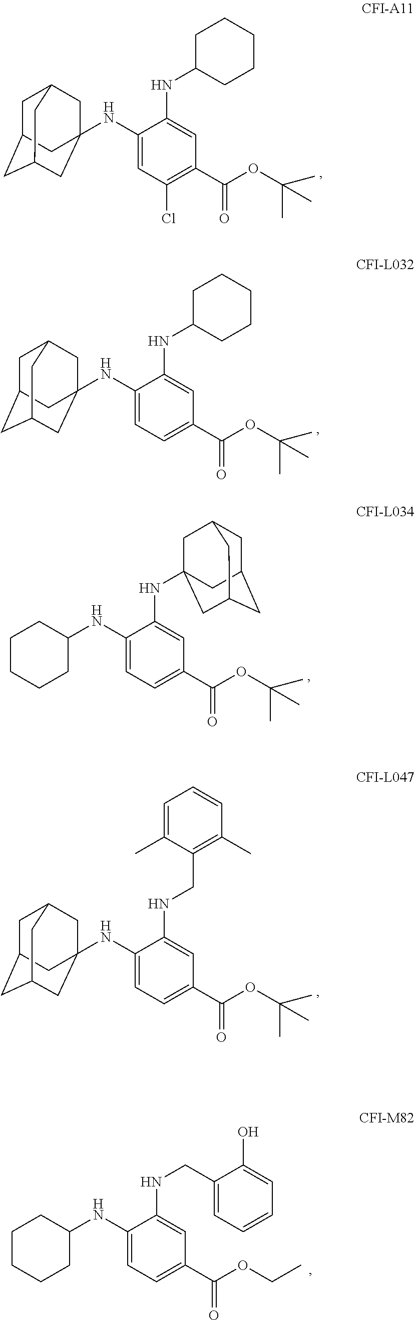 [SMILES: CC(C)(C)OC(=O)c1cc(NC2CCCCC2)c(NC23CC4CC(CC(C4)C2)C3)cc1Cl ; CC(C)(C)OC(=O)c1ccc(NC23CC4CC(CC(C4)C2)C3)c(NC2CCCCC2)c1 ; CC(C)(C)OC(=O)c1ccc(NC2CCCCC2)c(NC23CC4CC(CC(C4)C2)C3)c1 ; Cc1cccc(C)c1CNc1cc(C(=O)OC(C)(C)C)ccc1NC12CC3CC(CC(C3)C1)C2 ; CCOC(=O)c1ccc(NC2CCCCC2)c(NCc2ccccc2O)c1] 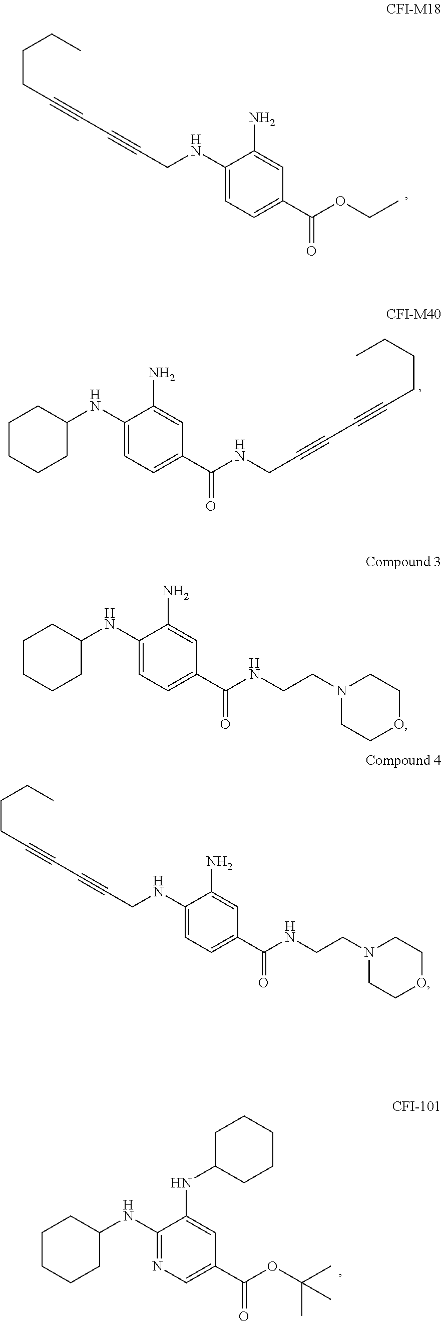 [SMILES: CCCCC#CC#CCNc1ccc(C(=O)OCC)cc1N ; CCCCC#CC#CCNC(=O)c1ccc(NC2CCCCC2)c(N)c1 ; Nc1cc(C(=O)NCCN2CCOCC2)ccc1NC1CCCCC1 ; CCCCC#CC#CCNc1ccc(C(=O)NCCN2CCOCC2)cc1N ; CC(C)(C)OC(=O)c1cnc(NC2CCCCC2)c(NC2CCCCC2)c1] 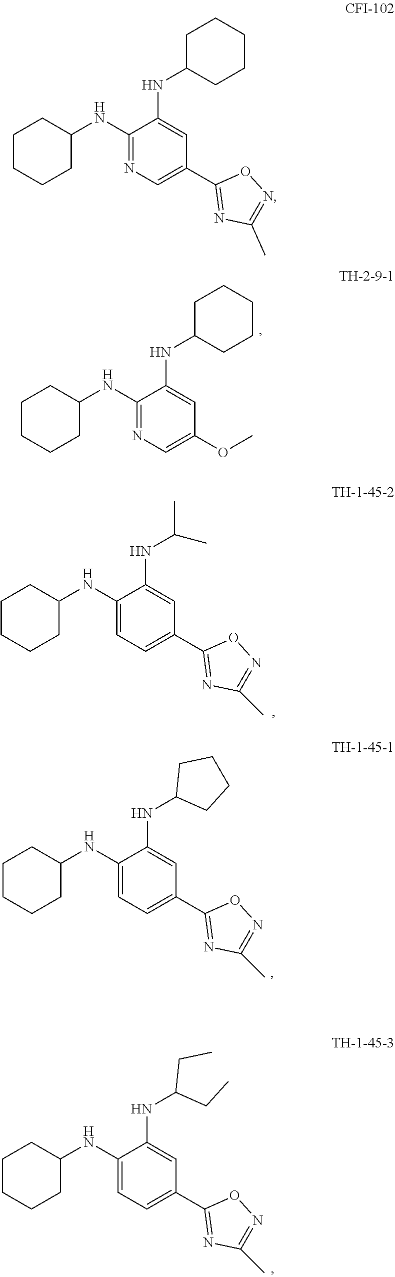 [SMILES: Cc1noc(-c2cnc(NC3CCCCC3)c(NC3CCCCC3)c2)n1 ; COc1cnc(NC2CCCCC2)c(NC2CCCCC2)c1 ; Cc1noc(-c2ccc(NC3CCCCC3)c(NC(C)C)c2)n1 ; Cc1noc(-c2ccc(NC3CCCCC3)c(NC3CCCC3)c2)n1 ; CCC(CC)Nc1cc(-c2nc(C)no2)ccc1NC1CCCCC1] 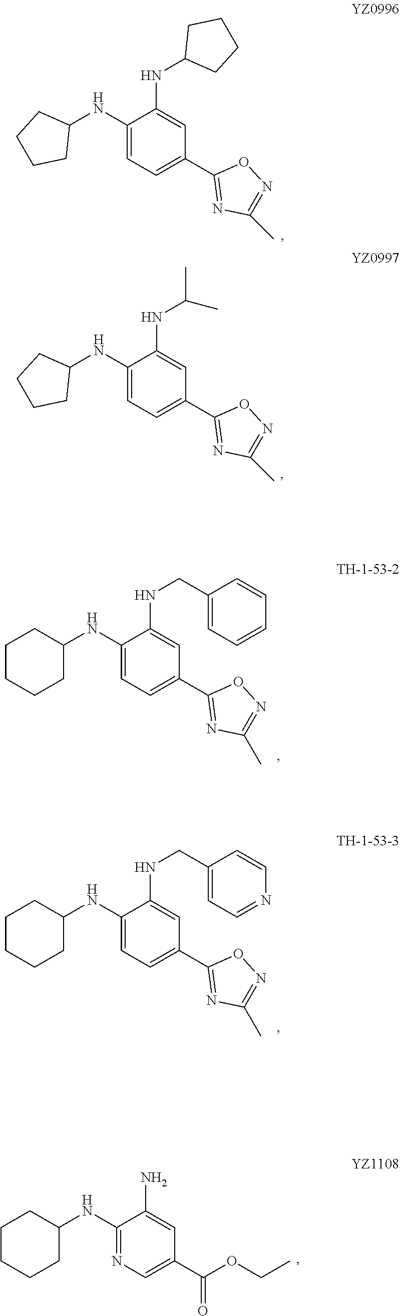 [SMILES: Cc1noc(-c2ccc(NC3CCCC3)c(NC3CCCC3)c2)n1 ; Cc1noc(-c2ccc(NC3CCCC3)c(NC(C)C)c2)n1 ; Cc1noc(-c2ccc(NC3CCCCC3)c(NCc3ccccc3)c2)n1 ; Cc1noc(-c2ccc(NC3CCCCC3)c(NCc3ccncc3)c2)n1 ; CCOC(=O)c1cnc(NC2CCCCC2)c(N)c1] 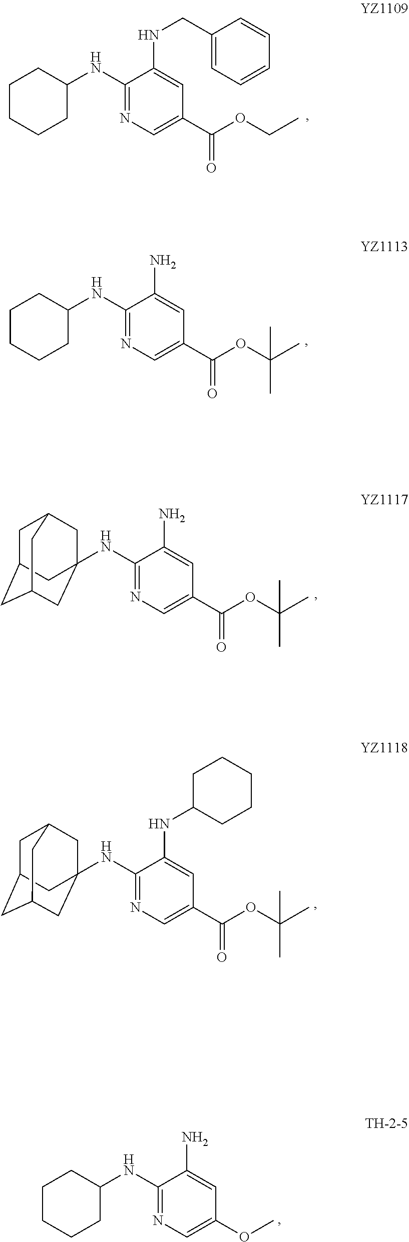 [SMILES: CCOC(=O)c1cnc(NC2CCCCC2)c(NCc2ccccc2)c1 ; CC(C)(C)OC(=O)c1cnc(NC2CCCCC2)c(N)c1 ; CC(C)(C)OC(=O)c1cnc(NC23CC4CC(CC(C4)C2)C3)c(N)c1 ; CC(C)(C)OC(=O)c1cnc(NC23CC4CC(CC(C4)C2)C3)c(NC2CCCCC2)c1 ; COc1cnc(NC2CCCCC2)c(N)c1] 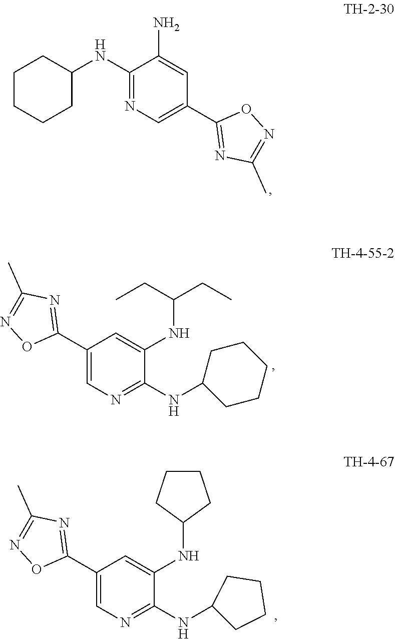 [SMILES: Cc1noc(-c2cnc(NC3CCCCC3)c(N)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
or an N-oxide, crystalline form, hydrate, or a pharmaceutically acceptable salt thereof.

5. A compound according to claim 4, which is selected from the group consisting of: 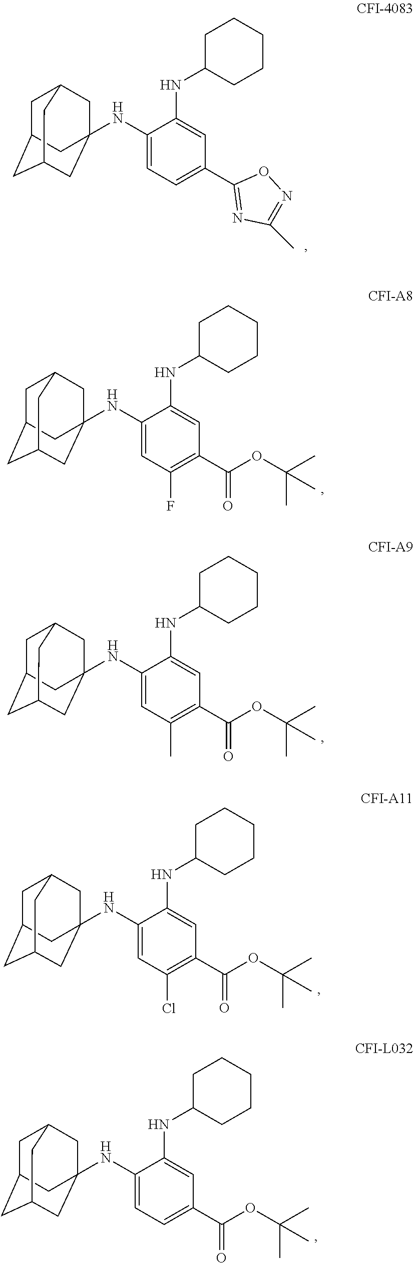 [SMILES: Cc1noc(-c2ccc(NC34CC5CC(CC(C5)C3)C4)c(NC3CCCCC3)c2)n1 ; CC(C)(C)OC(=O)c1cc(NC2CCCCC2)c(NC23CC4CC(CC(C4)C2)C3)cc1F ; Cc1cc(NC23CC4CC(CC(C4)C2)C3)c(NC2CCCCC2)cc1C(=O)OC(C)(C)C ; CC(C)(C)OC(=O)c1cc(NC2CCCCC2)c(NC23CC4CC(CC(C4)C2)C3)cc1Cl ; CC(C)(C)OC(=O)c1ccc(NC23CC4CC(CC(C4)C2)C3)c(NC2CCCCC2)c1] 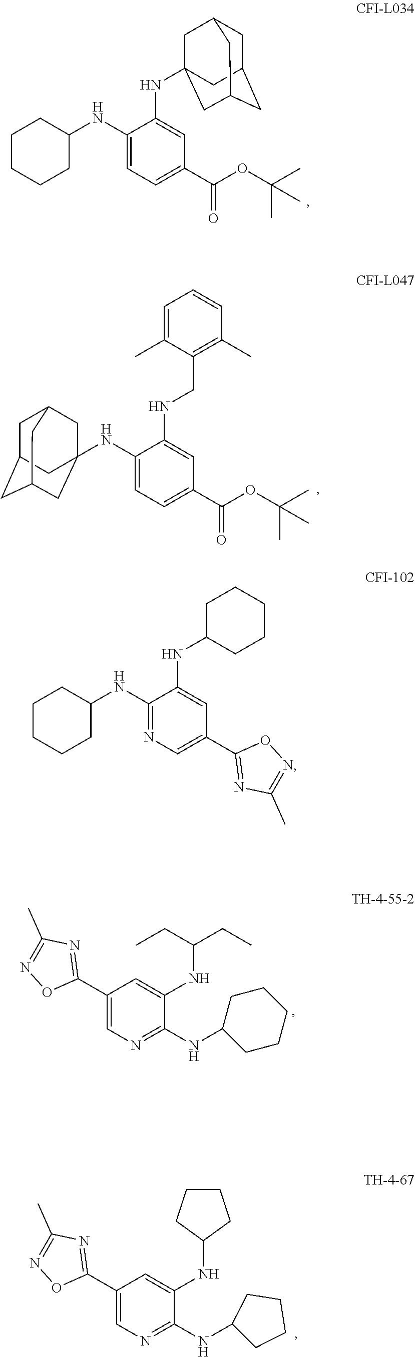 [SMILES: CC(C)(C)OC(=O)c1ccc(NC2CCCCC2)c(NC23CC4CC(CC(C4)C2)C3)c1 ; Cc1cccc(C)c1CNc1cc(C(=O)OC(C)(C)C)ccc1NC12CC3CC(CC(C3)C1)C2 ; Cc1noc(-c2cnc(NC3CCCCC3)c(NC3CCCCC3)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
or an N-oxide, crystalline form, hydrate, or a pharmaceutically acceptable salt thereof.

6. A compound according to claim 5, which is selected from the group consisting of: 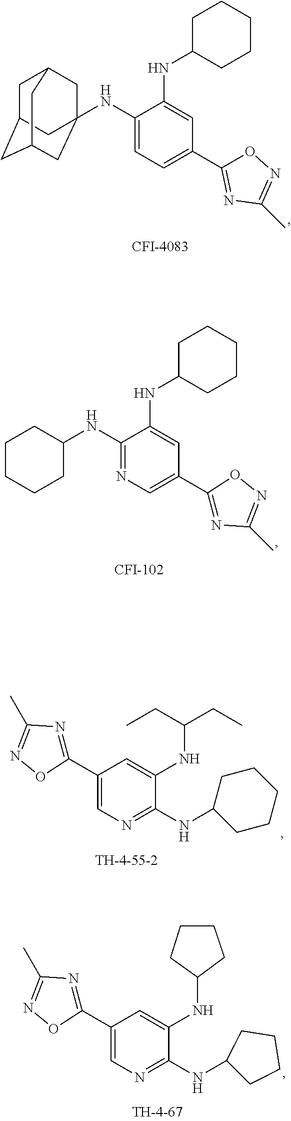
or an N-oxide, crystalline form, hydrate, or a pharmaceutically acceptable salt thereof.

7. A pharmaceutical composition comprising a pharmaceutically acceptable carrier or diluent and one or more compounds according to formula (1): 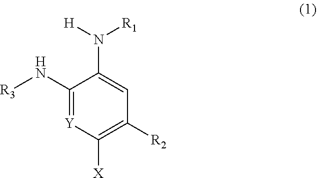
wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is 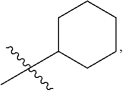 [SMILES: *C1CCCCC1]
R₂ cannot be 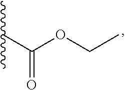 [SMILES: *C(=O)OCC]
the compound is not

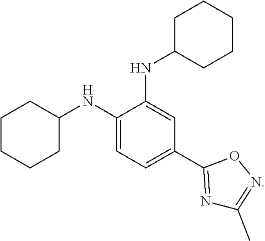 [SMILES: Cc1noc(-c2ccc(NC3CCCCC3)c(NC3CCCCC3)c2)n1]

8. A pharmaceutical composition according to claim 7, wherein the one or more compounds have the structure of formula (1a): 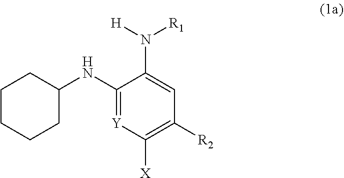
wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H and Y is —CH, R₂ cannot be 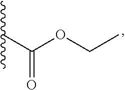 [SMILES: *C(=O)OCC]
the compound is not

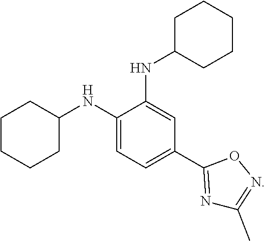 [SMILES: Cc1noc(-c2ccc(NC3CCCCC3)c(NC3CCCCC3)c2)n1]

9. A pharmaceutical composition according to claim 7, wherein the one or more compounds have the structure of formula (1b): 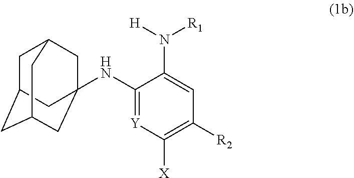
wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof.

10. A pharmaceutical composition according to claim 7, wherein the one or more compounds have a structure selected from the group consisting of: 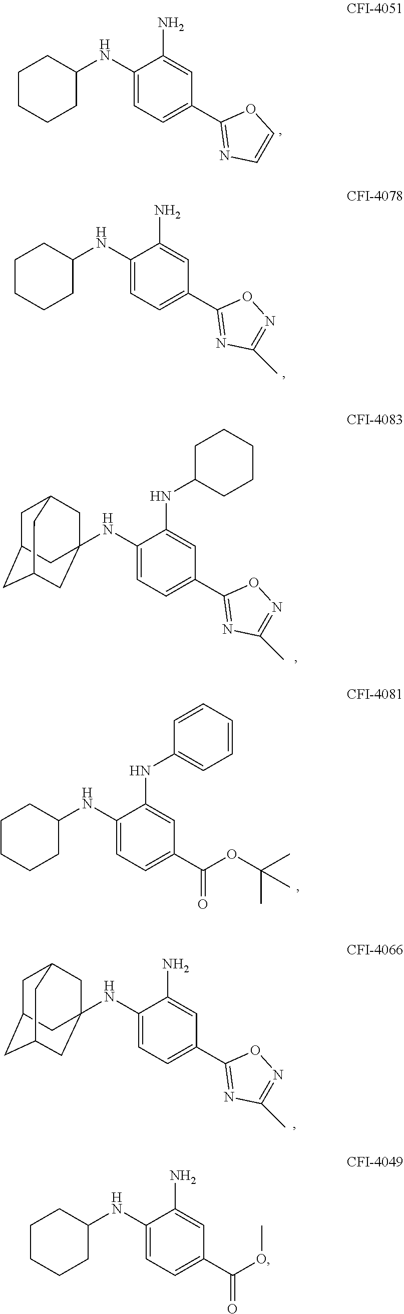 [SMILES: Nc1cc(-c2ncco2)ccc1NC1CCCCC1 ; Cc1noc(-c2ccc(NC3CCCCC3)c(N)c2)n1 ; Cc1noc(-c2ccc(NC34CC5CC(CC(C5)C3)C4)c(NC3CCCCC3)c2)n1 ; CC(C)(C)OC(=O)c1ccc(NC2CCCCC2)c(Nc2ccccc2)c1 ; Cc1noc(-c2ccc(NC34CC5CC(CC(C5)C3)C4)c(N)c2)n1 ; COC(=O)c1ccc(NC2CCCCC2)c(N)c1] 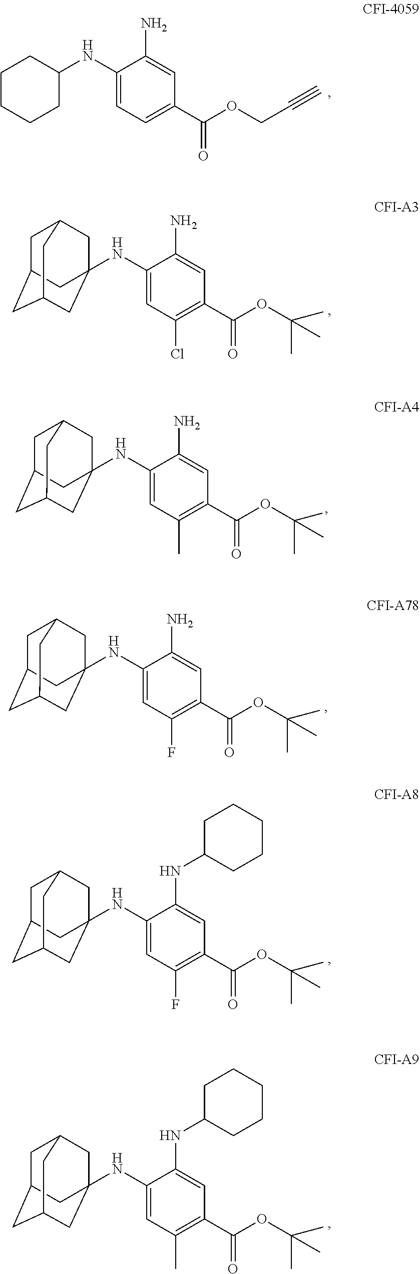 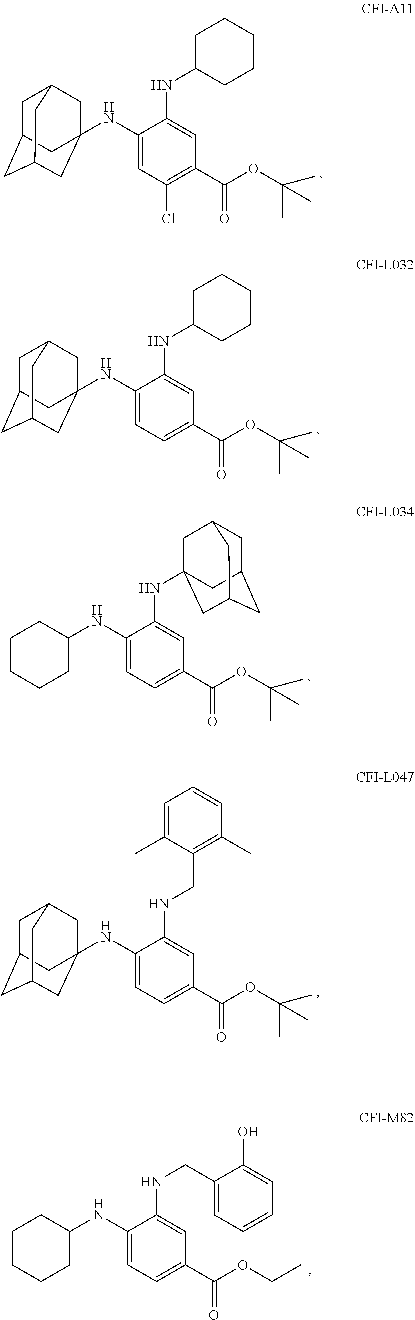 [SMILES: CC(C)(C)OC(=O)c1cc(NC2CCCCC2)c(NC23CC4CC(CC(C4)C2)C3)cc1Cl ; CC(C)(C)OC(=O)c1ccc(NC23CC4CC(CC(C4)C2)C3)c(NC2CCCCC2)c1 ; CC(C)(C)OC(=O)c1ccc(NC2CCCCC2)c(NC23CC4CC(CC(C4)C2)C3)c1 ; Cc1cccc(C)c1CNc1cc(C(=O)OC(C)(C)C)ccc1NC12CC3CC(CC(C3)C1)C2 ; CCOC(=O)c1ccc(NC2CCCCC2)c(NCc2ccccc2O)c1] 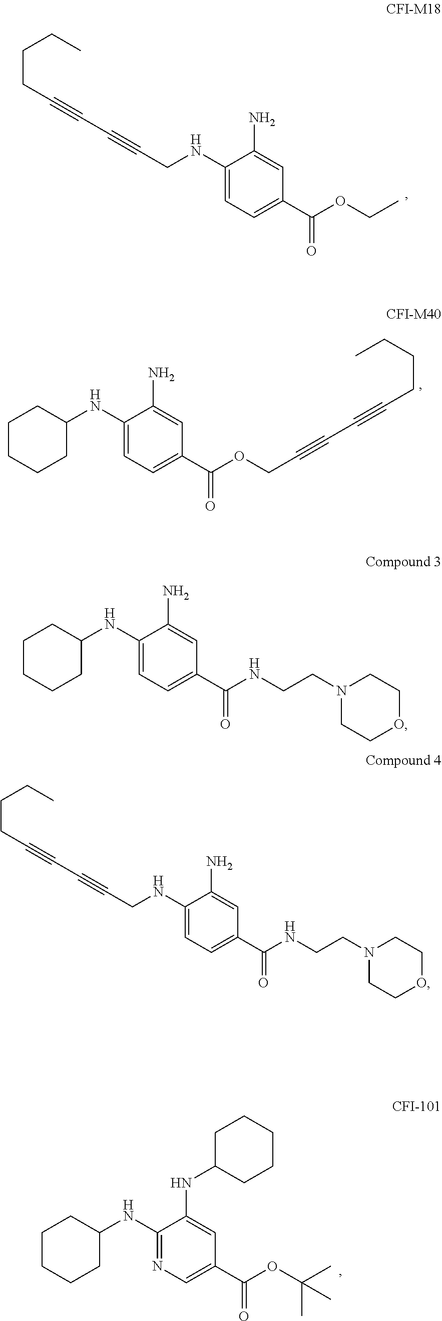 [SMILES: CCCCC#CC#CCNc1ccc(C(=O)OCC)cc1N ; CCCCC#CC#CCOC(=O)c1ccc(NC2CCCCC2)c(N)c1 ; Nc1cc(C(=O)NCCN2CCOCC2)ccc1NC1CCCCC1 ; CCCCC#CC#CCNc1ccc(C(=O)NCCN2CCOCC2)cc1N ; CC(C)(C)OC(=O)c1cnc(NC2CCCCC2)c(NC2CCCCC2)c1] 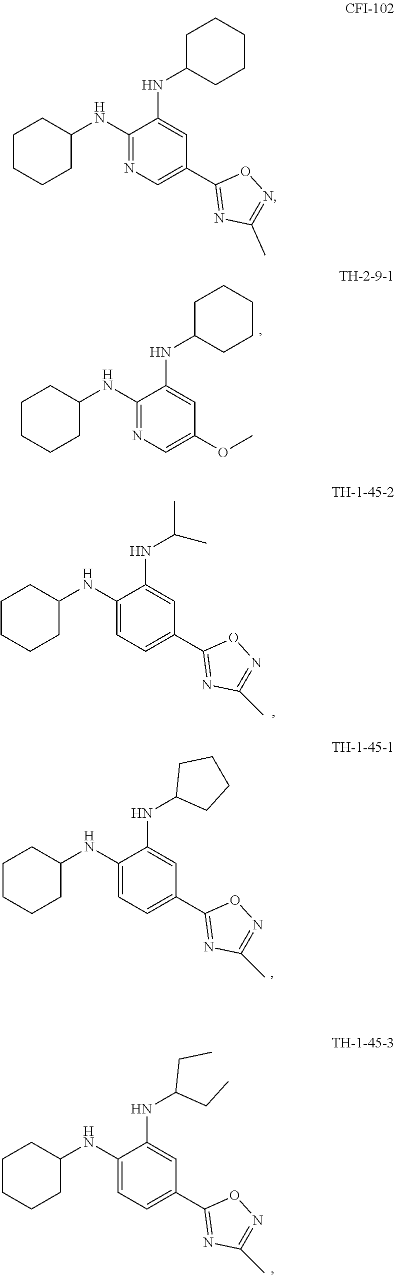 [SMILES: Cc1noc(-c2cnc(NC3CCCCC3)c(NC3CCCCC3)c2)n1 ; COc1cnc(NC2CCCCC2)c(NC2CCCCC2)c1 ; Cc1noc(-c2ccc(NC3CCCCC3)c(NC(C)C)c2)n1 ; Cc1noc(-c2ccc(NC3CCCCC3)c(NC3CCCC3)c2)n1 ; CCC(CC)Nc1cc(-c2nc(C)no2)ccc1NC1CCCCC1] 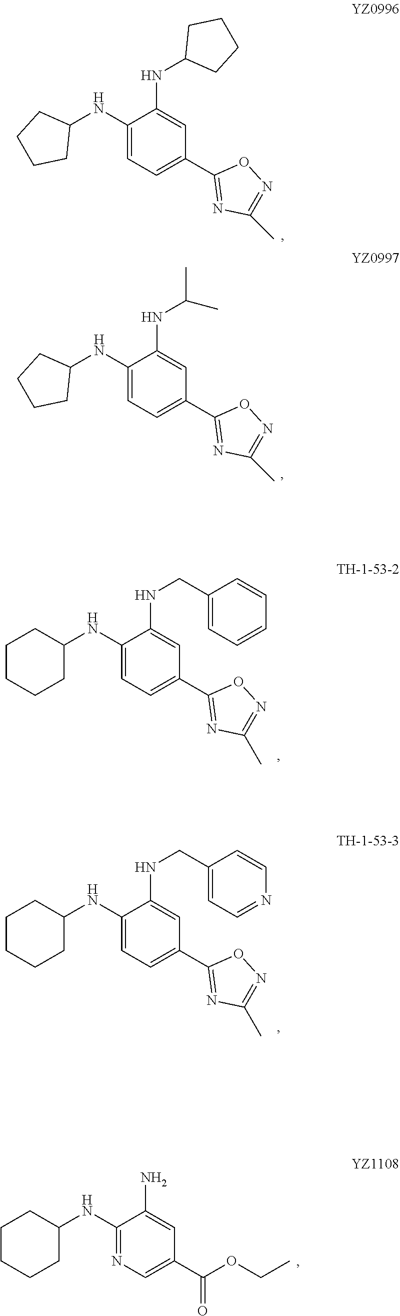 [SMILES: Cc1noc(-c2ccc(NC3CCCC3)c(NC3CCCC3)c2)n1 ; Cc1noc(-c2ccc(NC3CCCC3)c(NC(C)C)c2)n1 ; Cc1noc(-c2ccc(NC3CCCCC3)c(NCc3ccccc3)c2)n1 ; Cc1noc(-c2ccc(NC3CCCCC3)c(NCc3ccncc3)c2)n1 ; CCOC(=O)c1cnc(NC2CCCCC2)c(N)c1] 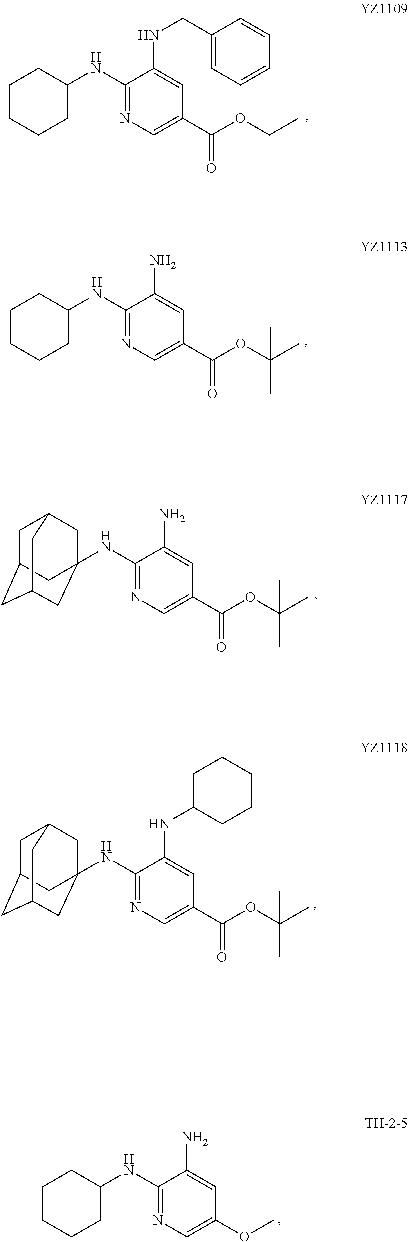 [SMILES: CCOC(=O)c1cnc(NC2CCCCC2)c(NCc2ccccc2)c1 ; CC(C)(C)OC(=O)c1cnc(NC2CCCCC2)c(N)c1 ; CC(C)(C)OC(=O)c1cnc(NC23CC4CC(CC(C4)C2)C3)c(N)c1 ; CC(C)(C)OC(=O)c1cnc(NC23CC4CC(CC(C4)C2)C3)c(NC2CCCCC2)c1 ; COc1cnc(NC2CCCCC2)c(N)c1] 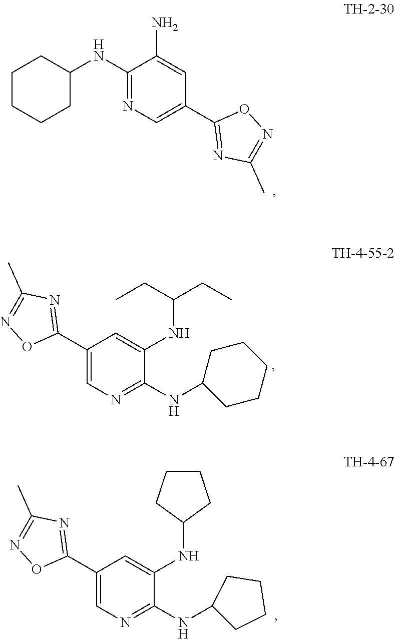 [SMILES: Cc1noc(-c2cnc(NC3CCCCC3)c(N)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
or an N-oxide, crystalline form, hydrate, or a pharmaceutically acceptable salt thereof.

11. A pharmaceutical composition according to claim 7, wherein the one or more compounds have a structure selected from the group consisting of: 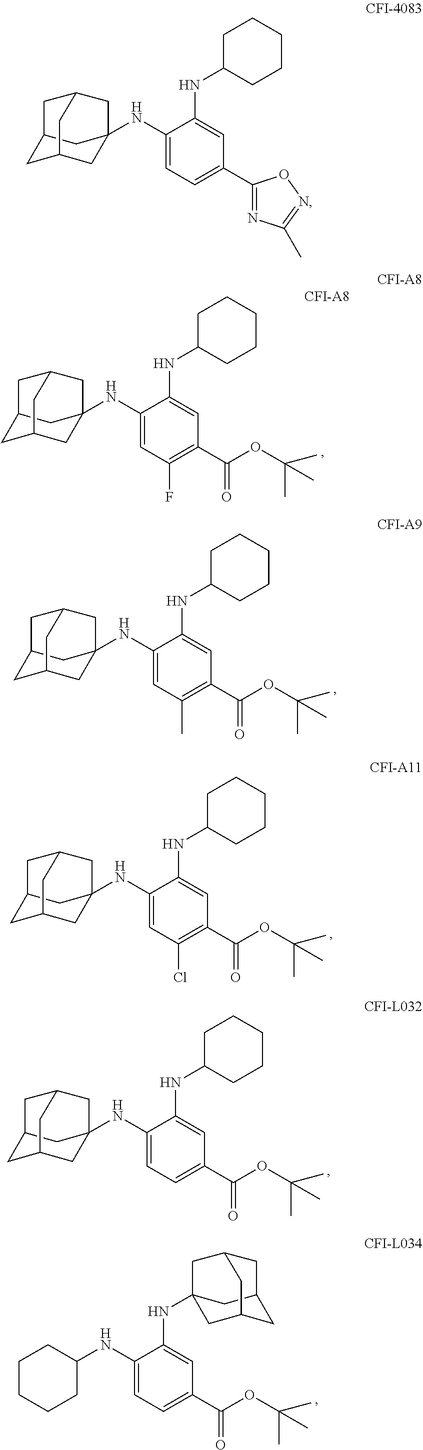 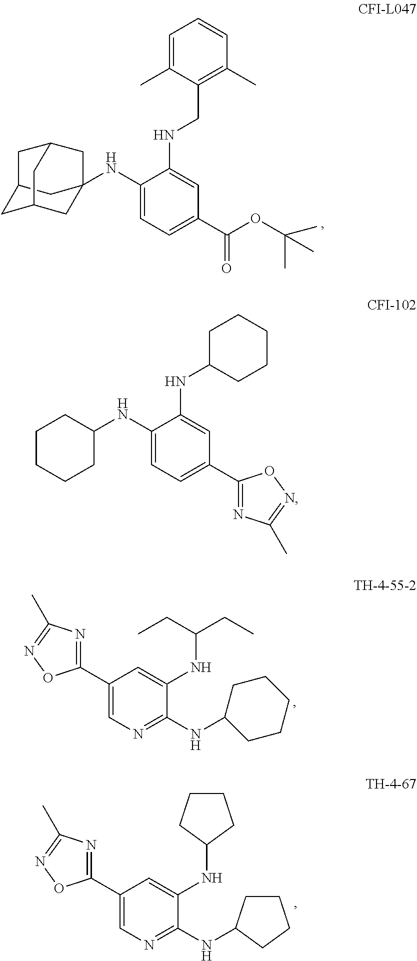 [SMILES: Cc1cccc(C)c1CNc1cc(C(=O)OC(C)(C)C)ccc1NC12CC3CC(CC(C3)C1)C2 ; Cc1noc(-c2ccc(NC3CCCCC3)c(NC3CCCCC3)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
or an N-oxide, crystalline form, hydrate, or a pharmaceutically acceptable salt thereof.

12. A pharmaceutical composition according to claim 7, wherein the one or more compounds have a structure selected from the group consisting of: 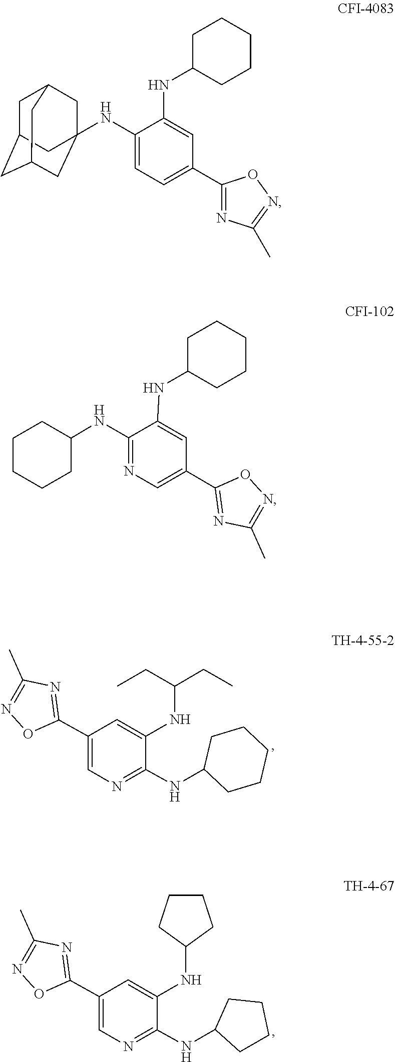 [SMILES: Cc1noc(-c2ccc(NC34CC5CC(CC(C5)C3)C4)c(NC3CCCCC3)c2)n1 ; Cc1noc(-c2cnc(NC3CCCCC3)c(NC3CCCCC3)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
or an N-oxide, crystalline form, hydrate, or a pharmaceutically acceptable salt thereof.

13. A kit comprising a compound according to any one of claims 1-6 together with instructions for the use of the compound.

14. A kit comprising a pharmaceutical composition according to any one of claims 7-12 together with instructions for the use of the pharmaceutical composition.

15. A method for treating or ameliorating the effects of a disorder in a subject in need thereof comprising administering to the subject an effective amount of one or more compounds having the structure of formula (1): 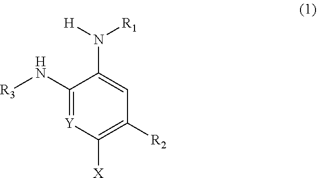
wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is 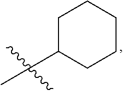 [SMILES: *C1CCCCC1]
R₂ cannot be 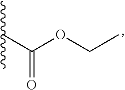 [SMILES: *C(=O)OCC]
the compound is not

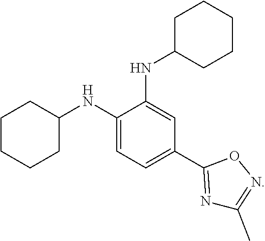 [SMILES: Cc1noc(-c2ccc(NC3CCCCC3)c(NC3CCCCC3)c2)n1]

16. The method according to claim 15, wherein the one or more compounds have the structure of formula (1a): 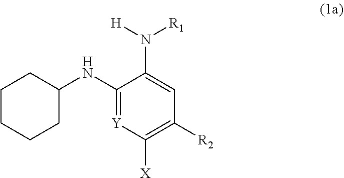
wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H and Y is —CH, R₂ cannot be 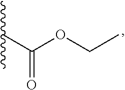 [SMILES: *C(=O)OCC]
the compound is not

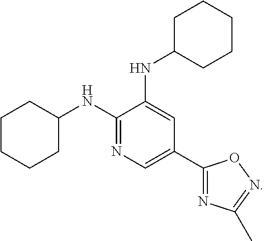 [SMILES: Cc1noc(-c2cnc(NC3CCCCC3)c(NC3CCCCC3)c2)n1]

17. The method according to claim 15, wherein the one or more compounds have the structure of formula (1b): 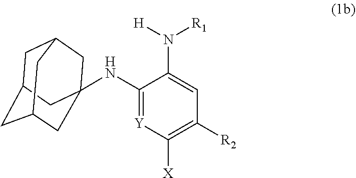
wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof.

18. The method according to claim 15, wherein the one or more compounds are selected from the group consisting of: 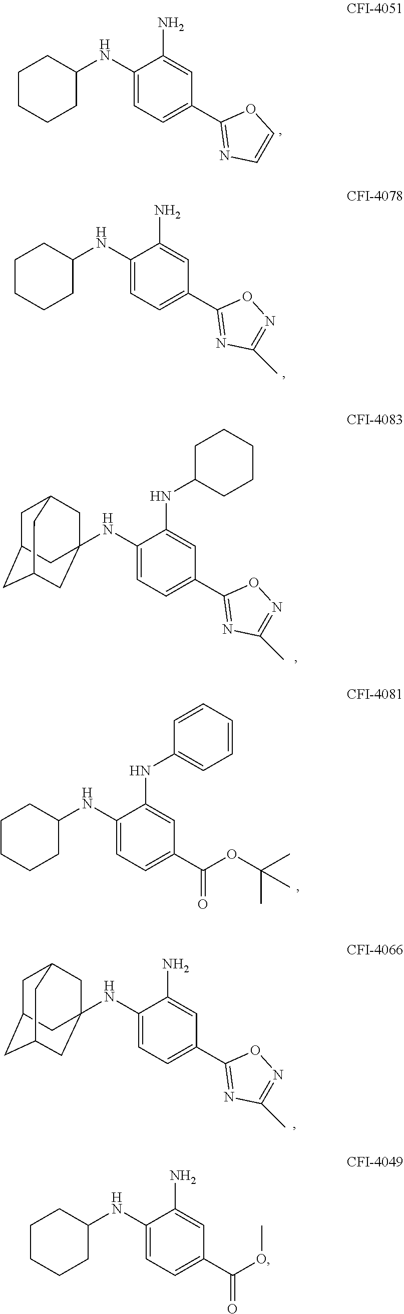 [SMILES: Nc1cc(-c2ncco2)ccc1NC1CCCCC1 ; Cc1noc(-c2ccc(NC3CCCCC3)c(N)c2)n1 ; Cc1noc(-c2ccc(NC34CC5CC(CC(C5)C3)C4)c(NC3CCCCC3)c2)n1 ; CC(C)(C)OC(=O)c1ccc(NC2CCCCC2)c(Nc2ccccc2)c1 ; Cc1noc(-c2ccc(NC34CC5CC(CC(C5)C3)C4)c(N)c2)n1 ; COC(=O)c1ccc(NC2CCCCC2)c(N)c1] 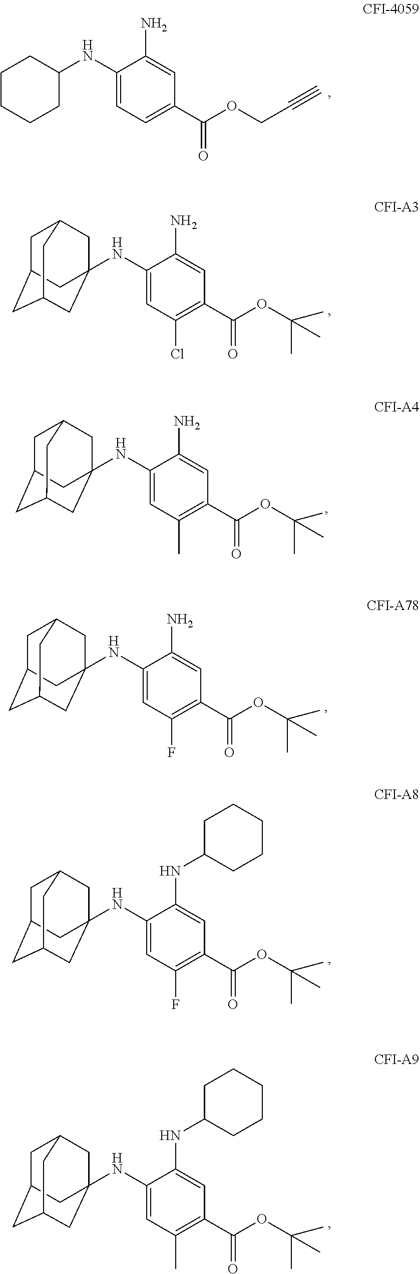 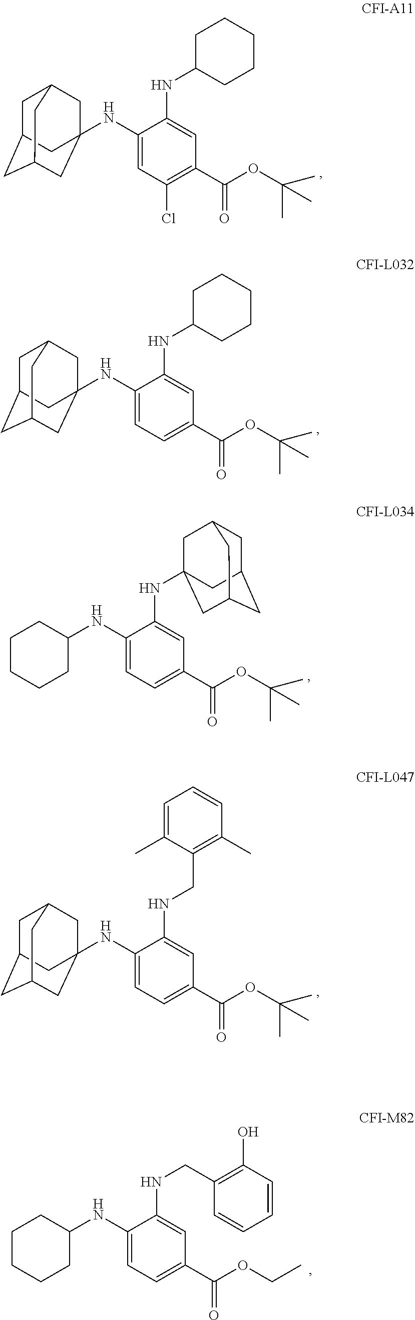 [SMILES: CC(C)(C)OC(=O)c1cc(NC2CCCCC2)c(NC23CC4CC(CC(C4)C2)C3)cc1Cl ; CC(C)(C)OC(=O)c1ccc(NC23CC4CC(CC(C4)C2)C3)c(NC2CCCCC2)c1 ; CC(C)(C)OC(=O)c1ccc(NC2CCCCC2)c(NC23CC4CC(CC(C4)C2)C3)c1 ; Cc1cccc(C)c1CNc1cc(C(=O)OC(C)(C)C)ccc1NC12CC3CC(CC(C3)C1)C2 ; CCOC(=O)c1ccc(NC2CCCCC2)c(NCc2ccccc2O)c1] 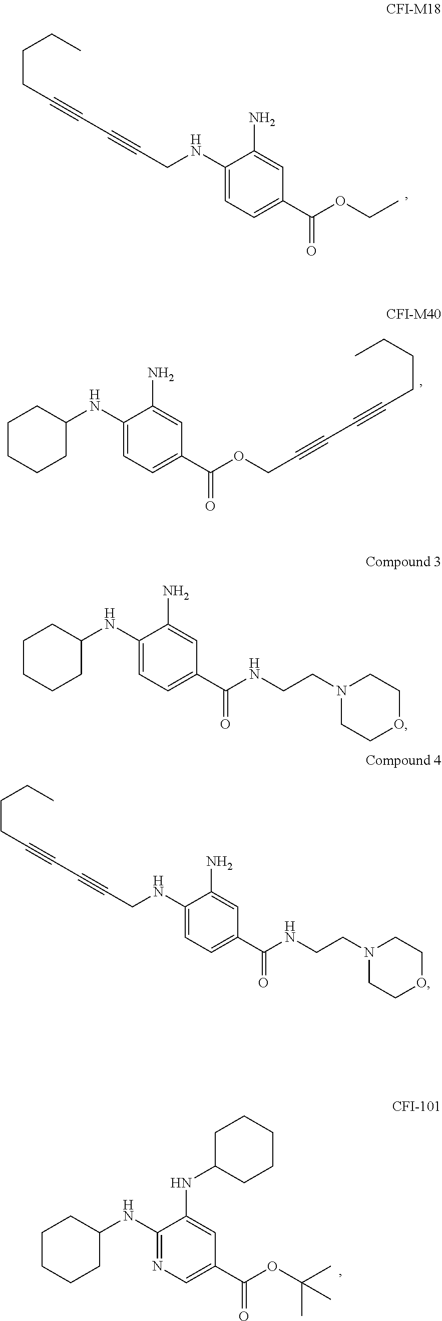 [SMILES: CCCCC#CC#CCNc1ccc(C(=O)OCC)cc1N ; CCCCC#CC#CCOC(=O)c1ccc(NC2CCCCC2)c(N)c1 ; Nc1cc(C(=O)NCCN2CCOCC2)ccc1NC1CCCCC1 ; CCCCC#CC#CCNc1ccc(C(=O)NCCN2CCOCC2)cc1N ; CC(C)(C)OC(=O)c1cnc(NC2CCCCC2)c(NC2CCCCC2)c1] 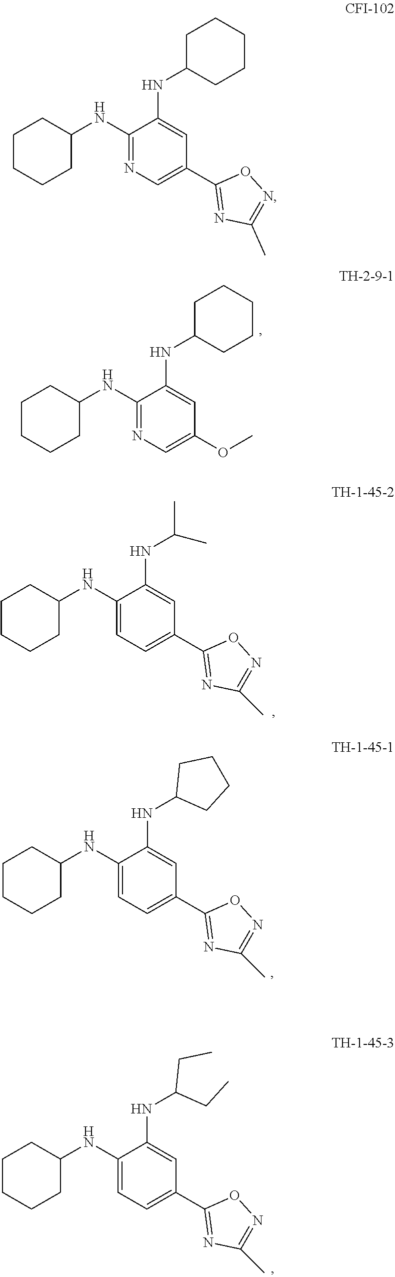 [SMILES: Cc1noc(-c2cnc(NC3CCCCC3)c(NC3CCCCC3)c2)n1 ; COc1cnc(NC2CCCCC2)c(NC2CCCCC2)c1 ; Cc1noc(-c2ccc(NC3CCCCC3)c(NC(C)C)c2)n1 ; Cc1noc(-c2ccc(NC3CCCCC3)c(NC3CCCC3)c2)n1 ; CCC(CC)Nc1cc(-c2nc(C)no2)ccc1NC1CCCCC1] 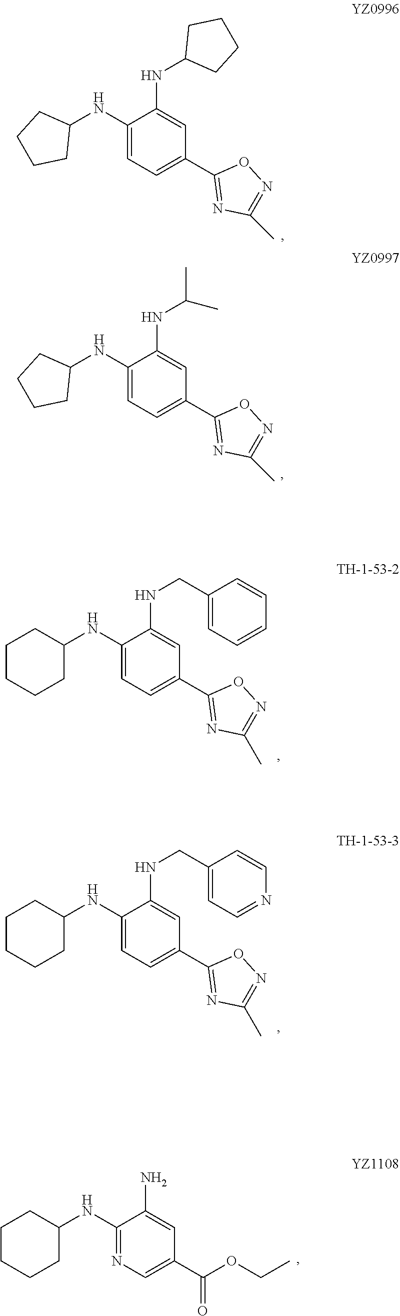 [SMILES: Cc1noc(-c2ccc(NC3CCCC3)c(NC3CCCC3)c2)n1 ; Cc1noc(-c2ccc(NC3CCCC3)c(NC(C)C)c2)n1 ; Cc1noc(-c2ccc(NC3CCCCC3)c(NCc3ccccc3)c2)n1 ; Cc1noc(-c2ccc(NC3CCCCC3)c(NCc3ccncc3)c2)n1 ; CCOC(=O)c1cnc(NC2CCCCC2)c(N)c1] 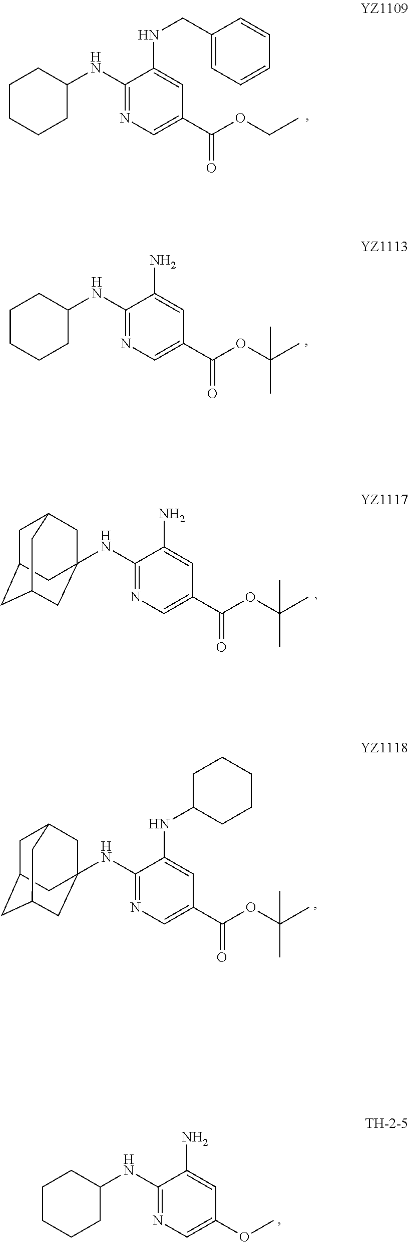 [SMILES: CCOC(=O)c1cnc(NC2CCCCC2)c(NCc2ccccc2)c1 ; CC(C)(C)OC(=O)c1cnc(NC2CCCCC2)c(N)c1 ; CC(C)(C)OC(=O)c1cnc(NC23CC4CC(CC(C4)C2)C3)c(N)c1 ; CC(C)(C)OC(=O)c1cnc(NC23CC4CC(CC(C4)C2)C3)c(NC2CCCCC2)c1 ; COc1cnc(NC2CCCCC2)c(N)c1] 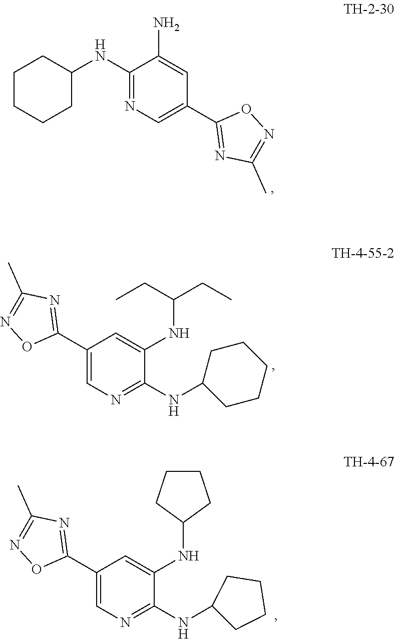 [SMILES: Cc1noc(-c2cnc(NC3CCCCC3)c(N)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
or an N-oxide, crystalline form, hydrate, or a pharmaceutically acceptable salt thereof.

19. The method according to claim 15, wherein the one or more compounds are selected from the group consisting of: 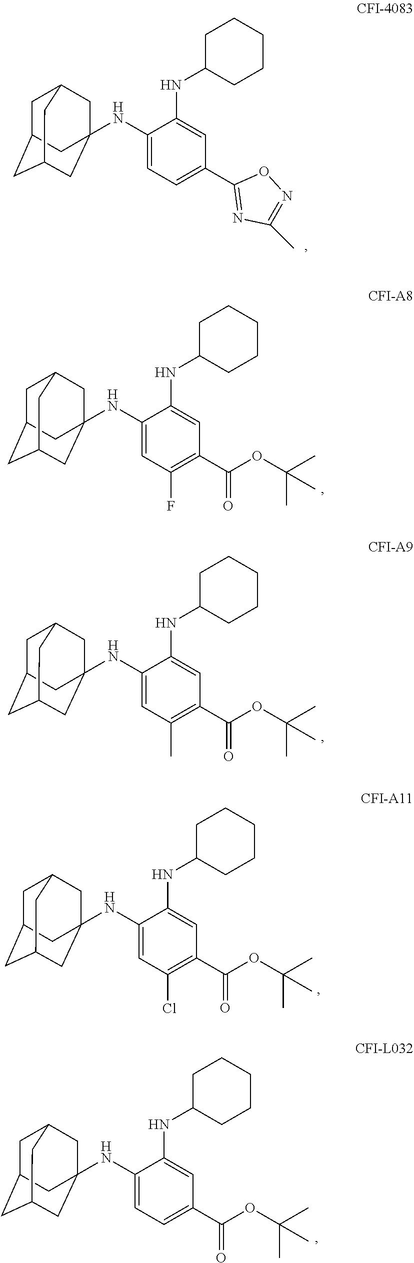 [SMILES: Cc1noc(-c2ccc(NC34CC5CC(CC(C5)C3)C4)c(NC3CCCCC3)c2)n1 ; CC(C)(C)OC(=O)c1cc(NC2CCCCC2)c(NC23CC4CC(CC(C4)C2)C3)cc1F ; Cc1cc(NC23CC4CC(CC(C4)C2)C3)c(NC2CCCCC2)cc1C(=O)OC(C)(C)C ; CC(C)(C)OC(=O)c1cc(NC2CCCCC2)c(NC23CC4CC(CC(C4)C2)C3)cc1Cl ; CC(C)(C)OC(=O)c1ccc(NC23CC4CC(CC(C4)C2)C3)c(NC2CCCCC2)c1] 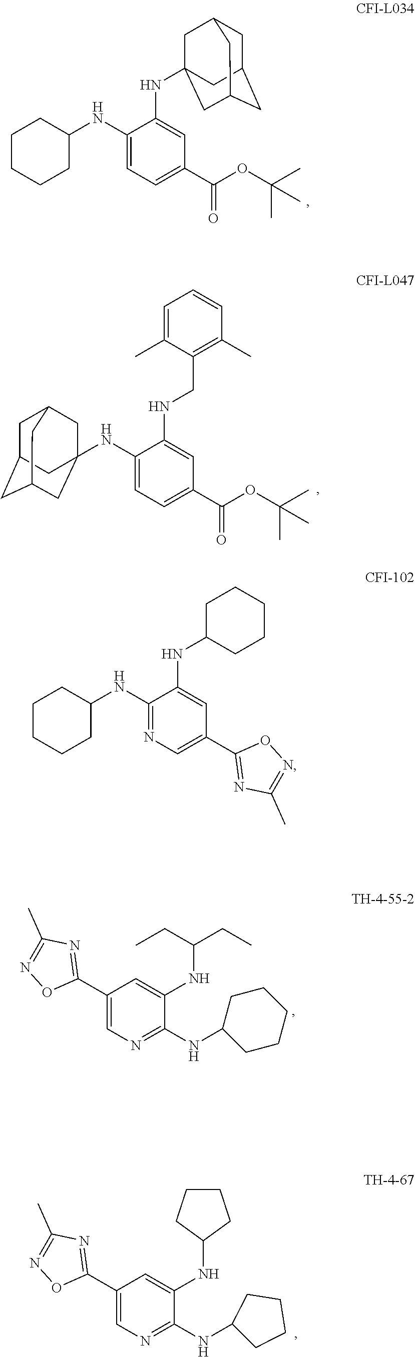 [SMILES: CC(C)(C)OC(=O)c1ccc(NC2CCCCC2)c(NC23CC4CC(CC(C4)C2)C3)c1 ; Cc1cccc(C)c1CNc1cc(C(=O)OC(C)(C)C)ccc1NC12CC3CC(CC(C3)C1)C2 ; Cc1noc(-c2cnc(NC3CCCCC3)c(NC3CCCCC3)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
or an N-oxide, crystalline form, hydrate, or a pharmaceutically acceptable salt thereof.

20. The method according to claim 15, wherein the one or more compounds are selected from the group consisting of: 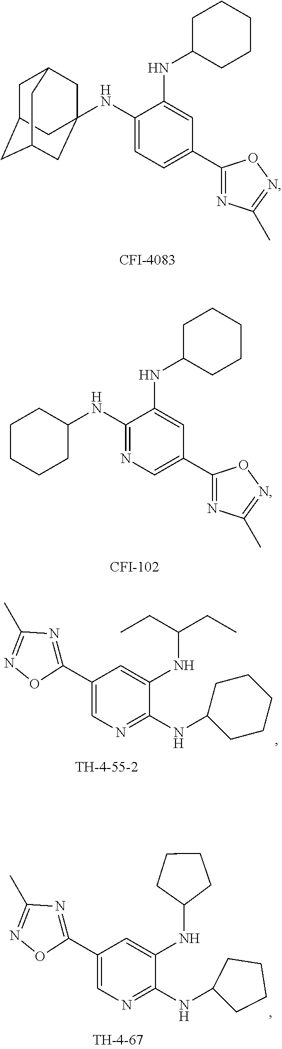 [SMILES: Cc1noc(-c2ccc(NC34CC5CC(CC(C5)C3)C4)c(NC3CCCCC3)c2)n1 ; Cc1noc(-c2cnc(NC3CCCCC3)c(NC3CCCCC3)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
or an N-oxide, crystalline form, hydrate, or a pharmaceutically acceptable salt thereof.

21. The method according to claim 15, wherein the disorder is a degenerative disease that involves lipid peroxidation.

22. The method according to claim 15, wherein the disorder is an excitotoxic disease involving oxidative cell death.

23. The method according to claim 15, wherein the disorder is selected from the group consisting of epilepsy, kidney disease, stroke, myocardial infarction, type I diabetes, TBI, PVL, and neurodegenerative disease.

24. The method according to claim 23, wherein the neurodegenerative disease is selected from the group consisting of Alzheimer's, Parkinson's, Amyotrophic lateral sclerosis, Friedreich's ataxia, Multiple sclerosis, Huntington's Disease, Transmissible spongiform encephalopathy, Charcot-Marie-Tooth disease, Dementia with Lewy bodies, Corticobasal degeneration, Progressive supranuclear palsy, Chronic Traumatic Encephalopathy (CTE), and Hereditary spastic paraparesis.

25. The method according to any one of claims 15-20 further comprising co-administering to the subject an effective amount of one or more additional therapeutic agents selected from the group consisting of 5-hydroxytryptophan, Activase, AFQ056 (Novartis), Aggrastat, Albendazole, alpha-lipoic acid/L-acetyl carnitine, Alteplase, Amantadine (Symmetrel), amlodipine, Ancrod, Apomorphine (Apokyn), Arimoclomol, Arixtra, Armodafinil, Ascorbic acid, Ascriptin, Aspirin, atenolol, Avonex, baclofen (Lioresal), Banzel, Benztropine (Cogentin), Betaseron, BGG492 (Novartis Corp.), Botulinum toxin, Bufferin, Carbatrol®, Carbidopa/levodopa immediate-release (Sinemet), Carbidopa/levodopa oral disintegrating (Parcopa), Carbidopa/levodopa/Entacapone (Stalevo), CERE-110: Adeno-Associated Virus Delivery of NGF (Ceregene), cerebrolysin, CinnoVex, citalopram, citicoline, Clobazam, Clonazepam, Clopidogrel, clozapine (Clozaril), Coenzyme Q, Creatine, dabigatran, dalteparin, Dapsone, Davunetide, Deferiprone, Depakene®, Depakote ER®, Depakote®, Desmoteplase, Diastat, Diazepam, Digoxin, Dilantin®, Dimebon, dipyridamole, divalproex (Depakote), Donepezil (Aricept), EGb 761, Eldepryl, ELND002 (Elan Pharmaceuticals), Enalapril, enoxaparin, Entacapone (Comtan), epoetin alfa, Eptifibatide, Erythropoietin, Escitalopram, Eslicarbazepine acetate, Esmolol, Ethosuximide, Ethyl-EPA (Miraxion™), Exenatide, Extavia, Ezogabine, Felbamate, Felbatol®, Fingolimod (Gilenya), fluoxetine (Prozac), fondaparinux, Fragmin, Frisium, Gabapentin, Gabitril®, Galantamine, Glatiramer (Copaxone), haloperidol (Haldol), Heparin, human chorionic gonadotropin (hCG), Idebenone, Inovelon®, insulin, Interferon beta 1a, Interferon beta 1 b, ioflupane 1231 (DATSCAN®), IPX066 (Impax Laboratories Inc.), JNJ-26489112 (Johnson and Johnson), Keppra®, Klonopin, Lacosamide, L-Alpha glycerylphosphorylcholine, Lamictal®, Lamotrigine, Levetiracetam, liraglutide, Lisinopril, Lithium carbonate, Lopressor, Lorazepam, losartan, Lovenox, Lu AA24493, Luminal, LY450139 (Eli Lilly), Lyrica, Masitinib, Mecobalamin, Memantine, methylprednisolone, metoprolol tartrate, Minitran, Minocycline, mirtazapine, Mitoxantrone (Novantrone), Mysoline®, Natalizumab (Tysabri), Neurontin®, Niacinamide, Nitro-Bid, Nitro-Dur, nitroglycerin, Nitrolingual, Nitromist, Nitrostat, Nitro-Time, Norepinephrine (NOR), Carbamazepine, octreotide, Onfi®, Oxcarbazepine, Oxybutinin chloride, PF-04360365 (Pfizer), Phenobarbital, Phenytek®, Phenytoin, piclozotan, Pioglitazone, Plavix, Potiga, Pram ipexole (Mirapex), pramlintide, Prednisone, Prim idone, Prinivil, probenecid, Propranolol, PRX-00023 (EPIX Pharmaceuticals Inc.), PXT3003, Quinacrine, Ramelteon, Rasagiline (Azilect), Rebif, ReciGen, remacemide, Resveratrol, Retavase, reteplase, riluzole (Rilutek), Rivastigmine (Exelon), Ropinirole (Requip), Rotigotine (Neupro), Rufinamide, Sabril, safinamide (EMD Serono), Salagen, Sarafem, Selegiline (1-deprenyl, Eldepryl), SEN0014196 (Siena Biotech), sertraline (Zoloft), Simvastatin, Sodium Nitroprussiate (NPS), sodium phenylbutyrate, Stanback Headache Powder, Tacrine (Cognex), Tamoxifen, tauroursodeoxycholic acid (TUDCA), Tegretol®, Tenecteplase, Tenormin, Tetrabenazine (Xenazine), THR-18 (Thrombotech Ltd.), Tiagabine, Tideglusib, tirofiban, tissue plasminogen activator (tPA), tizanidine (Zanaflex), TNKase, Tolcapone (Tasmar), Tolterodine, Topamax®, Topiramate, Trihexyphenidyl (formerly Artane), Trileptal®, ursodiol, Valproic Acid, valsartan, Varenicline (Pfizer), Vimpat, Vitamin E, Warfarin, Zarontin®, Zestril, Zonegran®, Zonisamide, Zydis selegiline HCL Oral disintegrating (Zelapar), and combinations thereof.

26. The method according to claim 15, wherein the subject is a mammal.

27. The method according to claim 26, wherein the mammal is selected from the group consisting of humans, veterinary animals, and agricultural animals.

28. The method according to claim 15, wherein the subject is a human.

29. A method for treating or ameliorating the effects of a disorder in a subject in need thereof comprising administering to the subject an effective amount of a pharmaceutical composition comprising a pharmaceutically acceptable carrier or diluent and one or more compounds having the structure of formula (1): 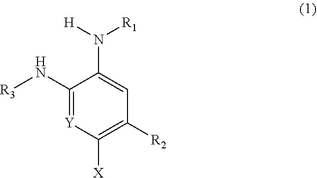
wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂carbocycle, or a polyyne, wherein each of the C₃₋₁₂carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is 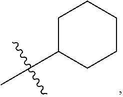 [SMILES: *C1CCCCC1]
R₂ cannot be 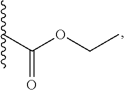 [SMILES: *C(=O)OCC]
the compound is not

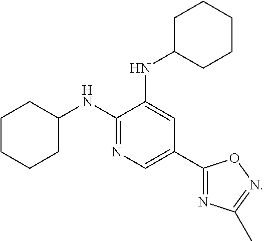 [SMILES: Cc1noc(-c2cnc(NC3CCCCC3)c(NC3CCCCC3)c2)n1]

30. The method according to claim 29, wherein the one or more compounds have the structure of formula (1a): 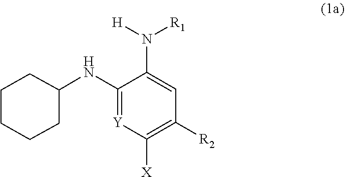
wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H and Y is —CH, R₂ cannot be 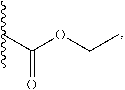 [SMILES: *C(=O)OCC]
the compound is not

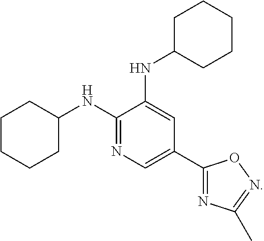 [SMILES: Cc1noc(-c2cnc(NC3CCCCC3)c(NC3CCCCC3)c2)n1]

31. The method according to claim 29, wherein the one or more compounds have the structure of formula (1b): 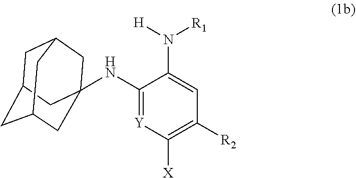
wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof.

32. The method according to claim 29, wherein the one or more compounds are selected from the group consisting of: 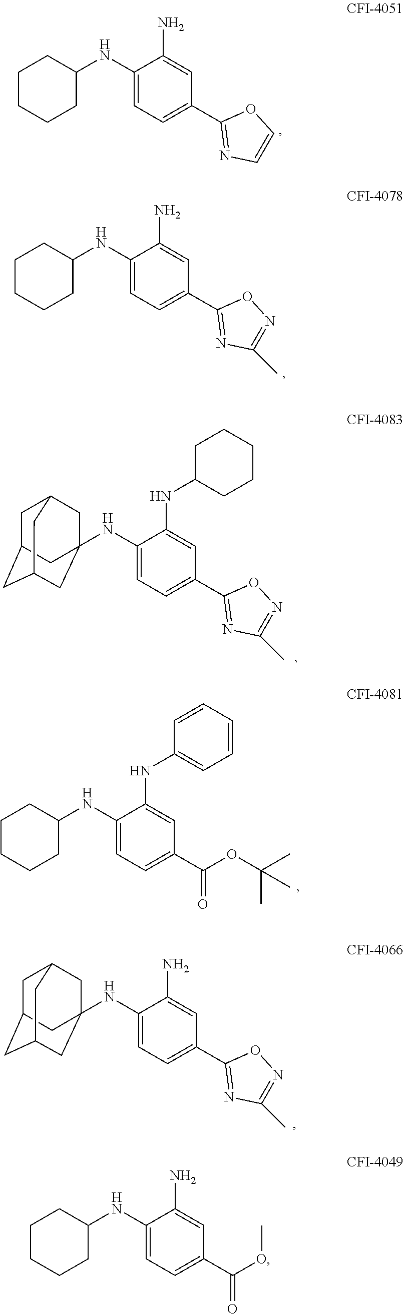 [SMILES: Nc1cc(-c2ncco2)ccc1NC1CCCCC1 ; Cc1noc(-c2ccc(NC3CCCCC3)c(N)c2)n1 ; Cc1noc(-c2ccc(NC34CC5CC(CC(C5)C3)C4)c(NC3CCCCC3)c2)n1 ; CC(C)(C)OC(=O)c1ccc(NC2CCCCC2)c(Nc2ccccc2)c1 ; Cc1noc(-c2ccc(NC34CC5CC(CC(C5)C3)C4)c(N)c2)n1 ; COC(=O)c1ccc(NC2CCCCC2)c(N)c1] 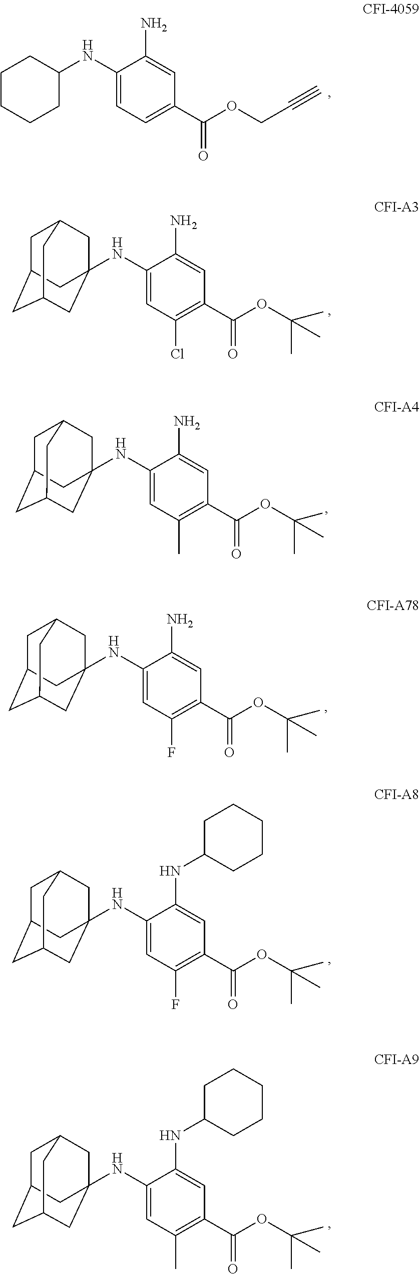 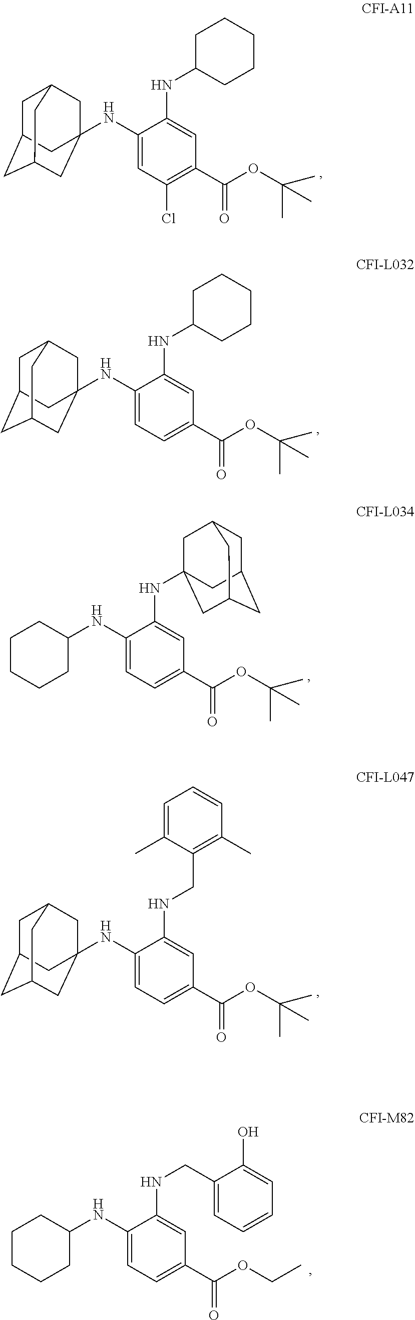 [SMILES: CC(C)(C)OC(=O)c1cc(NC2CCCCC2)c(NC23CC4CC(CC(C4)C2)C3)cc1Cl ; CC(C)(C)OC(=O)c1ccc(NC23CC4CC(CC(C4)C2)C3)c(NC2CCCCC2)c1 ; CC(C)(C)OC(=O)c1ccc(NC2CCCCC2)c(NC23CC4CC(CC(C4)C2)C3)c1 ; Cc1cccc(C)c1CNc1cc(C(=O)OC(C)(C)C)ccc1NC12CC3CC(CC(C3)C1)C2 ; CCOC(=O)c1ccc(NC2CCCCC2)c(NCc2ccccc2O)c1] 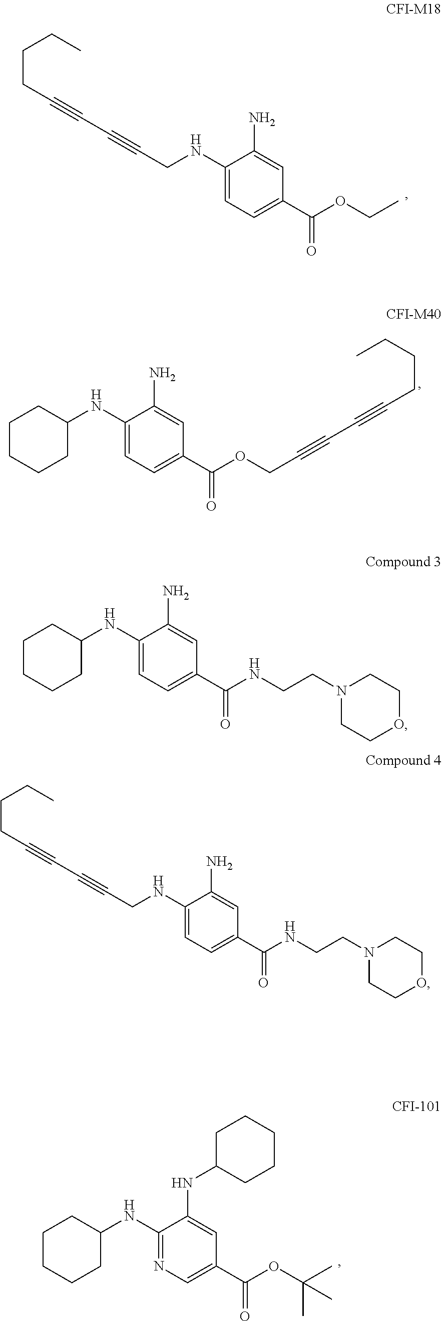 [SMILES: CCCCC#CC#CCNc1ccc(C(=O)OCC)cc1N ; CCCCC#CC#CCOC(=O)c1ccc(NC2CCCCC2)c(N)c1 ; Nc1cc(C(=O)NCCN2CCOCC2)ccc1NC1CCCCC1 ; CCCCC#CC#CCNc1ccc(C(=O)NCCN2CCOCC2)cc1N ; CC(C)(C)OC(=O)c1cnc(NC2CCCCC2)c(NC2CCCCC2)c1] 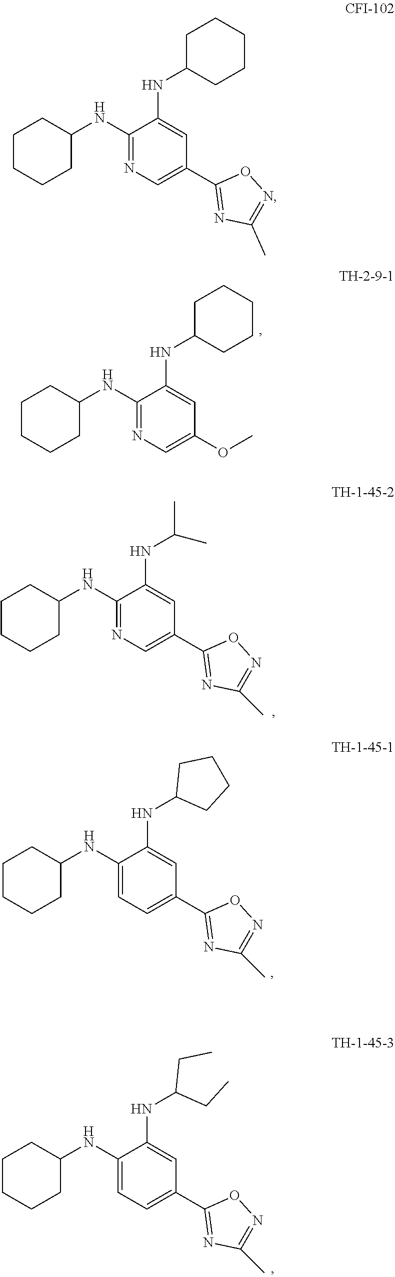 [SMILES: Cc1noc(-c2cnc(NC3CCCCC3)c(NC3CCCCC3)c2)n1 ; COc1cnc(NC2CCCCC2)c(NC2CCCCC2)c1 ; Cc1noc(-c2cnc(NC3CCCCC3)c(NC(C)C)c2)n1 ; Cc1noc(-c2ccc(NC3CCCCC3)c(NC3CCCC3)c2)n1 ; CCC(CC)Nc1cc(-c2nc(C)no2)ccc1NC1CCCCC1] 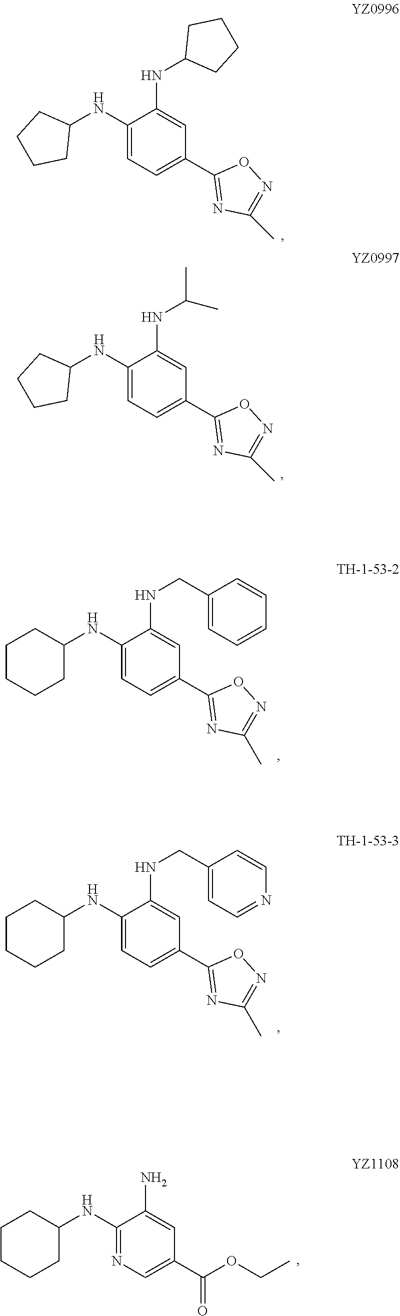 [SMILES: Cc1noc(-c2ccc(NC3CCCC3)c(NC3CCCC3)c2)n1 ; Cc1noc(-c2ccc(NC3CCCC3)c(NC(C)C)c2)n1 ; Cc1noc(-c2ccc(NC3CCCCC3)c(NCc3ccccc3)c2)n1 ; Cc1noc(-c2ccc(NC3CCCCC3)c(NCc3ccncc3)c2)n1 ; CCOC(=O)c1cnc(NC2CCCCC2)c(N)c1] 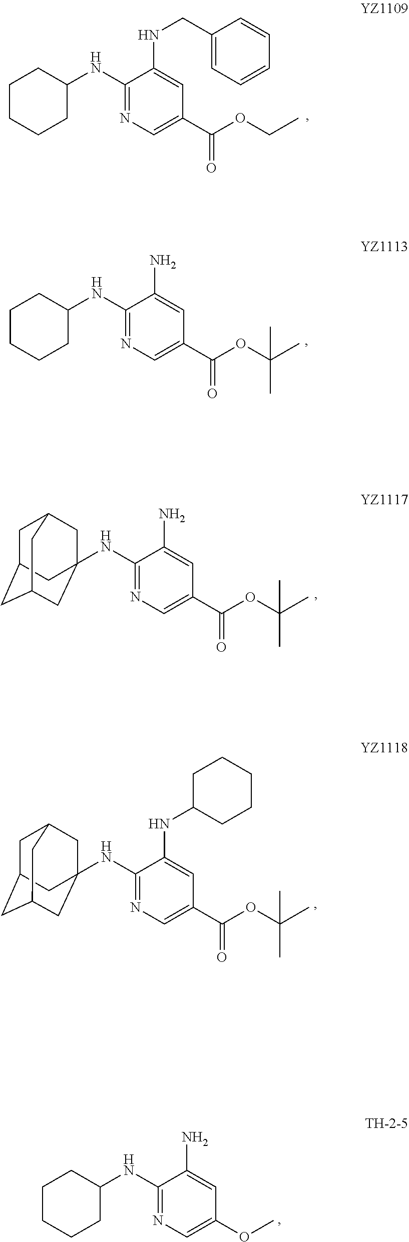 [SMILES: CCOC(=O)c1cnc(NC2CCCCC2)c(NCc2ccccc2)c1 ; CC(C)(C)OC(=O)c1cnc(NC2CCCCC2)c(N)c1 ; CC(C)(C)OC(=O)c1cnc(NC23CC4CC(CC(C4)C2)C3)c(N)c1 ; CC(C)(C)OC(=O)c1cnc(NC23CC4CC(CC(C4)C2)C3)c(NC2CCCCC2)c1 ; COc1cnc(NC2CCCCC2)c(N)c1] 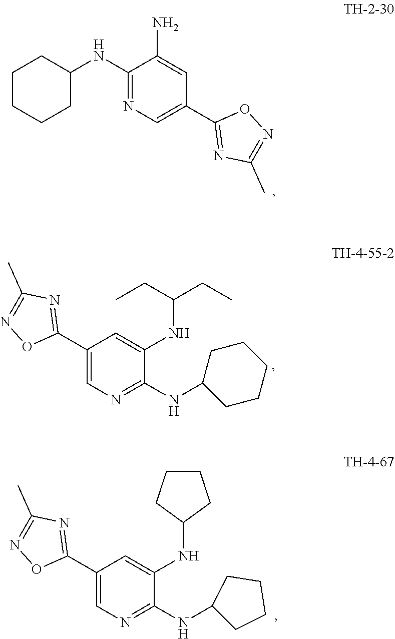 [SMILES: Cc1noc(-c2cnc(NC3CCCCC3)c(N)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
or an N-oxide, crystalline form, hydrate, or a pharmaceutically acceptable salt thereof.

33. The method according to claim 29, wherein the one or more compounds are selected from the group consisting of: 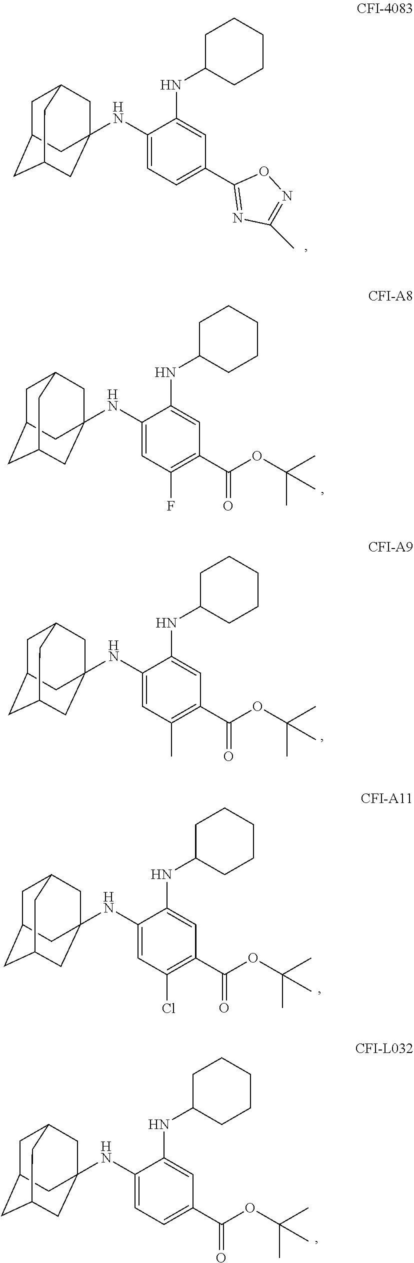 [SMILES: Cc1noc(-c2ccc(NC34CC5CC(CC(C5)C3)C4)c(NC3CCCCC3)c2)n1 ; CC(C)(C)OC(=O)c1cc(NC2CCCCC2)c(NC23CC4CC(CC(C4)C2)C3)cc1F ; Cc1cc(NC23CC4CC(CC(C4)C2)C3)c(NC2CCCCC2)cc1C(=O)OC(C)(C)C ; CC(C)(C)OC(=O)c1cc(NC2CCCCC2)c(NC23CC4CC(CC(C4)C2)C3)cc1Cl ; CC(C)(C)OC(=O)c1ccc(NC23CC4CC(CC(C4)C2)C3)c(NC2CCCCC2)c1] 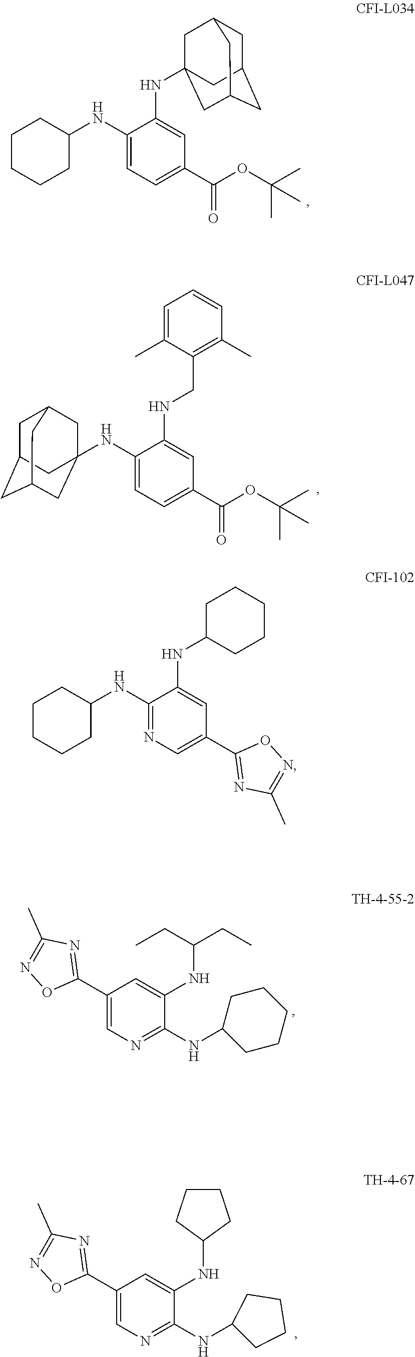 [SMILES: CC(C)(C)OC(=O)c1ccc(NC2CCCCC2)c(NC23CC4CC(CC(C4)C2)C3)c1 ; Cc1cccc(C)c1CNc1cc(C(=O)OC(C)(C)C)ccc1NC12CC3CC(CC(C3)C1)C2 ; Cc1noc(-c2cnc(NC3CCCCC3)c(NC3CCCCC3)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
or an N-oxide, crystalline form, hydrate, or a pharmaceutically acceptable salt thereof.

34. The method according to claim 29, wherein the one or more compounds are selected from the group consisting of: 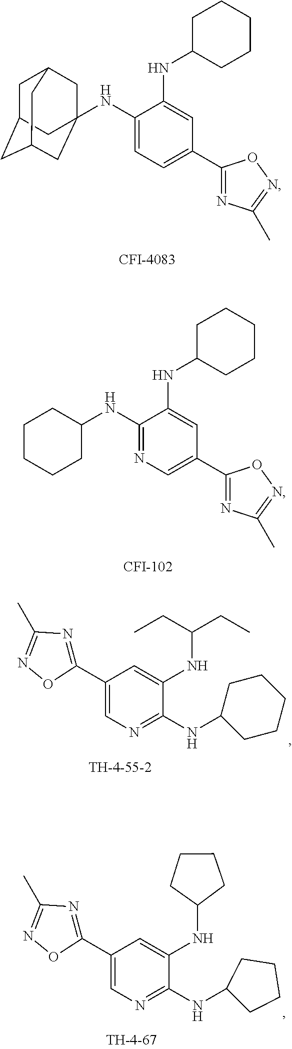 [SMILES: Cc1noc(-c2ccc(NC34CC5CC(CC(C5)C3)C4)c(NC3CCCCC3)c2)n1 ; Cc1noc(-c2cnc(NC3CCCCC3)c(NC3CCCCC3)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
or an N-oxide, crystalline form, hydrate, or a pharmaceutically acceptable salt thereof.

35. The method according to claim 29, wherein the disorder is a degenerative disease that involves lipid peroxidation.

36. The method according to claim 29, wherein the disorder is an excitotoxic disease involving oxidative cell death.

37. The method according to claim 29, wherein the disorder is selected from the group consisting of epilepsy, kidney disease, stroke, myocardial infarction, type I diabetes, TBI, PVL, and neurodegenerative disease.

38. The method according to claim 37, wherein the neurodegenerative disease is selected from the group consisting of Alzheimer's, Parkinson's, Amyotrophic lateral sclerosis, Friedreich's ataxia, Multiple sclerosis, Huntington's Disease, Transmissible spongiform encephalopathy, Charcot-Marie-Tooth disease, Dementia with Lewy bodies, Corticobasal degeneration, Progressive supranuclear palsy, Chronic Traumatic Encephalopathy (CTE), and Hereditary spastic paraparesis.

39. The method according to any one of claims 29-34 further comprising co-administering to the subject an effective amount of one or more therapeutic agents selected from the group consisting of: 5-hydroxytryptophan, Activase, AFQ056 (Novartis), Aggrastat, Albendazole, alpha-lipoic acid/L-acetyl carnitine, Alteplase, Amantadine (Symmetrel), amlodipine, Ancrod, Apomorphine (Apokyn), Arimoclomol, Arixtra, Armodafinil, Ascorbic acid, Ascriptin, Aspirin, atenolol, Avonex, baclofen (Lioresal), Banzel, Benztropine (Cogentin), Betaseron, BGG492 (Novartis Corp.), Botulinum toxin, Bufferin, Carbatrol®, Carbidopa/levodopa immediate-release (Sinemet), Carbidopa/levodopa oral disintegrating (Parcopa), Carbidopa/levodopa/Entacapone (Stalevo), CERE-110: Adeno-Associated Virus Delivery of NGF (Ceregene), cerebrolysin, CinnoVex, citalopram, citicoline, Clobazam, Clonazepam, Clopidogrel, clozapine (Clozaril), Coenzyme Q, Creatine, dabigatran, dalteparin, Dapsone, Davunetide, Deferiprone, Depakene®, Depakote ER®, Depakote®, Desmoteplase, Diastat, Diazepam, Digoxin, Dilantin®, Dimebon, dipyridamole, divalproex (Depakote), Donepezil (Aricept), EGb 761, Eldepryl, ELND002 (Elan Pharmaceuticals), Enalapril, enoxaparin, Entacapone (Comtan), epoetin alfa, Eptifibatide, Erythropoietin, Escitalopram, Eslicarbazepine acetate, Esmolol, Ethosuximide, Ethyl-EPA (Miraxion™), Exenatide, Extavia, Ezogabine, Felbamate, Felbatol®, Fingolimod (Gilenya), fluoxetine (Prozac), fondaparinux, Fragmin, Frisium, Gabapentin, Gabitril®, Galantamine, Glatiramer (Copaxone), haloperidol (Haldol), Heparin, human chorionic gonadotropin (hCG), Idebenone, Inovelon®, insulin, Interferon beta 1a, Interferon beta 1 b, ioflupane 1231 (DATSCAN®), IPX066 (Impax Laboratories Inc.), JNJ-26489112 (Johnson and Johnson), Keppra®, Klonopin, Lacosamide, L-Alpha glycerylphosphorylcholine, Lamictal®, Lamotrigine, Levetiracetam, liraglutide, Lisinopril, Lithium carbonate, Lopressor, Lorazepam, losartan, Lovenox, Lu AA24493, Luminal, LY450139 (Eli Lilly), Lyrica, Masitinib, Mecobalamin, Memantine, methylprednisolone, metoprolol tartrate, Minitran, Minocycline, mirtazapine, Mitoxantrone (Novantrone), Mysoline®, Natalizumab (Tysabri), Neurontin®, Niacinamide, Nitro-Bid, Nitro-Dur, nitroglycerin, Nitrolingual, Nitromist, Nitrostat, Nitro-Time, Norepinephrine (NOR), Carbamazepine, octreotide, Onfi®, Oxcarbazepine, Oxybutinin chloride, PF-04360365 (Pfizer), Phenobarbital, Phenytek®, Phenytoin, piclozotan, Pioglitazone, Plavix, Potiga, Pram ipexole (Mirapex), pramlintide, Prednisone, Prim idone, Prinivil, probenecid, Propranolol, PRX-00023 (EPIX Pharmaceuticals Inc.), PXT3003, Quinacrine, Ramelteon, Rasagiline (Azilect), Rebif, ReciGen, remacemide, Resveratrol, Retavase, reteplase, riluzole (Rilutek), Rivastigmine (Exelon), Ropinirole (Requip), Rotigotine (Neupro), Rufinamide, Sabril, safinamide (EMD Serono), Salagen, Sarafem, Selegiline (1-deprenyl, Eldepryl), SEN0014196 (Siena Biotech), sertraline (Zoloft), Simvastatin, Sodium Nitroprussiate (NPS), sodium phenylbutyrate, Stanback Headache Powder, Tacrine (Cognex), Tamoxifen, tauroursodeoxycholic acid (TUDCA), Tegretol®, Tenecteplase, Tenormin, Tetrabenazine (Xenazine), THR-18 (Thrombotech Ltd.), Tiagabine, Tideglusib, tirofiban, tissue plasminogen activator (tPA), tizanidine (Zanaflex), TNKase, Tolcapone (Tasmar), Tolterodine, Topamax®, Topiramate, Trihexyphenidyl (formerly Artane), Trileptal®, ursodiol, Valproic Acid, valsartan, Varenicline (Pfizer), Vimpat, Vitamin E, Warfarin, Zarontin®, Zestril, Zonegran®, Zonisamide, Zydis selegiline HCL Oral disintegrating (Zelapar), and combinations thereof.

40. The method according to claim 29, wherein the subject is a mammal.

41. The method according to claim 40, wherein the mammal is selected from the group consisting of humans, veterinary animals, and agricultural animals.

42. The method according to claim 29, wherein the subject is a human.

43. A method of modulating ferroptosis in a subject in need thereof comprising administering to the subject an effective amount of a ferroptosis inhibitor, which comprises one or more compounds having the structure of formula (1): 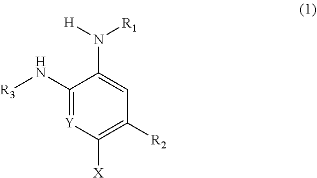
wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is 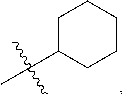 [SMILES: *C1CCCCC1]
R₂ cannot be 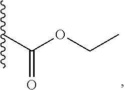 [SMILES: *C(=O)OCC]
the compound is not

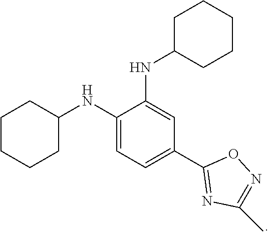 [SMILES: Cc1noc(-c2ccc(NC3CCCCC3)c(NC3CCCCC3)c2)n1]

44. A method of reducing reactive oxygen species (ROS) in a cell comprising contacting a cell with a ferroptosis modulator, which comprises one or more compounds having the structure of formula (1): 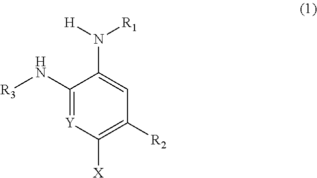
wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is 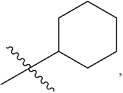 [SMILES: *C1CCCCC1]
R₂ cannot be 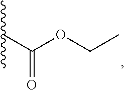 [SMILES: *C(=O)OCC]
the compound is not

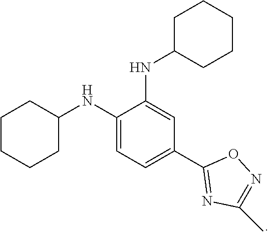 [SMILES: Cc1noc(-c2ccc(NC3CCCCC3)c(NC3CCCCC3)c2)n1]

45. A method for treating or ameliorating the effects of a neurodegenerative disease in a subject in need thereof comprising administering to the subject an effective amount of one or more compounds having the structure of formula (1): 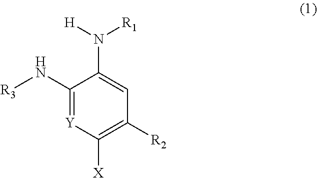
wherein:
R₁ is selected from the group consisting of H, alkyl, aryl, C₁₋₆ alkyl-aryl, C₁₋₆ alkyl-phenolyl, and C₃₋₁₀carbocycle, wherein each of the alkyl, aryl, C₁₋₆ alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂ carbocycle, or a polyyne, wherein each of the C₃₋₁₂ carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is 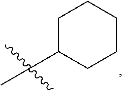 [SMILES: *C1CCCCC1]
R₂ cannot be 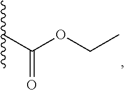 [SMILES: *C(=O)OCC]
the compound is not

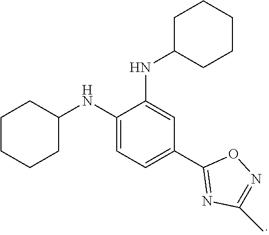 [SMILES: Cc1noc(-c2ccc(NC3CCCCC3)c(NC3CCCCC3)c2)n1]

46. The method according to claim 45, wherein the one or more compounds are selected from the group consisting of: 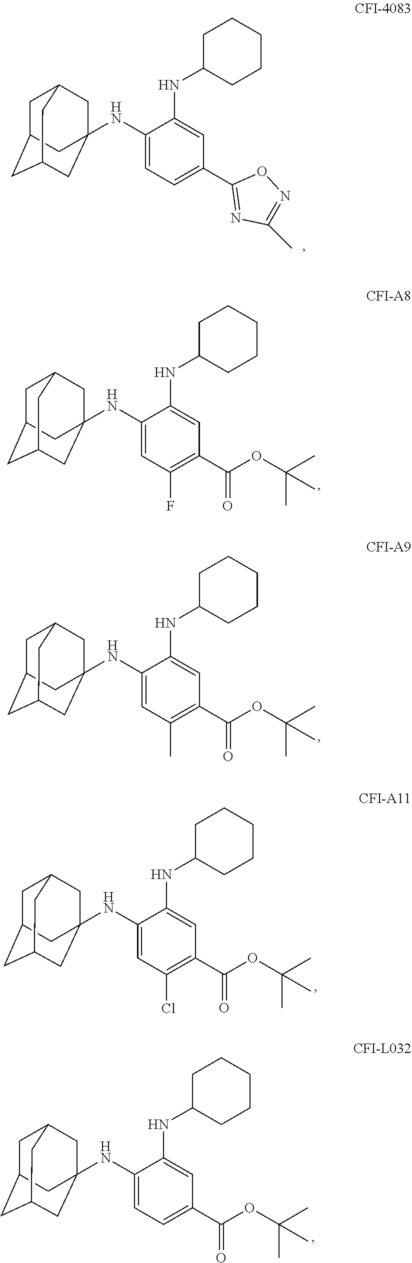 [SMILES: Cc1noc(-c2ccc(NC34CC5CC(CC(C5)C3)C4)c(NC3CCCCC3)c2)n1 ; CC(C)(C)OC(=O)c1cc(NC2CCCCC2)c(NC23CC4CC(CC(C4)C2)C3)cc1F ; Cc1cc(NC23CC4CC(CC(C4)C2)C3)c(NC2CCCCC2)cc1C(=O)OC(C)(C)C ; CC(C)(C)OC(=O)c1cc(NC2CCCCC2)c(NC23CC4CC(CC(C4)C2)C3)cc1Cl ; CC(C)(C)OC(=O)c1ccc(NC23CC4CC(CC(C4)C2)C3)c(NC2CCCCC2)c1] 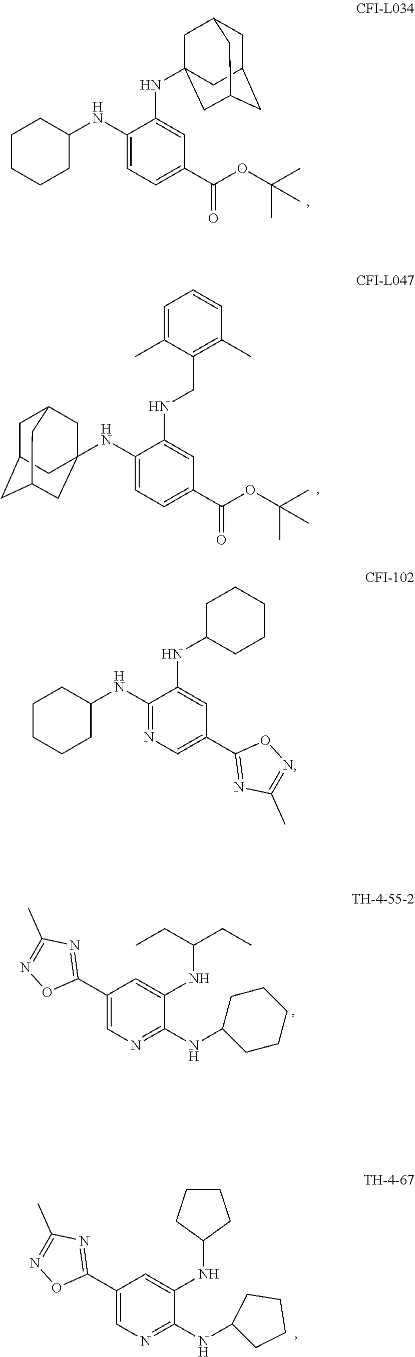 [SMILES: CC(C)(C)OC(=O)c1ccc(NC2CCCCC2)c(NC23CC4CC(CC(C4)C2)C3)c1 ; Cc1cccc(C)c1CNc1cc(C(=O)OC(C)(C)C)ccc1NC12CC3CC(CC(C3)C1)C2 ; Cc1noc(-c2cnc(NC3CCCCC3)c(NC3CCCCC3)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
or an N-oxide, crystalline form, hydrate, or a pharmaceutically acceptable salt thereof.

47. The method according to claim 46, wherein the one or more compounds are selected from the group consisting of: 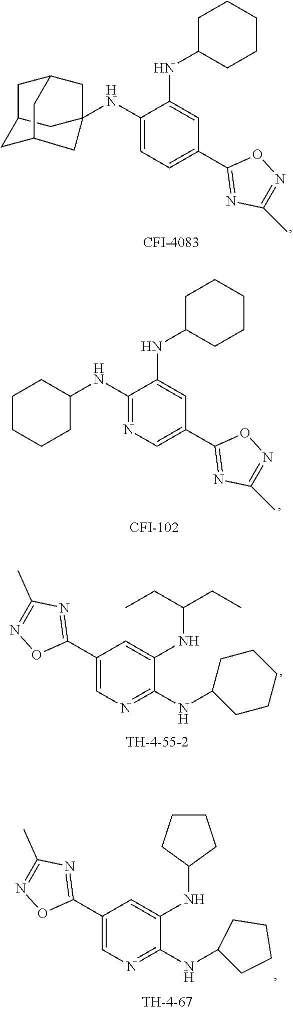 [SMILES: Cc1noc(-c2ccc(NC34CC5CC(CC(C5)C3)C4)c(NC3CCCCC3)c2)n1 ; Cc1noc(-c2cnc(NC3CCCCC3)c(NC3CCCCC3)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
or an N-oxide, crystalline form, hydrate, or a pharmaceutically acceptable salt thereof.

48. The method according to claim 45, wherein the neurodegenerative disease is selected from the group consisting of Alzheimer's, Parkinson's, Amyotrophic lateral sclerosis, Friedreich's ataxia, Multiple sclerosis, Huntington's Disease, Transmissible spongiform encephalopathy, Charcot-Marie-Tooth disease, Dementia with Lewy bodies, Corticobasal degeneration, Progressive supranuclear palsy, Chronic Traumatic Encephalopathy (CTE), and Hereditary spastic paraparesis.

49. The method according to any one of claims 45-47 further comprising co-administering to the subject an effective amount of one or more additional therapeutic agents selected from the group consisting of Donepezil (Aricept), Rivastigmine (Exelon), Galantamine (Razadyne), Tacrine (Cognex), Memantine (Namenda), Vitamin E, CERE-110: Adeno-Associated Virus Delivery of NGF (Ceregene), LY450139 (Eli Lilly), Exenatide, Varenicline (Pfizer), PF-04360365 (Pfizer), Resveratrol, Carbidopa/levodopa immediate-release (Sinemet), Carbidopa/levodopa oral disintegrating (Parcopa), Carbidopa/levodopa/Entacapone (Stalevo), Ropinirole (Requip), Pram ipexole (Mirapex), Rotigotine (Neupro), Apomorphine (Apokyn), Selegiline (1-deprenyl, Eldepryl), Rasagiline (Azilect), Zydis selegiline HCL Oral disintegrating (Zelapar), Entacapone (Comtan), Tolcapone (Tasmar), Amantadine (Symmetrel), Trihexyphenidyl (formerly Artane), Benztropine (Cogentin), IPX066 (Impax Laboratories Inc.), ioflupane 1231 (DATSCAN®), safinamide (EMD Serono), Pioglitazone, riluzole (Rilutek), Lithium carbonate, Arimoclomol, Creatine, Tamoxifen, Mecobalam in, tauroursodeoxycholic acid (TUDCA), Idebenone, Coenzyme Q, 5-hydroxytryptophan, Propranolol, Enalapril, Lisinopril, Digoxin, Erythropoietin, Lu AA24493, Deferiprone, IVIG, EGb 761, Avonex, Betaseron, Extavia, Rebif, Glatiramer (Copaxone), Fingolimod (Gilenya), Natalizumab (Tysabri), Mitoxantrone (Novantrone), baclofen (Lioresal), tizanidine (Zanaflex), methylprednisolone, CinnoVex, ReciGen, Masitinib, Prednisone, Interferon beta 1a, Interferon beta 1 b, ELND002 (Elan Pharmaceuticals), Tetrabenazine (Xenazine), haloperidol (Haldol), clozapine (Clozaril), clonazepam (Klonopin), diazepam (Valium), escitalopram (Lexapro), fluoxetine (Prozac, Sarafem), sertraline (Zoloft), valproic acid (Depakene), divalproex (Depakote), lamotrigine (Lamictal), Dimebon, AFQ056 (Novartis), Ethyl-EPA (Miraxion™), SEN0014196 (Siena Biotech), sodium phenylbutyrate, citalopram, ursodiol, minocycline, remacemide, mirtazapine, Quinacrine, Ascorbic acid, PXT3003, Armodafinil, Ramelteon, Davunetide, Tideglusib, alpha-lipoic acid/L-acetyl carnitine, Niacinamide, Oxybutinin chloride, Tolterodine, Botulinum toxin, and combinations thereof.

50. The method according to claim 45, wherein the subject is a mammal.

51. The method according to claim 50, wherein the mammal is selected from the group consisting of humans, veterinary animals, and agricultural animals.

52. The method according to claim 45, wherein the subject is a human.

53. A compound having the structure selected from the group consisting of: 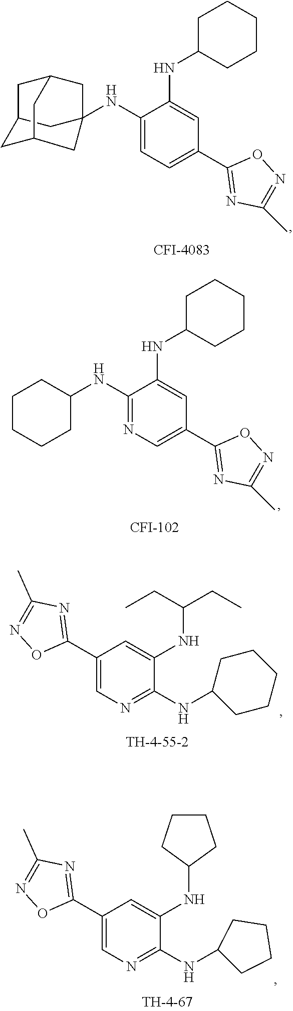 [SMILES: Cc1noc(-c2ccc(NC34CC5CC(CC(C5)C3)C4)c(NC3CCCCC3)c2)n1 ; Cc1noc(-c2cnc(NC3CCCCC3)c(NC3CCCCC3)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
and combinations thereof,
or an N-oxide, crystalline form, hydrate, or a pharmaceutically acceptable salt thereof.

54. A pharmaceutical composition comprising a pharmaceutically acceptable carrier or diluent and a compound having the structure selected from the group consisting of: 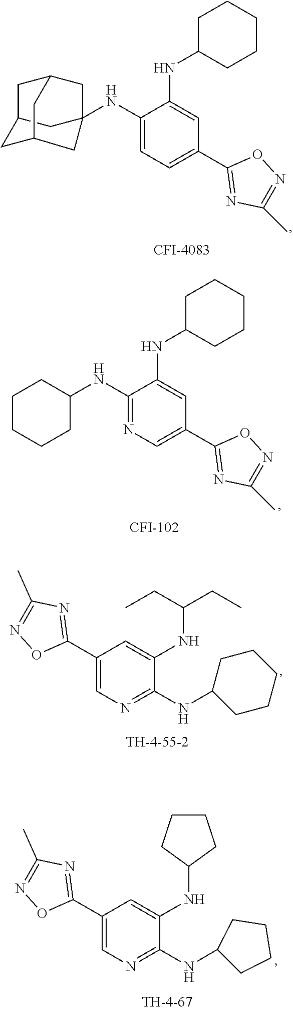 [SMILES: Cc1noc(-c2ccc(NC34CC5CC(CC(C5)C3)C4)c(NC3CCCCC3)c2)n1 ; Cc1noc(-c2cnc(NC3CCCCC3)c(NC3CCCCC3)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
and combinations thereof,
or an N-oxide, crystalline form, hydrate, or a pharmaceutically acceptable salt thereof.

55. A method for treating or ameliorating the effects of a disorder in a subject in need thereof comprising administering to the subject an effective amount of a compound having the structure selected from the group consisting of: 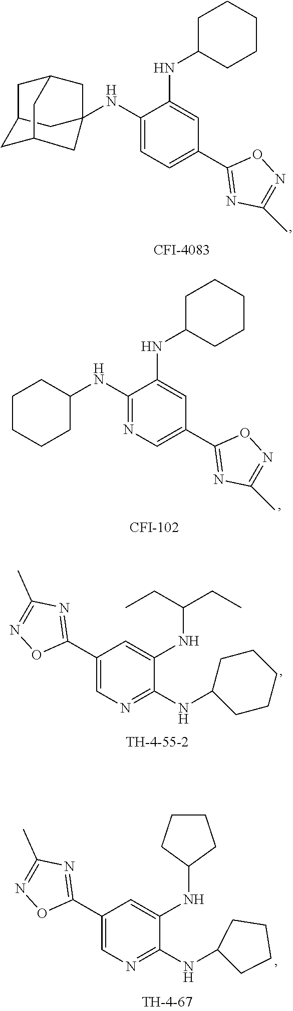 [SMILES: Cc1noc(-c2ccc(NC34CC5CC(CC(C5)C3)C4)c(NC3CCCCC3)c2)n1 ; Cc1noc(-c2cnc(NC3CCCCC3)c(NC3CCCCC3)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
and combinations thereof,
or an N-oxide, crystalline form, hydrate, or a pharmaceutically acceptable salt thereof.

56. A method of modulating ferroptosis in a subject in need thereof comprising administering to the subject an effective amount of a ferroptosis inhibitor, which comprises a compound having the structure selected from the group consisting of: 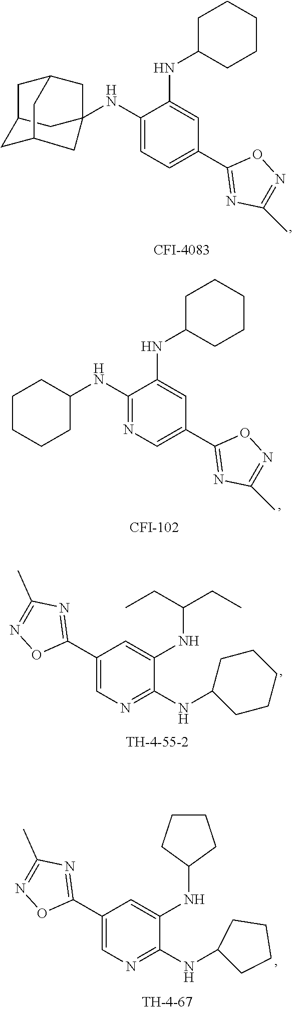 [SMILES: Cc1noc(-c2ccc(NC34CC5CC(CC(C5)C3)C4)c(NC3CCCCC3)c2)n1 ; Cc1noc(-c2cnc(NC3CCCCC3)c(NC3CCCCC3)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
and combinations thereof,
or an N-oxide, crystalline form, hydrate, or a pharmaceutically acceptable salt thereof.

57. A method of reducing reactive oxygen species (ROS) in a cell comprising contacting a cell with a ferroptosis modulator, which comprises a compound having the structure selected from the group consisting of: 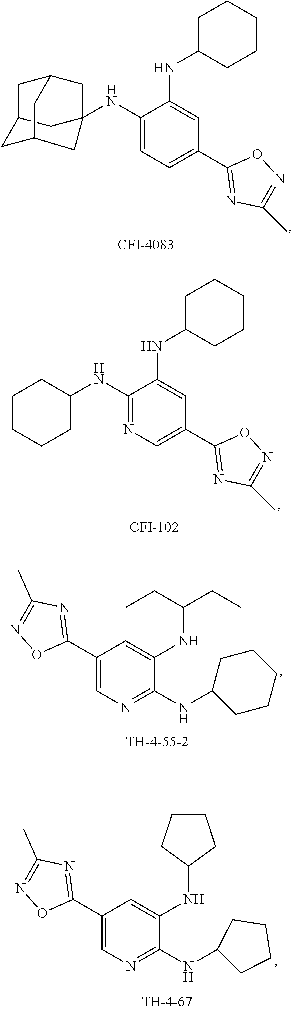 [SMILES: Cc1noc(-c2ccc(NC34CC5CC(CC(C5)C3)C4)c(NC3CCCCC3)c2)n1 ; Cc1noc(-c2cnc(NC3CCCCC3)c(NC3CCCCC3)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
and combinations thereof,
or an N-oxide, crystalline form, hydrate, or a pharmaceutically acceptable salt thereof.

58. A method for treating or ameliorating the effects of a neurodegenerative disease in a subject in need thereof comprising administering to the subject an effective amount of a compound having the structure selected from the group consisting of: 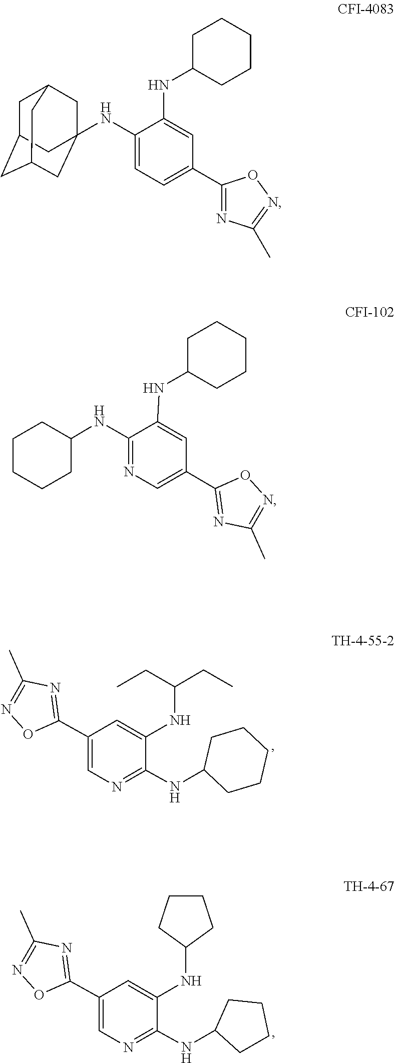 [SMILES: Cc1noc(-c2ccc(NC34CC5CC(CC(C5)C3)C4)c(NC3CCCCC3)c2)n1 ; Cc1noc(-c2cnc(NC3CCCCC3)c(NC3CCCCC3)c2)n1 ; CCC(CC)Nc1cc(-c2nc(C)no2)cnc1NC1CCCCC1 ; Cc1noc(-c2cnc(NC3CCCC3)c(NC3CCCC3)c2)n1]
and combinations thereof, or an N-oxide, crystalline form, hydrate, or a pharmaceutically acceptable salt thereof.

59. A compound according to formula (2): 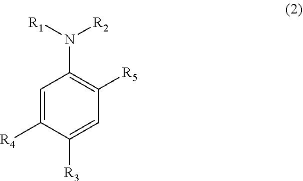
wherein:
R₁ and R₂ are independently selected from the group consisting of H, aryl, C₁₋₆alkyl-aryl, C₁₋₆ alkyl-phenolyl, C₁₋₆ alkyl-bicycle, and C₃₋₁₀carbocycle, wherein each of the aryl, C₁₋₆ alkyl-aryl, C₁₋₆ alkyl-bicycle, and C₃₋₁₀carbocycle are optionally substituted with one or more atoms or groups; or together, with the nitrogen attached, form a cyclic or bicyclic structure, wherein the cyclic or bicyclic structure is optionally substituted with one or more atoms or groups;
R₃ is selected from the group consisting of hydroxyl, alkoxy, and alcohol, wherein each of the hydroxyl, alkoxy, and alcohol are optionally substituted with one or more atoms or groups;
R₄ is selected from the group consisting of H, alkyl, and alkoxy; or together with R₃, form a ring structure, wherein the ring structure is optionally substituted with one or more atoms or groups; and
R₅ is selected from the group consisting of H, and alkoxy;
or an N-oxide, crystalline form, hydrate, or a pharmaceutically acceptable salt thereof.

60. The compound according to claim 59, which is selected from the group consisting of: 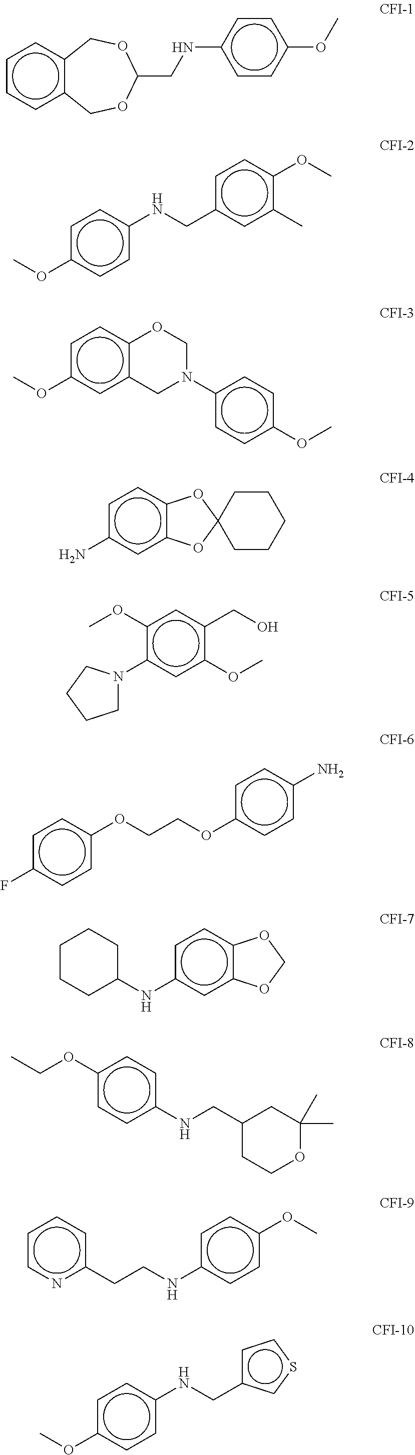 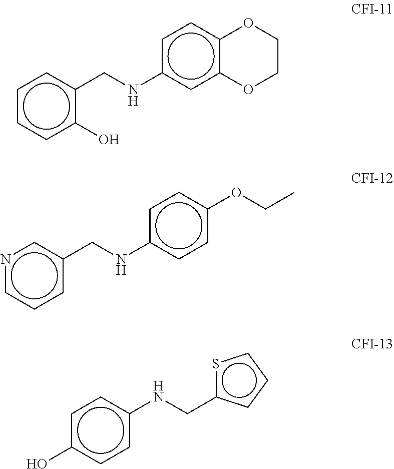
or an N-oxide, crystalline form, hydrate, or a pharmaceutically acceptable salt thereof.

61. A compound according to formula (3): 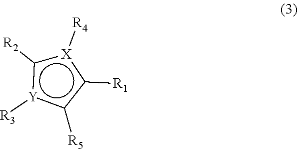
wherein:
X is selected from N, O, and S;
Y is C or N;
R₁ and R₅ are independently selected from the group consisting of H, alkenyl, ester, amino, and aryl, wherein each of the alkenyl, ester, amino, and aryl are optionally substituted with one or more atoms or groups; or together form a ring structure, wherein the ring structure is optionally substituted with one or more atoms or groups;
R₂ and R₃ together form a saturated or unsaturated ring structure, wherein the ring structure is optionally substituted with one or more atoms or groups; and
R₄ is selected from the group consisting of no atom, H, alkyl, alkenyl, and ketone;
or an N-oxide, crystalline form, hydrate, or a pharmaceutically acceptable salt thereof.

62. The compound according to claim 61 having the structure of formula (3a): 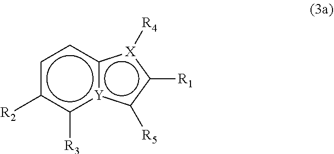
wherein:
X is selected from N, O, and S;
Y is C or N;
R₁ and R₅ are independently selected from the group consisting of H, alkenyl, ester, amino, and aryl, wherein each of the alkenyl, ester, amino, and aryl are optionally substituted with one or more atoms or groups; or together form a ring structure, wherein the ring structure is optionally substituted with one or more atoms or groups;
R₂ and R₃ are independently selected from the group consisting of H, alkyl, amino, and halo; and
R₄ is selected from the group consisting of no atom, H, alkyl, alkenyl, and ketone;
or an N-oxide, crystalline form, hydrate, or a pharmaceutically acceptable salt thereof.

63. The compound according to claim 62, which is selected from the group consisting of: 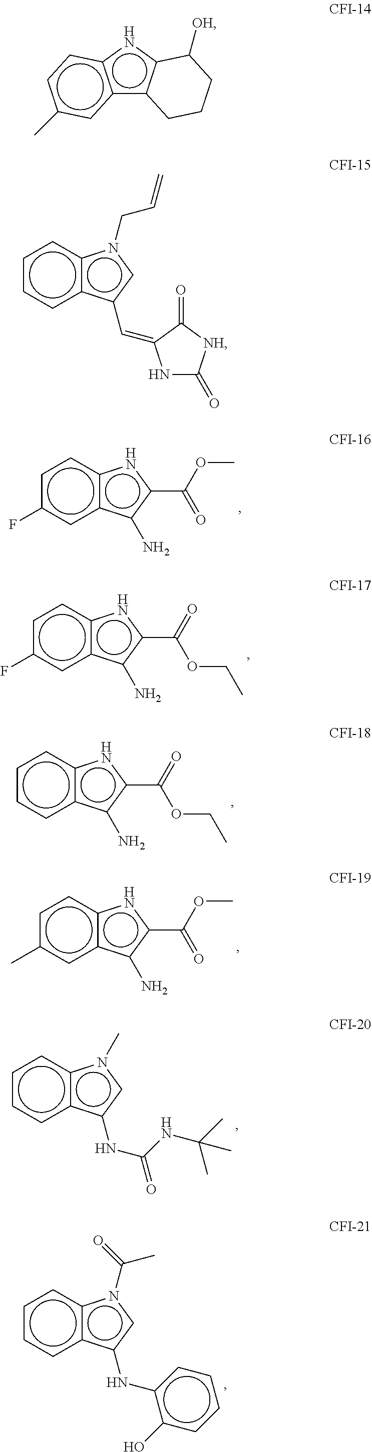 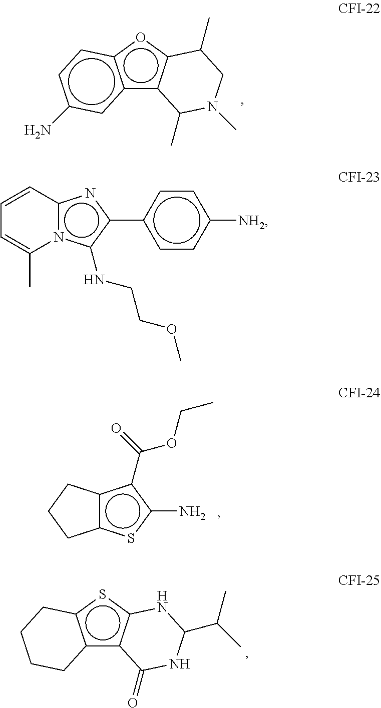
or an N-oxide, crystalline form, hydrate, or a pharmaceutically acceptable salt thereof.

64. The compound according to claim 63, which is selected from the group consisting of: 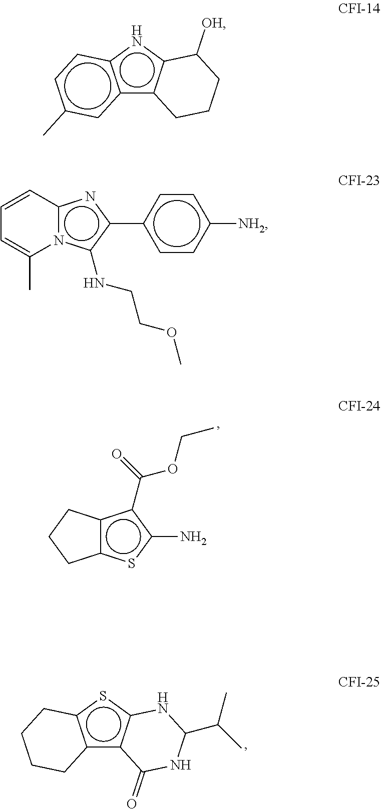
or an N-oxide, crystalline form, hydrate, or a pharmaceutically acceptable salt thereof.

65. A pharmaceutical composition comprising a pharmaceutically acceptable carrier or diluent and one or more compounds according to formula (2): 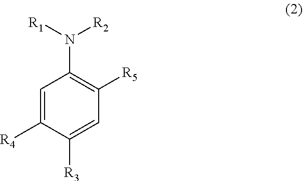
wherein:
R₁ and R₂ are independently selected from the group consisting of H, aryl, C₁₋₆alkyl-aryl, C₁₋₆ alkyl-phenolyl, C₁₋₆ alkyl-bicycle, and C₃₋₁₀carbocycle, wherein each of the aryl, C₁₋₆ alkyl-aryl, C₁₋₆ alkyl-phenolyl, C₁₋₆ alkyl-bicycle, and C₃₋₁₀carbocycle are optionally substituted with one or more atoms or groups; or together, with the nitrogen attached, form a cyclic or bicyclic structure, wherein the cyclic or bicyclic structure is optionally substituted with one or more atoms or groups;
R₃ is selected from the group consisting of hydroxyl, alkoxy, and alcohol, wherein each of the hydroxyl, alkoxy, and alcohol are optionally substituted with one or more atoms or groups;
R₄ is selected from the group consisting of H, alkyl, and alkoxy; or together with R₃, form a ring structure, wherein the ring structure is optionally substituted with one or more atoms or groups; and
R₅ is selected from the group consisting of H, and alkoxy;
or an N-oxide, crystalline form, hydrate, or a pharmaceutically acceptable salt thereof.

66. The pharmaceutical composition according to claim 65, wherein the one or more compounds have a structure selected from the group consisting of: 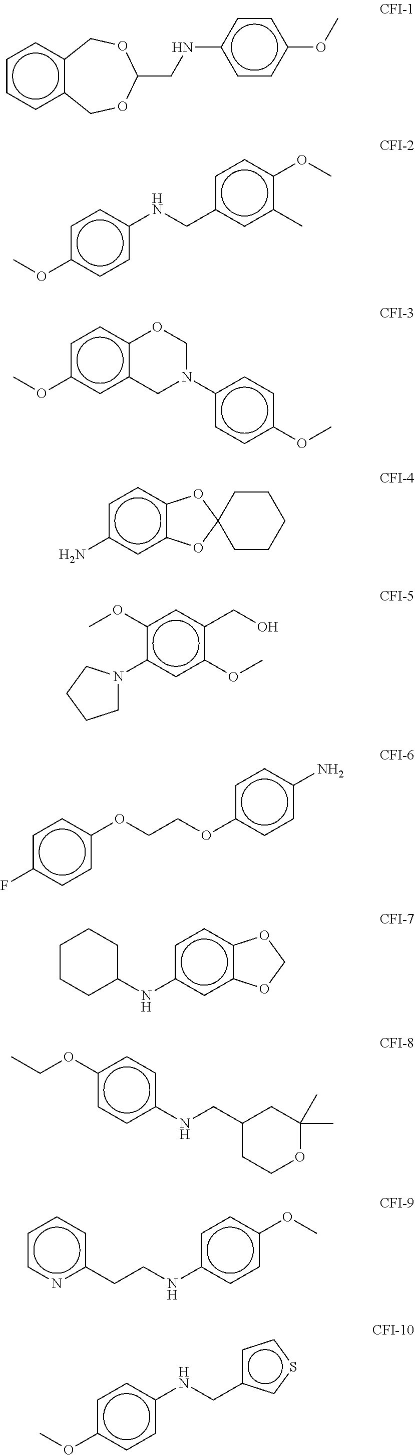 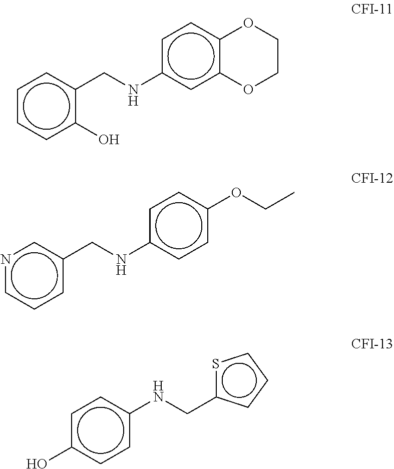
or an N-oxide, crystalline form, hydrate, or a pharmaceutically acceptable salt thereof.

67. A pharmaceutical composition comprising a pharmaceutically acceptable carrier or diluent and one or more compounds according to formula (3): 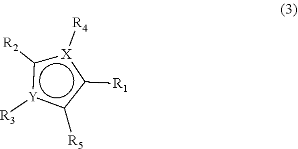
wherein:
X is selected from N, O, and S;
Y is C or N;
R₁ and R₅ are independently selected from the group consisting of H, alkenyl, ester, amino, and aryl, wherein each of the alkenyl, ester, amino, and aryl are optionally substituted with one or more atoms or groups; or together form a ring structure, wherein the ring structure is optionally substituted with one or more atoms or groups;
R₂ and R₃ together form a saturated or unsaturated ring structure, wherein the ring structure is optionally substituted with one or more atoms or groups; and
R₄ is selected from the group consisting of no atom, H, alkyl, alkenyl, and ketone;
or an N-oxide, crystalline form, hydrate, or a pharmaceutically acceptable salt thereof.

68. A pharmaceutical composition comprising a pharmaceutically acceptable carrier or diluent and one or more compounds according to formula (3a): 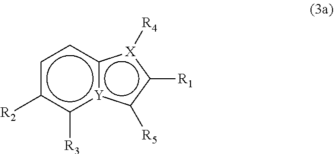
wherein:
X is selected from N, O, and S;
Y is C or N;
R₁ and R₅ are independently selected from the group consisting of H, alkenyl, ester, amino, and aryl, wherein each of the alkenyl, ester, amino, and aryl are optionally substituted with one or more atoms or groups; or together form a ring structure, wherein the ring structure is optionally substituted with one or more atoms or groups;
R₂ and R₃ are independently selected from the group consisting of H, alkyl, amino, and halo; and
R₄ is selected from the group consisting of no atom, H, alkyl, alkenyl, and ketone; or an N-oxide, crystalline form, hydrate, or a pharmaceutically acceptable salt thereof.

69. The pharmaceutical composition according to claim 67, wherein the one or more compounds have a structure selected from the group consisting of: 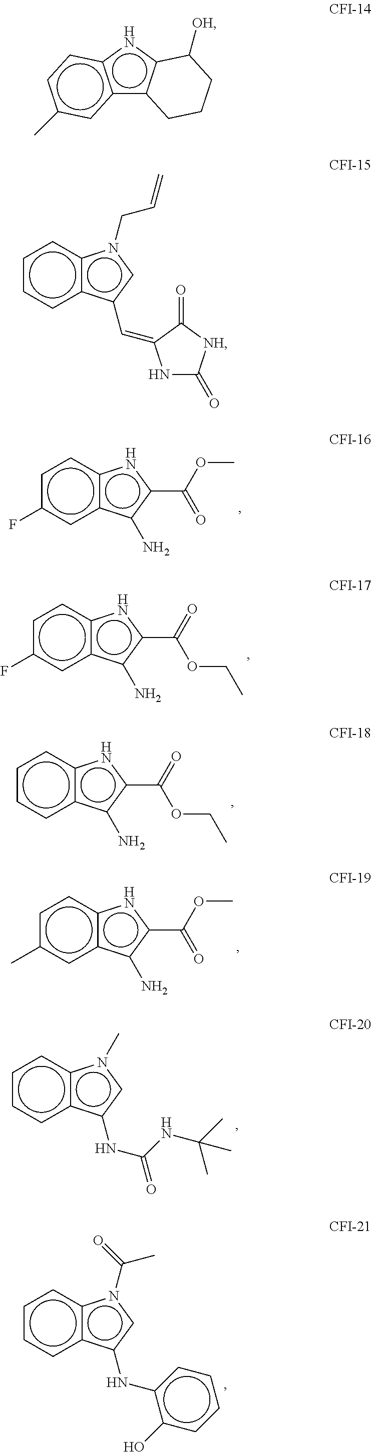 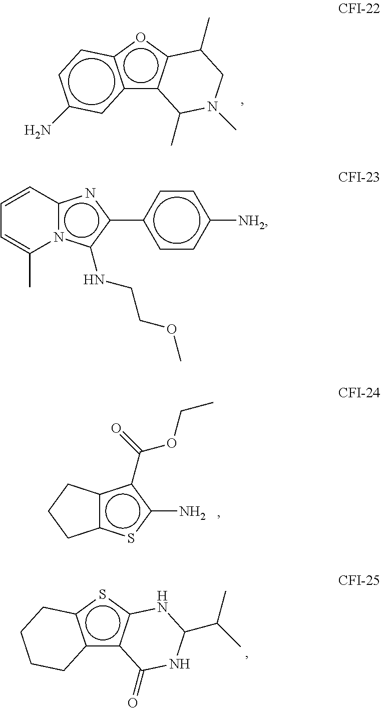
or an N-oxide, crystalline form, hydrate, or a pharmaceutically acceptable salt thereof.

70. The pharmaceutical composition according to claim 67, wherein the one or more compounds have a structure selected from the group consisting of: 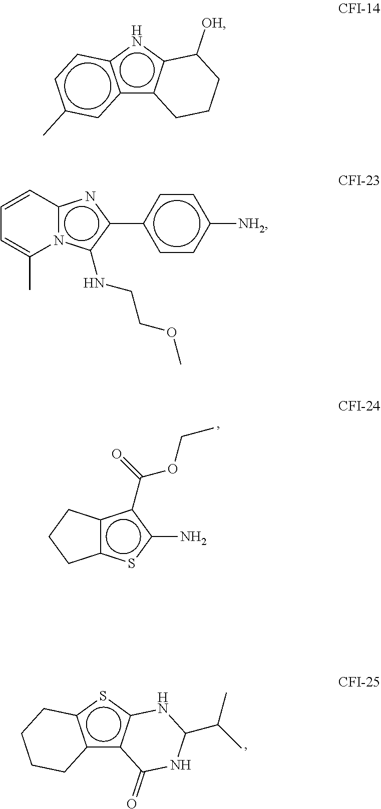
or an N-oxide, crystalline form, hydrate, or a pharmaceutically acceptable salt thereof.

71. A compound having the structure selected from the group consisting of: 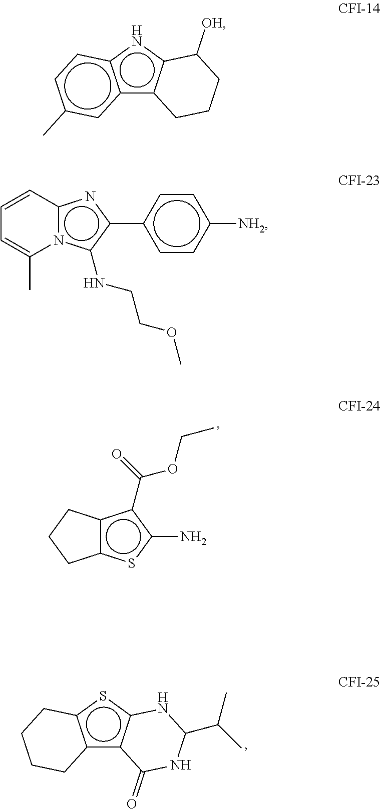
and combinations thereof, or an N-oxide, crystalline form, hydrate, or a pharmaceutically acceptable salt thereof.

72. A pharmaceutical composition comprising a pharmaceutically acceptable carrier or diluent and a compound having the structure selected from the group consisting of: 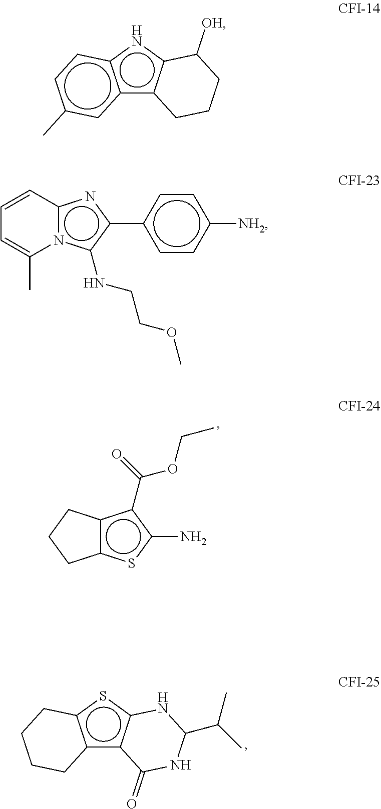
and combinations thereof, or an N-oxide, crystalline form, hydrate, or a pharmaceutically acceptable salt thereof.

73. A method for treating or ameliorating the effects of a disorder in a subject in need thereof comprising administering to the subject an effective amount of a compound having the structure selected from the group consisting of: 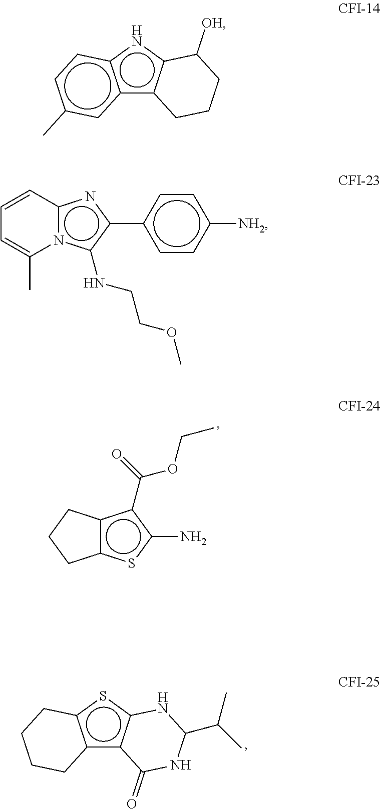
and combinations thereof, or an N-oxide, crystalline form, hydrate, or a pharmaceutically acceptable salt thereof.

74. A method of modulating ferroptosis in a subject in need thereof comprising administering to the subject an effective amount of a ferroptosis inhibitor, which comprises a compound having the structure selected from the group consisting of: 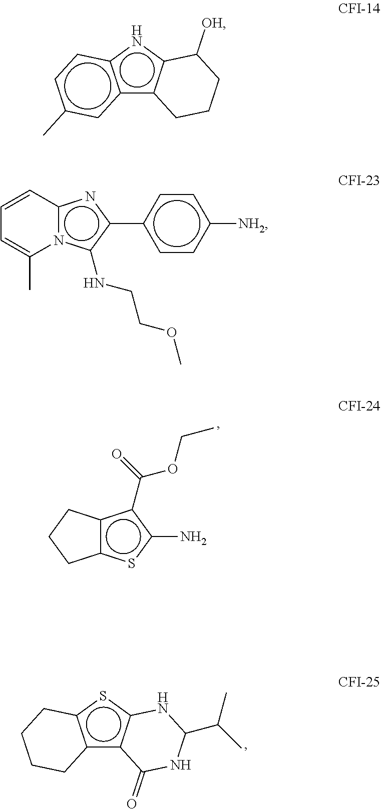
and combinations thereof, or an N-oxide, crystalline form, hydrate, or a pharmaceutically acceptable salt thereof.

75. A method of reducing reactive oxygen species (ROS) in a cell comprising contacting a cell with a ferroptosis modulator, which comprises a compound having the structure selected from the group consisting of: 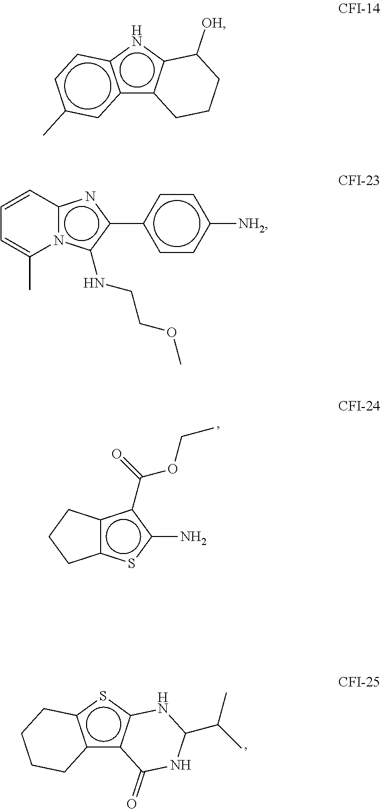
and combinations thereof, or an N-oxide, crystalline form, hydrate, or a pharmaceutically acceptable salt thereof.

76. A method for treating or ameliorating the effects of a neurodegenerative disease in a subject in need thereof comprising administering to the subject an effective amount of a compound having the structure selected from the group consisting of: 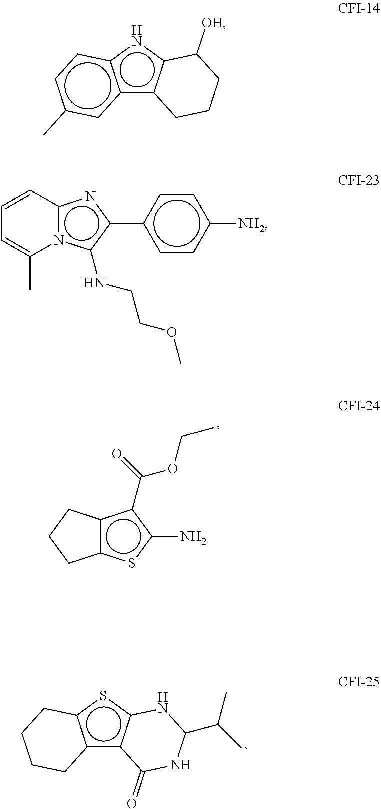
and combinations thereof, or an N-oxide, crystalline form, hydrate, or a pharmaceutically acceptable salt thereof.

77. A method for alleviating side effects in a subject undergoing radiotherapy and/or immunotherapy, comprising administering to the subject an effective amount of one or more compounds according to any one of claims 1-6 and 59-64.

78. A method for treating or ameliorating the effects of an infection associated with ferroptosis in a subject, comprising administering to the subject an effective amount of one or more compounds according to any one of claims 1-6 and 59-64.

79. The method according to claim 78, wherein the infection is caused by Mycobacterium tuberculosis.
